(19) United States
(12) Patent Application Publication
(10) Pub. No.: US 2023/0263729 A1
(43) Pub. Date: Aug. 24, 2023

(54) POLY(ETHYLENE GLYCOL)-BLOCK-POLY(PROPYLENE SULFIDE) NANOCARRIER PLATFORM FOR ENHANCED EFFICACY OF IMMUNOSUPPRESSIVE AGENTS

(71) Applicant: Northwestern University, Evanston, IL (US)

(72) Inventors: Evan A. Scott, Northfield, IL (US); Guillermo A. Ameer, Chicago, IL (US); Jacqueline A. Burke, Chicago, IL (US); Sean D. Allen, Chicago, IL (US); Sijia Yi, Evanston, IL (US)

(21) Appl. No.: 18/177,694

(22) Filed: Mar. 2, 2023

Related U.S. Application Data
(62) Division of application No. 16/891,391, filed on Jun. 3, 2020.
(60) Provisional application No. 62/856,512, filed on Jun. 3, 2019.

(51) Int. Cl. A61K 9/127; A61K 31/593; A61K 31/436; A61K 31/192; A61P 41/00; A61K 47/60; A61K 47/64; A61K 47/54; A61P 9/10

(52) U.S. Cl. CPC A61K 9/1273; A61K 31/593; A61K 31/436; A61K 31/192; A61P 41/00; A61K 47/60; A61K 47/64; A61K 47/543; A61P 9/10; A61K 9/0019

(57) ABSTRACT

Provided herein are nanocarriers for delivery of immunosuppressive agents. In some embodiments, provided herein are nanocarriers comprising a core comprising a poly(ethylene glycol)-block-poly(propylene sulfide) copolymer and least one therapeutic agent. In some embodiments, the nanocarriers may further comprise a targeting ligand displayed on a surface of the nanocarrier. The at least one therapeutic agent may be an anti-inflammatory agent. The disclosed nanocarriers may be incorporated into pharmaceutical compositions for use in methods of treating an inflammatory condition or preventing transplantation rejection in a subject.

### CROSS-REFERENCE TO RELATED APPLICATIONS

This application is a divisional application of U.S. application Ser. No. 16/891,391, filed Jun. 3, 2020, which claims priority to U.S. Provisional Application No. 62/856,512, filed Jun. 3, 2019, each of which is incorporated herein by reference in its entirety.

### STATEMENT REGARDING FEDERALLY SPONSORED RESEARCH

This invention was made with government support under grant number CBET-1453576 awarded by the National Science Foundation and under grant number HL132390 awarded by the National Institutes of Health. The government has certain rights in this invention.

### REFERENCE TO A SEQUENCE LISTING SUBMITTED VIA EFS-WEB

The contents of the electronic sequence listing “702581.02281.xml; Size: 24,972 bytes; and Date of Creation: Mar. 2, 2023” is herein incorporated by reference in its entirety.

### FIELD

Provided herein are nanocarriers for targeted delivery of immunosuppressive agents. In some embodiments, provided herein are nanocarriers comprising a core comprising a poly(ethylene glycol)-block-poly(propylene sulfide) copolymer and least one therapeutic agent. In some embodiments, the nanocarriers may further comprise a targeting ligand displayed on a surface of the nanocarrier. The disclosed nanocarriers enable delivery of immunosuppressants outside of typically reported therapeutic ranges. For example, the disclosed nanocarriers may enable delivery of immunosuppressants at lower dosages, thus achieving the same therapeutic effect at a fraction of the dosage with minimized adverse side effects.

### BACKGROUND

Immunosuppressive and immunomodulatory therapy is commonly used clinically for a variety of conditions. Applications include organ transplantation and inflammatory disorders such as atherosclerosis and arthritis. While this type of therapy can be highly beneficial to patients—often lifesaving, many immunosuppressive agents are associated with debilitating side effects. It is highly desirable to be able to achieve the targeted effect of the therapy by using a lower dose of the immunosuppressive or immunomodulatory agent. Thus, negative off-target effects can be reduced.

### SUMMARY OF THE INVENTION

Disclosed herein are nanocarriers comprising a poly(ethylene glycol)-block-poly(propylene sulfide) copolymer and least one therapeutic agent. The disclosed nanocarriers allow for the delivery of a wide range of immunosuppressive and immunomodulatory agents. The disclosed nanocarriers enable delivery of immunosuppressants outside of typically reported therapeutic ranges. For example, the disclosed nanocarriers may be used to safely lower or increase the dose of the therapeutic agent while minimizing negative side effects. For example, therapeutic agents can be easily loaded into the disclosed nanocarriers and are able to achieve the same immunomodulatory effects seen with the free therapeutic agent at fractions of the dose and with minimized side effects. The nanocarriers may comprise any suitable therapeutic agent, including 25-Dihydroxyvitamin D3 (aVD), celastrol, or rapamycin.

In some embodiments, the nanocarrier may further comprise a targeting ligand displayed on a surface of the nanocarrier. In some embodiments, the targeting ligand may target dendritic cells. For example, the targeting ligand may be a P-D2 peptide.

The disclosed nanocarriers may be used in methods for treating an inflammatory condition in a subject. For example, the disclosed nanocarriers may be used in methods for treating atherosclerosis in a subject. As another example, the disclosed nanocarriers may be used for methods of preventing transplant rejection in a subject. For example, the disclosed nanocarriers may be used in methods of preventing rejection of islet transplantation in a subject.

### BRIEF DESCRIPTION OF DRAWINGS

The patent or patent application file contains at least one drawing in color. Copies of this patent or patent application publication with color drawings will be provided by the Office upon request and payment of the necessary fee.

FIGS. 1A-1B show design and characterization of DC-targeting nanocarriers. (FIG. 1A) The vesicular morphology of poly(ethylene glycol)-block-poly(propylene sulfide) (PEG-bl-PPS) polymersome nanocarriers (PS) enhances the targeting of dendritic cells (DCs) in both mice and non-human primates. Mouse CD8a⁺ DCs and CD11b⁺ DCs were considered analogous to primate cDC1s and cDC2s. Cryogenic electron microscopy (CryoTEM) image scale bar=250 nm. (FIG. 1B) Schematic of multi-component PEG-bl-PPS PS consisting of the P-D2 targeting peptide (SEQ ID NO:1) construct, 1, 25-Dihydroxyvitamin D3 (aVD), and the ApoB-100 derived P210 peptide (SEQ ID NO:2), which respectively provided enhanced targeting, NF-kB suppression, and tolerogenic responses of DCs. The PS were self-assembled from PEG-bl-PPS containing a PEG hydrophilic mass fraction of 25% (schematic 1A). A targeting peptide construct composed of the P-D2 peptide, a PEG spacer, and a palmitoleic acid lipid tail was synthesized for optimization of the peptide surface display (schematic 1B).

FIGS. 1C-1D show design and characterization of DC-targeting nanocarriers. (FIG. 1C) Representative MALDI-TOF spectrum of the P-D2-PEG5-Lys-PA construct, and the mass indicated as 2562. (FIG. 1D) The nanostructure morphology of PS, P-D2-PEG5-PS, and P210/P-D2-PEG5-PS (SEQ ID NO:2) were shown to be consistent by CryoTEM images, scale bar=100 nm.

FIGS. 2A-2B show optimized surface display of the P-D2 peptide enhances intracellular delivery of PS to DCs. Flow cytometric analysis of bone marrow-derived DCs (BMDCs) incubated for 1 h with nile red-loaded PS incorporating P-D2 constructs (FIG. 2A) with different spacer lengths (0, 5, 11, or 15 units of PEG) and (FIG. 2B) at different surface densities (1%, 2%, and 5% molar ratio of P-D2 construct to PEG-bl-PPS copolymer).

FIGS. 2C-2G show optimized surface display of the P-D2 peptide enhances intracellular delivery of PS to DCs. Uptake of Dylight 650 labeled PS and P-D2-PEG5-PS by BMDCs pretreated with (FIGS. 2C and 2D) different concentrations of EIPA (0, 25 and 50 μM) or (FIGS. 2E and 2 F) with and without chlorpromazine (CPZ, 15 μg/ml). (FIG. 2G) Confocal images of BMDCs incubated with Dylight650 labeled PS and P-D2-PEG5-PS (red) at 37° C. for 5 min and 20 min, and then stained with DAPI (blue) and an antibody against clathrin heavy chain (green). Scale bar=5 μm·N=3 for each group and all results are representative of two independent experiments. Two-tailed t-tests were used for statistical analysis: **p<0.01, ***p<0.001.

FIG. 3A shows P-D2 decorated PS enhance a VD-dependent inhibition of pro-inflammatory DC activation. (FIG. 3A) The staining profiles of control mature DCs (thin line) and mature DCs treated with free aVD, PS-aVD or P-D2-PEG5-PS-aVD (grey) were shown for the expression of DC surface marker expression (MHCII, CD80 and CD86). The maturation of DCs was induced by LPS and IFN-γ. A aVD concentration of 10⁻⁸ M was used for each condition. Live cells were gated on CD11c⁺.

FIGS. 3B-3F show P-D2 decorated PS enhance aVD-dependent inhibition of pro-inflammatory DC activation. (FIGS. 3B-3D) Quantification of mean fluorescence intensity of MHCII (FIG. 3B), CD80 (FIG. 3C) and CD86 (FIG. 3D) for stimulated DCs with LPS in the presence of free aVD, PS-aVD or P-D2-PEG5-PS-aVD. iDCs, immature DCs without LPS stimulation; mDCs, mature DCs following LPS stimulation. (FIG. 3E) Quantification of mean fluorescence intensity in MHCII, CD80 and CD86 expression of immature DCs in the presence of PS or P-D2-PEG5-PS. (FIG. 3F) IL-12 secretion in the supernatant of mature DCs treated with free aVD, PS-aVD or P-D2-PEG5-PS-aVD, as determined by ELISA. N=3 for each group and all results are representative of two independent experiments. Two-tailed t-tests were used for statistical significance: **p<0.01,***p<0.001.

FIGS. 4A-4D show P-D2-PEG5-PS enhance targeting of atheroma-resident and splenic DCs. (FIG. 4A) IVIS images revealed that P-D2-PEG5-PS labeled with Dylight 680 preferentially accumulated in atherosclerotic lesions of ApoE^{-/-} mice 24 h after i.v. injection. (FIG. 4B) Flow cytometric analysis showed significantly higher uptake of P-D2-PEG5-PS than PS and P-D2-PS in atheroma of ApoE^{-/-} 24 h after i.v. injection. Histograms indicate the percentages of DCs that were positive for Dylight680 (NC). (FIG. 4C) P-D2-PEG5-PS associated with significantly higher levels of DCs compared to other cell populations in atheroma. (FIG. 4D) Significantly higher uptake of P-D2-PEG5-PS than PS and P-D2-PS was also observed in spleen. The control mice were injected i.v. with the same amount of PBS. DC: CD45⁺CD11c⁺; Macrophages: CD45₊F4/80⁺; Monocytes: CD45⁺Ly6G⁻CD11c⁻CD11b⁺; neutrophils: CD45⁺CD11c⁻Ly6G⁺CD11b⁺; CD4 T cells: CD45⁺CD3⁺CD4⁺; CD8 T cells: CD45⁺CD3⁺CD8⁺. Refer to gating strategies in FIGS. 19-20. N=4-6 mice for each group and data are representative of two independent experiments. Two-tailed t-tests were used for statistical significance.

FIGS. 5A-5B show P210/P-D2-PEG5-PS-aVD reduces atherosclerosis in ApoE^{-/-} mice. (FIG. 5A) Schematic diagram outlining the experimental design for the treatment of atherosclerosis in ApoE^{-/-} mice. 10-week old mice were fed a high fat diet for 4 weeks and then administrated intravenously with different treatments once per week for 8 weeks. (FIG. 5B) Representative images of heart and aorta collected from treated mice at 24 weeks.

FIGS. 5C-5E show P210/P-D2-PEG5-PS-aVD reduces atherosclerosis in ApoE^{-/-} mice. (FIG. 5C) Histologic sections of the aortic sinus were stained with Oil red O (ORO) (red) to detect lipid within lesions. Representative immunofluorescence staining of CD68 (green) in aortic sinus of ApoE^{-/-} mice, indicating decreased levels of lesion-resident inflammatory macrophages. Scale bar=500 μm. ORO area (FIG. 5D) and CD68 area (FIG. 5E) in ˜200 cross sections were quantified for lesion area and macrophage content by an in-house developed software (FIG. 16). N=5-6 mice per group and data are representative of two independent experiments. The unpaired Mann-Whitney test was used for statistical significance: *p<0.05; **p<0.01.

FIGS. 6A-6D show P210/P-D2-PEG5-PS-aVD decreases systemic inflammation and arterial stiffness in ApoE^{-/-} mice. (FIG. 6A) Representative flow cytometric plots of Ly6C″ monocytes gated on CD45⁺ live cells in blood of ApoE^{-/-} mice. (FIG. 6B) The quantification of inflammatory Ly6C^{hi} monocytes in total circulating CD45⁺ lymphocytes. Monocytes: Ly6G⁻ CD11b⁺. N=5 mice per group in A, B. (FIG. 6C) Quantitative RT-PCR analysis of VCAM-1 and ICAM-1 mRNA expression in aortas of control, free aVD, P-D2-PEG5-PS-aVD and P210/P-D2-PEG5-PS-aVD groups. The mRNA expression was quantified relative to GAPDH. Data was normalized to the control group and expressed as the mean±s.e.m. (FIG. 6D) Representative force-distance curves obtained from the aortic arch of ApoE^{-/-} mice (blue dots), fitted by Hertzian contact elastic model (red line).

FIGS. 6E-6G show P210/P-D2-PEG5-PS-aVD decreases systemic inflammation and arterial stiffness in ApoE^{-/-} mice. (FIG. 6E) Young's moduli were determined from the force-indentation data collected over 30 nonlesioned regions of 5 different mouse aortic arch tissue areas. The mean Young's modulus was calculated for each mouse sample. N=3 mice per group. (FIG. 6F) The levels of cytokine IL-6 in serum of ApoE^{-/-} mice was determined with Luminex-based multiplex. N=4-5 mice per group. (FIG. 6G) mRNA expression of IL-10 and IL-6 in aortas of control, free aVD, P-D2-PEG5-PS-aVD and P210/P-D2-PEG5-PS-aVD groups, as determined by quantitative RT-PCR. N=5-6 mice per group. Two-tailed t-tests were used for statistical significance: *p<0.05, **p<0.01.

FIGS. 7A-7C show P-D2-PEG5-PS-aVD inhibits DC maturation in lymphoid organs and increases Treg levels in the aorta. (FIG. 7A) Representative flow cytometric plots of mature DCs as CD80⁺CD86⁺CD11c⁺ gated on CD45⁺ live cells in both spleen and DLNs of ApoE^{-/-} mice with and without PS-aVD or P-D2-PEG5-PS-aVD treatment. The percentages of mature DCs (CD80⁺CD86⁺CD11c⁺) within the total DC population (CD11c⁺) were quantified in both spleen (FIG. 7B) and DLNs (FIG. 7C).

FIGS. 7D-7G show P-D2-PEG5-PS-aVD inhibits DC maturation in lymphoid organs and increases Treg levels in the aorta. (FIG. 7D) The expression of CD80 and CD86 genes in aortas of control or P-D2-PEG5-PS-aVD treated groups, as determined by quantitative RT-PCR. (FIG. 7E) Representative flow cytometric plots of Tregs (Foxp3⁺CD25⁺CD4⁺) gated on CD45⁺CD3⁺ live cells in spleen of ApoE^{-/-} mice. (FIG. 7F) Immunofluorescence staining of Foxp3 (red) in aortic sinus of ApoE^{-/-} mice was (FIG. 7G) quantified in cross sections to assess Treg content in aorta. Scale bar=200 μm. N=5 mice per group in A, B, C, E; N=5-6 mice per group in D, F, G. All results are representative of two independent experiments. The unpaired Mann-Whitney test was used for statistical significance in G; two-tailed t-tests were used for statistical significance in A, B, C, D, E: *p<0.05, **p<0.01, ***p<0.001.

FIGS. 8A-8C show Representative MALDI-TOF spectra of P-D2 conjugation with different PEG lengths (n=0, 11, and 15).

FIG. 9 shows CryoTEM images of PS decorated with P-D2 constructs containing different PEG spacer lengths (P-D2-PS, P-D2-PEG11-PS, and P-D2-PEG15-PS), which were assembled in PBS solution.

FIGS. 10A-10C. (FIG. 10A) The amount of P-D2 peptide decorated on PS was measured by the 3-(4-carbpxubemzpul)quinoline-2-carboxaldyhyde (CBQCA) assay (right). The red dots show the amount of P-D2-PEG5-Lys-PA initially added, while the black dots show the amount of P-D2-PEG5-Lys-PA conjugated on PS after purification by size exclusion chromatography (SEC). The left figure shows the standard curve of P-D2 peptide by CBQCA assay, which measured the amount of primary amines on peptides via a specific reaction between CBQCA and primary amine with cyanide. (FIG. 10B) The effect of different molar ratios of P-D2-PEG5-Lys-PA (from 1% to 5%) to PEG-bl-PPS copolymers on cellular uptake of PS by BMDCs. No significant increase was observed beyond a 4% molar ratio of the P-D2 construct. (FIG. 10C) The aVD-loaded P-D2-PEG5-PS suppressed the expression of costimulatory molecules (MHC-II, CD80, and CD86) on BMDCs in a dose dependent manner (aVD concentration=0, 10⁻¹⁰, and 10⁻⁹ M). N=3 for each group.

FIGS. 11A-11B (FIG. 11A) MTT cell viability assay of polymersomes on BMDCs. (FIG. 11B) BMDC viability assay following polymersomes treatment was performed by Zombie Aqua fixable cell viability dye. The concentration of PS and P-D2-PEG5-PS was 0.3 mg/ml. N=3-4 per group.

FIG. 12 shows Confocal images of BMDCs incubated with Dylight650 labeled PS and P-D2-PEG5-PS (red) at 4° C. for 5 min and 20 min, and then stained with DAPI (blue) and an antibody against clathrin heavy chain (green). Scale bar=5 μm.

FIGS. 13A-13C. (FIG. 13A) Flow cytometry analysis showed that PS associated with significantly higher levels of DCs compared to other cell populations in atheroma. (FIG. 13B) Biodistribution of PS within different cell populations of spleen. (FIG. 13C) Flow cytometry analysis showed that P-D2-PEG5-PS associated with significantly higher percentage of DCs compared to other cell populations, such as macrophages, monocytes, neutrophils, nature killer cells (NKs), CD4 T cells, CD8 T cells and CD45⁻ cells in spleen of ApoE^{-/-} mice. N=4-5 mice for each group and data are representative of two independent experiments. Two-tailed t-tests were used for statistical significance: *p<0.05, **p<0.01,***p<0.001.

FIGS. 14A-14B (FIG. 14A) Mouse body weight was measured every week during treatment. (FIG. 14B) Quantification of serum total cholesterol in ApoE^{-/-} mice after 8 weeks treatment. N=5-6 mice for each group.

FIGS. 15A-15B (FIG. 15A) Representative images of mouse heart and aorta of ApoE^{-/-} mice after 8 weeks of treatment. (FIG. 15B) Histology images of mouse aortic sinus from different groups as shown in bright-field. Scale bar=500 μm.

FIGS. 15C-15D show total fluorescence intensity of ORO (FIG. 15C) and CD68 (FIG. 15D) in 200 cross sections were quantified for lesion size and macrophage content. N=5-6 mice per group and data are representative of two independent experiments. The two tailed t-tests were used for statistical significance: *p<0.05; **p<0.01; ***p<0.001.

FIG. 16 shows an in-house developed software written in Python was used to quantify areas and fluorescence in histological samples.

FIGS. 17A-17B show quantification of IFN-γ (FIG. 17A) and IL-10 (FIG. 17B) cytokine production in serum of ApoE^{-/-} mice after 8 weeks treatment. N=4-5 for each group. The two tailed t-tests were used for statistical significance: **p<0.01.

FIGS. 18A-18B show the percentages of Tregs (Foxp3⁺CD25⁺CD4⁺) within the total T cell population (CD45⁺ CD3⁺) were quantified in (FIG. 18A) spleen and (FIG. 18B) DLNs. N=5 mice per group and data are representative of two independent experiments

FIG. 19 shows flow cytometry gating strategy for the analysis of polymersome distribution in aortic immune cells of ApoE^{-/-}. Immune cells are determined as below: (a) Macrophages: CD45⁺F4/80⁺; (b) dendritic cells: CD45⁺CD11c⁺; (c) monocytes: CD45⁺Ly6G⁻ CD11b⁺.

FIG. 20A shows flow cytometry gating strategy for the analysis of polymersome distribution in splenic immune cells of ApoE^{-/-} mice. Immune cells are determined as below: I. (a); (b) dendritic cells: CD45⁺CD11c⁺; (b) monocytes: CD45⁺CD11c⁻Ly6G⁻CD11b⁺; (c) neutrophils: CD45⁺CD11c⁻Ly6G⁺CD11b⁺.

FIG. 20B show flow cytometry gating strategy for the analysis of polymersome distribution in splenic immune cells of ApoE^{-/-} mice. II. (d) Macrophages: CD45⁺F4/80⁺; (e) natural killer cells: CD45⁺NK1.1⁺; (f) CD4 T cells: CD45⁺CD3⁺CD4⁺; (g) CD8 T cells: CD45⁺CD3⁺CD8⁺.

FIGS. 21A-21C show characterization of rapamycin-loaded polymersomes. (FIG. 21A) Size distribution shown via dynamic-light scattering. (FIG. 21B) Cryogenic transmission electron micrograph (cryoTEM) of rapamycin-loaded polymersomes. (FIG. 21C) Small angle x-ray scattering (SAXS) transformed data and polymer vesicle model fits.

FIG. 22 shows experimental overview for in vivo allogenic islet transplantation.

FIGS. 23A-23C show rapamycin-Loaded Polymersomes prevent islet transplantation rejection. (FIG. 23A) Average blood glucose concentration. (FIG. 23B) Average body weight. (FIG. 23C) Diabetes incidence (defined is blood glucose concentration ≥200 mg/dl).

FIGS. 24A-24B show recipients treated with Low Dose Rapamycin-Loaded Polymersomes have improved islet function over those treated with Low Dose Free Rapamycin. (FIG. 24A) Glycemic profile and (FIG. 24B) area under the curve (AUC) of the profile during intraperitoneal glucose tolerance test (IPGTT) performed 1 month post-transplantation.

FIG. 25A shows size and morphological characterization of Blank MC and Cel-MC. (FIG. 25A) Schematic of polymer and celastrol chemical structures and a cartoon figure of an assembled micelle loaded with celastrol.

FIGS. 25B-25C show size and morphological characterization of Blank MC and Cel-MC. (FIG. 25B) Cryogenic transmission electron micrographs of Blank MC and Cel-MC, scale bars=50 nm. (FIG. 25C) Small angle x-ray scattering transformed data and polymer micelle model fits. Graphs are vertically offset for ease of visualization.

FIGS. 26A-26C show encapsulation efficiency, loading capacity, and release of celastrol from micelles. (FIG. 26A) Encapsulation efficiency of celastrol in micelles when loaded at different starting amounts of celastrol. ‘Celastrol Added’ represents the amount of celastrol initially available to be loaded into 10 mg of polymer. All data points on graph, n=3. (FIG. 26B) Loading capacity of celastrol in micelles. ‘Celastrol Added’ and ‘Celastrol Loaded’ represent the amount of celastrol initially available to be loaded into micelles and the amount of celastrol actually loaded into micelles, respectively, per 10 mg of polymer. All data points on graph, n=3. (FIG. 26C) Cumulative release of celastrol from celastrol micelles into 1×PBS. Average values plotted on graph, error bars (S.D.) not visible due to low variability compared to y-axis scale, n=3.

FIGS. 27A-27E show subcellular localization of Cel-MC in RAW 264.7 cells and inhibition of NF-κB by and cytotoxicity of free celastrol and Cel-MC. (FIG. 27A) Confocal images of live RAW 264.7 cells incubated with a nuclear stain (blue) and a lysosomal stain (green). Cells were also incubated overnight with blank MC (top row) or Cel-MC (1 μg/mL celastrol, bottom row) labelled with DiI, a lipophilic dye. Composite and brightfield images are included to demonstrate colocalization of micelle and lysosome signal and cell morphology, respectively. (FIG. 27B) RAW Blue colorimetric assay of NF-κB expression at varying concentrations of celastrol. Y-axis is normalized such that 0% represents cells untreated with LPS and 100% represents cells treated with LPS but not treated with any celastrol. X-axis is on a log scale. n=4 (FIG. 27C) ELISA results for TNF-α secretion by RAW 264.7 cells treated with LPS and either free celastrol or Cel-MC. Celastrol treatments were at 10 ng/mL or 1 μg/mL concentrations. All data points shown on graph, n=5 for treatment conditions, n=12 for the LPS control. P values shown on graph are from Tukey's multiple comparison test. (FIG. 27D) RAW 264.7 cell viability with either free celastrol or Cel-MC treatment at varying concentrations of celastrol. Y-axis represents viability normalized by delivery vehicle or formulation, with 100% representing the mean viability of cells treated with vehicle but no celastrol, and 0% representing methanol-treated cells. X-axis is on a log scale. n=4. (FIG. 27E) Stacked bar graph of RAW 264.7 viability split into three categories: live, dead, or apoptotic. Cells were either LPS treated (+) or not (−). n=5 for each treatment group. For FIGS. 27B-27E, error bars represent standard deviation.

FIGS. 28A-28C show RNAseq analysis of transcriptional effects of free celastrol and Cel-MC treatment of RAW 264.7 cells. Free celastrol and Cel-MC have similar anti-inflammatory effects on the transcriptomes of LPS-treated RAW 264.7 cells. (FIG. 28A) Heatmap analysis of genes significantly affected by free celastrol. DE-Seq2 analysis identified 2649 genes significantly altered by free celastrol treatment of LPS-treated RAW 264.7 cells after 2 hours (p_{adj}<0.1). This gene set was used to generate a heatmap with the following conditions: LPS-treated RAW 264.7 cells (LPS), LPS+celastrol vehicle (V), LPS+blank micelles (Blank MC), LPS+free celastrol (Free Cel), and LPS+Cel-MC (Cel-MC). Red represents genes that are overexpressed in that sample compared to the other cohorts. Blue, underexpressed. (FIG. 28B) Fold Change and (FIG. 28C) Adjusted P-values of the NF-κB gene set. Gene set variation analysis of the NF-κB pathway (Hinata NF-κB Matrix Gene Set) in LPS-treated RAW 264.7 cells treated for 2 hours with vehicle (V), blank MC, free celastrol, or Cel-MC. Fold change is relative to RAW 264.7 cells treated with only LPS.

FIGS. 29A-29E show flow cytometric analysis of changes in cell populations in ldlr−/− mice treated with free celastrol or Cel-MC. (FIG. 29A) Heatmap of fold change in cell populations. Each row represents an immune cell population, each column represents the organ from which the cells were isolated. Heatmap is on a log 2 scale, with yellow representing a fold increase and blue representing a fold decrease in that cell population, compared to the Blank MC control. Cell population as a percent of all immune cells for a given population in a given organ are also provided for: (FIG. 29B) aortic neutrophils, (FIG. 29C) aortic NK cells, (FIG. 29D) blood monocytes, and (FIG. 29E) blood neutrophils. All significant p-values are displayed on their graphs, calculated using Dunn's multiple comparisons test. Cells were identified as follows: B cells—CD45+ CD19+, NK cells—CD45+ NK1.1+, T cells—CD45+ CD3+, Neutrophils—CD45+ Ly-6G+, Macrophages—CD45+ CD3− NK1.1− CD19− Ly-6G− F4/80+, Dendritic cells—CD45+CD3− NK1.1− CD19− Ly-6G− F4/80− CD11c+, Monocytes—CD45+CD3− NK1.1− CD19− Ly-6G− F4/80− CD11c− CD11b+ Ly-6C+.

FIGS. 30A-30B show Oil Red O (ORO) analysis of plaque area in ldlr−/− mice treated with free celastrol or Cel-MC. (FIG. 30A) Representative fluorescence microscopy images of ORO stained, frozen aorta sections from free celastrol, blank MC or Cel-MC treated ldlr−/− mice. Top images represent brightfield microscopy, while bottom images were obtained with fluorescence microscopy of DAPI-stained nuclei (blue) and lipid-rich plaques (red). All images were acquired at 20× magnification. (FIG. 30B) Quantification of ORO staining area for free celastrol, Blank MC, and Cel MC treated aorta sections. P-value was calculated using Dunn's multiple comparisons test. Data points represent imaged sections from discrete portions along the length of the aorta, all data points shown on graph. Bars represent the mean and standard deviation, n=12 for free celastrol, n=11 for Blank MC, and n=14 for Cel-MC.

FIGS. 31A-31B show flow cytometry gating strategy contour plots. Gating strategy for flow cytometry for (FIG. 31A) both panels and (FIG. 31B) panel 1.

FIG. 31C shows flow cytometry gating strategy contour plots. Gating strategy for flow cytometry for (FIG. 31C) panel 2.

FIG. 32 shows confocal images of RAW 264.7 cells not treated with Dil-labeled micelles. RAW 264.7 cells stained with Hoechst 33342 for nuclei and LysoTracker Green for lysosomes. As cells were not treated with Dil-labeled micelles, the red channel is devoid of signal, demonstrating low bleed through of LysoTracker Green signal.

FIGS. 33A-33B show mouse body weight and food consumption analysis. (FIG. 33A) Average mouse body weights in the weeks before and after the initiation of treatment with free celastrol, Blank MC, and Cel-MC. n=8 for free celastrol and Blank MC groups and n=9 for Cel-MC group. Error bars are standard deviation, x-axis time 0 is the initiation of treatment. (FIG. 33B) Average food consumed during treatment by mice within the three treatment groups, n=7 for all treatment groups, bars represent the mean and standard deviation.

FIGS. 34A-34C show additional flow cytometric cell population comparisons between treatment groups. Comparison of each cell population as a percent of CD45+ cells in that organ between free celastrol, Blank MC, and Cel-MC treatments for (FIG. 34A) splenic NK cells, (FIG. 34B) aortic dendritic cells, and (FIG. 34C) splenic T cells. P-values obtained using Dunn's multiple comparison test, n=6. Bars represent the mean and standard deviation. All data points are shown on graphs.

FIG. 35. Right: Rapamycin can be easily loaded into the hydrophobic membrane of polymersomes to form rPS. Top left: When injected subcutaneously into mice, the rPS drain into the brachial lymph nodes where they are uptaken by antigen-presenting cells. As a result, these antigen presenting cells develop an anti-inflammatory, semi-mature phenotype, in which they express high levels of MHC II to present to CD4+ T cell receptors, but they do not express costimulatory molecules, thus preventing costimulation. Without activation, the acute rejection causing CD4+ T cells go into a state of anergy or become tolerogenic CD8+ regulatory T cells. Bottom left: The resulting tolerogenic state allows for fully-major compatibility mismatched allogeneic islet graft survival at the clinically relevant intraportal (liver) transplantation site.

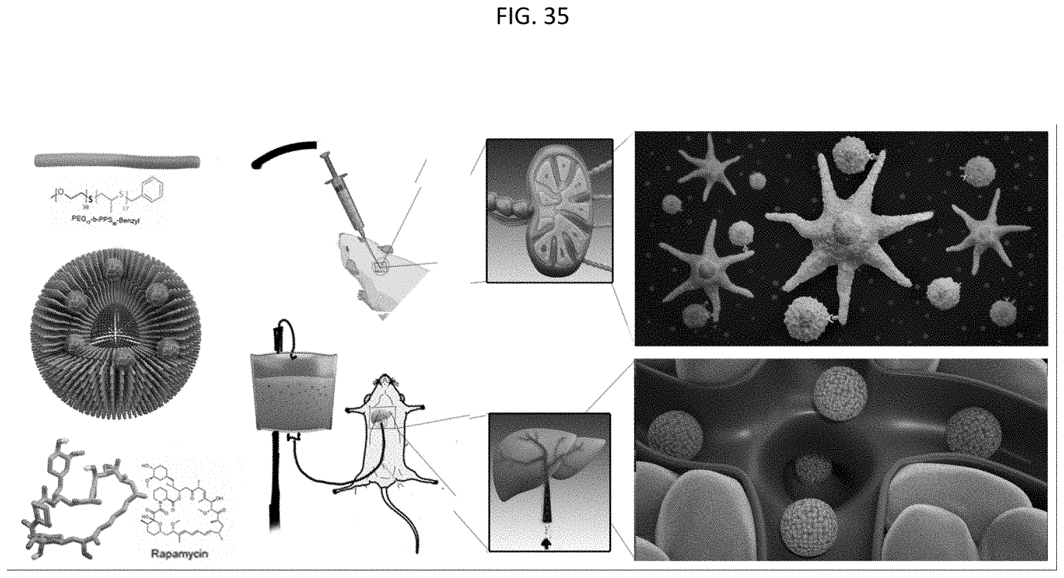

FIGS. 36A-36E show morphological and functional characterization of PS and rPS relative to PLGA and rPLGA. Cryogenic transmission electron micrograph (cryoTEM) of PS (FIG. 36A), rPS (FIG. 36B), PLGA (supplement), and rPLGA (supplement) with overlay of size distribution by dynamic light scattering (DLS) (n=3). Scale bars represent 100 nm. (FIG. 36C) Small angle X-ray scattering (SAXS) transformed data of PS (black) and rPS (purple) with polymer vesicular model fit (--). (FIGS. 36D and 36E) rPS show superior encapsulation efficiency (FIG. 36D) and stability of loaded rapamycin (FIG. 36E) as compared to rPLGA (n=3-5). All data is presented as mean±SD with *p<0.05; **p<0.01; ***p<0.001; ****p<0.0001.

FIG. 36F shows flow cytometry analysis of mice subcutaneously injected with PBS or blank formulations of PLGA or PS for 11 days reveal that PS are non-immunogenic as compared to PLGA. As compared to PLGA nanocarriers, PS cause minimal alterations in immune cell populations and inflammatory cell phenotypes. Fold changes greater than 5 are shown in white. Macrophages were not assessed in blood as indicated by a black “x.” (n=3 mice). All data is presented as mean fold change.

FIGS. 37A-37B show Polymersomes alter organ-level biodistribution. (FIG. 37A) Biodistribution of indocyanine green (ICG) dye by formulation 2 hours after subcutaneous injection (n=5 mice). ICG-PS dye drains to the brachial lymph nodes (right side) 24 and 48 h post subcutaneous injection. (FIG. 37B) Biodistribution of rapamycin by formulation. Rapamycin concentration in the blood, axial and brachial lymph nodes, spleen, and liver over time (0.5 h, 2 h, 8 h, 16 h, 24 h and 48 h). (n=3 mice). All data is presented as mean±SD with *p<0.05; **p<0.01; ***p<0.001; ****p<0.0001.

FIGS. 38A-38C show polymersomes alter drug mechanism. (FIG. 38A) tSNE analysis of CD45+ cells from the axial and brachial lymph nodes shows clustering of cell populations after standard dosage rapamycin or rPS treatment. Two distinct T cell populations are observed for both rapamycin and rPS treatment. For the rPS treatment group, there is an overall reduction in T cells and an increase in monocytes and DCs (FIG. 38B). One of the T cell populations clusters with the enhanced monocyte and DC populations (FIG. 38A; circled). (FIG. 38C) Monocytes are anti-inflammatory as significant reductions in Ly-6C and macrophage markers (F4/80 and CD169) are observed. The tolerogenic monocyte phonotype is enhanced by significant reductions in costimulatory markers (CD40, CD80 and CD86). Data is shown from the axial and brachial lymph nodes. (n=3 mice). All data are presented as mean±SD with *p<0.05; **p<0.01; ***p<0.001; ****p<0.0001.

FIGS. 38D-38E show polymersomes alter drug mechanism. (FIG. 38D) DCs have a similar phenotype to monocytes with greatly reduced stimulatory markers, but enhanced MHC II expression. The pDC population is reduced, while enhancement is observed in a unique double CD8+ and CD11b+ cDC population. (FIG. 38E) When specific T cell populations are mapped on to the tSNE plot of CD45+ cells, it is observed that there is clustering between CD4+ and double CD4+ CD8+ T cells with monocytes and DCs (circled). Data is shown from the axial and brachial lymph nodes. (n=3 mice). All data are presented as mean±SD with *p<0.05; **p<0.01; ***p<0.001; ****p<0.0001.

FIG. 38F shows polymersomes alter drug mechanism. (FIG. 38F) The CD4+ CD8− population is reduced with rPS treatment while CD4− CD8+ and double positive CD4+ CD8+ populations are bolstered. Furthermore, with rPS treatment there is enhancement in tolerogenic NK T cells while tend to cluster with CD8+ T cells and CD8+ regulatory T cells. Data is shown from the axial and brachial lymph nodes. (n=3 mice). All data are presented as mean±SD with *p<0.05; **p<0.01; ***p<0.001; ****p<0.0001.

FIGS. 39A-39C show polymersomes reduce effective drug dose and mitigate side effects in vivo. (FIG. 39A) Standard dosage and low dosage schemes for rapamycin during allogeneic islet transplantation (day 0) experiment. Diabetes is induced (day −5) via streptozotocin injection. The standard dosage protocol consists of 11 injections, given daily starting at day −1. The low dosage protocol consists of 6 injections, given every 3 days, starting at day −1. (FIG. 39B) First month post-transplantation blood glucose concentrations. (n≥5 mice). (FIG. 39C) Post-transplantation normoglycemia (%) (BG<200 mg/dl). No treatment (red circle); Standard dosage rapamycin (black square); Low dosage rapamycin (white box, black outline); Low dosage rPS (purple upside down triange). (n≥5 mice). All data is presented as mean±SD with *p<0.05; **p<0.01; ***p<0.001; ****p<0.0001.

FIGS. 39D-39E show polymersomes reduce effective drug dose and mitigate side effects in vivo. (FIG. 39D) rPS eliminate dorsal injection site alopecia during allogeneic islet transplantation. Hematoxylin and eosin histology shows lack of mature hair follicles in standard dosage rapamycin group, weaning hair follicles in low dosage rapamycin group and fully mature hair follicles in the low dosage rPS group. Scale bars represent 100 μm. (n≥5 mice). (FIG. 39E) Single cell RNA sequencing analysis of macrophages and regulatory T cells from the liver and spleen reveals that rPS treatment causes less perturbation in genes associated with known side effects of rapamycin, including impaired wound healing, malignancy, metabolic syndrome and enhanced predisposition to viral infection. (n=3 mice). All data is presented as mean±SD with *p<0.05; **p<0.01; ***p<0.001; ****p<0.0001.

FIGS. 40A-40F shows biodistribution of rapamycin by formulation. Rapamycin concentration in the a, blood, b, kidneys, c, liver, d, axial and brachial lymph nodes, e, spleen and f, urine over time (0.5 h, 2 h, 8 h, 16 h, 24 h and 48 h) when given as rapamycin (circle) or rPS (square). Rapamycin concentration was also analyzed in the lungs, brain, heart, and fat; however, levels were below 1 ng/mg for both rapamycin and rPS at all timepoints. (n=3 mice).

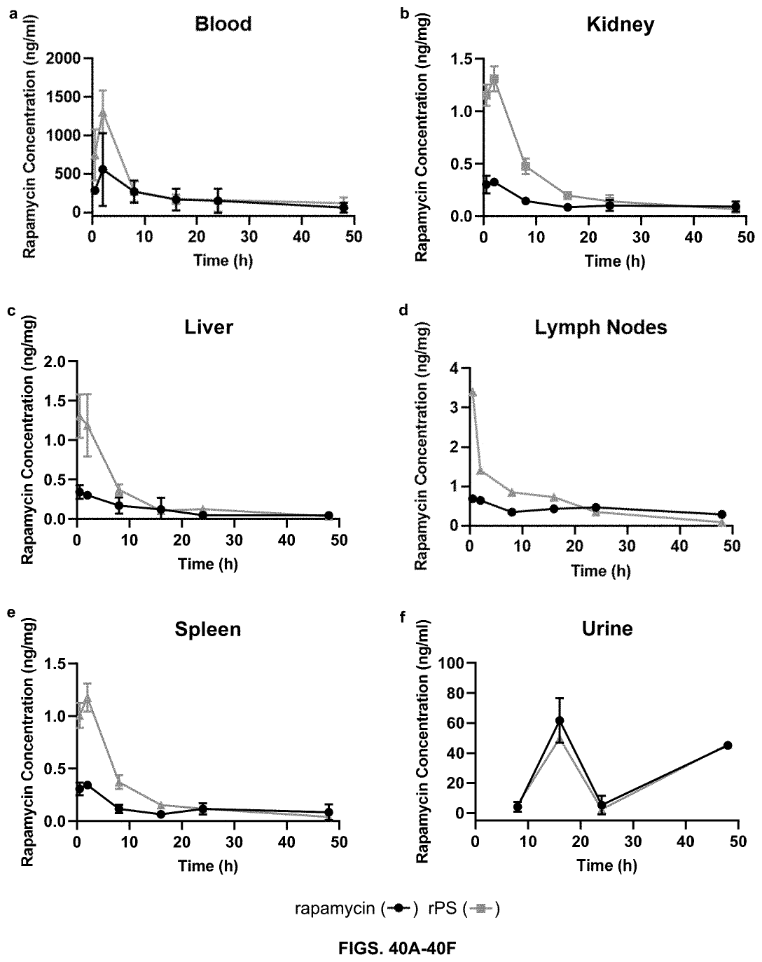

FIG. 41 shows an overview of CD45+ cell populations via tSNE from various tissues. The horizontal axis represents tSNE 1 and the vertical axis represents tSNE 2 of the CD45+ cell population. B cell are shown in orange; dendritic cells (DCs) are shown in blue; monocytes are shown in purple; neutrophils are shown in light blue; natural killer (NK) cells are shown in green; and T cells are shown in red. (n=3 mice).

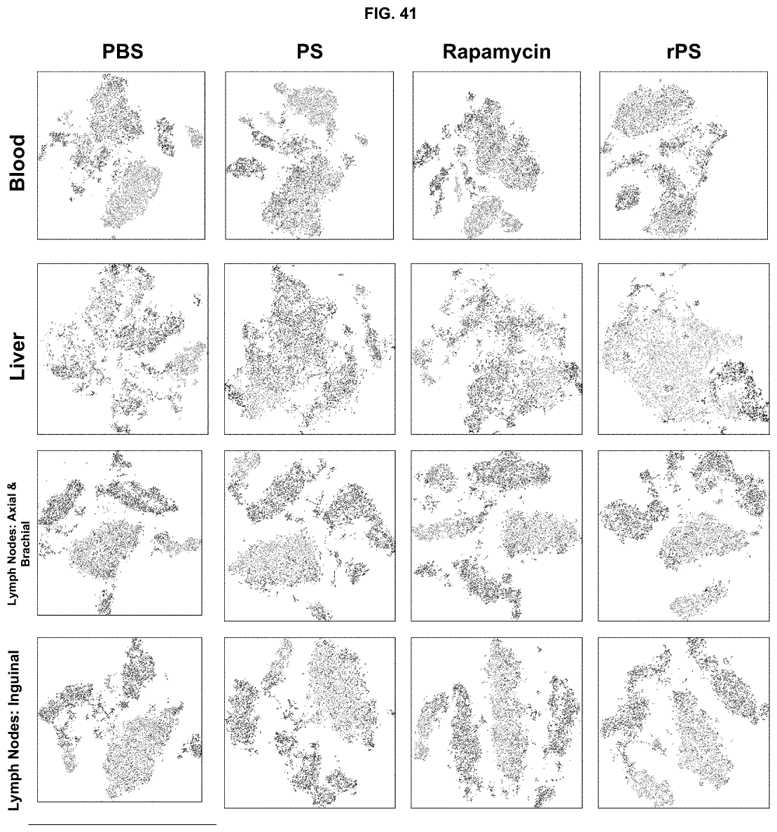

FIG. 42 shows an overview of CD45+ cell populations in blood. rPS treatment significantly increased NK cell populations relative to control treatments. (n=3 mice). All data is presented as mean±SD with *p<0.05; **p<0.01; ***p<0.001; ****p<0.0001.

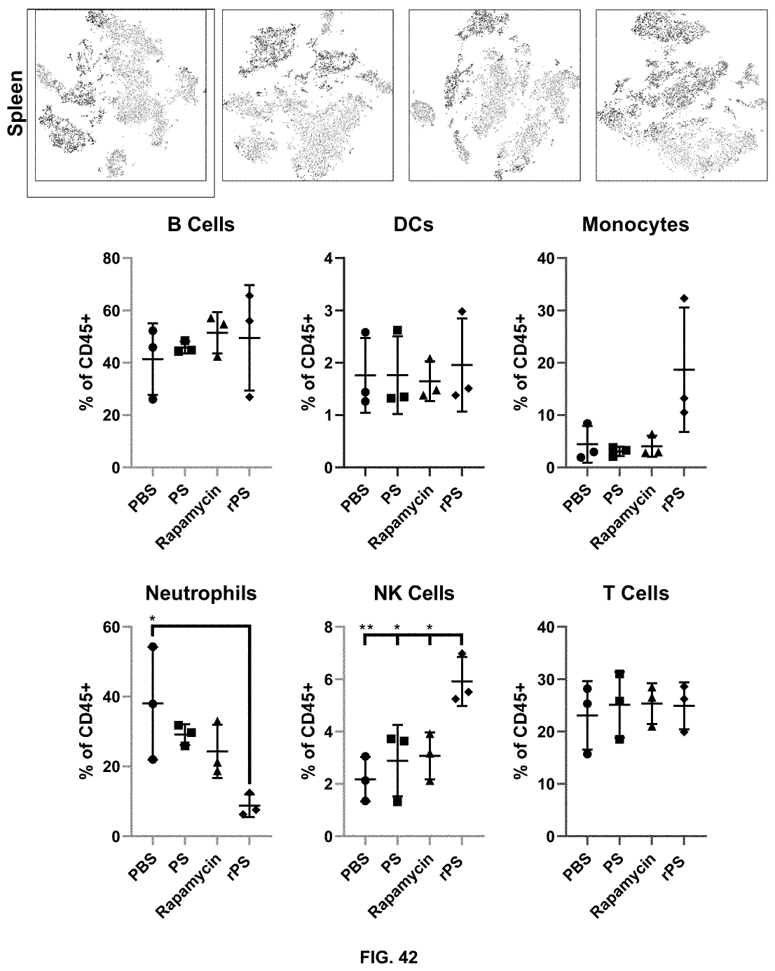

FIG. 43 shows an overview of CD45+ cell populations in liver. rPS treatment significantly increased B cells and decreased neutrophils and T cells relative to control treatments. (n=3 mice). All data is presented as mean±SD with *p<0.05; **p<0.01; ***p<0.001; ****p<0.0001.

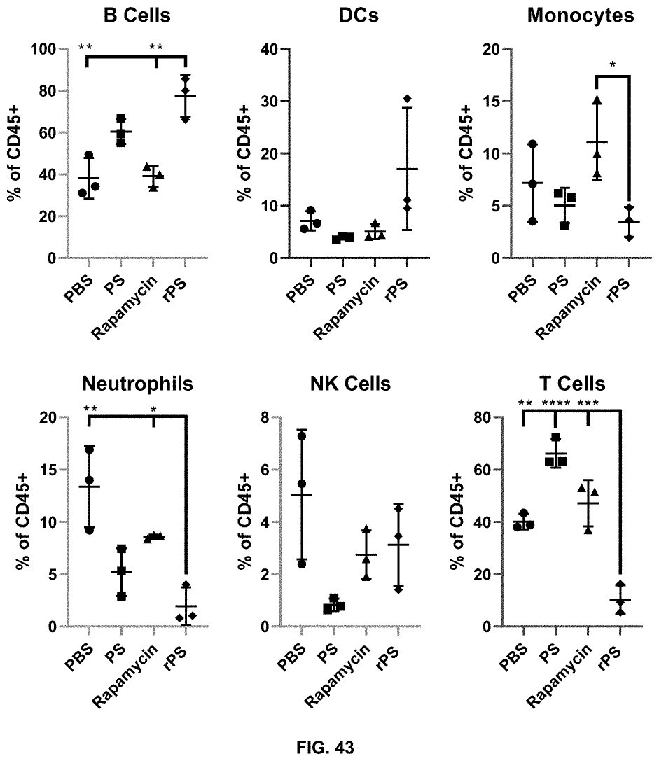

FIG. 44 shows an overview of CD45+ cell populations in axial and brachial lymph nodes. rPS treatment significantly increased DCs and monocytes and reduced T cells relative to control treatments. (n=3 mice). All data is presented as mean±SD with *p<0.05; **p<0.01; ***p<0.001; ****p<0.0001.

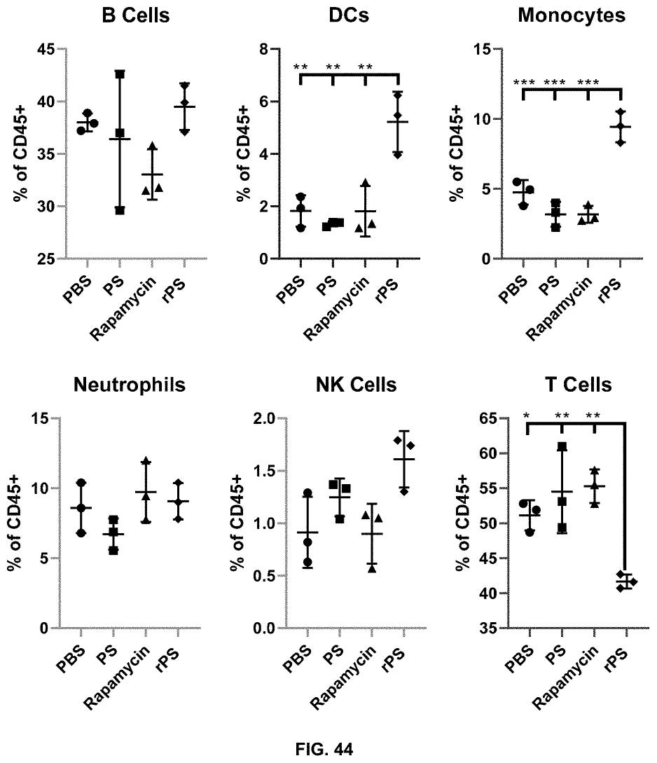

FIG. 45 shows an overview of CD45+ cell populations in inguinal lymph nodes. rPS treatment significantly increased DCs, monocytes, and neutrophils, and NK cells and reduced T cells relative to control treatments. (n=3 mice). All data is presented as mean±SD with *p<0.05; **p<0.01; ***p<0.001; ****p<0.0001.

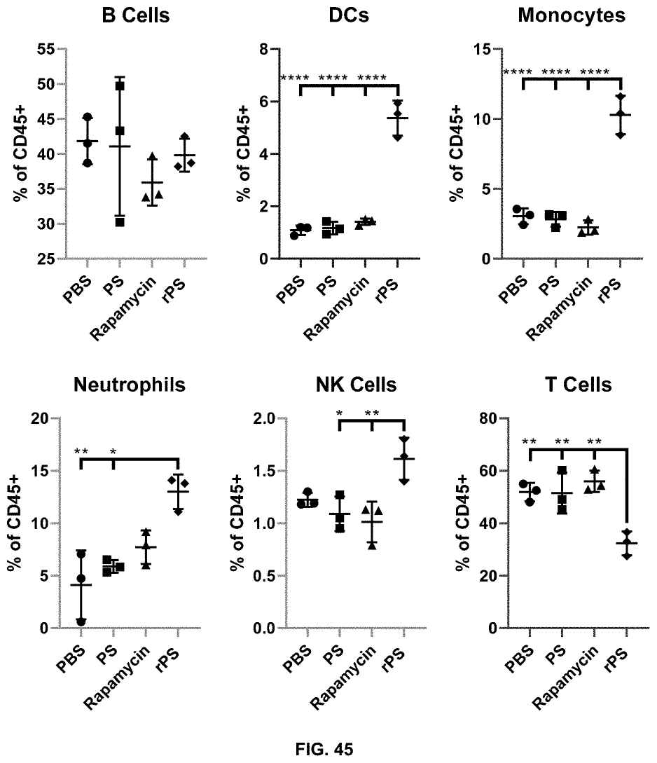

FIG. 46 shows an overview of CD45+ cell populations in spleen. rPS treatment significantly increased monocytes relative to control treatments. (n=3 mice). All data is presented as mean±SD with *p<0.05; **p<0.01; ***p<0.001; ****p<0.0001.

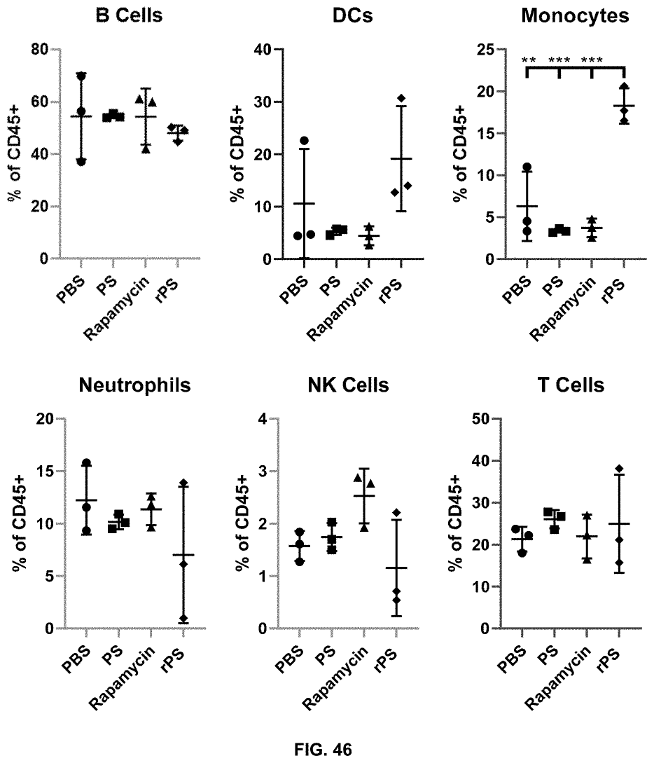

FIG. 47 shows costimulation of B Cells in blood. rPS treatment significantly reduced CD40 and CD80 costimulation of B cells in blood. (n=3 mice). For tSNE plots (top), the horizontal axis represents tSNE 1 and the vertical axis represents tSNE 2 of the CD19+ cell population. CD40+ B cell are shown in light blue; CD80+ B cells are shown in orange; CD86+ B cells are shown in green. For graphs (bottom), all data is presented as mean±SD with *p<0.05; **p<0.01; ***p<0.001; ****p<0.0001.

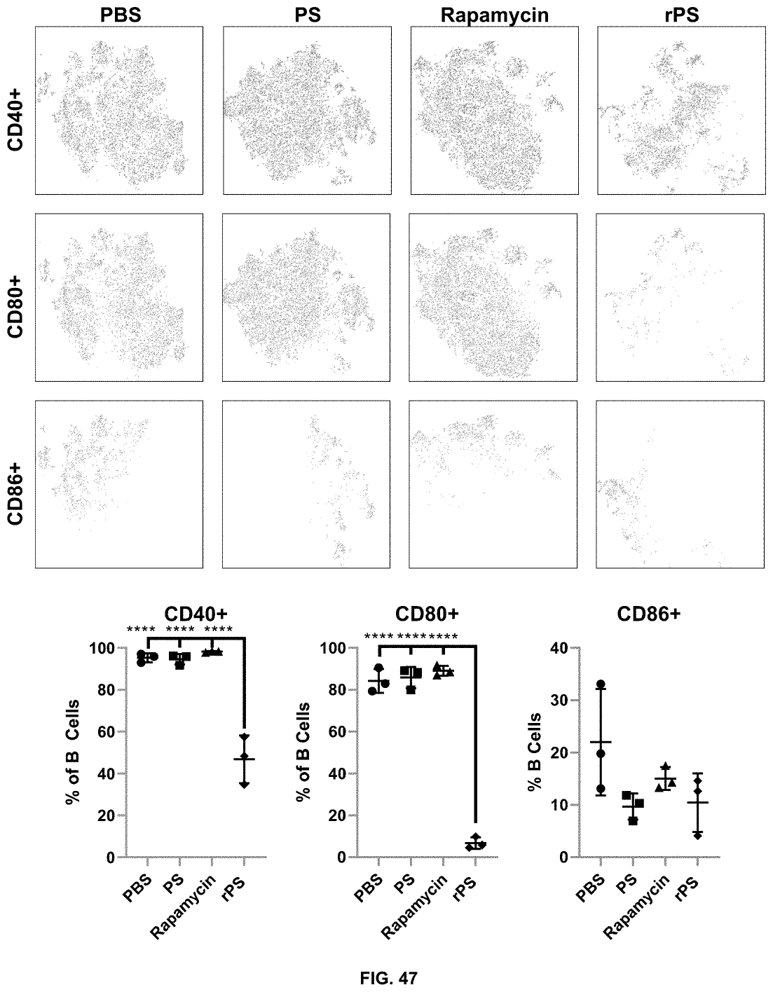

FIG. 48 shows DCs in blood. rPS treatment significantly reduced plasmacytoid DCs (pDCs) in blood. (n=3 mice). For tSNE plots (top), the horizontal axis represents tSNE 1 and the vertical axis represents tSNE 2 of the CD11c+ cell population. Conventional DCs (cDCs) Type I (CD8+CD11b−) are shown in dark purple; cDCs Type II (CD8− CD11b+) are shown in pink; pDCs are shown in green. For graphs (bottom), all data is presented as mean±SD with *p<0.05; **p<0.01; ***p<0.001; ****p<0.0001.

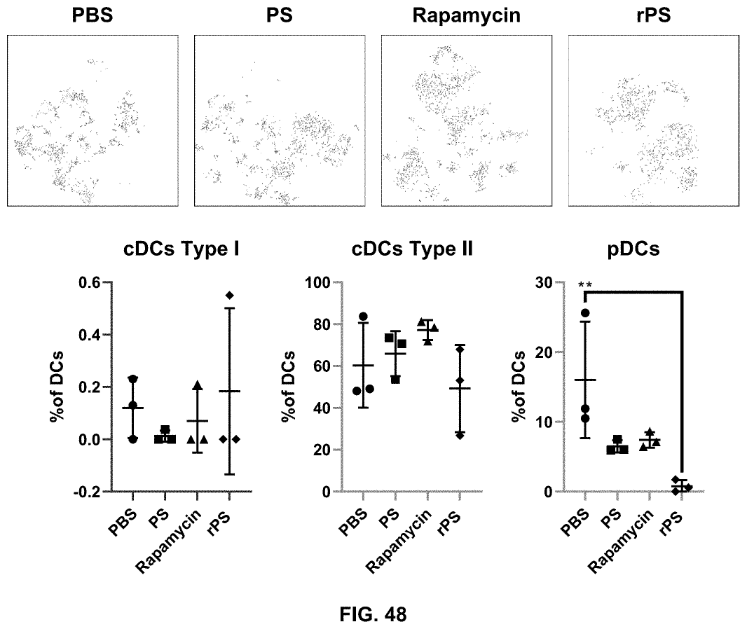

FIG. 49 shows maturation of DCs in blood. rPS treatment significantly increased maturation of MEW II in DCs in blood. (n=3 mice). For tSNE plots (top), the horizontal axis represents tSNE 1 and the vertical axis represents tSNE 2 of the CD11c+ cell population. Pre-DCs (MEW II−) are shown in blue; mature DCs (MEW II+) are shown in red. For graphs (bottom), all data is presented as mean±SD with *p<0.05; **p<0.01; ***p<0.001; ****p<0.0001.

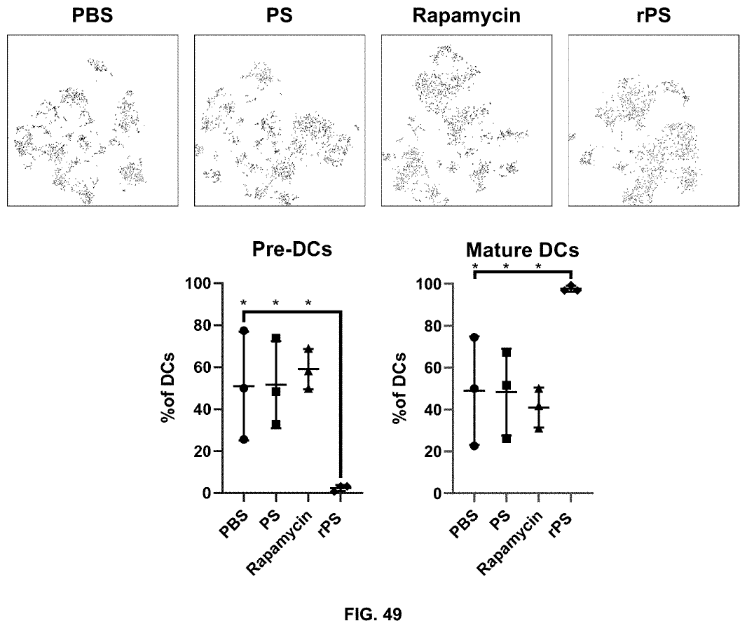

FIG. 50 shows costimulation of DCs in blood. rPS treatment significantly reduced CD80 costimulation of DCs in blood. (n=3 mice). For tSNE plots (top), the horizontal axis represents tSNE 1 and the vertical axis represents tSNE 2 of the CD11c+ cell population. CD40+ B cell are shown in light blue; CD80+ B cells are shown in orange; CD86+ B cells are shown in green. For graphs (bottom), all data is presented as mean±SD with *p<0.05; **p<0.01; ***p<0.001; ****p<0.0001.

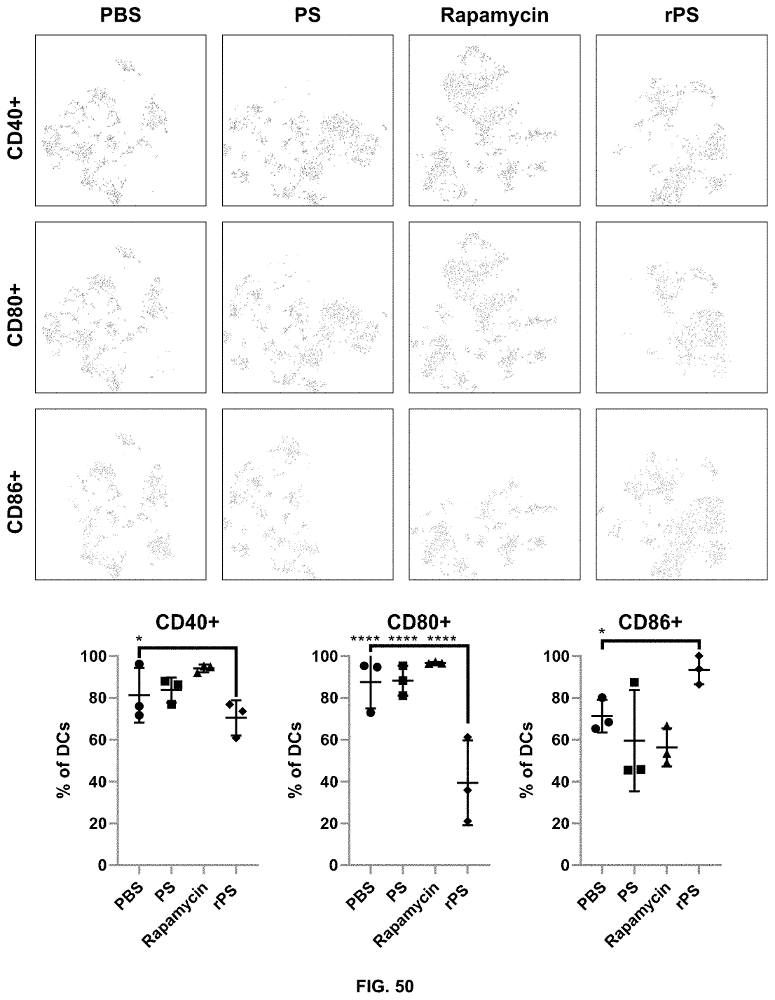

FIG. 51 shows MEW II+ monocytes in blood. rPS treatment significantly increased MEW II presentation in monocytes in blood. (n=3 mice). For tSNE plots (top), the horizontal axis represents tSNE 1 and the vertical axis represents tSNE 2 of the CD11b+ cell population. Pre-DCs (MEW II−) are shown in blue; mature DCs (MEW II+) are shown in red. For graphs (bottom), all data is presented as mean±SD with *p<0.05; **p<0.01; ***p<0.001; ****p<0.0001.

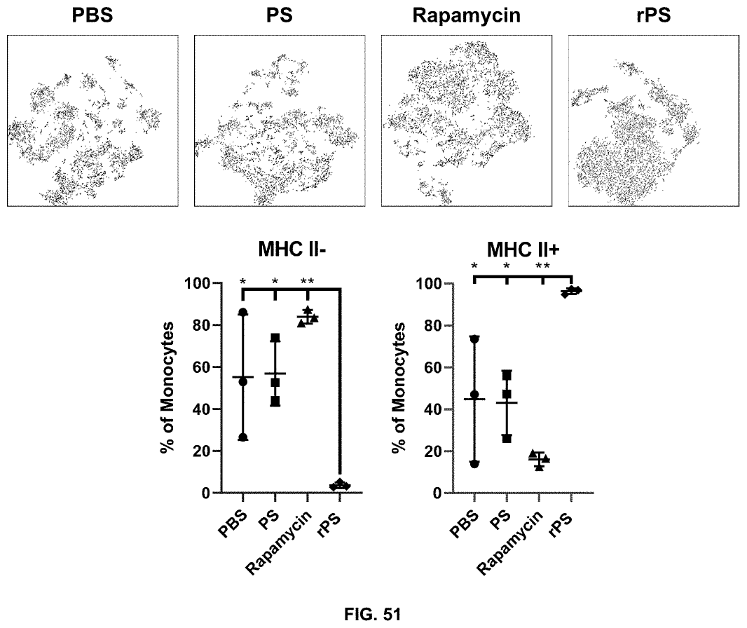

FIG. 52 shows Ly-6C^{Hi} monocytes in blood. rPS treatment significantly decreased Ly-6C presentation in monocytes in blood. (n=3 mice). For tSNE plots (top), the horizontal axis represents tSNE 1 and the vertical axis represents tSNE 2 of the CD11b+ cell population. Ly-6C^{Hi} monocytes are shown in pink; Ly-6C^{Lo} monocytes are shown in light blue. For graphs (bottom), all data is presented as mean±SD with *p<0.05; **p<0.01; ***p<0.001; ****p<0.0001.

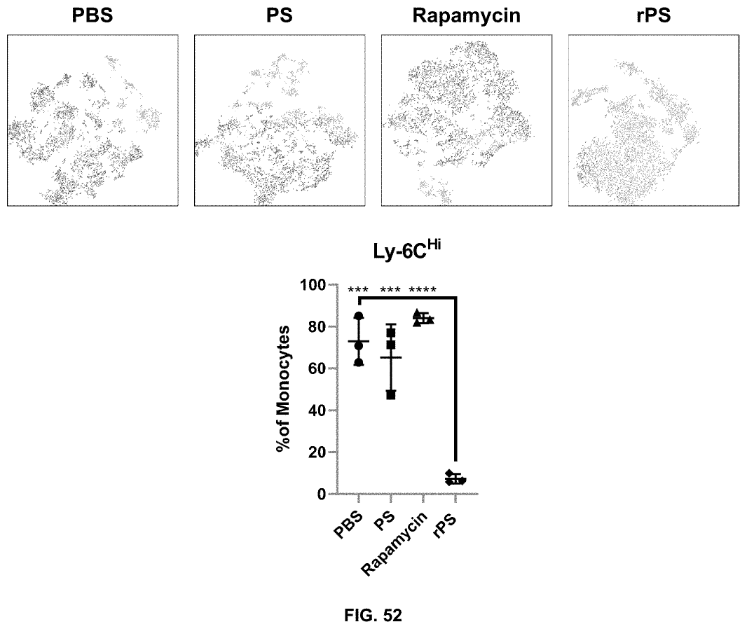

FIG. 53 shows costimulation of monocytes in blood. rPS treatment significantly reduced CD40 costimulation of monocytes in blood. (n=3 mice). For tSNE plots (top), the horizontal axis represents tSNE 1 and the vertical axis represents tSNE 2 of the CD11b+ cell population. CD40+ B cell are shown in light blue; CD80+ B cells are shown in orange; CD86+ B cells are shown in green. For graphs (bottom), all data is presented as mean±SD with *p<0.05; **p<0.01; ***p<0.001; ****p<0.0001.

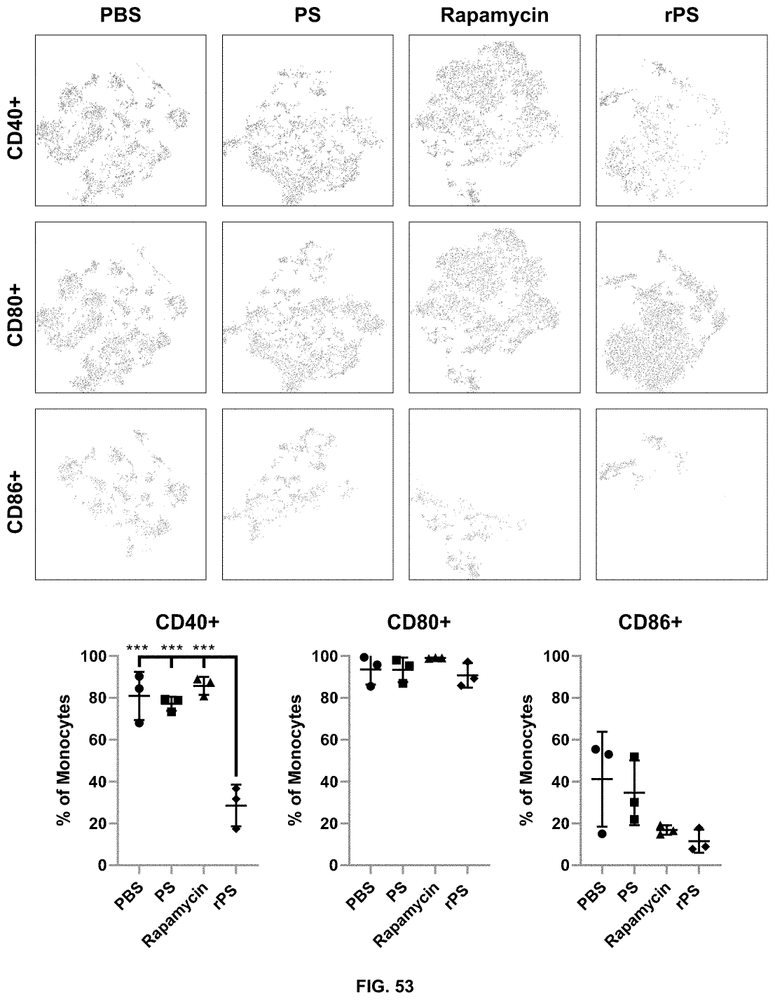

FIG. 54 shows T Cells in blood. rPS treatment significantly reduced CD4+ regulatory T cells and increased NK T cells in blood. (n=3 mice). For tSNE plots (top), the horizontal axis represents tSNE 1 and the vertical axis represents tSNE 2 of the CD3+ cell population. CD4+ T cell are shown in orange; CD8+ T cells are shown in blue; NK T cells (NK1.1+) are shown in yellow; CD4+ regulatory T cells (CD25+ FoxP3+) are shown in pink; CD8+ regulatory T cells are shown in light blue. For graphs (bottom), all data is presented as mean±SD with *p<0.05; **p<0.01; ***p<0.001; ****p<0.0001.

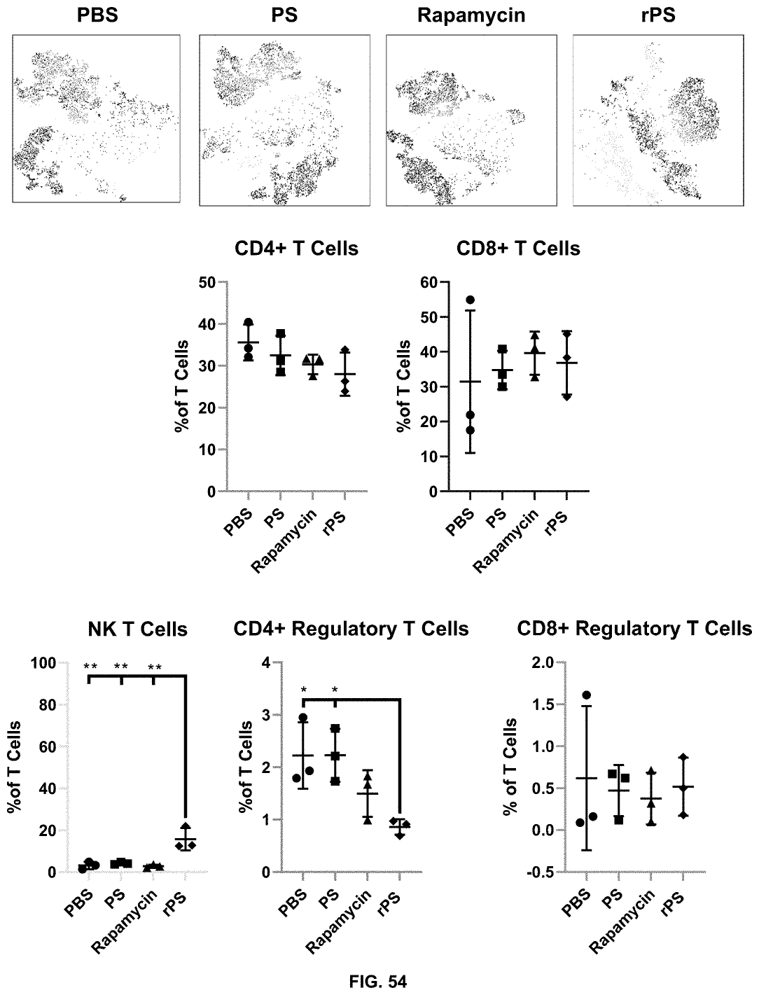

FIG. 55 shows T Cells in blood. Fig. S15 T Cells in blood. (n=3 mice). For tSNE plots (top), the horizontal axis represents tSNE 1 and the vertical axis represents tSNE 2 of the CD3+ cell population. Double negative (CD4− CD8−) T cells are shown in red; double positive (CD4+ CD8+) T cells are shown in green; CD4+ CD8− T cell are shown in orange; CD4+ CD8+ T cells are shown in blue. NK T cells (NK1.1+) are shown in yellow; CD4+ regulatory T cells (CD25+FoxP3+) are shown in pink; CD8+ regulatory T cells are shown in light blue. For graphs (bottom), all data is presented as mean±SD with *p<0.05; **p<0.01; ***p<0.001; ****p<0.0001.

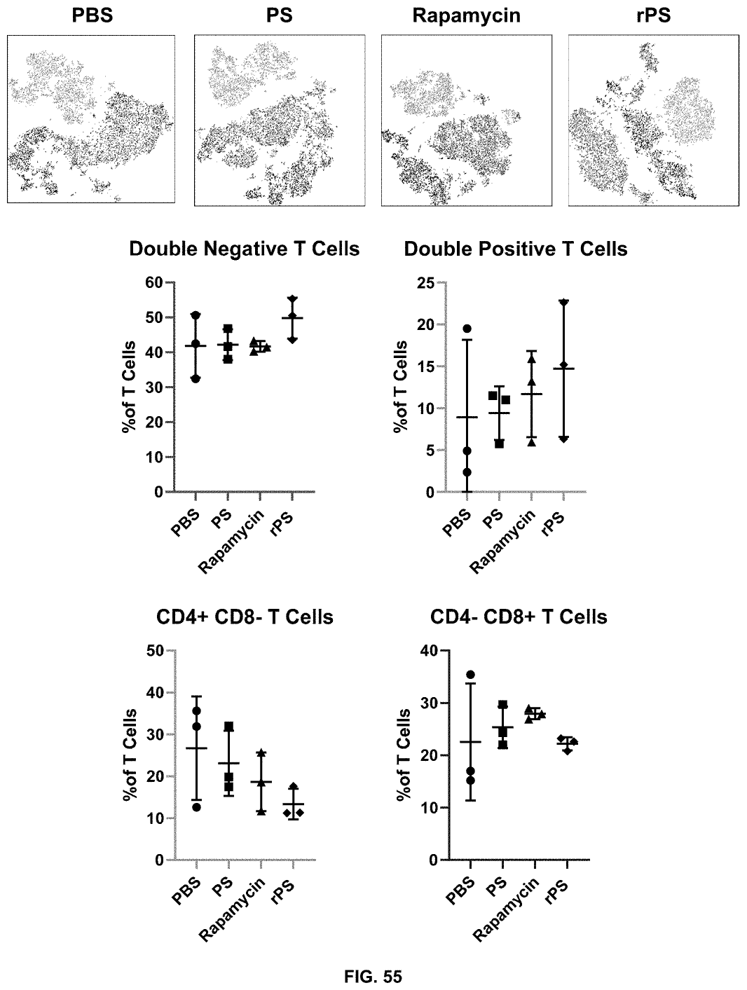

FIG. 56 shows costimulation of B cells in the liver. rPS treatment significantly reduced CD80 costimulation of monocytes in the liver. (n=3 mice). For tSNE plots (top), the horizontal axis represents tSNE 1 and the vertical axis represents tSNE 2 of the CD19+ cell population. CD40+ B cell are shown in light blue; CD80+ B cells are shown in orange; CD86+ B cells are shown in green. For graphs (bottom), all data is presented as mean±SD with *p<0.05; **p<0.01; ***p<0.001; ****p<0.0001.

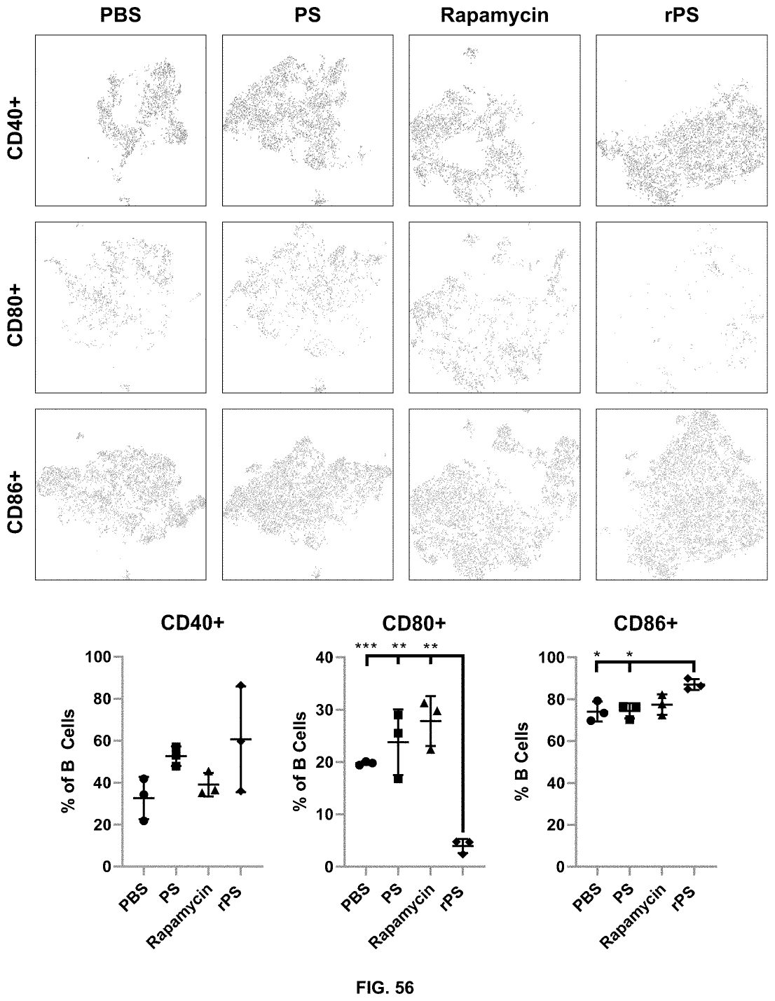

FIG. 57 shows DCs in the liver. (n=3 mice). For tSNE plots (top), the horizontal axis represents tSNE 1 and the vertical axis represents tSNE 2 of the CD11c+ cell population. cDCs Type I (CD8+ CD11b−) are shown in dark purple; cDCs Type II (CD8− CD11b+) are shown in pink; pDCs are shown in green. For graphs (bottom), all data is presented as mean±SD with *p<0.05; **p<0.01; ***p<0.001; ****p<0.0001.

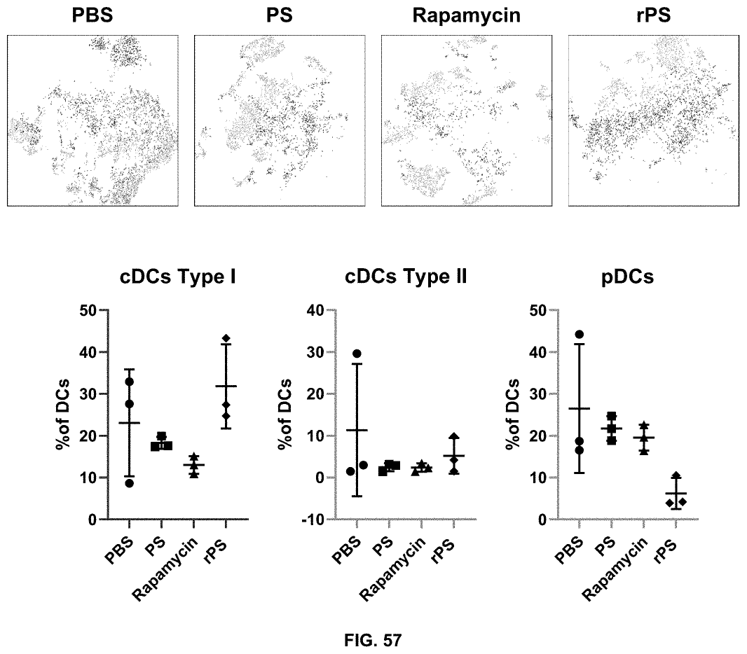

FIG. 58 shows maturation of DCs in the liver. (n=3 mice). For tSNE plots (top), the horizontal axis represents tSNE 1 and the vertical axis represents tSNE 2 of the CD11c+ cell population. Pre-DCs (MEW II−) are shown in blue; mature DCs (MEW II+) are shown in red. For graphs (bottom), all data is presented as mean±SD with *p<0.05; **p<0.01; ***p<0.001; ****p<0.0001.

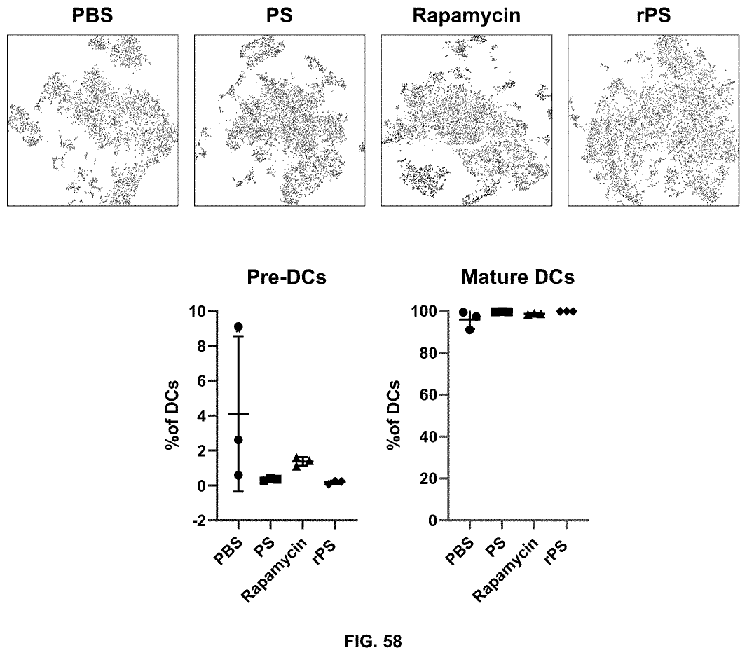

FIG. 59 shows costimulation of DCs in the liver. (n=3 mice). For tSNE plots (top), the horizontal axis represents tSNE 1 and the vertical axis represents tSNE 2 of the CD11c+ cell population. CD40+ DCs are shown in light blue; CD80+ DCs are shown in orange; CD86+ DCs are shown in green. For graphs (bottom), all data is presented as mean±SD with *p<0.05; **p<0.01; ***p<0.001; ****p<0.0001.

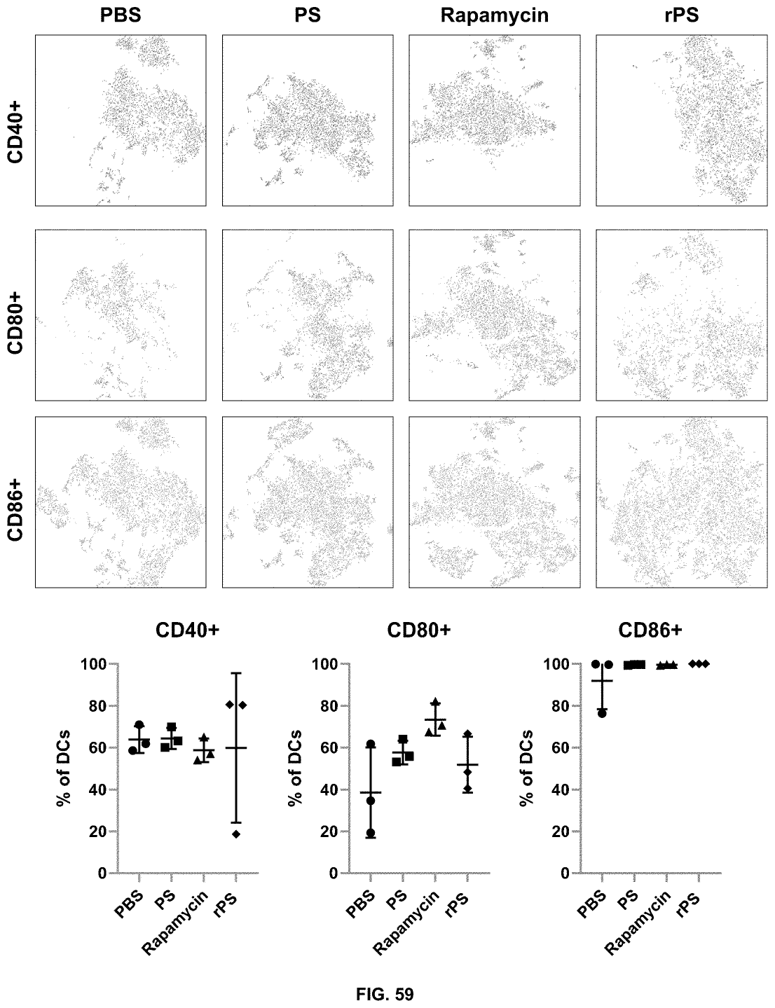

FIG. 60 shows macrophages in the liver. (n=3 mice). For tSNE plots (top), the horizontal axis represents tSNE 1 and the vertical axis represents tSNE 2 of the CD11b+ cell population. Macrophages (F4/80+ and/or CD169+) are shown in pink; Non-macrophage (F4/80− CD169−) monocytes are shown in green. For graphs (bottom), all data is presented as mean±SD with *p<0.05; **p<0.01; ***p<0.001; ****p<0.0001.

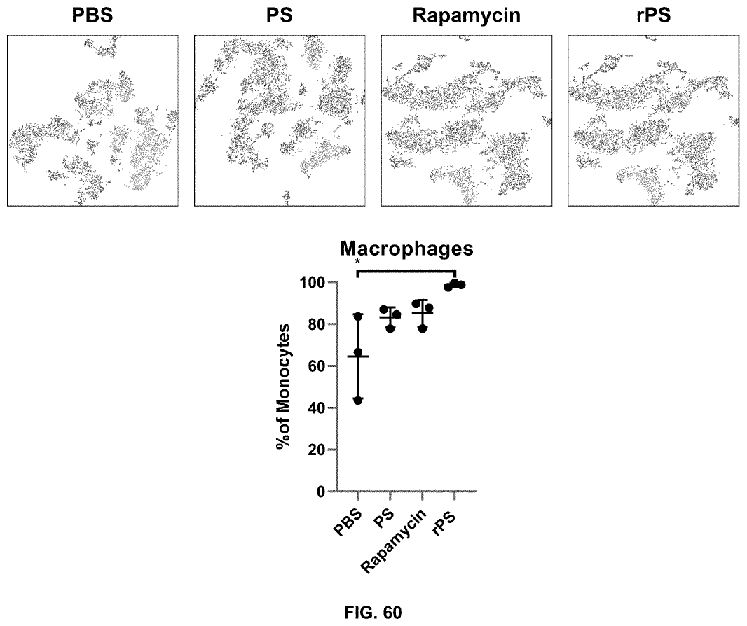

FIG. 61 shows MEW II+ monocytes in the liver. (n=3 mice). For tSNE plots (top), the horizontal axis represents tSNE 1 and the vertical axis represents tSNE 2 of the CD11b+ cell population. MEW II− monocytes are shown in blue; MEW II+ monocytes are shown in red. For graphs (bottom), all data is presented as mean±SD with *p<0.05; **p<0.01; ***p<0.001; ****p<0.0001.

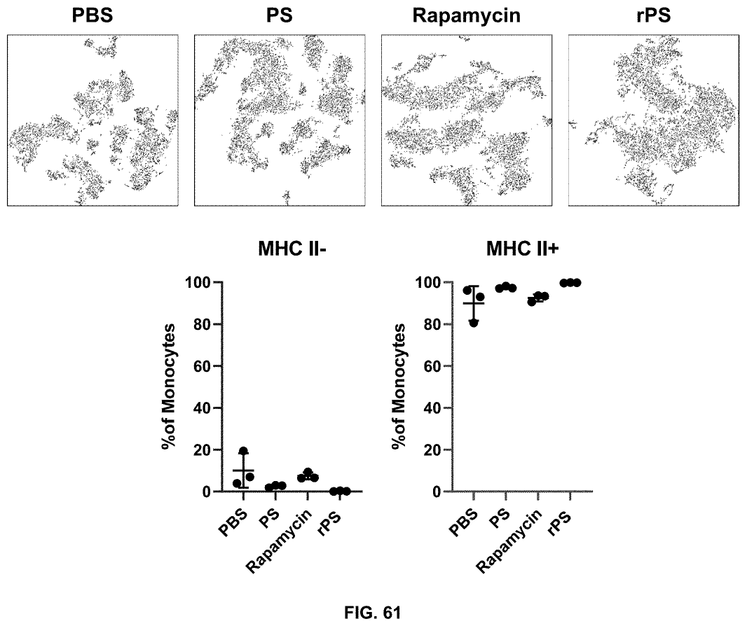

FIG. 62 shows Ly-6C+ monocytes in the liver. (n=3 mice). For tSNE plots (top), the horizontal axis represents tSNE 1 and the vertical axis represents tSNE 2 of the CD11b+ cell population. Ly-60^{Hi} monocytes are shown in pink; Ly-6C^{Lo} monocytes are shown in light blue. For graphs (bottom), all data is presented as mean±SD with *p<0.05; **p<0.01; ***p<0.001; ****p<0.0001.

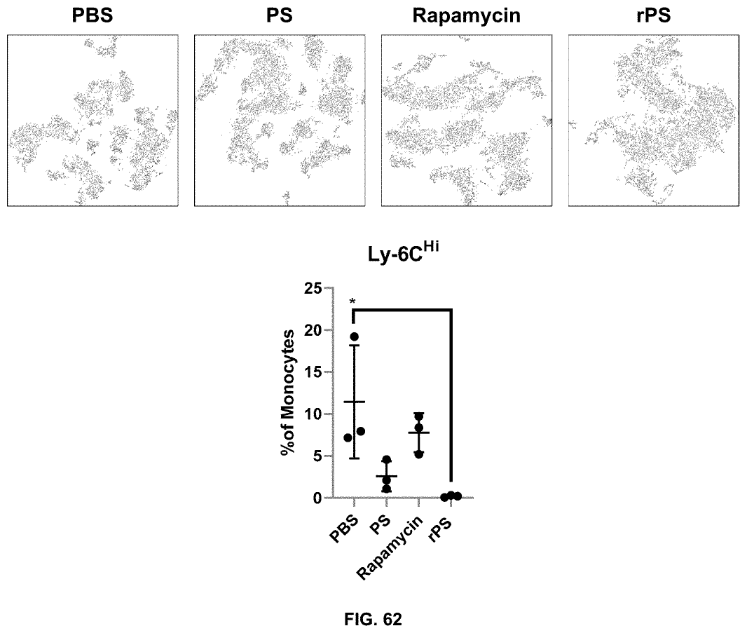

FIG. 63 show costimulation of monocytes in the liver. (n=3 mice). For tSNE plots (top), the horizontal axis represents tSNE 1 and the vertical axis represents tSNE 2 of the CD11b+ cell population. CD40+ monocytes are shown in light blue; CD80+ monocytes are shown in orange; CD86+ monocytes are shown in green. For graphs (bottom), all data is presented as mean±SD with *p<0.05; **p<0.01; ***p<0.001; ****p<0.0001.

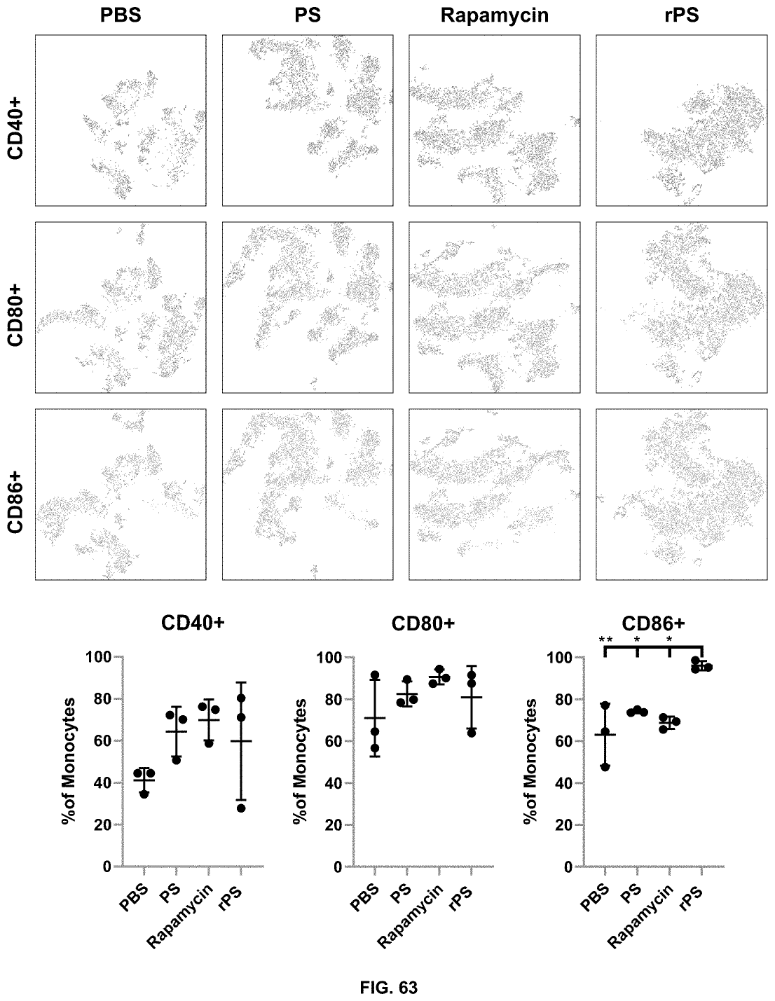

FIG. 64 shows T cells in the liver. rPS treatment significantly reduced CD8+ T cells and CD8+ regulatory T cells and increased NK T cells in the liver. (n=3 mice). For tSNE plots (top), the horizontal axis represents tSNE 1 and the vertical axis represents tSNE 2 of the CD3+ cell population. CD4+ T cell are shown in orange; CD8+ T cells are shown in blue; NK T cells (NK1.1+) are shown in yellow; CD4+ regulatory T cells (CD25+ FoxP3+) are shown in pink; CD8+ regulatory T cells are shown in light blue. For graphs (bottom), all data is presented as mean±SD with *p<0.05; **p<0.01; ***p<0.001; ****p<0.0001.

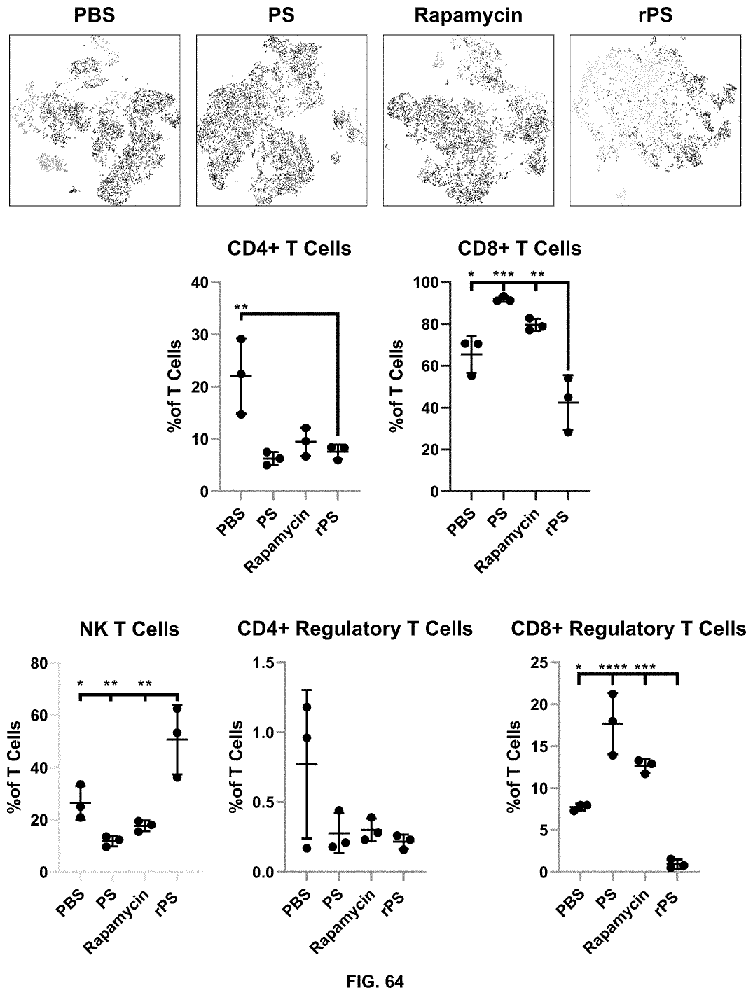

FIG. 65 shows T Cells in the liver. rPS treatment significantly increased double positive T cells in the liver. (n=3 mice). For tSNE plots (top), the horizontal axis represents tSNE 1 and the vertical axis represents tSNE 2 of the CD3+ cell population. Double negative (CD4− CD8−) T cells are shown in red; double positive (CD4+ CD8+) T cells are shown in green; CD4+ CD8− T cell are shown in orange; CD4+ CD8+ T cells are shown in blue. NK T cells (NK1.1+) are shown in yellow; CD4+ regulatory T cells (CD25+ FoxP3+) are shown in pink; CD8+ regulatory T cells are shown in light blue. For graphs (bottom), all data is presented as mean±SD with *p<0.05; **p<0.01; ***p<0.001; ****p<0.0001.

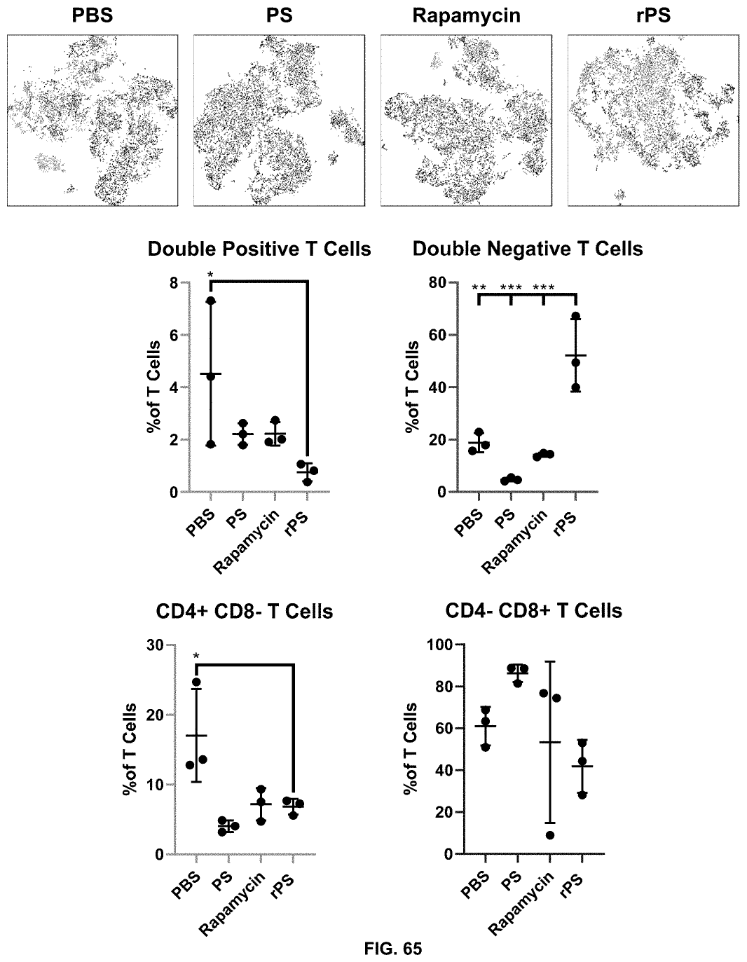

FIG. 66 shows costimulation of B cells in the axial and brachial lymph nodes. rPS treatment significantly reduced CD40 and CD80 costimulation of B cells in the axial and branchial lymph nodes. (n=3 mice). For tSNE plots (top), the horizontal axis represents tSNE 1 and the vertical axis represents tSNE 2 of the CD19+ cell population. CD40+ B cells are shown in light blue; CD80+ B cells are shown in orange; CD86+ B cells are shown in green. For graphs (bottom), all data is presented as mean±SD with *p<0.05; **p<0.01; ***p<0.001; ****p<0.0001.

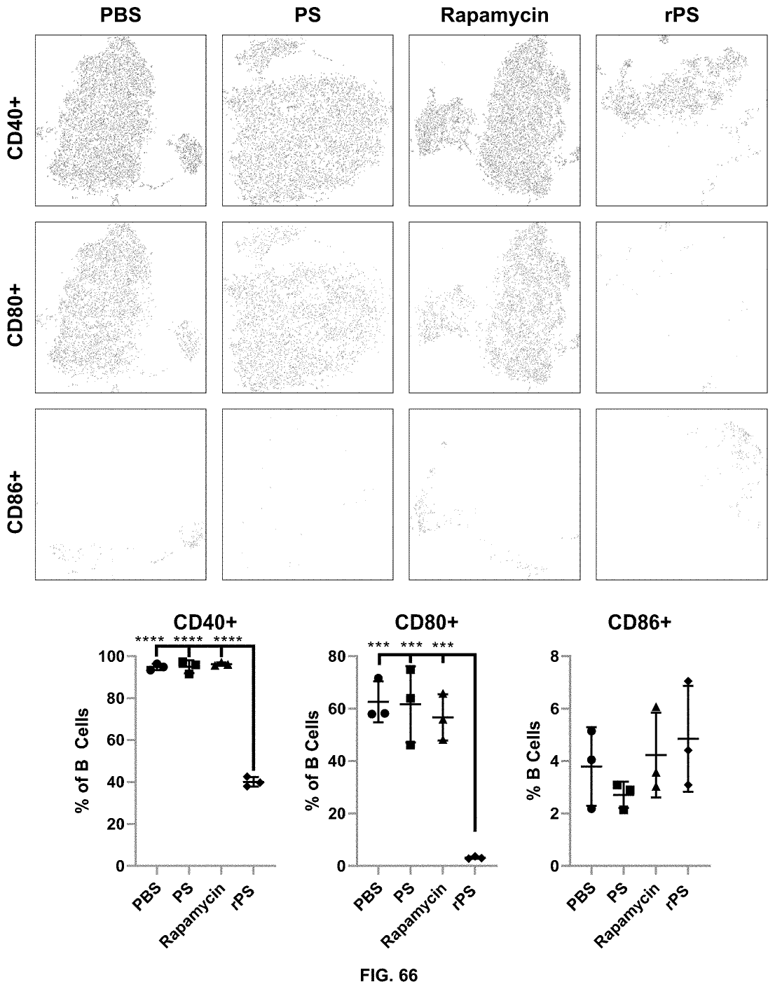

FIG. 67 shows DCs in the axial and brachial lymph nodes. rPS treatment significantly reduced pDCs in the axial and brachial lymph nodes. (n=3 mice). For tSNE plots (top), the horizontal axis represents tSNE 1 and the vertical axis represents tSNE 2 of the CD11c+ cell population. cDCs Type I (CD8+ CD11b−) are shown in dark purple; cDCs Type II (CD8− CD11b+) are shown in pink; pDCs are shown in green. For graphs (bottom), all data is presented as mean±SD with *p<0.05; **p<0.01; ***p<0.001; ****p<0.0001.

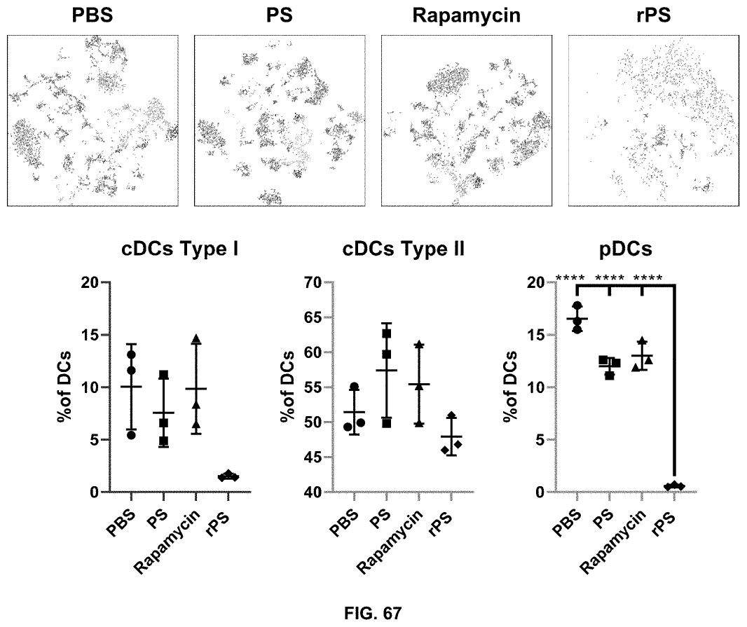

FIG. 68 shows maturation of DCs the axial and brachial lymph nodes. rPS treatment significantly increased DC maturation in the axial and brachial lymph nodes. (n=3 mice). For tSNE plots (top), the horizontal axis represents tSNE 1 and the vertical axis represents tSNE 2 of the CD11c+ cell population. Pre-DCs (MHC II−) are shown in blue; mature DCs (MHC II+) are shown in red. For graphs (bottom), all data is presented as mean±SD with *p<0.05; **p<0.01; ***p<0.001; ****p<0.0001.

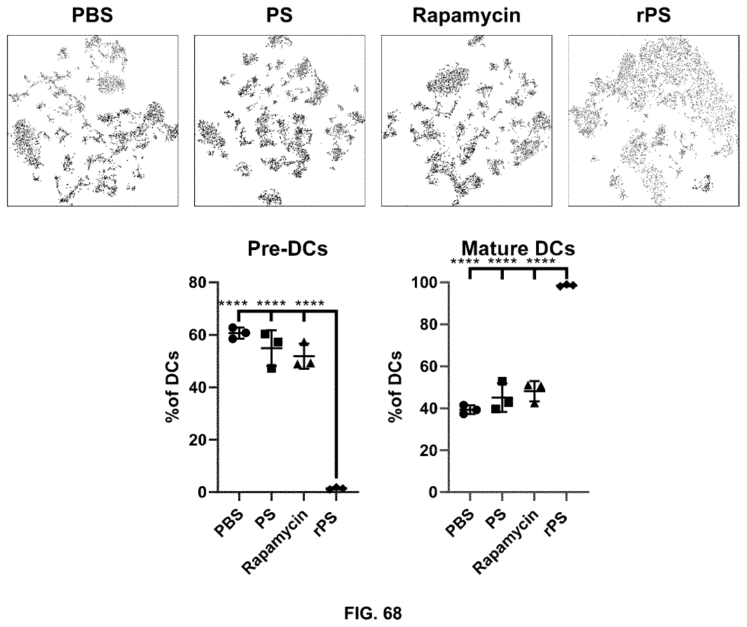

FIG. 69 shows costimulation of DCs in the axial and brachial lymph nodes. rPS treatment significantly reduced CD40, CD80 and CD86 costimulation of DCs in the axial and brachial lymph nodes. (n=3 mice). For tSNE plots (top), the horizontal axis represents tSNE 1 and the vertical axis represents tSNE 2 of the CD11c+ cell population. CD40+ DCs are shown in light blue; CD80+ DCs are shown in orange; CD86+ DCs are shown in green. For graphs (bottom), all data is presented as mean±SD with *p<0.05; **p<0.01; ***p<0.001; ****p<0.0001.

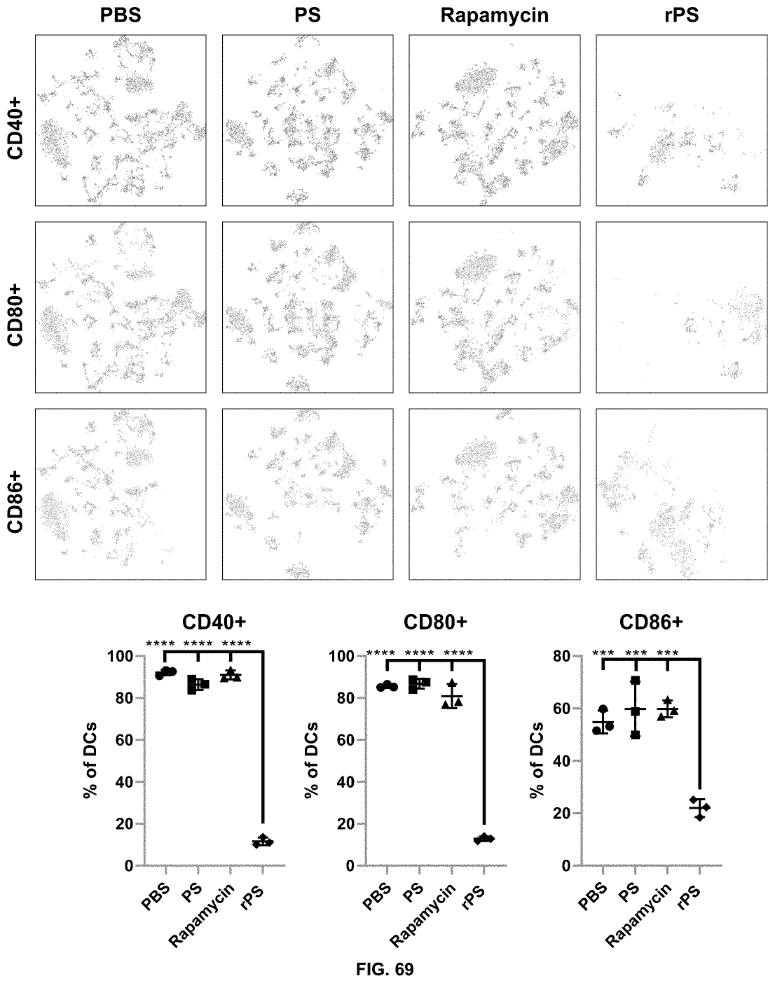

FIG. 70 shows macrophages in the axial and brachial lymph nodes. rPS treatment significantly reduced the macrophage population in the axial and brachial lymph nodes. (n=3 mice). For tSNE plots (top), the horizontal axis represents tSNE 1 and the vertical axis represents tSNE 2 of the CD11b+ cell population. Macrophages (F4/80+ and/or CD169+) are shown in pink; Non-macrophage (F4/80− CD169−) monocytes are shown in green. For graphs (bottom), all data is presented as mean±SD with *p<0.05; **p<0.01; ***p<0.001; ****p<0.0001.

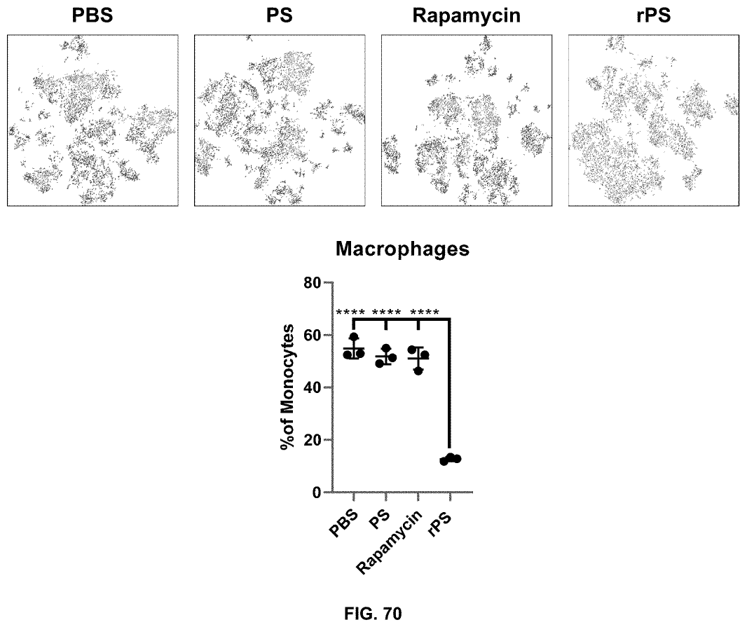

FIG. 71 shows MHC II+ monocytes in the axial and brachial lymph nodes. rPS treatment significantly increased the MHC II+ monocyte population in the axial and brachial lymph nodes. (n=3 mice). For tSNE plots (top), the horizontal axis represents tSNE 1 and the vertical axis represents tSNE 2 of the CD11b+ cell population. MHC II− monocytes are shown in blue; MHC II+ monocytes are shown in red. For graphs (bottom), all data is presented as mean±SD with *p<0.05; **p<0.01; ***p<0.001; ****p<0.0001.

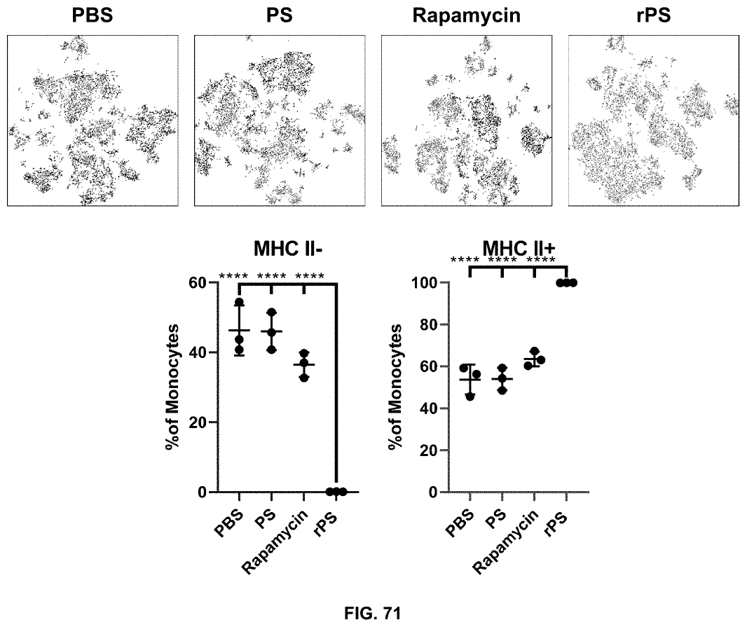

FIG. 72 shows Ly-6C+ monocytes in the axial and brachial lymph nodes. rPS treatment significantly reduced the Ly-6C^{Hi} monocyte population in the axial and brachial lymph nodes. (n=3 mice). For tSNE plots (top), the horizontal axis represents tSNE 1 and the vertical axis represents tSNE 2 of the CD11b+ cell population. Ly-6C^{Hi} monocytes are shown in pink; Ly-6C^{Lo} monocytes are shown in light blue. For graphs (bottom), all data is presented as mean±SD with *p<0.05; **p<0.01; ***p<0.001; ****p<0.0001.

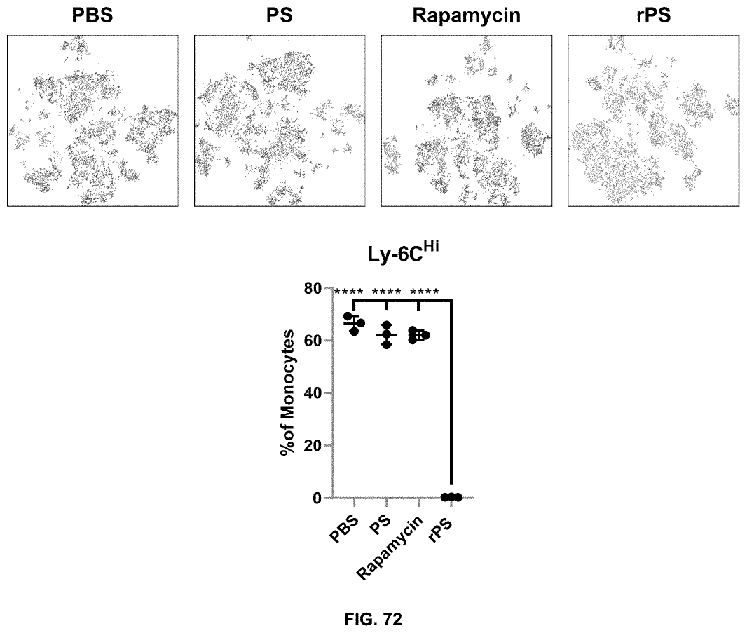

FIG. 73 shows costimulation of monocytes in the axial and brachial lymph nodes. rPS treatment significantly reduced CD40, CD80 and CD86 costimulation of monocytes in the axial and brachial lymph nodes. (n=3 mice). For tSNE plots (top), the horizontal axis represents tSNE 1 and the vertical axis represents tSNE 2 of the CD11b+ cell population. CD40+ monocytes are shown in light blue; CD80+ monocytes are shown in orange; CD86+ monocytes are shown in green. For graphs (bottom), all data is presented as mean±SD with *p<0.05; **p<0.01; ***p<0.001; ****p<0.0001.

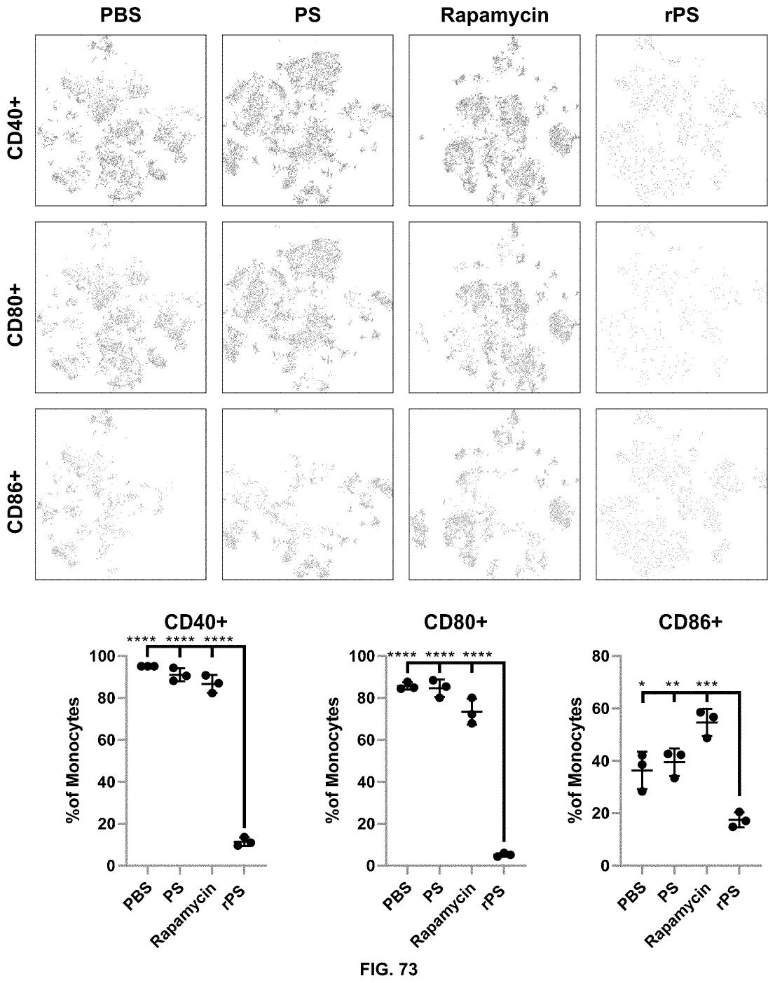

FIG. 74 shows T cells in the axial and brachial lymph nodes. rPS treatment significantly reduced CD4+ T cells and CD4+ regulatory T cells and increased CD8+ T cells, NK T cells, and CD8+ regulatory T cells in the axial and brachial lymph nodes. (n=3 mice). For tSNE plots (top), the horizontal axis represents tSNE 1 and the vertical axis represents tSNE 2 of the CD3+ cell population. CD4+ T cell are shown in orange; CD8+ T cells are shown in blue; NK T cells (NK1.1+) are shown in yellow; CD4+ regulatory T cells (CD25+ FoxP3+) are shown in pink; CD8+ regulatory T cells are shown in light blue. For graphs (bottom), all data is presented as mean±SD with *p<0.05; **p<0.01; ***p<0.001; ****p<0.0001.

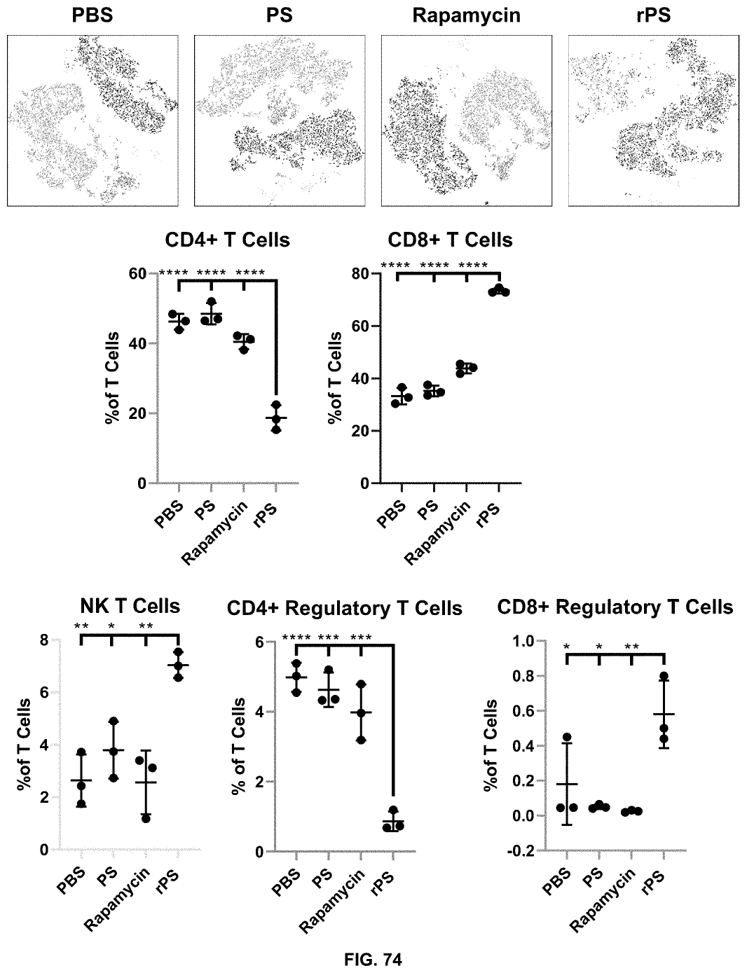

FIG. 75 shows T cells in the axial and brachial lymph nodes. rPS treatment significantly increased double positive T cells, CD4− CD8+ T cells and reduced CD4+ CD8− T cells in the axial and brachial lymph nodes. (n=3 mice). For tSNE plots (top), the horizontal axis represents tSNE 1 and the vertical axis represents tSNE 2 of the CD3+ cell population. Double negative (CD4− CD8−) T cells are shown in red; double positive (CD4+ CD8+) T cells are shown in green; CD4+CD8− T cell are shown in orange; CD4+ CD8+ T cells are shown in blue. NK T cells (NK1.1+) are shown in yellow; CD4+ regulatory T cells (CD25+ FoxP3+) are shown in pink; CD8+ regulatory T cells are shown in light blue. For graphs (bottom), all data is presented as mean±SD with *p<0.05; **p<0.01; ***p<0.001; ****p<0.0001.

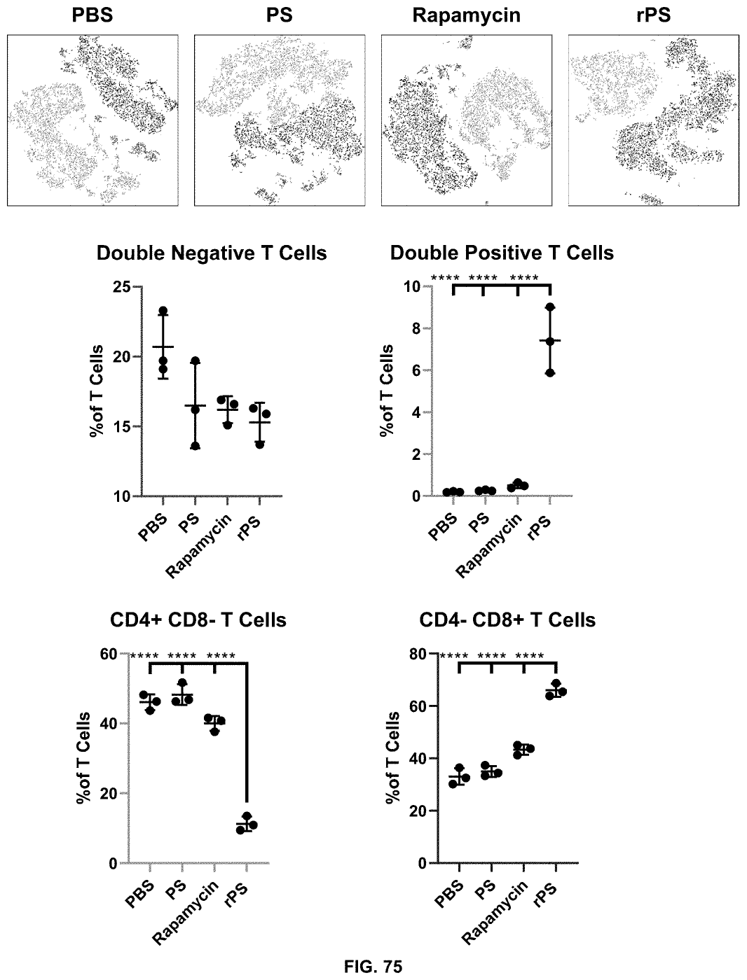

FIG. 76 shows costimulation of B cells in the inguinal lymph nodes. rPS treatment significantly reduced CD40 and CD80 costimulation of B cells in the inguinal lymph nodes. (n=3 mice). For tSNE plots (top), the horizontal axis represents tSNE 1 and the vertical axis represents tSNE 2 of the CD19+ cell population. CD40+ B cells are shown in light blue; CD80+ B cells are shown in orange; CD86+ B cells are shown in green. For graphs (bottom), all data is presented as mean±SD with *p<0.05; **p<0.01; ***p<0.001; ****p<0.0001.

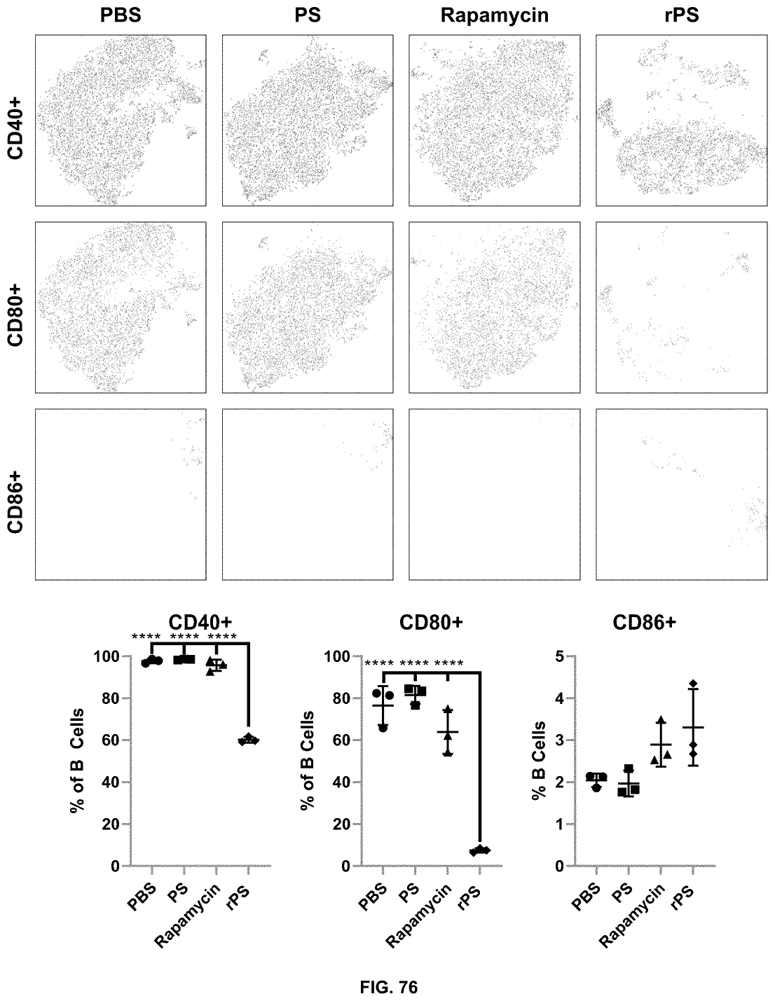

FIG. 77 shows DCs in the inguinal lymph nodes. rPS treatment significantly reduced cDCS Type II and pDCs in the inguinal lymph nodes. (n=3 mice). For tSNE plots (top), the horizontal axis represents tSNE 1 and the vertical axis represents tSNE 2 of the CD11c+ cell population. cDCs Type I (CD8+ CD11b−) are shown in dark purple; cDCs Type II (CD8− CD11b+) are shown in pink; pDCs are shown in green. For graphs (bottom), all data is presented as mean±SD with *p<0.05; **p<0.01; ***p<0.001; ****p<0.0001.

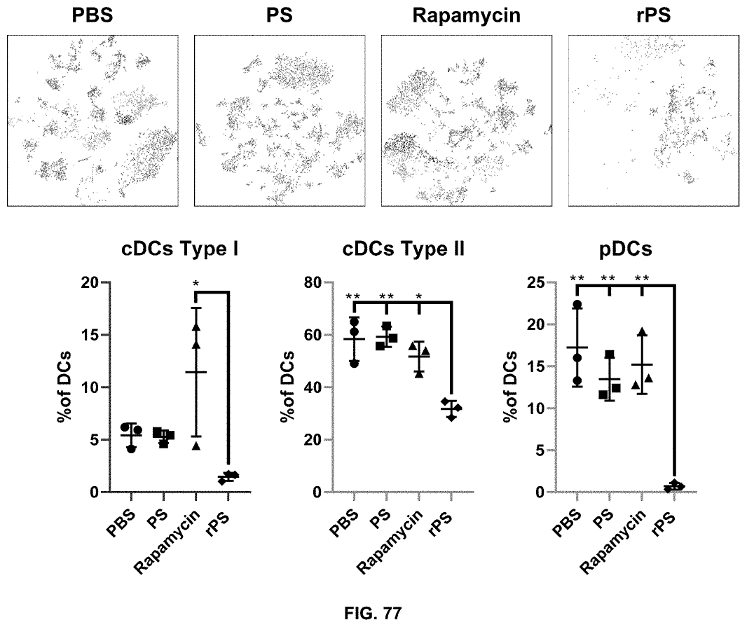

FIG. 78 shows maturation of DCs in the inguinal lymph nodes. rPS treatment significantly increased DC maturation in the inguinal lymph nodes. (n=3 mice). For tSNE plots (top), the horizontal axis represents tSNE 1 and the vertical axis represents tSNE 2 of the CD11c+cell population. Pre-DCs (MEW II−) are shown in blue; mature DCs (MEW II+) are shown in red. For graphs (bottom), all data is presented as mean±SD with *p<0.05; **p<0.01; ***p<0.001; ****p<0.0001.

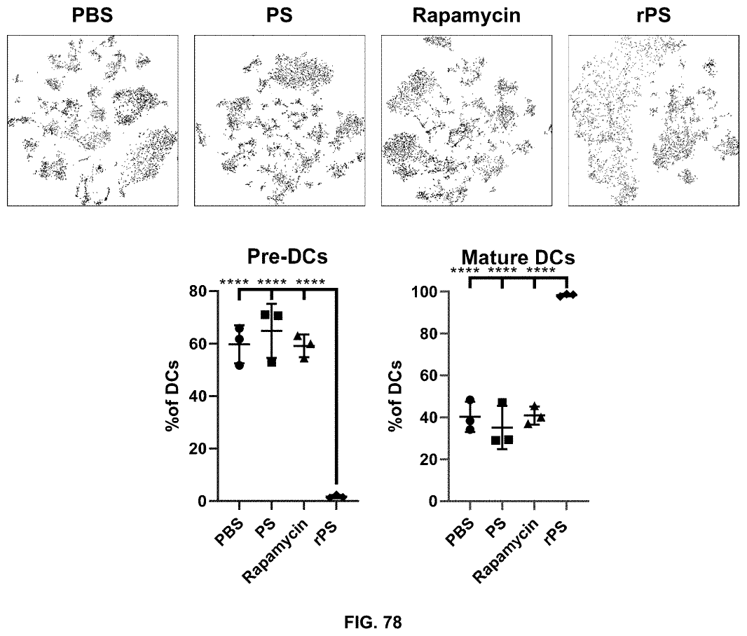

FIG. 79 shows costimulation of DCs in the inguinal lymph nodes. rPS treatment significantly reduced CD40 and CD80 costimulation of DCs in the inguinal lymph nodes. (n=3 mice). For tSNE plots (top), the horizontal axis represents tSNE 1 and the vertical axis represents tSNE 2 of the CD11b+ cell population. CD40+ DCs are shown in light blue; CD80+ DCs are shown in orange; CD86+ DCs are shown in green. For graphs (bottom), all data is presented as mean±SD with *p<0.05; **p<0.01; ***p<0.001; ****p<0.0001.

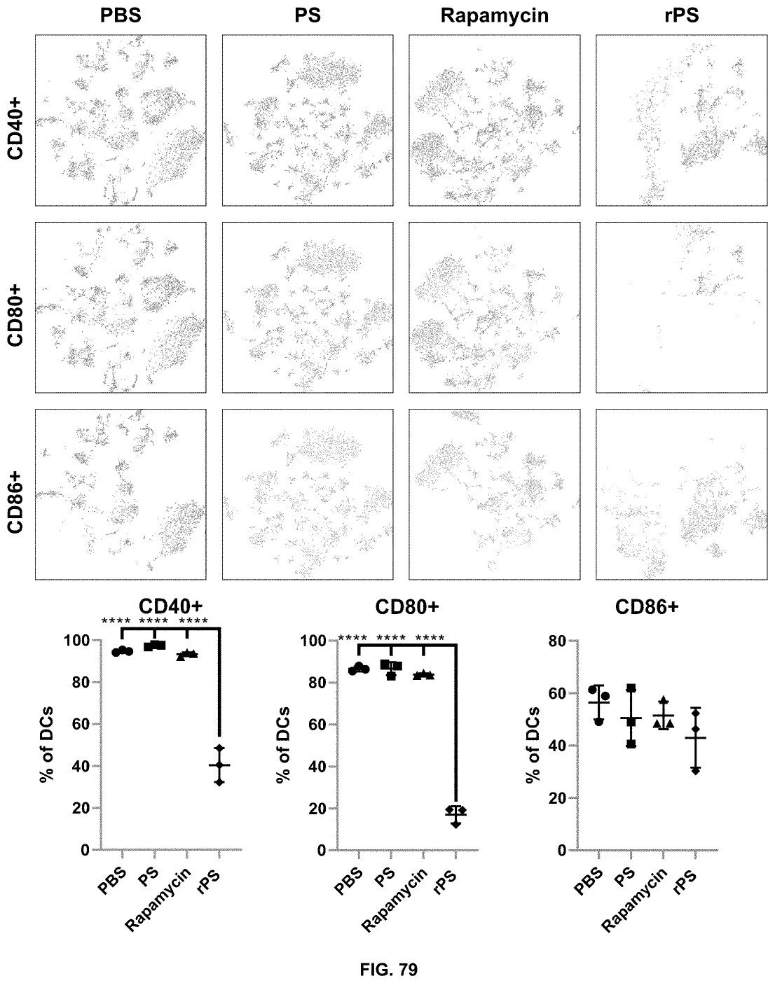

FIG. 80 shows macrophages in the inguinal lymph nodes. rPS treatment significantly reduced the macrophage population in the inguinal lymph nodes. (n=3 mice). For tSNE plots (top), the horizontal axis represents tSNE 1 and the vertical axis represents tSNE 2 of the CD11b+ cell population. Macrophages (F4/80+ and/or CD169+) are shown in pink; Non-macrophage (F4/80− CD169−) monocytes are shown in green. For graphs (bottom), all data is presented as mean±SD with *p<0.05; **p<0.01; ***p<0.001; ****p<0.0001.

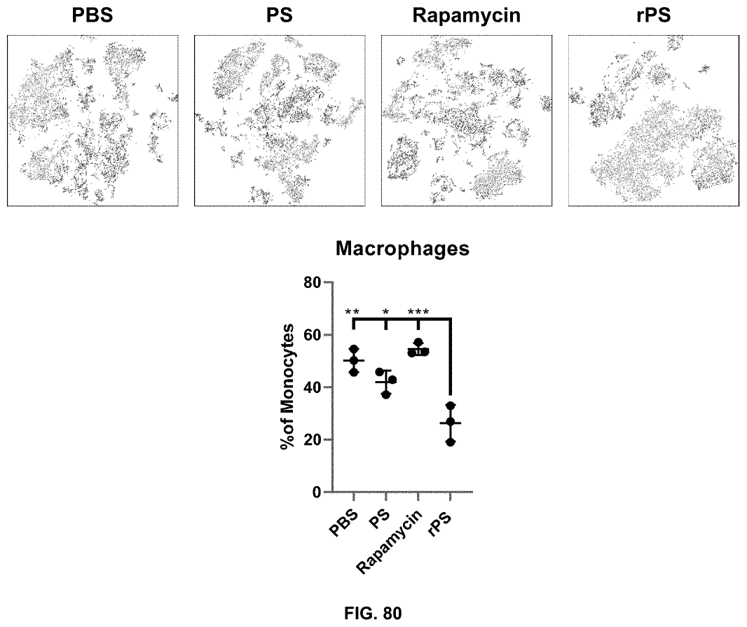

FIG. 81 shows MEW II+ monocytes in inguinal lymph nodes. rPS treatment significantly increased the MEW II+ monocyte population in the inguinal lymph nodes. (n=3 mice). For tSNE plots (top), the horizontal axis represents tSNE 1 and the vertical axis represents tSNE 2 of the CD11b+ cell population. MEW II− monocytes are shown in blue; MEW II+ monocytes are shown in red. For graphs (bottom), all data is presented as mean±SD with *p<0.05; **p<0.01; ***p<0.001; ****p<0.0001.

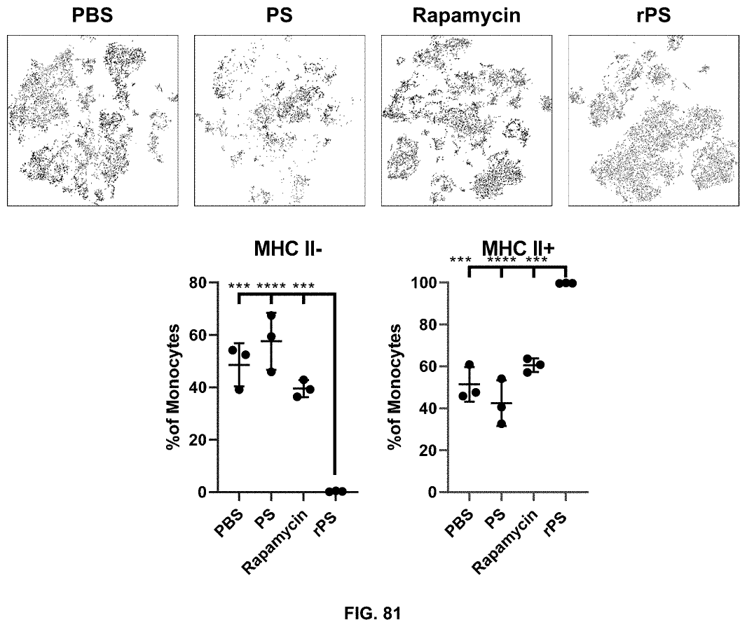

FIG. 82 shows Ly-6C^{Hi} monocytes in the inguinal lymph nodes. rPS treatment significantly reduced the Ly-6C^{Hi} monocyte population in the inguinal lymph nodes. (n=3 mice). For tSNE plots (top), the horizontal axis represents tSNE 1 and the vertical axis represents tSNE 2 of the CD11b+ cell population. Ly-6C^{Hi} monocytes are shown in pink; Ly-6C^{Lo} monocytes are shown in light blue. For graphs (bottom), all data is presented as mean±SD with *p<0.05; **p<0.01; ***p<0.001; ****p<0.0001.

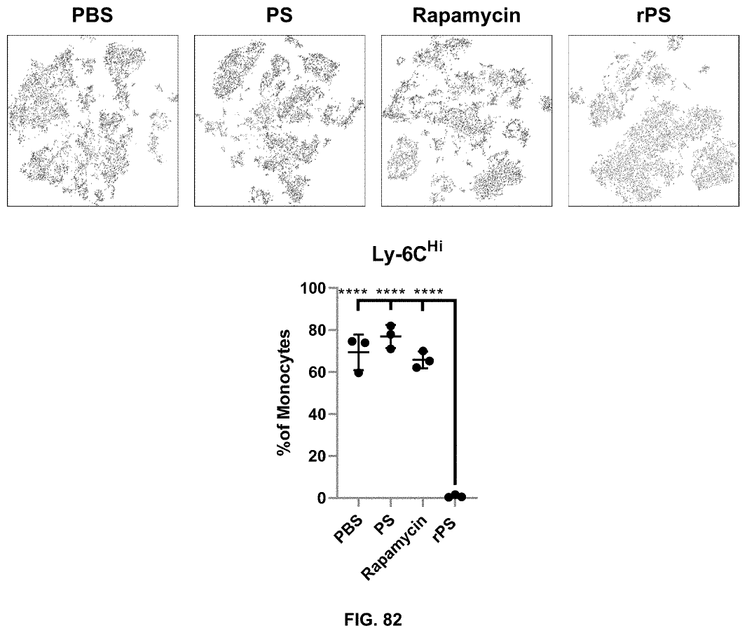

FIG. 83 shows costimulation of monocytes in the inguinal lymph nodes. rPS treatment significantly reduced CD40 and CD80 costimulation of monocytes in the inguinal lymph nodes. (n=3 mice). For tSNE plots (top), the horizontal axis represents tSNE 1 and the vertical axis represents tSNE 2 of the CD11b+ cell population. CD40+ monocytes are shown in light blue; CD80+ monocytes are shown in orange; CD86+ monocytes are shown in green. For graphs (bottom), all data is presented as mean±SD with *p<0.05; **p<0.01; ***p<0.001; ****p<0.0001.

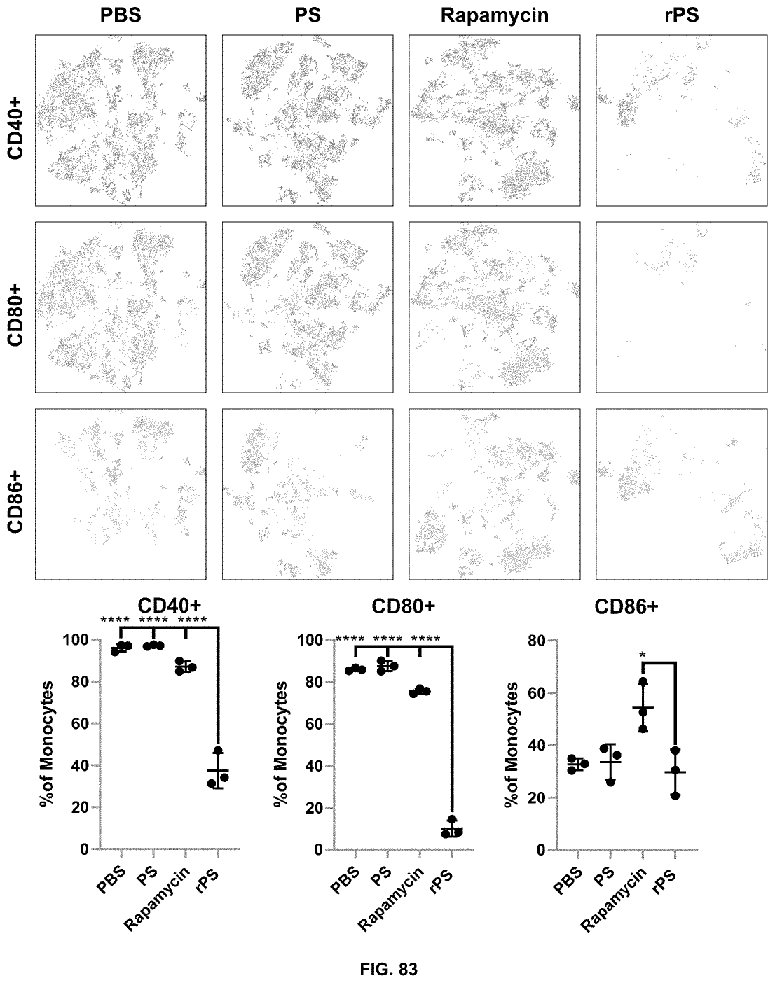

FIG. 84 shows T cells in the inguinal lymph nodes. rPS treatment significantly reduced CD4+ T cells and CD4+ regulatory T cells and increased CD8+ T cells, NK T cells, and CD8+ regulatory T cells in the inguinal lymph nodes. (n=3 mice). For tSNE plots (top), the horizontal axis represents tSNE 1 and the vertical axis represents tSNE 2 of the CD3+ cell population. CD4+ T cell are shown in orange; CD8+ T cells are shown in blue; NK T cells (NK1.1+) are shown in yellow; CD4+ regulatory T cells (CD25+ FoxP3+) are shown in pink; CD8+ regulatory T cells are shown in light blue. For graphs (bottom), all data is presented as mean±SD with *p<0.05; **p<0.01; ***p<0.001; ****p<0.0001.

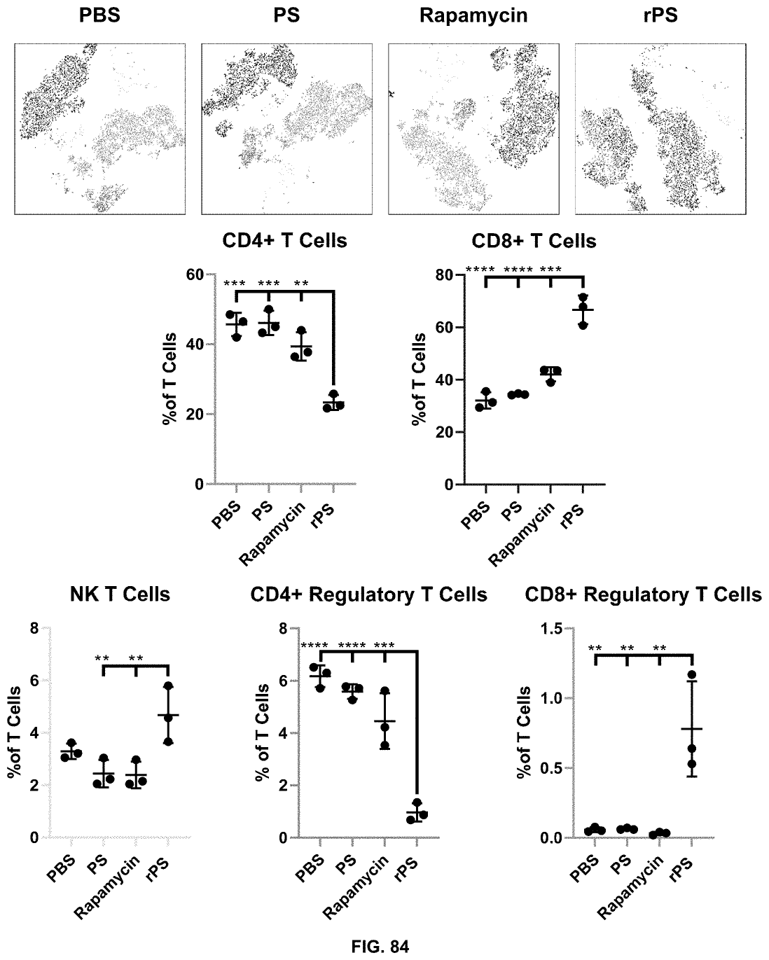

FIG. 85 shows T cells in the inguinal lymph nodes. rPS treatment significantly increased double positive T cells, CD4− CD8+ T cells and reduced CD4+ CD8− T cells in the inguinal lymph nodes. (n=3 mice). For tSNE plots (top), the horizontal axis represents tSNE 1 and the vertical axis represents tSNE 2 of the CD3+ cell population. Double negative (CD4− CD8−) T cells are shown in red; double positive (CD4+ CD8+) T cells are shown in green; CD4+ CD8− T cell are shown in orange; CD4+ CD8+ T cells are shown in blue. NK T cells (NK1.1⁺) are shown in yellow; CD4+ regulatory T cells (CD25+ FoxP3+) are shown in pink; CD8+ regulatory T cells are shown in light blue. For graphs (bottom), all data is presented as mean±SD with *p<0.05; **p<0.01; ***p<0.001; ****p<0.0001.

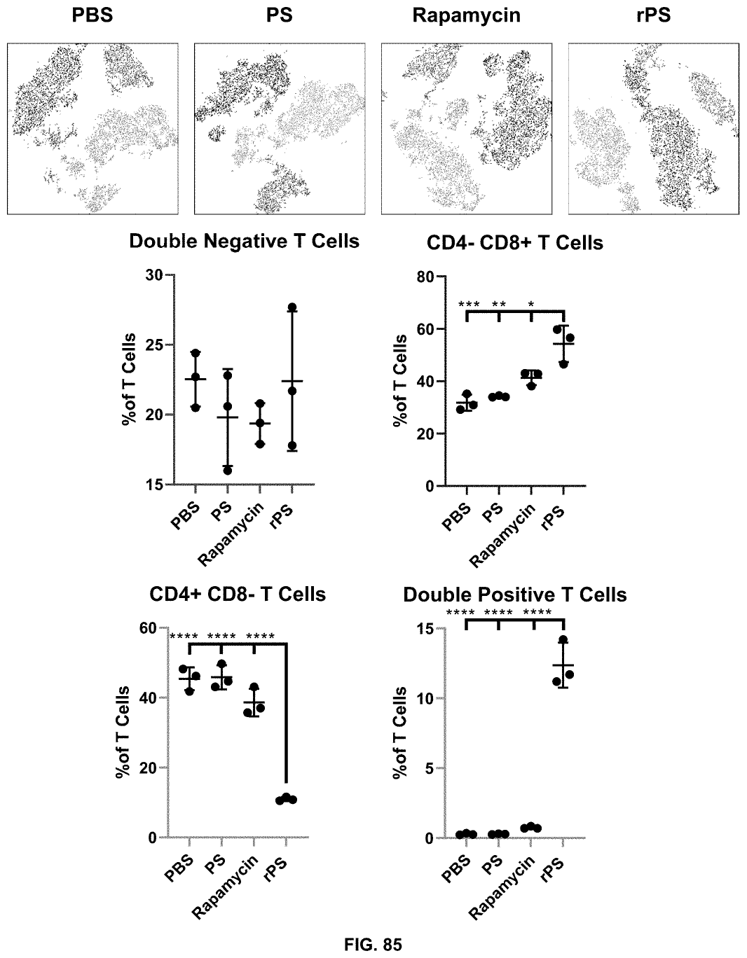

FIG. 86 shows costimulation of B cells in the spleen. (n=3 mice). For tSNE plots (top), the horizontal axis represents tSNE 1 and the vertical axis represents tSNE 2 of the CD19+ cell population. CD40+ B cells are shown in light blue; CD80+ B cells are shown in orange; CD86+ B cells are shown in green. For graphs (bottom), all data is presented as mean±SD with *p<0.05; **p<0.01; ***p<0.001; ****p<0.0001.

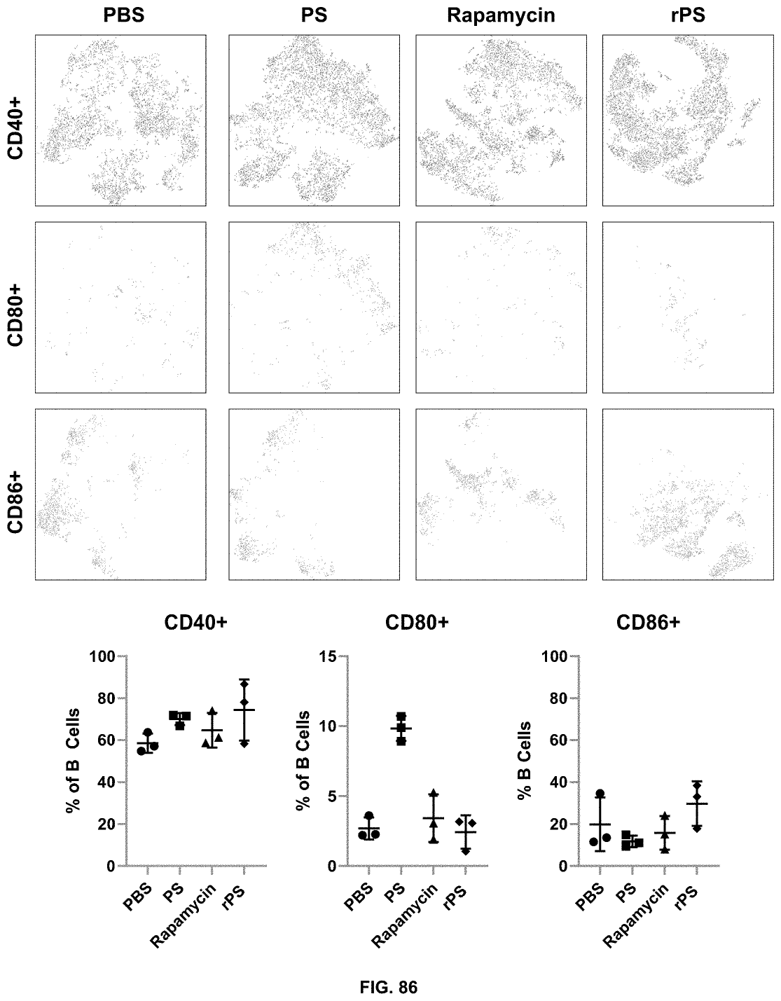

FIG. 87 shows DCs in the spleen rPS treatment significantly increased cDCs Type I and reduced pDCs in the spleen. (n=3 mice). For tSNE plots (top), the horizontal axis represents tSNE 1 and the vertical axis represents tSNE 2 of the CD11c+ cell population. cDCs Type I (CD8+ CD11b−) are shown in dark purple; cDCs Type II (CD8− CD11b+) are shown in pink; pDCs are shown in green. For graphs (bottom), all data is presented as mean±SD with *p<0.05; **p<0.01; ***p<0.001; ****p<0.0001.

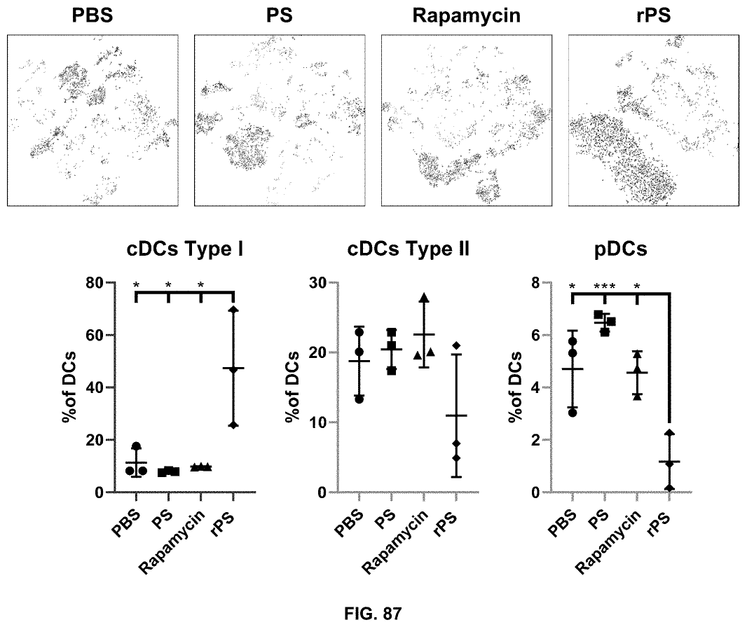

FIG. 88 shows maturation of DCs in the spleen. rPS treatment significantly increased DC maturation in the spleen. (n=3 mice). For tSNE plots (top), the horizontal axis represents tSNE 1 and the vertical axis represents tSNE 2 of the CD11c+ cell population. Pre-DCs (MHC II−) are shown in blue; mature DCs (MHC II+) are shown in red. For graphs (bottom), all data is presented as mean±SD with *p<0.05; **p<0.01; ***p<0.001; ****p<0.0001.

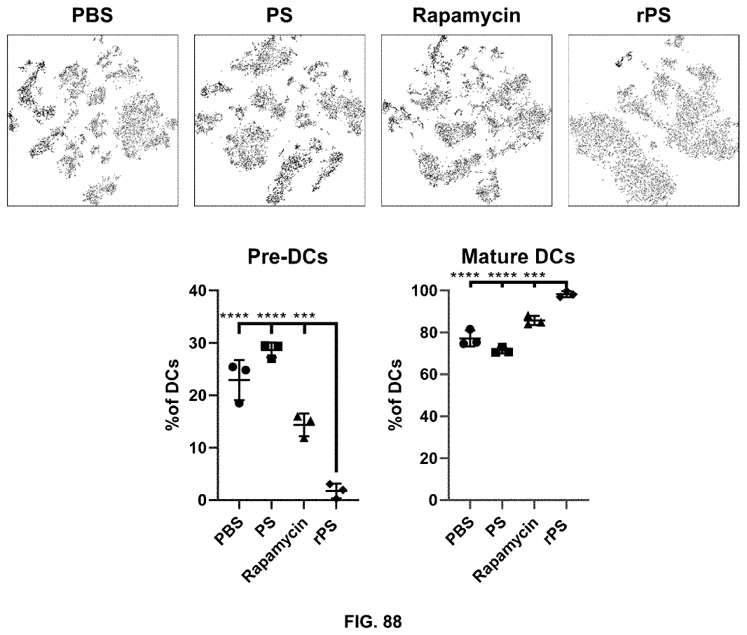

FIG. 89 shows costimulation of DCs in the spleen. rPS treatment significantly reduced CD80 and increased CD86 costimulation of DCs in the spleen. (n=3 mice). For tSNE plots (top), the horizontal axis represents tSNE 1 and the vertical axis represents tSNE 2 of the CD11b+ cell population. CD40+ DCs are shown in light blue; CD80+ DCs are shown in orange; CD86+ DCs are shown in green. For graphs (bottom), all data is presented as mean±SD with *p<0.05; **p<0.01; ***p<0.001; ****p<0.0001.

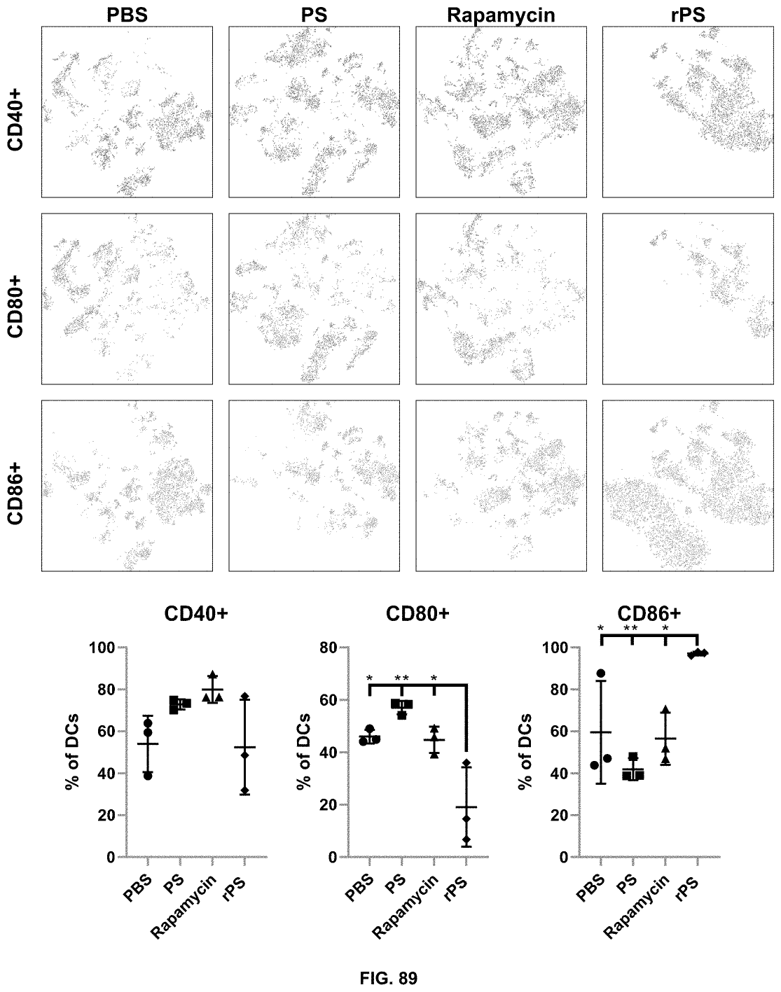

FIG. 90 shows macrophages in the spleen. rPS treatment significantly increased the macrophage population in the spleen. (n=3 mice). For tSNE plots (top), the horizontal axis represents tSNE 1 and the vertical axis represents tSNE 2 of the CD11b+ cell population. Macrophages (F4/80+ and/or CD169+) are shown in pink; Non-macrophage (F4/80− CD169−) monocytes are shown in green. For graphs (bottom), all data is presented as mean±SD with *p<0.05; **p<0.01; ***p<0.001; ****p<0.0001.

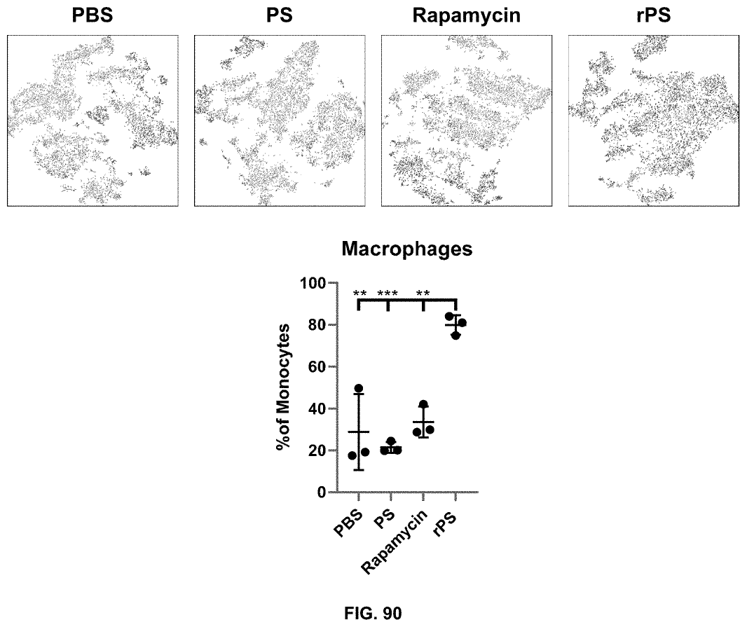

FIG. 91 shows MHC II+ monocytes in the spleen. rPS treatment significantly increased the MHC II+ monocyte population in the spleen (n=3 mice). For tSNE plots (top), the horizontal axis represents tSNE 1 and the vertical axis represents tSNE 2 of the CD11b+ cell population. MHC II− monocytes are shown in blue; MHC II+ monocytes are shown in red. For graphs (bottom), all data is presented as mean±SD with *p<0.05; **p<0.01; ***p<0.001; ****p<0.0001.

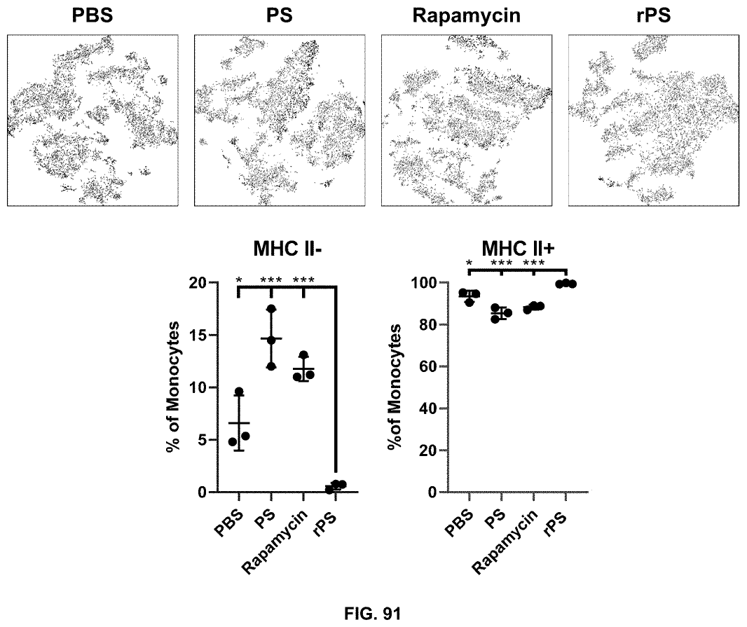

FIG. 92 shows Ly-6C^{Hi} monocytes in the spleen. rPS treatment significantly reduced the Ly-6C^{Hi} monocyte population in the spleen. (n=3 mice). For tSNE plots (top), the horizontal axis represents tSNE 1 and the vertical axis represents tSNE 2 of the CD11b+ cell population. Ly-6C^{Hi} monocytes are shown in pink; Ly-6C^{Lo} monocytes are shown in light blue. For graphs (bottom), all data is presented as mean±SD with *p<0.05; **p<0.01; ***p<0.001; ****p<0.0001.

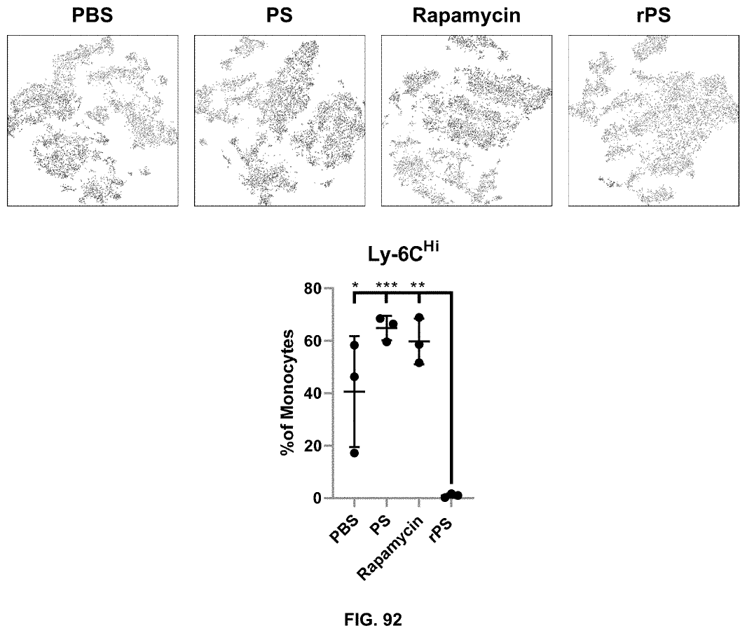

FIG. 93 shows costimulation of monocytes in the spleen. rPS treatment significantly reduced CD80 costimulation and increased CD86 costimulation of monocytes in the spleen. (n=3 mice). For tSNE plots (top), the horizontal axis represents tSNE 1 and the vertical axis represents tSNE 2 of the CD11b+ cell population. CD40+ monocytes are shown in light blue; CD80+ monocytes are shown in orange; CD86+ monocytes are shown in green. For graphs (bottom), all data is presented as mean±SD with *p<0.05; **p<0.01; ***p<0.001; ****p<0.0001.

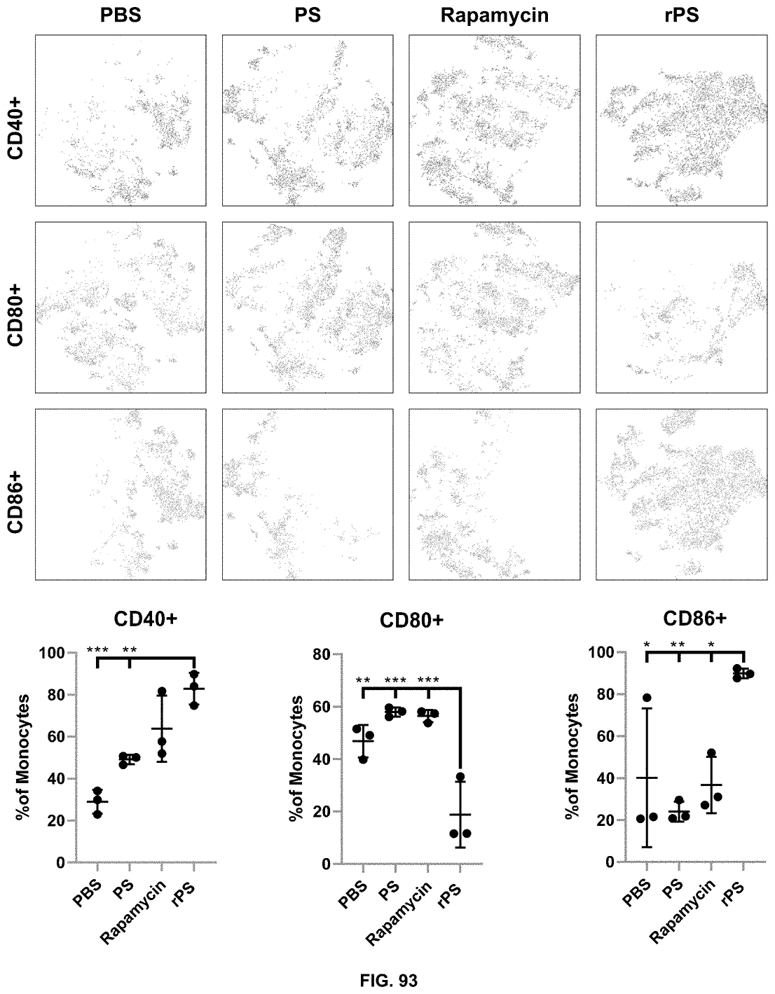

FIG. 94 shows T cells in the spleen. rPS treatment significantly reduced CD4+ T cells and CD4+ regulatory T cells and increased CD8+ T cells in the spleen. (n=3 mice). For tSNE plots (top), the horizontal axis represents tSNE 1 and the vertical axis represents tSNE 2 of the CD3+ cell population. CD4+ T cell are shown in orange; CD8+ T cells are shown in blue; NK T cells (NK1.1+) are shown in yellow; CD4+ regulatory T cells (CD25+ FoxP3+) are shown in pink; CD8+ regulatory T cells are shown in light blue. For graphs (bottom), all data is presented as mean±SD with *p<0.05; **p<0.01; ***p<0.001; ****p<0.0001.

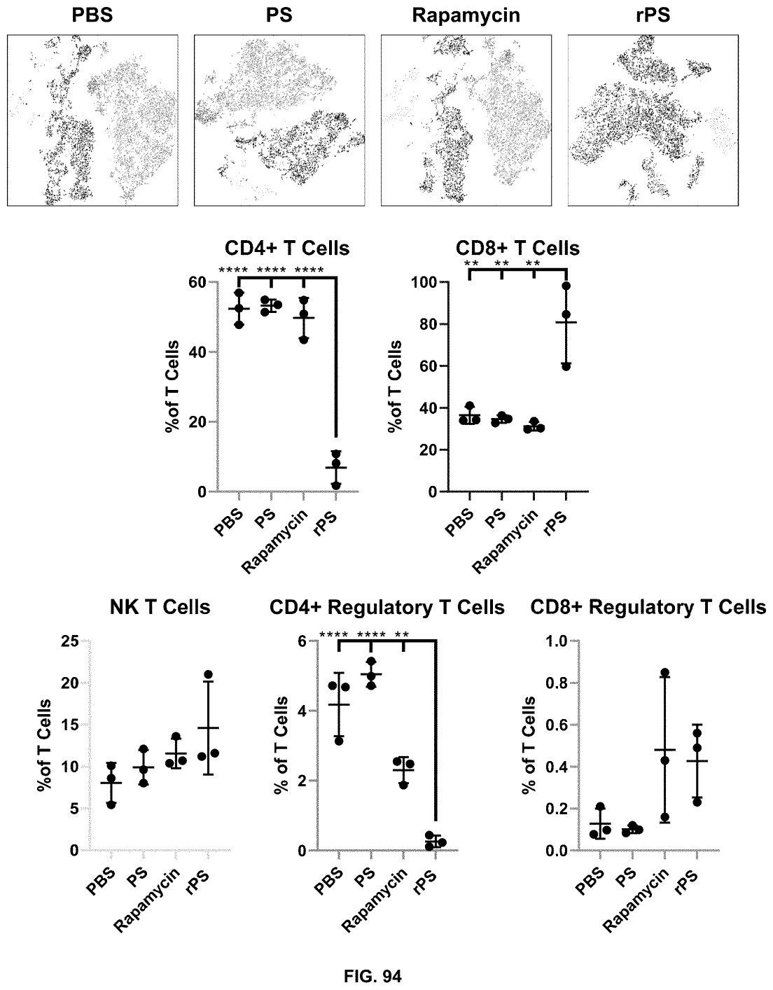

FIG. 95 shows T cells in the spleen. rPS treatment significantly increased double positive T cells, CD4− CD8+ T cells and reduced CD4⁺ CD8− T cells in the spleen. (n=3 mice). For tSNE plots (top), the horizontal axis represents tSNE 1 and the vertical axis represents tSNE 2 of the CD3+ cell population. Double negative (CD4− CD8−) T cells are shown in red; double positive (CD4⁺ CD8⁺) T cells are shown in green; CD4⁺ CD8− T cell are shown in orange; CD4+ CD8+ T cells are shown in blue. NK T cells (NK1.1+) are shown in yellow; CD4+ regulatory T cells (CD25+ FoxP3+) are shown in pink; CD8+ regulatory T cells are shown in light blue. For graphs (bottom), all data is presented as mean±SD with *p<0.05; **p<0.01; ***p<0.001; ****p<0.0001.

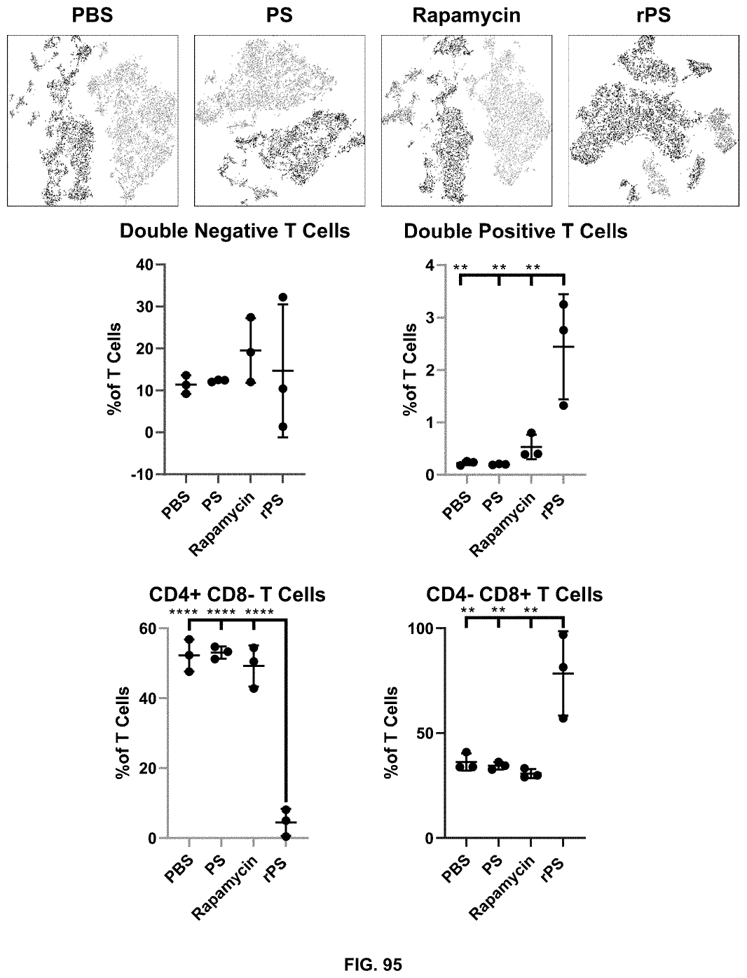

FIG. 96A shows Gating Strategy for Cell Populations in Flow Cytometry Studies. Representative pseudocolor plots and histograms are displayed from an example mouse lymph node.

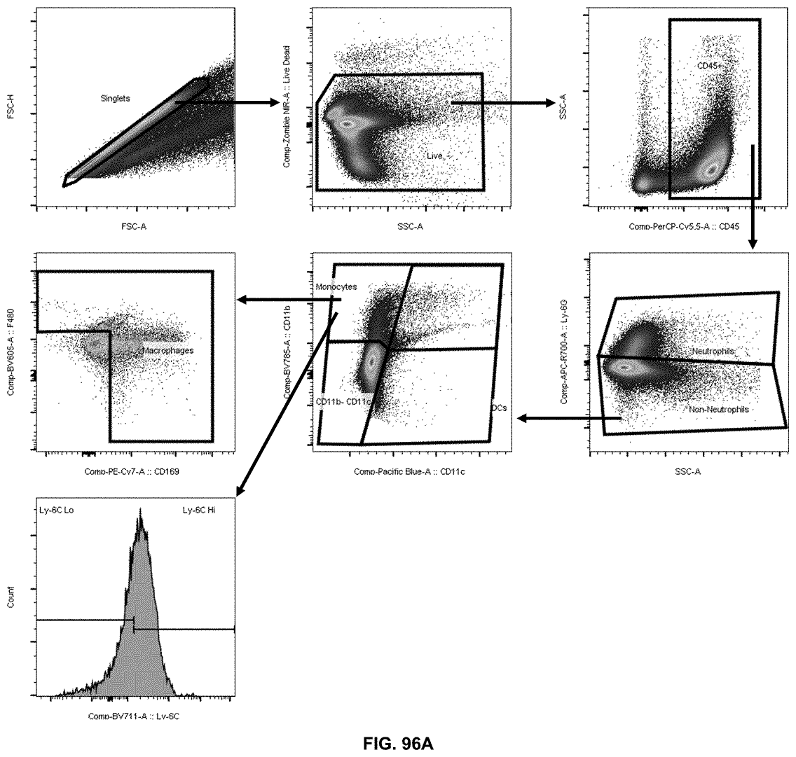

FIG. 96B shows Gating Strategy for Cell Populations in Flow Cytometry Studies. Representative pseudocolor plots and histograms are displayed from an example mouse lymph node.

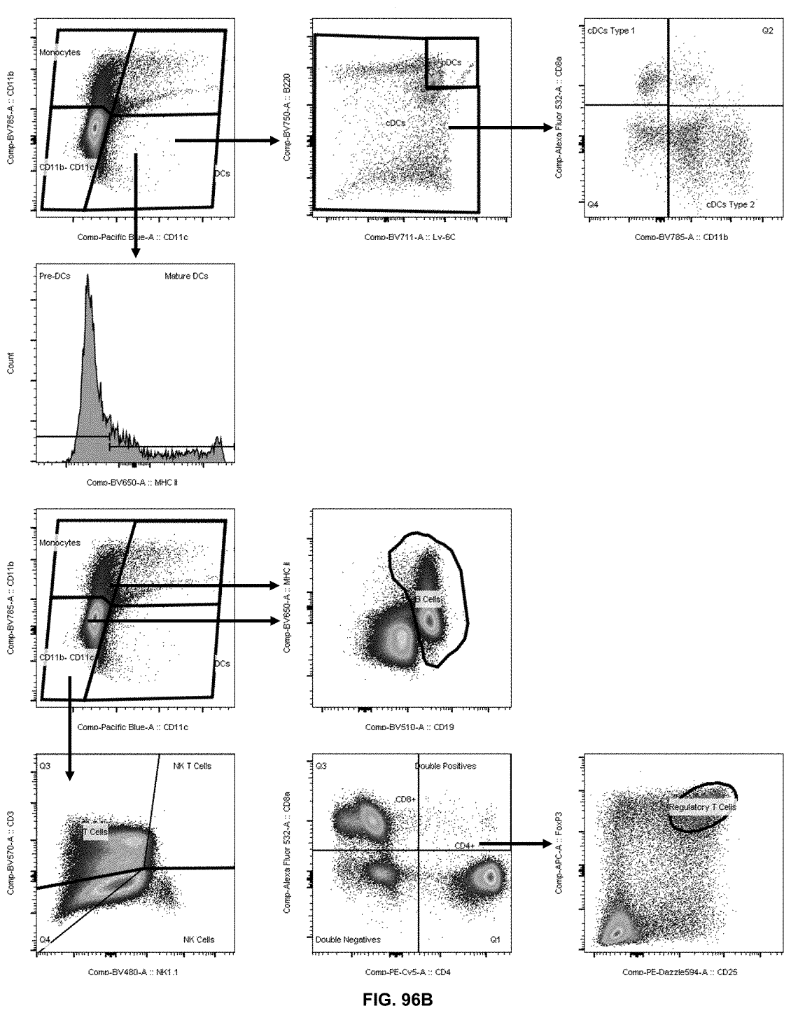

FIG. 96C shows Gating Strategy for Cell Populations in Flow Cytometry Studies. Representative pseudocolor plots and histograms are displayed from an example mouse lymph node.

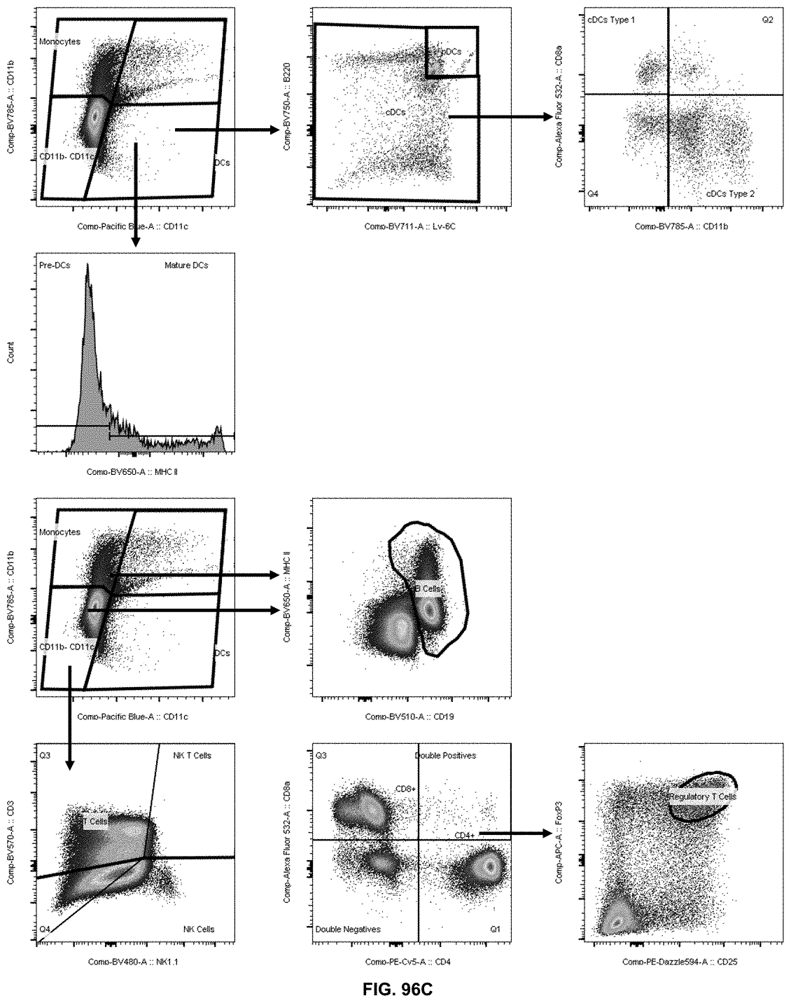

FIGS. 97A-97B shows Intraperitoneal glucose tolerance test. a, Blood glucose concentration over time after intraperitoneal glucose challenge. b, Area under the curve from IPGTT. (n≥5 mice). All data is presented as mean±SD.

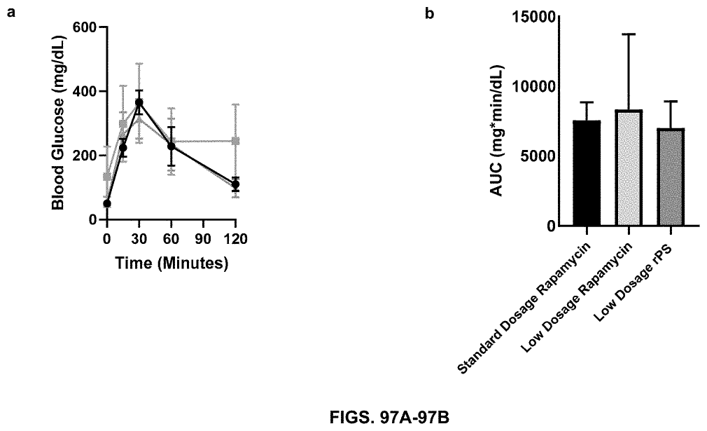

FIG. 98 shows rPS reduce injection site alopecia associated with rapamycin. (n≥5 mice).

FIG. 99 shows low dosage rPS enhance allogenic islet transplantation graft survival to the kidney capsule, as indicated by blood glucose concentration of the individual animals, normoglycemia (%), and intraperitoneal glucose tolerance test (IPGTT). (N=5).

FIG. 100 shows biodistribution of indocyanine green (ICG) dye and ICG loaded in to polymersomes (ICG-PS) by formulation 2, 24 and 48 hours after subcutaneous injection (n=5 mice).

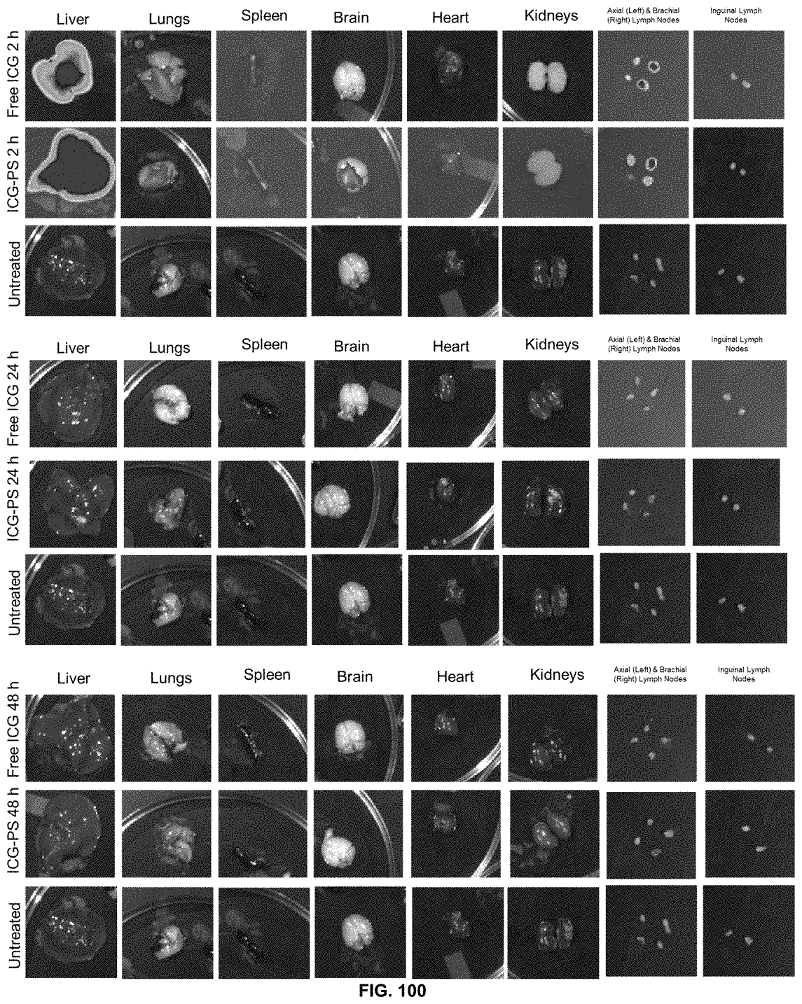

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### Definitions

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a nanocarrier” is a reference to one or more nanocarriers and equivalents thereof known to those skilled in the art, and so forth.

As used herein, the term “about,” when referring to a value is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “artificial” refers to compositions and systems that are designed or prepared by man, and are not naturally occurring. For example, an artificial peptide, peptoid, or nucleic acid is one comprising a non-natural sequence (e.g., a peptide without 100% identity with a naturally-occurring protein or a fragment thereof).

As used herein, the term “biocompatible” refers to materials and agents that are not toxic to cells or organisms. In some embodiments, a substance is considered to be “biocompatible” if its addition to cells in vitro results in less than or equal to approximately 10% cell death, usually less than 5%, more usually less than 1%.

As used herein, “biodegradable” as used to describe the polymers, hydrogels, and/or wound dressings herein refers to compositions degraded or otherwise “broken down” under exposure to physiological conditions. In some embodiments, a biodegradable substance is a broken down by cellular machinery, enzymatic degradation, chemical processes, hydrolysis, etc.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (or basic) (histidine (H), lysine (K), and arginine (R)); polar negative (or acidic) (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

The term “dendritic cell” or “DC” refers to the antigen presenting cells of the mammalian immune system. DCs function to process antigen material and present it on their surface to T cells of the immune systems and act as a messenger between the innate and the adaptive immune system. DCs express high levels of the molecules that are required for antigen presentation such as the MHC II, CD80, and CD86 on activation and are highly effective in initiating an immune response. DCs are distributed throughout the body, including the mucosal tissues, where they are found below the epithelial cell barrier. DCs have been found to play roles in progressive decline in adaptive immune responses, loss of tolerance and development of chronic inflammation. Dendritic cells may be present in the normal arterial wall and within atherosclerotic lesions.

The term “islet” or “pancreatic islet” as used interchangeably herein refers to the regions of the pancreas that contain endocrine (hormone-producing) cells.

The term “nanocarrier” refers to a nanomaterial used as a transport module for another substance. For example, the nanocarriers disclosed herein may be used as a transport module for one or more therapeutic agents. The nanocarriers disclosed herein are also referred to as “polymersomes” or “PS” or micelles, depending on their structure. Polymersomes are a class of artificial vesicle nanocarriers composed of amphiphilic synthetic block copolymers and having an aqueous core. Micelles are a class of artificial vesicle nanocarriers having a hydrophobic/lipophilic core and a hydrophilic exterior. In particular embodiments, the nanocarriers disclosed herein are composed of a poly(ethylene glycol)-block-poly(propylene sulfide) copolymer.

As used herein, “nanodrug” refers to a nanocarrier formulation of a drug or therapeutic compound. Nanodrug formulations can be formed using any nanocarrier and any drug or therapeutic agent described herein. Nanodrugs may be formulated with a nanocarrier targeted to a specific cell or tissue.

As used herein, “non-tolerogenic” refers to a compound, composition, or carrier that does not produce or cause immunological tolerance when administered to a subject in the absence of in immunological compound such as an antigen or adjuvant. In some embodiments, the compound, composition, or carrier is less tolerogenic than other compounds, compositions, or carriers known in the art.

As used herein, the term “peptide” refers an oligomer to short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (artificial) sequence.

As used herein, the phrase “physiological conditions” relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

As used herein, the terms “prevent,” “prevention,” and preventing” refer to reducing the likelihood of a particular condition or disease state (e.g., inflammatory condition, transplantation rejection) from occurring in a subject not presently experiencing or afflicted with the condition or disease state. The terms do not necessarily indicate complete or absolute prevention. “Prevention,” encompasses any administration or application of a therapeutic or technique to reduce the likelihood of a disease developing (e.g., in a mammal, including a human). Such a likelihood may be assessed for a population or for an individual.

As used herein, the term “sequence identity” refers to the degree of which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y % sequence identity (e.g., 90%) with SEQ ID NO:Z (e.g., 100 amino acids) may have up to X substitutions (e.g., 10) relative to SEQ ID NO:Z, and may therefore also be expressed as “having X (e.g., 10) or fewer substitutions relative to SEQ ID NO:Z.”

As used herein, the terms “treat,” “treatment,” and “treating” refer to reducing the amount or severity of a particular condition, disease state (e.g., inflammatory condition), or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

### DETAILED DESCRIPTION OF THE INVENTION

The present disclosure describes nanocarriers comprising a core comprising poly(ethylene glycol)-block-poly(propylene sulfide) (PEG-bl-PPS) and least one therapeutic agent. PEG-bl-PPS nanocarriers are non-inflammatory and are therefore advantageous as vehicles for immunomodulatory therapeutic agents, as the elicited responses are dependent solely on the transported therapeutic agent.

PEG-bl-PPS block copolymers can be prepared via known methods, e.g., those described in Allen, S. et al., Facile assembly and loading of theranostic polymersomes via multi-impingement flash nanoprecipitation, i.e., J. Control. Release 2017. 262: p. 91-103 and in U.S. Pat. No. 10,633,493, each of which is incorporated herein by reference in its entirety. An exemplary synthesis is described in the Examples. For example, an appropriate methyl ether poly(ethylene glycol) with a mesylate leaving group can be reacted with thioacetic acid to form a protected PEG-thioacetate. Base activation of the thioacetate can result in the formation of a thiolate anion, which may be used as the initiator for ring opening polymerization of propylene sulfide. The reaction can be completed with the addition of an end-capping agent or functionalization agent. These block copolymers can be prepared with varying ratios of PEG and PPS by varying the degree of propylene sulfide polymerization.

Nanocarriers of the PEG-bl-PPS can be prepared, for example, by flash-nanoprecipitation (FNP) or thin film rehydration. To make nanocarriers via FNP, polymer and any hydrophobic agents can be dissolved in one or more organic solvents, while any hydrophilic agents can be dissolved in an aqueous solution (e.g., a buffer such as phosphate-buffered saline). The two solutions can be loaded into separate syringes and impinged against each other into a reservoir using a confined impingement jets (CIJ) mixer. Multiple impingements can be used to extrude polymersomes. To make nanocarriers via thin film rehydration, polymer and any hydrophobic agents can be dissolved in one or more organic solvents, and the resulting solution can be dessicated. Then an aqueous solution (e.g., a buffer such as phosphate-buffered saline) can be added to the mixture can be shaken overnight, followed by extrusion (e.g., using a syringe filter). Polymersomes were extruded using a 0.22 μm syringe filter. For both methods, unloaded agents can be removed either via exclusion column purification or dialysis.

Nanocarriers can be characterized for size distribution via dynamic light scattering (DLS) and nanoparticle tracking analysis (NTA), and for morphology via cryogenic transmission electron microscopy (cryoTEM). Agent loading can be characterized via fluorescence and absorbance measurements.

A variety of types of nanocarriers can be prepared by varying the degree of propylene sulfide polymerization. For example, nanocarriers may be in the form of bicontinuous nanospheres (e.g., PEG weight fraction of about 0.12), polymersomes (e.g., PEG weight fraction of about 0.19 to about 0.31), filomicelles (e.g., PEG weight fraction of about 0.31 to about 0.38), and micelles (e.g., PEG weight fraction of about 0.38 to about 0.69). In some embodiments, the block copolymer has a PEG weight fraction of about 0.25.

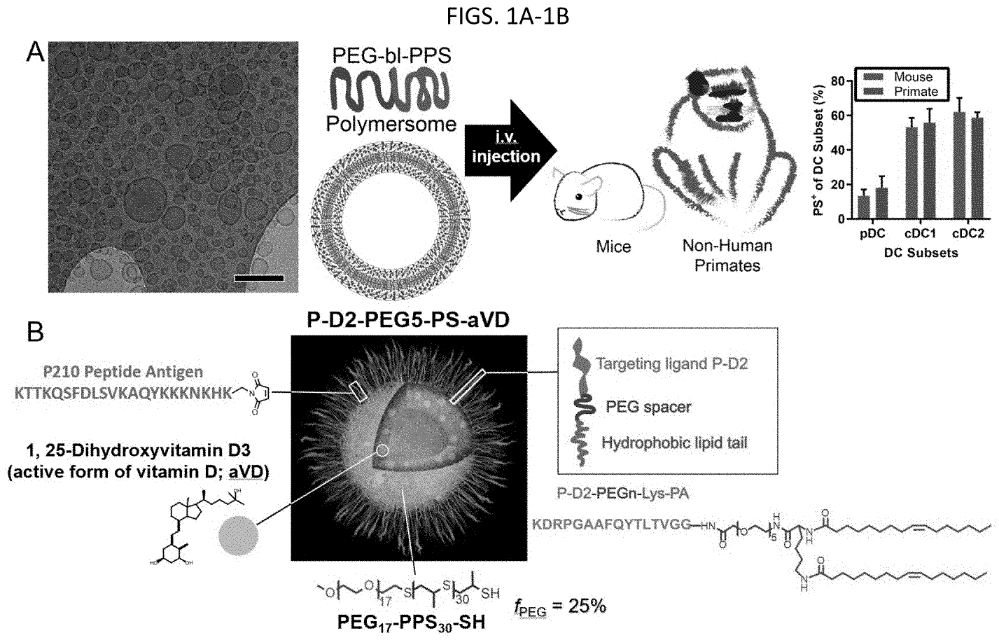

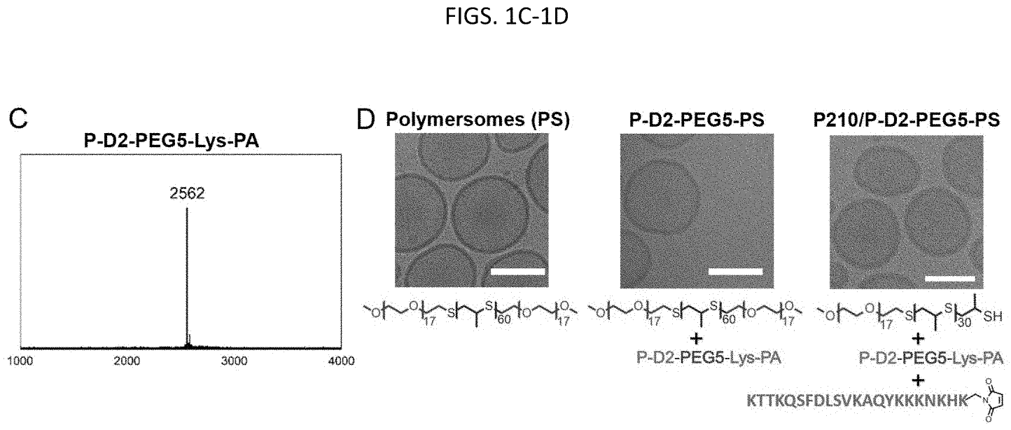

In some embodiments, the nanocarrier is a polymersome having an aqueous core and hydrophobic and hydrophilic regions of the lipid bilayer surrounding the aqueous core. See FIG. 1A for an example of the polymersome morphology. The polymersome nanocarrier can have a PEG weight fraction of about 0.19 to about 0.31, e.g., 0.25. The polymersome nanocarrier may have a diameter of about 90 nm to about 150 nm in diameter, alternatively from about 100 nm to about 150 nm, alternatively from about 100 nm to about 120 nm in diameter. The size of the polymersome may change when it is loaded with a target molecule or drug compared to the size of an unloaded polymersome made from the same copolymer. In one embodiment, the polymersome comprises a vesicular polymer membrane composed of PEG₁₇-bl-PPS₃₀.

In some embodiments, the nanocarrier is a bicontinuous nanosphere (BCN) characterized by two continuous phases; (i) a cubic lattice of aqueous channels that traverse (ii) an extensive hydrophobic interior volume. Based on small angle X-ray scattering (SAXS) analysis, BCN have primitive type cubic internal organization (Im3m) as confirmed by Bragg peaks with relative spacing ratios at √2, √4, and √6. BCNs are the polymeric equivalent of lipid cubosomes and are lyotropic. BCN can incorporate both hydrophobic and hydrophilic payload molecules.

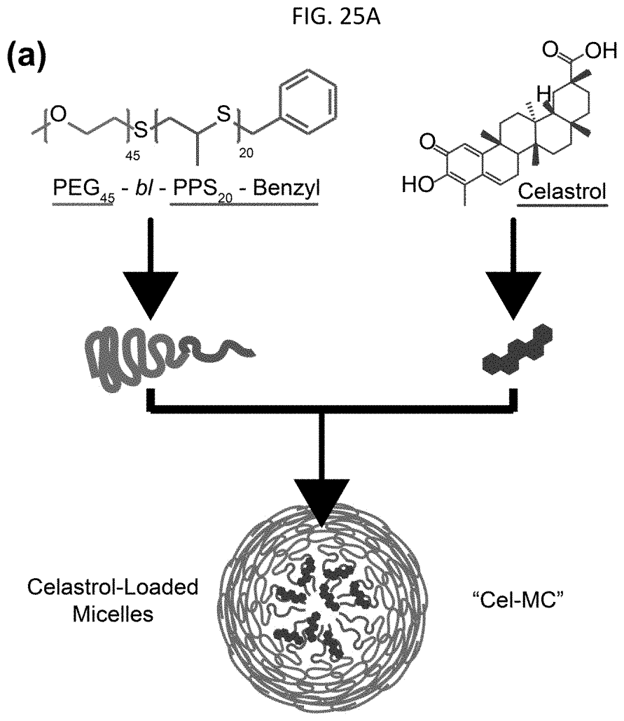

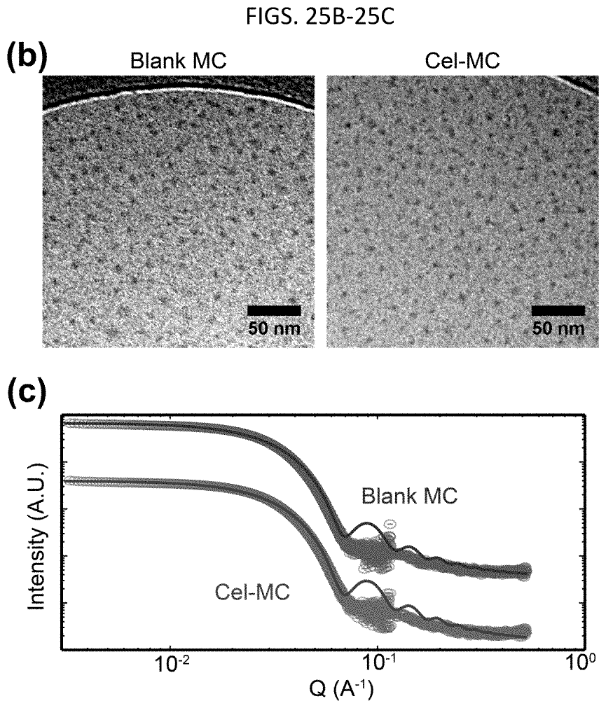

In some embodiments, the nanocarrier is a micelle having a hydrophobic/lipophilic core and a hydrophilic exterior. Micelle nanocarriers have a spherical morphology and are typically smaller (e.g., less than 50 nm) than polymersomes and the hydrophobic core can be loaded with a lipophilic payload molecule or therapeutic agent. The micelles suitably have a PEG weight fraction of about 0.38 to about 0.69. An example of the micelle nanocarrier morphology is shown in FIG. 25A.

The nanocarrier further comprises at least one therapeutic agent. The therapeutic agent may be any suitable therapeutic agent to achieve the desired therapeutic effect. The therapeutic agent may be hydrophilic or hydrophobic. In some embodiments, a nanocarriers comprising the at least one therapeutic agent are able to achieve the same immunomodulatory effects at a lower therapeutically effective dose compared the therapeutically effective dose required for free therapeutic agent (i.e. the therapeutic agent in the absence of the nanocarrier), therefore allowing therapeutic efficacy with minimized side effects in the subject. In other embodiments, the nanocarriers may enable a high dose of the therapeutic agent to be used safely without negative side effects typically associated with the same dose of the therapeutic agent in the absence of the nanocarrier. The disclosed nanocarriers may therefore improve the quality of life for patients, such as patients requiring immunosuppression for organ transplantation or inflammatory diseases, as the intended effect of the therapy will be achieved with reduced side effects.

Selection of the at least one therapeutic agent is dependent on the desired condition to be treated. For example, the at least one therapeutic agent may be an anti-inflammatory agent, an immunomodulatory agent, or an immunosuppressive agent. Suitable therapeutic agents include, for example, celastrol, rapamycin, 1, 25-Dihydroxyvitamin D3 (aVD), ApoB-100, and ApoB-100 derived P210 peptide. The condition to be treated may be any inflammatory condition, including atherosclerosis, arthritis, inflammatory bowel disease, and the like.

In some embodiments, the therapeutic agent may be selected to enable use of the nanocarrier for the treatment of atherosclerosis. Atherosclerosis is an immunologically complex inflammatory condition within the intima of arterial vessels and a primary source of cardiovascular disease (CVD), the leading cause of death worldwide. Immune cells are present in very early atherosclerotic lesions and remain for the duration of plaque progression. Immune cells play an active role in cholesterol efflux, plaque extracellular matrix restructuring, and plaque stability and size. Pro-inflammatory signaling can result in the recruitment of more immune cells to vascular lesions, and to the exacerbation of atherosclerosis.

In other embodiments, the at least one therapeutic agent may be an immunomodulatory agent or an immunosuppressive agent. Nanocarriers comprising an immunomodulatory or immunosuppressive agent may be useful for the treatment or prevention of cell, tissue, or organ transplant rejection. For example, nanocarriers may be useful for the prevention of islet transplantation rejection.

One of the core inflammatory signaling pathways within immune cells is the NF-κB signaling pathway. NF-κB is a transcription factor that is sequestered within the cytoplasm until upstream signaling results in its release and subsequent translocation into the nucleus. A number of receptors lie upstream of NF-κB, including Toll-like receptors (TLRs) TLR2 and TLR4. These receptors are known to recognize oxidized LDL, a marker of atherosclerosis and a key component of its development and progression. NF-κB can result in the expression of pro-inflammatory signaling molecules, such as the cytokine TNF-α, which can induce apoptosis in nearby cells and exacerbate oxidative stress. Mice lacking MyD88, an adaptor protein upstream of NF-κB in many TLR signaling pathways, have reduced atherosclerosis, highlighting the pro-atherogenic result of NF-kB activation.

In one embodiment, the therapeutic agent is an immunomodulatory agent, for example, the immunomodulatory agent is an inhibitor of NF-κB. In some embodiments, the therapeutic agent may be any suitable inhibitor of NF-κB. Suitable NF-κB inhibitors include, but are not limited to, celastrol, aVD, QNZ, SC75741, (−)-parthenolide, caffeic acid phenethyl ester, curcumin, CBL0137, andrographolide, pyrrolidinedithiocarbamate, SN50, sodium salicylate, and sodium 4-aminosalicylate. See, for example, Yi et al., Advanced Functional Materials, 2019, which is incorporated herein by reference in its entirety.

In some embodiments, the small molecule inhibitor of NF-κB is celastrol, a triterpene extracted from Tryptergium wilfordii. Celastrol has been used, in its herbal plant form, in Chinese folk medicine for a number of years before it was isolated and recognized as an inhibitor with a number of advantageous targets. One (or potentially several) of those targets is upstream of NF-κB, and inhibition by celastrol prevents the release and translocation of NF-κB. However, free Celastrol possesses numerous properties that hinder its use as a therapeutic agent. Celastrol is very hydrophobic, with correspondingly poor bioavailability and a relatively short serum half-life (T_{1/2β}) of 8-10 hours. Celastrol also has many targets unrelated to inflammatory signaling and effects a wide variety of cell types. It can reduce cell survival in some cells by inhibiting the HSP90 pathway, but can also promote cell survival in neuronal cells, potentially through its inhibition of the NF-κB pathway and upregulation of HSP70. Celastrol can resensitize the body to leptin in obese mice, most likely by affecting cells in the hypothalamus. In a recent study, celastrol's ability to reduce lipopolysaccharide (LPS)-induced inflammation in vivo was counterintuitively found to worse inflammation when administered via intraperitoneal (IP) injection. Perhaps related to celastrol's ability to induce apoptosis, celastrol can be cytotoxic to cells at concentrations relatively close to its EC₅₀.

In some embodiments, the therapeutic agent may be the small molecule hydrophobic therapeutic agent celastrol. Suitable nanocarriers containing the celastrol are described in Allen, S. et al., Celastrol-loaded PEG-bl-PPS nanocarriers as an anti-inflammatory treatment for atherosclerosis. Biomater. Sci. 2019 7: 657-668, the entire contents of which are incorporated herein by reference. In some embodiments, nanocarriers comprising the therapeutic agent celastrol may enable delivery of significantly lower therapeutically effective dosages of celastrol compared to the dosages required for therapeutic efficacy of celastrol alone. For example, a typical dose of celastrol may be about free celastrol demonstrates a steep decline in its efficacy between 1 μg/mL and 0.1 μg/mL concentrations, with a half maximal effective concentration (EC₅₀) of 0.2 μg/mL. Celastrol loaded in nanocarrier formulations has an estimated EC₅₀ of 4.2 pg/mL, a concentration nearly 50,000 times lower. In a subject, while celastrol may typically be administered at a dosage between about 0.5 mg/kg and about 10 mg/kg, nanocarrier celastrol formulations can be administered at a dosage between about 0.5 μg/kg and about 100 μg/kg. In some embodiments, the lower therapeutically effective dose of celastrol when administered in a nanocarrier formulation is at least 100, at least 500, at least 1000, at least 10,000, at least 25,000, or at least 50,000 times lower than the therapeutically effective dose of free celastrol. In accordance with such embodiments, nanocarriers comprising celastrol may be safely used in a subject with improved efficacy and safety.

In some embodiments, the therapeutic agent may be an immunosuppressive agent. Suitable immunosuppressive agents include, but are not limited to, rapamycin (sirolimus), tacrolimus, mycophenolate mofetil, cyclosporine, azathioprine, and prednisone.

In some embodiments, the at least one therapeutic agent may be the hydrophobic therapeutic agent rapamycin. Rapamycin is an FDA-approved immunosuppressant that inhibits the mechanistic target of rapamycin (mTOR) kinase, which is a key regulator of cell growth, metabolism and proliferation and elicits cellular responses that are highly dependent on the cell type. In the case of T cells, mTOR inhibition is known to decrease proliferation, migration and overall population levels for T cells, particularly CD4+ CD25− T cell and effector CD8+ T cell subsets. For dendritic cells, rapamycin has a suppressive effect on maturation and differentiation by inhibiting expression of co-stimulatory molecules and inflammatory cytokines. Suitable nanocarriers containing rapamycin are described in Allen, S. et al., J. Control. Release 2017. 262: p. 91-103, the entire contents of which are incorporated herein by reference. In some embodiments, nanocarriers comprising the therapeutic agent rapamycin may enable delivery of significantly lower therapeutically effective dosages of rapamycin compared to the dosages required for therapeutic efficacy of rapamycin alone. In accordance with such embodiments, nanocarriers comprising rapamycin may be safely used in a subject with improved efficacy and safety.

The nanocarrier may comprise any suitable number of therapeutic agents to achieve the desired effect. For example, the nanocarrier may comprise one therapeutic agent. In other embodiments, the nanocarrier may comprise two therapeutic agents. In other embodiments, the nanocarrier may comprise more than three or more therapeutic agents.

The nanocarrier may comprise any suitable amount of the one or more therapeutic agents to achieve the desired effect. The disclosed nanocarriers may enable loading of low doses of the therapeutic agent with enhanced therapeutic efficacy and minimized side effects compared to the same dose of the therapeutic agent in the absence of the disclosed nanocarriers (i.e. the free therapeutic agent). In other embodiments, the nanocarriers may enable a high dose of the therapeutic agent to be used safely without negative side effects typically associated with the same dose of the therapeutic agent in the absence of the nanocarrier. The nanocarrier may comprise about 1 ng therapeutic agent/mg PEG-bl-PPS to about 1 mg therapeutic agent/mg PEG-bl-PPS. For example, the nanocarrier may comprise about 1 ng therapeutic agent/mg PEG-bl-PPS to about 1 mg therapeutic agent/m PEG-bl-PPS, about 10 ng/mg to about 900 μg/mg, about 100 ng/mg to about 800 μg/mg, about 500 ng/mg to about 700 μg/mg, about 750 ng/mg to about 600 μg/mg, about 1000 ng/mg to about 500 μg/mg, about 10 μg/mg to about 400 μg/mg, about 100 μg/mg, to about 300 μg/mg, or about 200 μg therapeutic agent/mg PEG-bl-PPS.

In some embodiments, the nanocarrier may further comprise a targeting ligand displayed on a surface of the nanocarrier. The targeting ligand may target any desired cell type. In some embodiments, the targeting ligand may selectively target dendritic cells. Nanocarriers comprising a targeting ligand may be useful for the administration of, for example, aVD, ApoB-100, or ApoB-100 derived antigenic peptide P210 to a subject. In some embodiments, the nanocarrier comprises 1, 25-Dihydroxyvitamin D3 and P210 peptide. In particular embodiments, the P210 peptide comprises the amino acid sequence of SEQ ID NO: 2.

As central nodes that can direct both the initiation and suppression of immune responses for respective atherogenesis and atheroprotection, dendritic cells (DCs) may serve as an advantageous target for immunomodulation of atherosclerotic inflammation. DC maturation and pro-inflammatory responses can be triggered by their increased uptake of oxidized LDL (oxLDL) under conditions of a high fat diet, resulting in presentation of apolipoprotein B100 (ApoB-100)-derived peptides for Th1-biased cell activation and differentiation. However, immature DCs with insufficient presentation of stimulatory CD80/CD86 co-receptors can induce naïve T cells to differentiate into regulatory T cells (Tregs), which suppress inflammation and proatherogenic immune responses.

The targeting ligand may comprise any suitable ligand that selectively targets dendritic cells. For example, the targeting ligand may comprise an antibody, antibody fragment, an aptamer, or a peptide. For example, the targeting ligand may be an anti-CD11c antibody or a fragment thereof. In other embodiments, the targeting ligand is a peptide. For example, the targeting ligand may be a P-D2 peptide. In some embodiments, the targeting ligand is a P-D2 peptide comprising the amino acid sequence GGVTLTYQFAAGPRDK (SEQ ID NO: 1).

Additional embodiments of nanocarriers suitable for targeting dendritic cells are described in U.S. Patent Publication No. 2018/0028446, which is incorporated herein by reference in its entirety.

The targeting ligand may further comprise a spacer. The spacer may be incorporated for adding solubility, flexibility, distance between segments, etc. The spacer may comprise peptide and/or non-peptide elements. The spacer may comprise one or more bioactive groups (e.g., alkene, alkyne, azide, thiol, etc.). In other embodiments, the spacer is a non-peptide spacer (e.g., alkyl, OEG, PEG, etc.) linkers). For example, the spacer may be a PEG spacer. The spacer may be any suitable length. For example, the spacer may comprise a PEG spacer with 1-20 repeating PEG units. For example, the spacer may comprise 1-20, 2-18, 4-16, 6-12, or 8-10 repeating PEG units. In some embodiments, the spacer comprises 5 repeating PEG units. In other embodiments, the spacer comprises 11 repeating PEG units. In other embodiments, the spacer comprises 15 repeating PEG units.

The targeting ligand may further comprise a lipid tail for insertion into the nanocarrier membrane. For example, the targeting ligand may comprise a palmitoleic acid (PA) lipid tail.

In particular embodiments, the targeting ligand comprises a P-D2 peptide, a PEG spacer, and a palmitoleic acid lipid tail.

Administration of 1, 25-Dihydroxyvitamin D3 (aVD) can promote the maintenance of immature tolerogenic DCs by interacting with the vitamin D nuclear receptor (VDR), which directly inhibits the pro-inflammatory transcription factor NF-kB to down-regulate expression of MHC-II, co-stimulatory receptors CD80/86, and a range of pro-inflammatory cytokines. aVD-induced tolerogenic DCs inhibit proinflammatory T cells (Th1 and Th17 cells) and are particularly relevant to the induction of tolerance and generation of Tregs in humans. However, due to the broad tissue distribution of the VDR and the wide range of cell-specific functions of NF-kB, systemic non-targeted administration of aVD can result in a host of side effects including systemic toxicity and severe immunosuppression.

In some embodiments, described herein are synthetic nanocarriers composed of poly(ethylene glycol)-bl-poly(propylene sulfide) copolymers with modified surface chemistry and morphology that selectively target and modulate DCs by transporting the anti-inflammatory agent (aVD; 1, 25-Dihydroxyvitamin D3) and ApoB-100 derived antigenic peptide P210. Polymersomes decorated with an optimized surface are shown herein which display an optimal density for the P-D2 peptide, which binds CD11c on the DC surface, and significantly enhances the cytosolic delivery and resulting immunomodulatory capacity of aVD. Intravenous administration of the optimized polymersomes is shown herein to achieve selective targeting of DCs in atheroma and spleen compared to all other cell populations, including both immune and CD45⁻ cells, and locally increased the presence of tolerogenic DCs and cytokines. aVD-loaded polymersomes is demonstrated herein to significantly inhibit atherosclerotic lesion development in high fat diet-fed ApoE^{-/-} mice following 8 weeks of administration. Incorporation of the P210 peptide is shown herein to generate the largest reductions in vascular lesion area (˜40%, p<0.01), macrophage content (˜57%, p<0.01), and vascular stiffness (4.8-fold). These results correlate with an −6.5-fold increase in levels of Foxp3⁺ regulatory T cells within atherosclerotic lesions. These results validate the key role of DC immunomodulation during aVD-dependent inhibition of atherosclerosis and demonstrate the therapeutic enhancement and dosage lowering capability of cell-targeted nanotherapy in the treatment of CVD.

The nanocarrier may comprise any suitable molar ratio of targeting peptide:core necessary to achieve the desired effect. For example, the nanocarrier may comprise a molar ratio of targeting peptide:poly(ethylene glycol)-block-poly(propylene sulfide) copolymer of 1%-10%. For example, the molar ratio may be 1%, 2%, 3%, 4%, 5%, 6%, 7%, 8%, 9%, or 10%. In particular embodiments, the molar ratio of targeting peptide:poly(ethylene glycol)-block-poly(propylene sulfide) copolymer is 4%.

The disclosed nanocarriers are advantageous over current therapies on the market for a variety of reasons. The disclosed nanocarriers are highly versatile—allowing for a diverse array of therapeutic agents to be loaded. Both hydrophilic and/or hydrophobic drugs can be incorporated. Additionally, the disclosed nanocarriers allow for enhanced cell targeting. By varying the nanocarrier morphology, the nanocarriers may be designed to selectively target any desired cell population or organ specific population. Additionally, the polymers used in the nanocarriers, poly(ethylene glycol) and poly(propylene sulfide) have been widely proven to be inert. Thus, the disclosed nanocarriers not induce any background inflammation that may exasperate to inflammatory conditions.

Additionally, nanodrug formulations including the nanocarrier and therapeutic agents described herein allow for administration of the therapeutic agent at doses significantly lower (i.e., reduction in the effective dose) than administration of the free therapeutic agent alone without the nanocarrier. In some embodiments, the effective dose the therapeutic agent, when administered in a nanodrug formulation with a nanocarrier, is at least 20%, 25%, 30%, 35%, 40%, 45%, 50%, 60%, 70%, 75%, 80%, or 90% lower than the effective dose of the therapeutic drug alone without the nanocarrier. In some embodiments, the effective dose the therapeutic agent, when administered in a nanodrug formulation with a nanocarrier, is at least 10 times, at least 50 times, at least 100 times, at least 250 times, at least 500 times, at least 1,000 times, at least 2,500 times, at least 5,000 times, at least 10,000 times, at least 15,000 times, at least 25,000, or at least 50,000 times lower than the effective dose of the therapeutic drug alone without the nanocarrier.

Although the disclosed nanocarriers are suitable for administration to a subject, the nanocarriers disclosed herein may also be incorporated into pharmaceutical compositions. The disclosed nanocarriers or pharmaceutical compositions comprising the same may be used in methods of treating inflammatory condition in a subject in need thereof. The pharmaceutical compositions may further comprise one or more pharmaceutically acceptable excipients. The pharmaceutically acceptable excipients will be dependent on the mode of administration to be used. Suitable modes of administration include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the disclosed pharmaceutical compositions are administered parenterally. In some embodiments, parenteral administration is by intrathecal administration, intracerebroventricular administration, or intraparenchymal administration. The disclosed pharmaceutical compositions herein can be administered as the sole active agent or in combination with other pharmaceutical agents such as other agents used in the treatment of inflammatory condition in a subject.

In some embodiments, the disclosed nanocarriers and pharmaceutical compositions comprising the same may be used in methods for treating or preventing an inflammatory or autoimmune condition in a subject in need thereof. The subject may be diagnosed with or at risk of developing any inflammatory or autoimmune. Inflammatory and autoimmune conditions include, but are not limited to, Rheumatoid arthritis, immunodyregulation polyendocrinopathy enteropathy X-linked syndrome, autoimmune lymphoproliferative syndrome, autoimmune polyendocrinopathy candidiasis ectodermal dystrophy, multiple sclerosis, systemic lupus erythematosus, osteoarthritis, spondyloarthropathies, gout, familial fever syndromes, systemic juvenile idiopathic arthritis, inflammatory bowel disease, arthritis, and atherosclerosis. In some embodiments, the inflammatory condition is atherosclerosis. In another embodiment, the inflammatory condition is inflammatory bowel disease. In a further embodiment, the inflammatory condition is arthritis.

In other embodiments, the disclosed nanocarriers and pharmaceutical compositions comprising the same may be used in methods for treating or preventing cell, tissue, or organ transplant rejection in a subject in need thereof. The methods comprise administering to the subject a therapeutically effective amount of the disclosed pharmaceutical composition prior to, concurrently with, or immediately following islet cell, kidney, liver, pancreas, heart, lung, intestine, bone marrow, limb, skin, stem cell, or other cell transplantation to prevent rejection thereof in the subject.

In some embodiments, treating or preventing cell, tissue, or organ transplant rejection may be monitored by evaluating one or more clinical signs or symptoms such as malignancy, susceptibility to infection, wound healing, thrombopenia, alopecia, gastrointestinal issues, gonadal dysfunction, hypertension, hyperlipidemia, nephrotoxicity, and peripheral edema.

The amount of the disclosed nanocarriers or pharmaceutical compositions comprising the same to be administered is dependent on a variety of factors, including the severity of the condition, the age, sex, and weight of the subject, the frequency of administration, the duration of treatment, and the like. The disclosed nanocarriers or pharmaceutical compositions may be administered at any suitable dosage, frequency, and for any suitable duration necessary to achieve the desired therapeutic effect. The disclosed nanocarriers or pharmaceutical compositions pharmaceutical compositions may be administered once per day or multiple times per day. For example, the nanocarriers or pharmaceutical compositions may be administered once per day, twice per day, or three or more times per day. The disclosed nanocarrier or pharmaceutical compositions may be administered daily, every other day, every three days, every four days, every five days, every six days, once per week, once every two weeks, or less than once every two weeks. The nanocarriers or pharmaceutical compositions may be administered for any suitable duration to achieve the desired therapeutic effect. For example, the nanocarriers or pharmaceutical compositions may be administered to the subject for one day, two days, three days, four days, five days, six days, seven days, eight days, nine days, ten days, eleven days, twelve days, thirteen days, two weeks, one month, two months, three months, six months, 1 year, or more than 1 year.

Any suitable dose of the disclosed nanocarriers or pharmaceutical compositions comprising the same may be used. Suitable doses will depend on the therapeutic agent, intended therapeutic effect, body weight of the individual, age of the individual, and the like. In general, suitable dosages of the disclosed nanocarriers or pharmaceutical compositions comprising the same may range from 1 ng nanocarrier/kg body weight to 100 g nanocarrier/kg body weight. For example, suitable dosages may be about 1 ng/kg to about 100 g/kg, about 100 ng/kg to about 50 g/kg, about 200 ng/kg to about 25 g/kg, about 300 ng/kg to about 10 g/kg, about 400 ng/kg to about 1 g/kg, about 500 ng/kg to about 900 mg/kg, about 600 ng/kg to about 800 mg/kg, about 700 ng/kg to about 700 mg/kg, about 800 ng/kg to about 600 mg/kg, about 900 ng/kg to about 500 mg/kg, about 1 μg/kg to about 400 mg/kg, about 10 μg/kg to about 300 mg/kg, about 100 μg/kg to about 200 mg/kg, about 200 μg/kg to about 100 mg/kg, about 300 μg/kg to about 10 mg/kg, about 400 μg/kg to about 1 mg/kg, about 500 μg/kg to about 900 μg/kg, about 600 μg/kg to about 800 μg/kg, or about 700 μg/kg.

For example, for the treatment or prevention of islet transplantation rejection the nanocarrier comprising rapamycin as the therapeutic agent or pharmaceutical composition comprising the same may be administered to the subject at a dose of 1 μg nanocarrier/kg body weight to about 100 mg/kg body weight. In general, rapamycin therapy maintains a whole blood concentration between about 1 ng/ml and about 20 ng/ml. Suitable does may be any dose that results in a maintained whole blood concentration between about 1 ng/ml and about 20 ng/ml. Dosage may be adjusted to increase or decrease the whole blood concentration after initial treatment to maintain the whole blood concentration between about 1 ng/ml and about 20 ng/ml. For example, suitable doses may be 1 μg/kg to about 100 mg/kg, about 10 μg/kg to about 90 mg/kg body weight, about 20 μg/kg to about 80 mg/kg, about 30 μg/kg to about 70 mg/kg, about 40 μg/kg to about 60 mg/kg, about 50 μg/kg to about 50 mg/kg, about 60 μg/kg to about 40 mg/kg, about 70 μg/kg to about 30 mg/kg, about 80 μg/kg to about 20 mg/kg, about 90 μg/kg to about 10 mg/kg, about 100 μg/kg to about 1 mg/kg, about 200 μg/kg to about 900 μg/kg, about 300 μg/kg to about 800 μg/kg, about 400 μg/kg to about 700 μg/kg, or about 500 μg/kg to about 600 μg/kg. For example, the dose may be about 1 μg/kg to about 1 mg/kg. For example, the dose may be about 1 μg/kg, about 10 μg/kg, about 20 μg/kg, about 30 μg/kg, about 40 μg/kg, about 50 μg/kg, about 60 μg/kg, about 70 μg/kg, about 80 μg/kg, about 90 μg/kg, about 100 μg/kg, about 150 μg/kg, about 200 μg/kg, about 250 μg/kg, about 300 μg/kg, about 350 μg/kg, about 400 μg/kg, about 450 μg/kg, about 500 μg/kg, about 550 μg/kg, about 600 μg/kg, about 650 μg/kg, about 700 μg/kg, about 750 μg/kg, about 800 μg/kg, about 850 μg/kg, about 900 μg/kg, about 950 μg/kg, or about 1 mg/kg.

Rapamycin nanocarrier formulations described herein have the advantage of higher bioavailability resulting in lower concentrations being required to maintain the required whole blood concentration of rapamycin for treatment. For example, given a dose of 1 mg rapamycin/kg body weight, by be administered to the subject fewer times (e.g., few daily injections or increased days between dosing, etc.). Alternatively, a smaller dose of rapamycin may be administered in the same number of injections at the same time interval as free rapamycin.

In particular embodiments, the disclosed nanocarrier comprising rapamycin as the therapeutic agent or compositions comprising the same may be administered to a subject for at least one day after transplantation to prevent islet transplantation rejection in the subject. For example, the nanocarrier or composition may be administered for at least 3 days after transplantation to prevent islet transplantation rejection in the subject. For example, the nanocarrier or composition may be administered for at least 5 days after transplantation. For example, the nanocarrier or composition may be administered for 5-20 days after transplantation. For example, the nanocarrier or composition may be administered for 5 days, 6 days, 7 days, 8 days, 9 days, 10 days, 11 days, 12 days, 13 days, 14 days, 15 days, 16 days, 17 days, 18 days, 19 days, or 20 days after transplantation. In some embodiments, the nanocarrier or composition may be administered for three weeks or more after transplantation (e.g. at least three weeks, at least one month, at least two months, at least three months, at least six months, at least one year). The nanocarrier or composition may be administered daily, every other day, every three days, every four days, every five days, every week, every two weeks, every month, or less than every month to the subject. The nanocarrier or composition may additionally be administered prior to and/or on the day of transplantation to prevent islet transplantation rejection in the subject.

The present invention has been described in terms of one or more preferred embodiments, and it should be appreciated that many equivalents, alternatives, variations, and modifications, aside from those expressly stated, are possible and within the scope of the invention.

### EXAMPLES

### Example 1

### Materials and Methods

Synthesis of PEG-bl-PPS copolymers and assembly of polymersomes-Polymersomes were fabricated from the controlled self-assembly of poly(ethylene glycol)-bl-poly(propylene sulfide) (PEG-bl-PPS) block copolymers with the hydrophilic PEG fraction of the total block copolymer molecular weight of 25% to 45%. PEG-bl-PPS block copolymers were synthesized using a PEG thioacetate initiated living polymerization of PPS that was end capped with PEG mesylate or CH₃COOH to create the PPS thiol-end groups for P210 peptide or fluorophore conjugation (Schematic 1). The obtained block copolymers (PEG₁₇-PPS₆₀-PEG₁₇ and PEG₁₇-bl-PPS₃₀-SH) were purified by double precipitation in methanol, and then characterized by ¹H NMR (CDCl₃) and gel permeation chromatography (GPC) (ThermoFisher Scientific) using Waters Styragel THF columns with refractive index and UV-Vis detectors in a tetrahydrofuran (THF) mobile phase. Polymersomes (PS) were self-assembled from PEG-bl-PPS block copolymers through thin film dehydration method in PBS. Briefly, 30 mg of PEG-PPS copolymer and fluorescent dye (Nile red) or aVD (1, 25-Dihydroxyvitamin D3, Sigma) were dissolved in 150 μl dichloromethane within 1.8 mL clear glass vials (ThermoFisher Scientific) and placed under vacuum to remove the solvent. The resulting thin films were hydrated in 1 ml of phosphate-buffered saline (PBS) under shaking at 1500 rpm overnight. The single layer PS were obtained by extrusion multiple times through 0.2 μm and then 0.1 μm nucleopore track-etched membranes (Whatman). For in vitro and in vivo imaging, PS suspensions at 30 mg/ml were covalently labeled with DyLight 633, DyLight 650 or DyLight 680 (ThermoFisher Scientific) via thiol-maleimide click reaction.

For P210 peptide conjugation, 50 ul of P210 peptide (KTTKQSFDLSVKAQYKKKNKHK, SEQ ID NO:2) with maleimide functional group (Peptide 2.0 Inc., Chantilly, Va.) (20 mg/ml in DMSO) was added to PS suspensions at 30 mg/ml and mixed overnight. The P210 peptide conjugated PS were purified by Zeba Spin Desalting Columns (14K MWCO, ThermoFisher Scientific) equilibrated with PBS solution. The conjugation efficiency of P210 on PS was determined by the 3-(4-carbpxubemzpul) quinoline-2-carboxaldyhyde (CBQCA) assay.

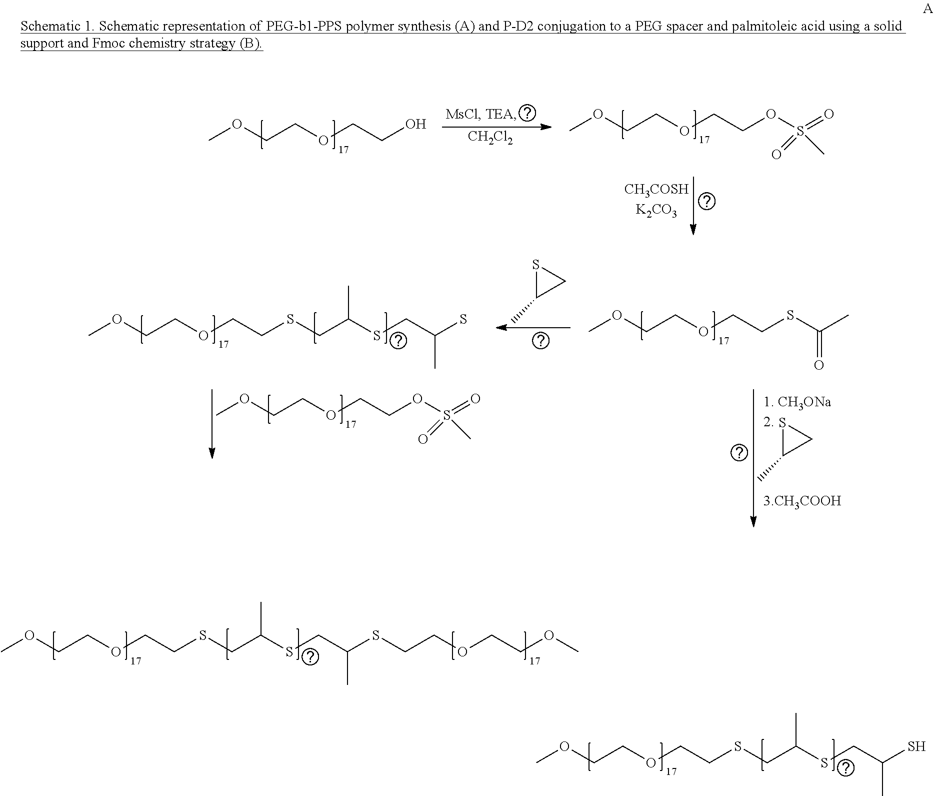

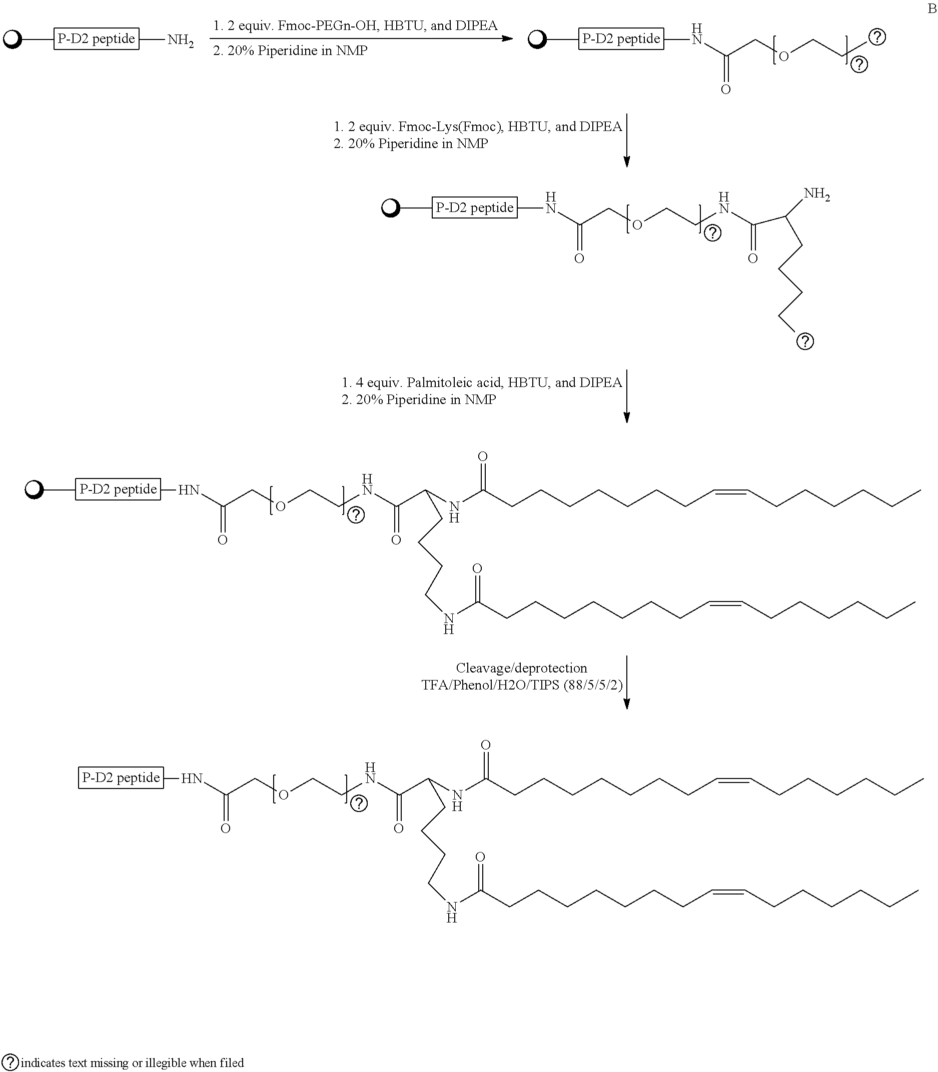

Synthesis of P-D2 targeting peptide constructs—P-D2 peptide (GGVTLTYQFAAGPRDK; SEQ ID NO:1) was synthesized in a 0.5 mmol scale on Wang resin (EMD Millipore) using a standard Fmoc solid phase peptide synthesis (SPPS) method and FastMoe™ chemistry (Applied Biosystems). The resins were firstly swelled in N-methyl-2-pyrrolidinon (NMP) for 1 h and the Fmoc residues were deprotected with 20% of piperidine in NMP for 20 min. Fmoc-amino acids (2-4 eq.) were coupled stepwise with (HBTU, 2-4 eq.) and N, N-diisopropylethylamine (DIPEA, 4 eq.) as a base by shaking for 3 h at room temperature. After the removal of the last Fmoc protecting group, Fmoc protected PEG spacers (Fmoc-PEG0/5/11/15-COOH, 2 eq.) were introduced to couple to the peptide amine groups with (HBTU, 2 eq.) and N, N-diisopropylethylamine (DIPEA, 4 eq.) by shaking for 4 h. The coupling and deprotecting efficiency were evaluated by Kaiser test kit (Sigma). Following the deprotection of the Fmoc from the peptide coupled PEG spacer, a Fmoc-Lys(Fmoc)-OH (2 eq.) was coupled with 2-(1H-Benzotriazol-1-ly)-1,1,3,3-tetramthyluronium hexafluorophosphate (HBTU, 2 eq.) and N, N-diisopropylethylamine (DIPEA, 4 eq.) by shaking 4 h at room temperature. After the Fmoc groups removal, 4 eq. of palmitoleic acid was then reacted with α- and ε-amines of lysine to produce two hydrophobic tails of the peptide conjugation in the presence of HBTU (2 eq.) and DIPEA (4 eq.) overnight. Deprotection and cleavage from the resin were performed by using Fmoc cleavage cocktails trifluoroacetic acid (TFA)/phenol/water/triisopropylsilane (TIPS) (88/5/5/2) twice for 2 h each time. The crude products were isolated by double precipitation in cold diethyl ether, and then purified by using reverse-phase high-pressure liquid chromatography (RP-HPLC) system (water-acetonitrile gradient, C18 column) in the SQI peptide synthesis core at Northwestern University. The purified molecules were mixed with α-cyano-4-hydroxycinnamic acid (CHCA) as the matrix prepared in 50:50 (v/v) acetonitrile/water containing 0.1% trifluoroacetic acid and determined by matrix-assisted laser desorption and ionization time-of-flight (MALDI-TOF) using a Bruker Autoflex III SmartBeam spectrometer (Bruker Daltonics Inc., Billerica, Mass.).

To achieve precise control over the density of peptide modifications on PS, the desired P-D2-PEGn-PA conjugations (1%, 2%, 3%, 4%, 5%, and 10% molar ratio of P-D2-PEGn-PA conjugations to PS) dissolved in DMSO were added to the suspension of aVD-loaded PS, P210 conjugated and aVD-loaded PS, Nile red-loaded PS or DyLight650-labeled PS and allowed to shake overnight. The fluorescent dye loaded and peptide modified PS were purified by Zeba Spin Desalting Columns (14K MWCO, ThermoFisher Scientific) equilibrated with PBS solution.

In vitro assessment of PS uptake by BMDCs and cellular uptake mechanism—Bone marrow-derived dendritic cells (BMDCs) were prepared. Bone marrow cells were harvested from femurs of naive C57BL/6 mice. The cells were then resuspended in primary media (RPMI 1640 medium supplemented with 10% FBS, 100 IU/ml Penicillin and 100 mg/ml streptomycin, 50 Um B-Me, 2 mM L-Gln, 20 ng/ml GM-CSF, and 10 ng/ml IL-4). Cells were cultured in 100 mm Petri dishes with the density of 1*10⁶/ml and incubated at 37 C with 5% CO₂ for 7 days. The culture media was refreshed on days 3 and 6. On day 7, BMDCs were seeded at 10⁵ cells/ml in 24-well plates and incubated for 24 h at 37 C with 5% CO₂. The NR-loaded PS with the same NR concentration in the presence or absence of different P-D2-PEGn-PA modifications were added into the wells and incubated for 1 h. The free PS were removed by repeat washing with PBS and centrifugation. The fluorescence intensity was quantitatively determined by flow cytometry (BD Biosciences).

For uptake inhibition studies, BMDCs were seeded at 2×10⁵ cells/ml in 12-well plates and incubated for 24 h at 37 C with 5% CO₂. After 30 min pre-incubation with various inhibitors: PBS (control), EIPA (0, 25, 50 μM), chlorpromazine (0 and 15m/ml), cells were treated with the same concentration of DyLight650-PS with or without P-D2-PEGn-PA modifications. After 1 h incubation, the BMDCs were collected and washed with PBS 3 times. The mean fluorescence intensity was determined by flow cytometry.

For immunofluorescence of PS uptake studies, μ-slide 8 well slides (ibidi) were treated with poly-L-Lysine (PLL, Sigma-Aldrich) at room temperature for 60 minutes, and then washed in PBS. BMDCs were seeded at 10⁵ cells/ml in PLL coated μ-slide 8 well slides and incubated for 12 h at 37° C. with 5% CO₂. Cells were incubated with DyLight 650-labeled PS or P-D2-PEG₅-PA for different time points (0 min, 5 min, and 20 min) at 4° C. and 37° C. Cells were fixed in 4% paraformaldehyde (PFA) and stained with clathrin heavy chain antibody (1:500 in blocking buffer, catalog #MA1-065, ThermoFisher Scientific). Images were acquired on a Leica TCS SP8 confocal microscope with a 63× oil immersion objective at Northwestern University.

Both MTT assay and flow cytometric assessment were performed to investigate BMDC viability following polymersome treatment. In MTT assay, BMDCs were seeded at 2.5×10⁵ cells/mL (200 μL; 50,000 cells/well) in 96-well tissue culture treated plates in complete RPMI. BMDCs received matched volume treatments of either PBS (n=3), PS (n=4), or P-D2-PEG5-PS (n=4) and were incubated for 18 h. Following this 18 h incubation, MTT (5 mg/ml in PBS; 20 μL) was added to each well and cells were incubated for an additional 8 h. Plates were centrifuged at 400×g for 5 min prior to media removal. Deposited formazan crystals were dissolved in 200 of dimethyl sulfoxide and assessed for absorbance at 560 nm using a Spectramax M3 Microplate Reader (Molecular Devices). The percentage cell viability was calculated in comparison to untreated BMDCs (n=3) using the formula: Cell viability=(OD of treated sample/OD of the untreated sample)*100%. BMDC viability following PS treatment was also completed using Zombie Aqua fixable cell viability dye. Following differentiation, cells were plated at 2.5×10⁵ cells/mL (200 μL; 50,000 cells/well) in 96-well tissue culture treated plates in complete RPMI. BMDCs received matched volume treatments of either PBS (n=3), PSs (n=4), or P-D2-PEG5-PS (n=4) and were incubated for 18 h. Following incubation, cells were collected and transferred to 1.2 mL microtiter tubes prior to staining with Zombie Aqua fixable viability dye (Biolegend) for 15 min. Once stained, cells were washed with cell staining buffer and fixed with intracellular (IC) cell fixation buffer (Biosciences). Flow cytometry data was acquired on an LSR Fortessa analyzer (BD Biosciences) and analyzed using cytobank software.

Assessment of BMDC activation and maturation in vitro—BMDCs were cultured at a density of 1×10⁶/ml and were incubated at 37° C. with 5% CO₂ for 10 days. Free aVD, aVD-loaded PS (PS-aVD) or aVD-loaded P-D2-PEG5-PS (P-D2-PEG5-PS-aVD) with the aVD concentration of 10⁻⁸ M were added when the media was refreshed on days 3 and 6. On day 7, 10 ng/ml murine recombinant interferon (IFN-γ) and 1 μg/ml LPS were added to stimulate dendritic cell maturation. At day 10, BMDCs were collected, washed with PBS and then incubated with anti-mouse CD16/CD32 to block FcRs and Zombie Aqua fixable viability dye to determine live/dead cells for 20 min. Cells were washed with PBS and stained with antibody cocktail (CD11c, WICK CD80, and CD86) (Table 1) for 35 min at 4° C. BMDC maturation was determined by characterizing the cell surface marker expression using FACSDiva on a LSRII flow cytometer (BD Biosciences). Over 20,000 events were recorded for each sample and data were analyzed with FlowJo software. Different concentrations of P-D2-PEG5-PS-aVD (aVD concentration=0, 10⁻⁹, and 10⁻⁸ M) were also investigated on BMDC maturation using the same procedure described above. To determine the ability of PS or P-D2-PEG5-PS to inhibit DCs activation, the same concentration of polymers as used in the above BMDC maturation experiments was added on days 3 and 6. Without stimulating DC maturation, the BMDCs were collected on day 8 and the expression of surface costimulatory receptors were characterized by flow cytometry as previously described. IL12p70 secretion in supernatant of the cell culture in the last 48 h were determined by enzyme-linked immunosorbent assay (ELISA) following the manufacturer's instructions (Fisher Thermo Scientific).

TABLE 1
Antibodies that were used for flow cytometry in this study

| Antigen | Fluorophore | Clone |
|---|---|---|
| CD45 | Percp/Cy5.5 | 30-F11 |
| CD45 | FITC | 30-F11 |
| CD3 | APC/Cy7 | 17A2 |
| CD4 | PE | GK1.5 |
| CD4 | Percp/Cy5.5 | RM4-4 |
| CD8a | APC | 53-6.7 |
| CD8a | PE/Cy7 | 53-6.7 |
| F4/80 | PE/CY7 | BM8 |
| NK1.1 | BV421 | PK136 |
| CD11b | PE/Cy7 | M1/70 |
| CD11b | FITC | M1/70 |
| CD11c | BV421 | N418 |
| CD11c | Pacific Blue | N418 |
| B220 | PE | RA3-6B2 |
| CD19 | Pacific Blue | 6D5 |
| Ly-6G | APC | 1A8 |
| Ly-6C | APC/Cy7 | HK1.4 |
| MHC-II | FITC | M5/114.15.2 |
| CD80 | APC | 16-10A1 |
| CD86 | PE | GL-1 |

Animals—The apolipoprotein E-deficient (ApoE^{-/-}) mice with C57BL/6 background were obtained from The Jackson Laboratory at 4-6 weeks old and fed a high-fat diet (HFD, Harlan Teklad TD.88137, 42% kcal from fat) starting at 7 weeks old for 18 weeks until sacrificed. All mice were housed and maintained in the Center for Comparative Medicine at Northwestern University. C57BL6/J mice were obtained from Jackson Laboratory at 4-6 weeks old and were fed a standard diet. All animal experimental procedures were performed according to protocols approved by the Northwestern University Institutional Animal Care and Use Committee (IACUC). For each experiment, mice were allocated randomly to each group. Female cynomolgus monkeys that originated from Mauritius and were on average 4.8 years of age (range 4.5-4.9) were housed in an AAALAC accredited facility at the University of Kentucky (UK) under the care of the UK Division of Laboratory Animal Resources, and all non-human primate studies were approved by the UK Institutional Animal Care and Use Committee.

Administration of PS to C57BL6/J mice and non-human primates—DyLight 633-labeled PS were administered to mice and non-human primates (NHP) at a dose of 20 mg/kg. Mouse treatments (n=3) were administered via tail-vein injection, while NHP treatments (n=2) were administered via saphenous vein with a Harvard syringe pump at 1 mL/minute. 24 hours after administration, animals were euthanized and the liver, kidneys, and spleen from each animal was collected and processed into single cell suspensions. For flow cytometric analysis, cells were stained using cocktails of fluorophore-conjugated anti-mouse antibodies. Mouse: BUV396 anti-CD45, BV605 anti-F4/80, FITC anti-NK1.1 anti-CD3 and anti-CD19, PerCP/Cy5.5 anti-CD11b, PE anti-B220, BV421 anti-CD11c, AlexaFluor 750 anti-CD8a. Non-human primate: BV450 anti-CD45, APC/Cy7 anti-HLA-DR, PE anti-CD1c, FITC anti-CD123, PerCP/Cy5.5 anti-CD3 anti-CD20. For subset comparisons between mice and NHP, mouse CD8a+ DCs and CD11b+ DCs were considered analogous to primate cDC1s and cDC2s, respectively. Plasmacytoid DCs from each species were also considered analogous.

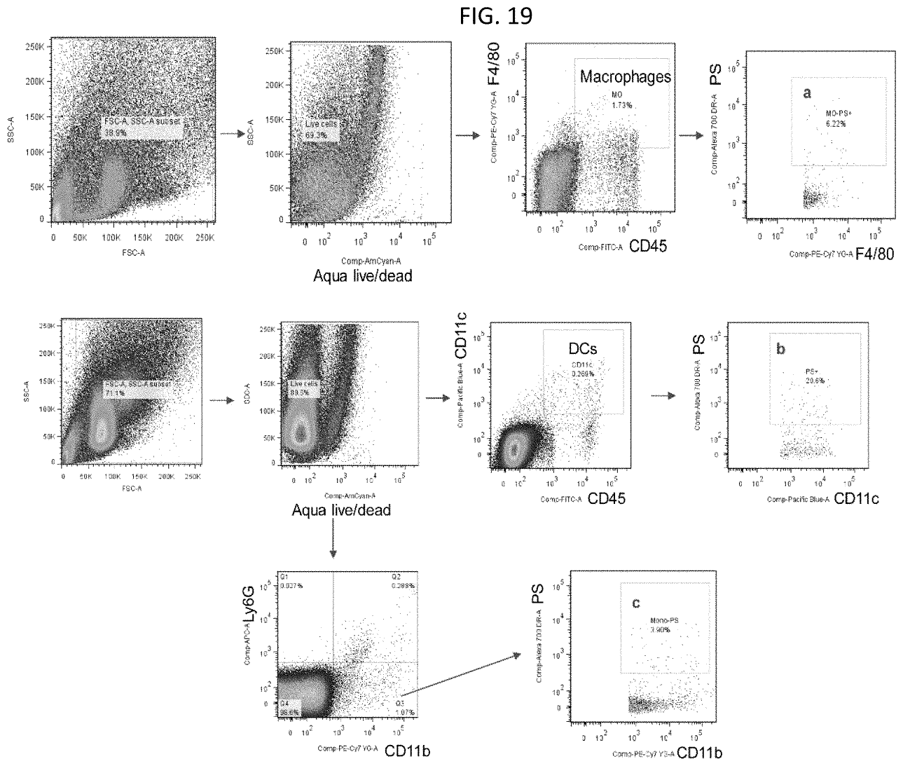

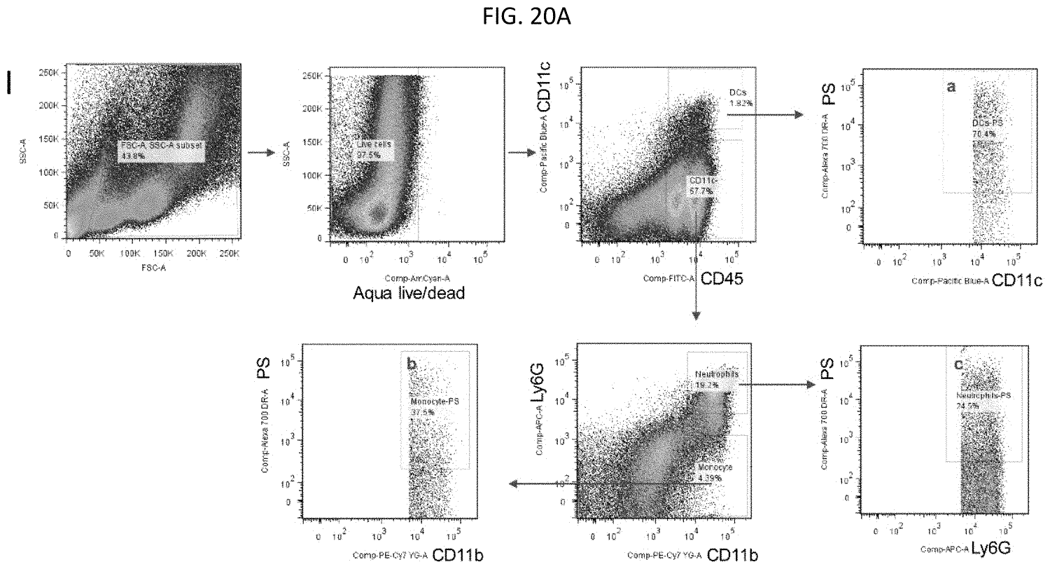

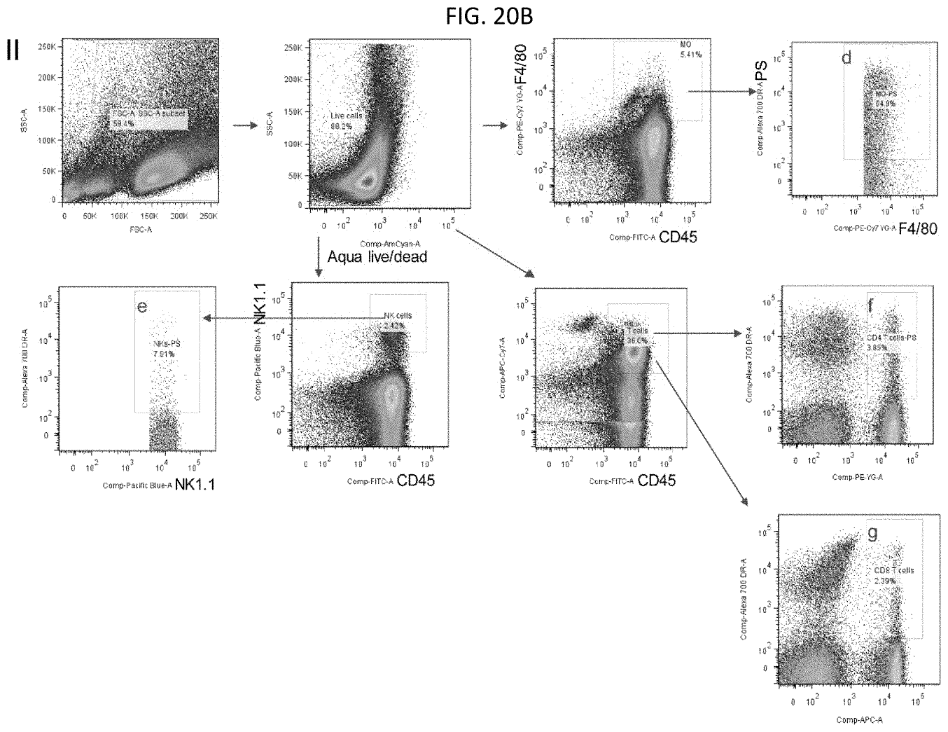

Measurement of immune cell biodistributions for P-D2 modified PS in ApoE^{-/-} mice—ApoE^{-/-} male mice (n=4-6), were injected i.v. with 150 μl of PS, P-D2-PS, or P-D2-PEG5-PS labeled with DyLight680 (block copolymer concentration of 15 mg/ml). After 24 h, mice were euthanized under CO₂ anesthesia and spleen and aorta were harvested and organ NIRF imaging was performed by an IVIS Lumina with filter of 680/800 nm. The single cell suspensions were then prepared from various organs. Cells were stained with anti-mouse CD16/CD32 to block FcRs and Zombie Aqua fixable viability dye prior to antibody staining. After wash, cells were then stained with multiple cocktails of fluorophore-conjugated anti-mouse antibodies (Table 1). Flow cytometry was performed with BD LSRFortessa 6-Laser flow cytometer (BD Biosciences) and data were analyzed with FlowJo software. The gating strategies are shown in FIGS. 19-20.

Treatment—Male ApoE^{-/-} mice at 8-weeks of age were fed a high-fat diet (HFD, Harlan Teklad TD.88137, 42% kcal from fat) for 4 weeks. Treatments were performed from week 12 and 100 μl of different treatments: 1, PBS (control); 2, free aVD (1 μg/ml); 3, PS-aVD; 4, P-D2-PEG5-PS-aVD; 5, P210/P-D2-PEG5-PS-aVD (2, 3, 4, 5 groups with the same aVD concentration of 1 μg/ml) were subsequently i.v. administrated every week for 8 weeks. During those weeks, mice were maintained on a high-fat diet, and the activities and body weight was monitored. In vivo experiments were performed with group sizes of N=5-6 mice per group and then independently repeated for a total of N=10-12 mice per group.

Measurements of serum lipid profiles and cytokines—After 8 weeks treatment, mice were euthanized, and blood was collected by retro-orbital puncture with BD Microtainer tubes and dipotassium EDTA (BD Biosciences). Serum was separated by centrifugation at 3000 rpm at 4° C. for 25 min and stored at −80 C. Total cholesterol was determined by HDL and LDL/VLDL Quantitation Kit (Sigma). Cytokines TGF-β (ThermoFisher Scientific) and IL-10 (Biolegend) were measured by ELISA assays (BioLegend) and cytokines (IL-6, IFN-γ) were measured by a customized Luminex Multiplex panel, according to the manufacturer's instructions (ThermoFisher Scientific).

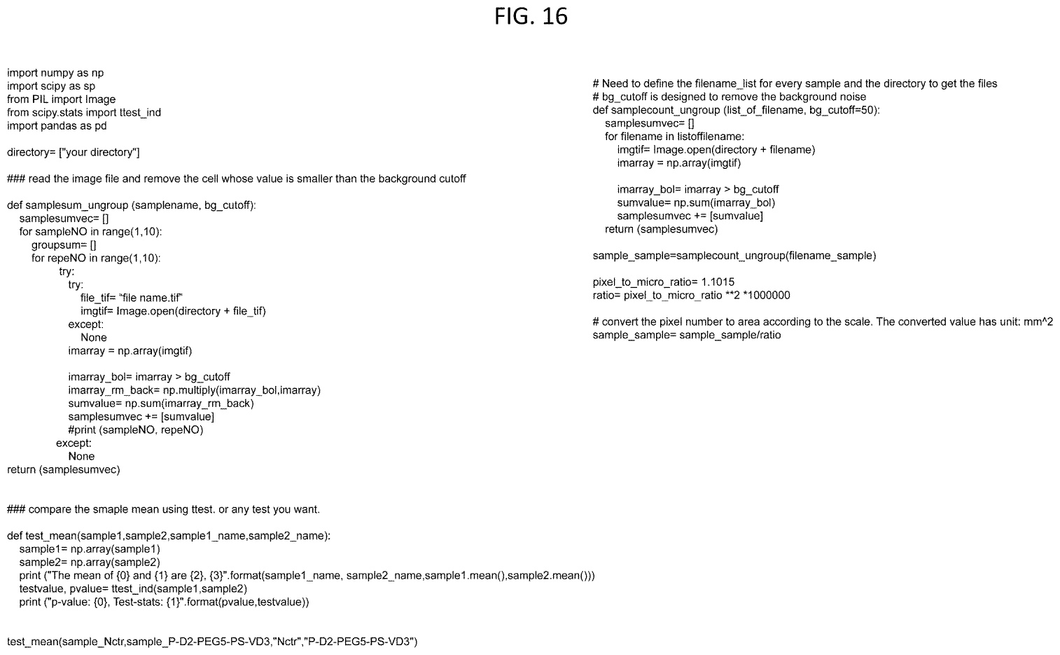

Atherosclerotic lesion quantification and immunohistochemistry—For atherosclerotic lesion analysis, mice were anesthetized and aortas were carefully harvested after perfusion with PBS under a microscope. The heart with ascending aorta was fixed with 4% paraformaldehyde (PFA)/5% sucrose in PBS solution 12 h at 4° C. The tissue samples were immersed in 15% sucrose solution for 12 h and then 30% sucrose solution for 24 h. The resulting specimens were embedded in Tissue-Tek OCT and frozen at −80° C. and then sectioned with a cryostat. Serial sections (10 μm thick) of the aortic roots were collected (5-7 sections per mouse) starting at the appearance of aortic valves. The distance between each section was 100 μm, and serial cross-sections were obtained. For quantitative analysis of atherosclerotic lesions, 5-7 separate cross-sections from each mouse were stained with Oil Red 0 (ORO) (Sigma) for 1 h at 37° C. and 4′,6-diamidino-2-phenylindole (DAPI) for 5 min. The presence of immune cells in aortic lesions was studied by immunohistochemistry. The slides with multiple frozen aortic root sections were fixed in acetone and twice with PBS. Specific antibodies were used on another consecutive cross-section for macrophages (anti-CD68, 1:500, Abcam) and Treg cells (anti-Foxp3, 1:500, Abcam). Slides were stained using the Tyramide Signal Amplification kits in MHPL core facility of Northwestern University. All slides containing the cross-sections were digitally imaged with Leica DM6B widefield fluorescent microscope. An in-house software written in Python was developed for automated and quantitative image analysis (FIG. 16)

Flow cytometry analysis—White blood cells were obtained after eliminating red blood cells by treatment three times with ammonium-chloride-potassium (ACK) lysis buffer (Invivogen). Splenocytes and LN cells were prepared. Anti-mouse CD16/CD32 was used to block FcRs and Zombie Aqua fixable viability dye was used to determine live/dead cells. For flow cytometric analysis, cells were stained using cocktails of fluorophore-conjugated anti-mouse antibodies (Table 1). After washes, cells were suspended in cell staining buffer (eBioscience) and fixed by IC cell fixation buffer (eBioscience). Intracellular staining of Foxp3 was performed using Foxp3 Fix/Perm Buffer Set (Biolegend). Flow cytometry was performed with BD LSRFortessa 6-Laser flow cytometer (BD Biosciences) and data were analyzed with FlowJo software.

qRT-PCR—Mice were anesthetized and spleen and aorta were isolated as described above at week 21. The obtained mouse tissues were immediately preserved in RNAlater solution (Sigma) and stored at −80° C. Intracellular cytokine gene expression analysis was performed by quantitative real-time reverse transcriptase polymerase chain reaction (qRT-PCR). Frozen mouse tissues (spleen and aorta) were homogenized by Tissuelyser-II (QIAGEN) and total RNA was isolated using RNeasy mini kit (Qiagen). RNA was then reverse transcribed into complementary deoxyribonucleic acid (cDNA) using High Capacity cDNA Reverse Transcription Kits (ThermoFisher Scientific) according to the manufacturer's instructions. The high-throughput PCR was performed in 384-well plates in triplicate by adding 1.2 μl cDNA (˜20 μg/μl) and 1.2 μl SsoAdvanced Universal SYBR Green Supermix (Bio-Rad) using a Mosquito robot (TTP Lab Tech), and then mixing with 12 nl primer mix (100 nM of each primer) via Echo 550 acoustic transfer robot (Labcyte). Quantitative RT-PCR was performed by BioRad CFX384 Real-Time PCR Detection System (Bio-Rad). All samples were normalized to the housekeeping gene (GAPDH). The primers used in this study were listed in Table 2.

TABLE 2
List of primers used for qRT-PCR in this study

| | Forward Primer | Reverse Primer |
|---|---|---|
| Gene | (5′-3′) | (5′-3′) |
| GAPDH | GGCACAGTCAAGGCCGAGA | GATGTTAGTGGGGTCTCGC |
| | ATGG | TCCTGG |
| | (SEQ ID NO: 3) | (SEQ ID NO: 4) |
| Foxp3 | CTC ATG ATA GTG CCT | AGG GCC AGC ATA GGT |
| | GTG TCC TCA A | GCA AG |
| | (SEQ ID NO: 5) | (SEQ ID NO: 6) |
| CD80 | AGT TTC CAT GTC CAA | TTG TAA CGG CAA GGC |
| | GGC TCA TTC | AGC AAT A |
| | (SEQ ID NO: 7) | (SEQ ID NO: 8) |
| CD86 | GTTCTGTACGAGCACTATT | GTGAAGTCGTAGAGTCCAG |
| | TGGGC | TTGTTCC |
| | (SEQ ID NO: 9) | (SEQ ID NO: 10) |
| IL-10 | GGGTTGCCAAGCCTTATCG | CTTCTCACCCAGGGAATTC |
| | (SEQ ID NO: 11) | AAATG |
| | | (SEQ ID NO: 12) |
| IL-6 | TTCCATCCAGTTGCCTTCT | GGGAGTGGTATCCTCTGTG |
| | TGGG | AAGTC |
| | (SEQ ID NO: 13) | (SEQ ID NO: 14) |
| ICAM-1 | CAA TTC ACA CTG | CAA GCA AGT CCG TCT |
| | AATGCC AGC TC | CGT CCA |
| | (SEQ ID NO: 16) | (SEQ ID NO: 17) |
| VCAM-1 | TGCCGG CAT ATA CGA | CCC GAT GGC AGG TAT |
| | GTG TGA | TAC CAAG |
| | (SEQ ID NO: 18) | (SEQ ID NO: 19) |

AFM measurement for arterial stiffness—The biomechanical properties of arteries from ApoE^{-/-} mice were measured on the aortic arch ex vivo using atomic force microscopy (AFM) (Dimension Icon, Bruker). Mice were anesthetized and perfused with PBS for 10 min after treatment. The fatty tissue around the aorta was carefully removed under microscopy, and the aorta was then isolated as described above. The aortic arch was opened and cut into a small piece (3×8 mm) and placed on the poly-L-lysine coated glass slides with PBS. Each end of the aortic tissue was firmly fixed on the slides by 1 ul cyanoacrylate adhesive glue (Krazy). After 1 min air-drying, PBS was added, and adequate hydration was maintained throughout the AFM measurement. Spherical silicon nitride probes (1 μm diameter, 0.06 N/m cantilever spring constant, Novascan) were used in all experiments. 5-10 measurements were captured from each area and 5 different areas were characterized per sample. The force-indentation curves were fit with the linearized Hertz model in the contact region to calculate the Young's Modulus, with the Poisson ratio assumed to be 0.5.

In vitro surface engineering of PS for an optimized display of DC targeting peptide constructs—It was previously found that vesicular PS with diameters between 120-150 nm were favored for endocytosis by splenic DCs in mice (Yi, S., et al., Tailoring Nanostructure Morphology for Enhanced Targeting of Dendritic Cells in Atherosclerosis. ACS Nano, 2016. 10(12): p. 11290-11303, incorporated herein by reference in its entirety). This effect was validated herein in a more clinically relevant non-human primate model. 24 h after intravenous (i.v.) administration, uptake of PEG₁₇-bl-PPS₃₀ PS by DC populations in the major organs of the mononuclear phagocyte system (spleen, liver, and kidneys) in cynomolgus monkeys was assessed by flow cytometry (FIG. 1A). Compared to mice, no significant differences in the percentages of PS positive (PS⁺) DC subsets in non-human primates were found, demonstrating that NSET of DCs via the PS nanocarrier morphology applies equally well in both animal models. These results confirm the relevancy of optimizing and validating nanocarrier-dependent DC targeting strategies in mice, which currently serve as the most established and economical models of both atherosclerosis and immune modulation. All further in vivo testing was therefore performed in the standard high fat diet-fed ApoE^{-/-} mouse model of atherosclerosis.

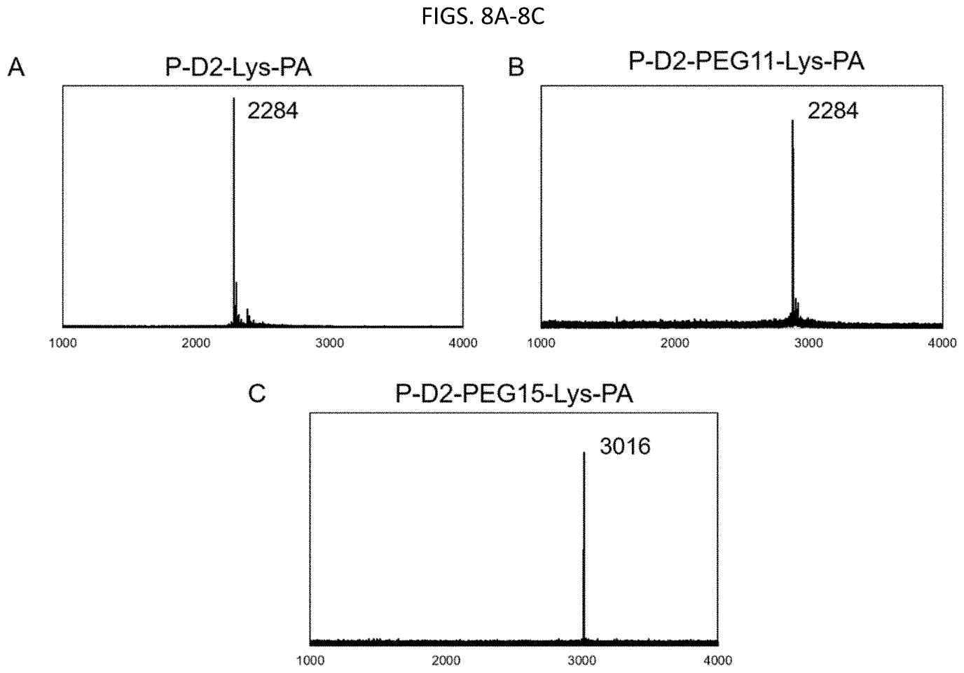

It was next sought to synergize this PS-enhanced targeting of DCs with an optimal surface density of the P-D2 peptide. Derived from the Ig-like domain 2 of intercellular adhesion molecule 4 (ICAM-4), the P-D2 peptide targets DCs, promotes intracellular delivery, and enhances tolerogenic responses. To optimize the peptide display, a construct was designed composed of three linked components: the P-D2 peptide, a PEG spacer, and a palmitoleic acid (PA) lipid tail for insertion into the lipophilic PS membrane (FIG. 1B). These constructs allowed precise control over the surface density of the displayed peptide by loading efficiently and stably into bilayers of PS, which were self-assembled from PEG-bl-PPS block copolymers (FIG. 1B, schematic 1A). The controllable synthetic approach for P-D2 conjugation was performed by Fmoc chemistry (schematic 1B). Multiple PEG spacers were introduced to present P-D2 peptide above the PEG corona of PS surface for efficient ligand-receptor interactions. Numerous lipids are atherogenic. Accordingly, palmitoleic acid was selected because it may be a therapeutic lipid that prevents macrophage ER stress and IL-1β production in atherosclerotic mice. After cleavage and purification by RP-HPLC, a purity of over 99% was determined by analytical HPLC and matrix-assisted laser desorption/ionization-time of flight (MALDI-TOF) (FIG. 1C, FIG. 8)

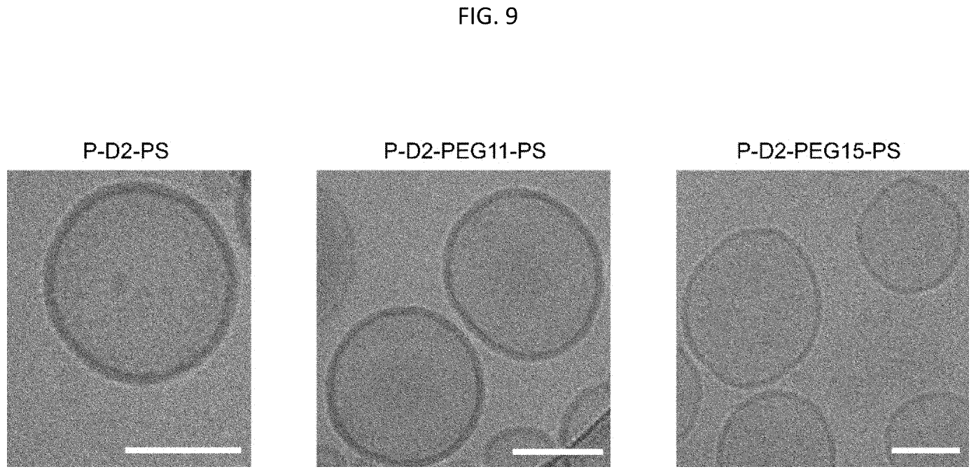

To study the effect of P-D2 conjugated PS with different PEG spacers, PS were prepared by a thin film dehydration method, adding different P-D2 constructs with or without PEG spacers composed of 0, 5, 11, or 15 PEG units (P-D2-Lys-PA; P-D2-PEG5-Lys-PA; P-D2-PEG11-Lys-PA; P-D2-PEG15-Lys-PA). The stable vesicular structures of the assembled PS after incorporation of the P-D2 peptide constructs was verified with cryogenic transmission electron microscopy (CryoTEM) (FIG. 1D, FIG. 9). The hydrodynamic size of P-D2 conjugated PS was 140 nm-160 nm and marginally increased with the increasing length of the PEG spacer as determined by dynamic light scattering (DLS) (Table 3). Zeta potential showed that the surface charge of P-D2 conjugated PS was slightly negative in PBS solution (Table 3).

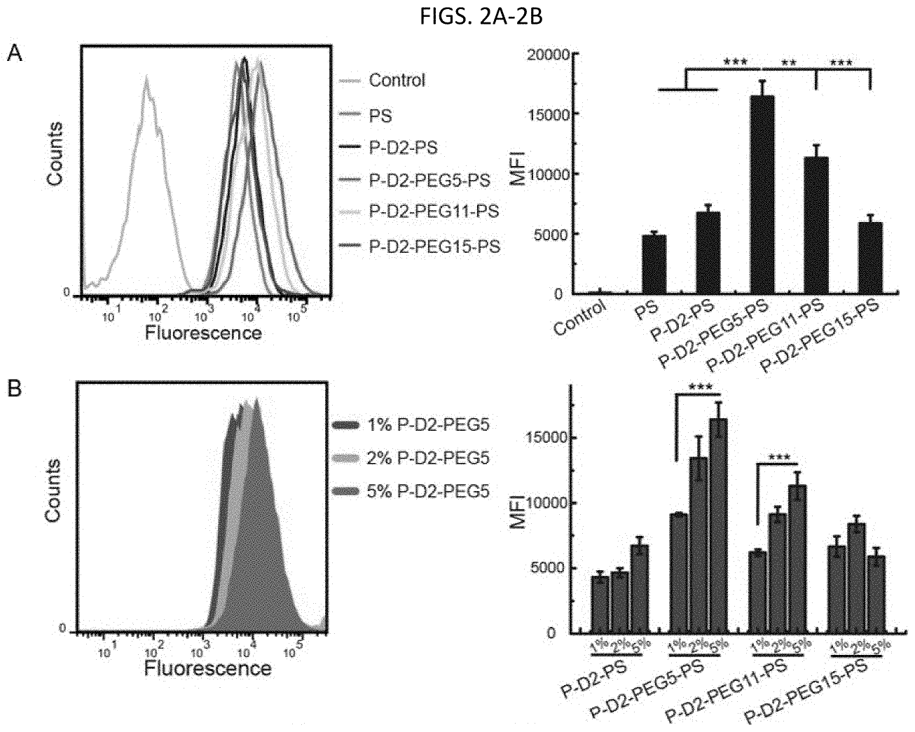

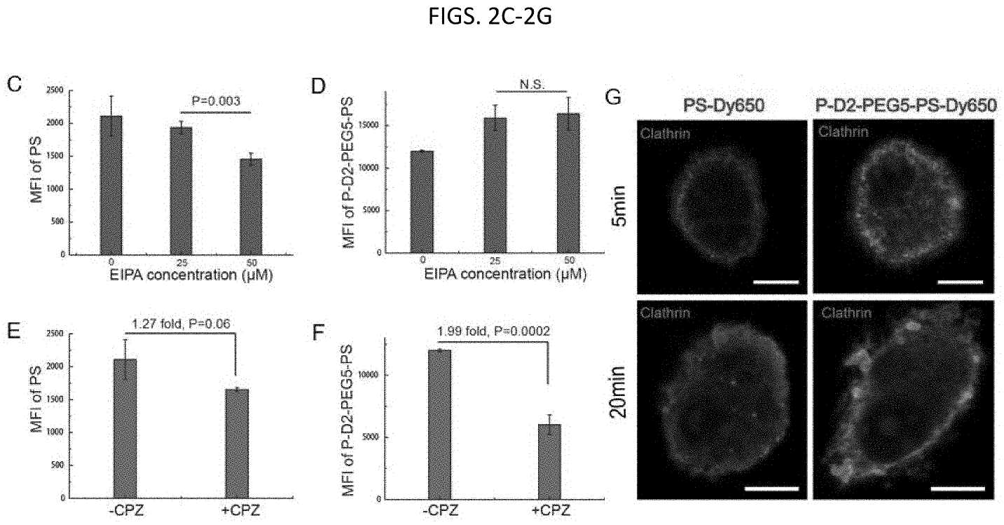

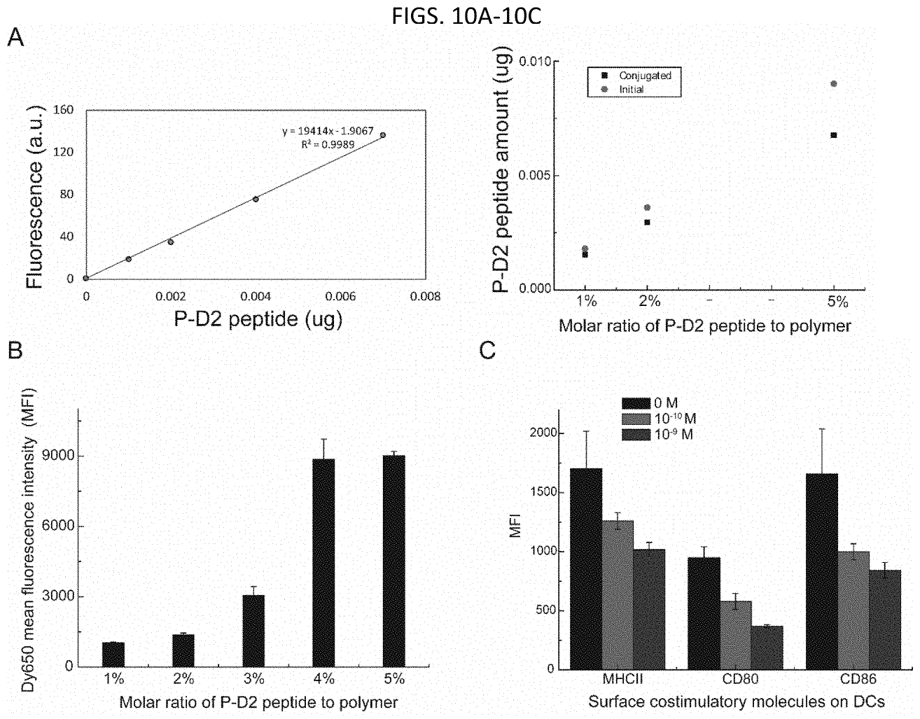

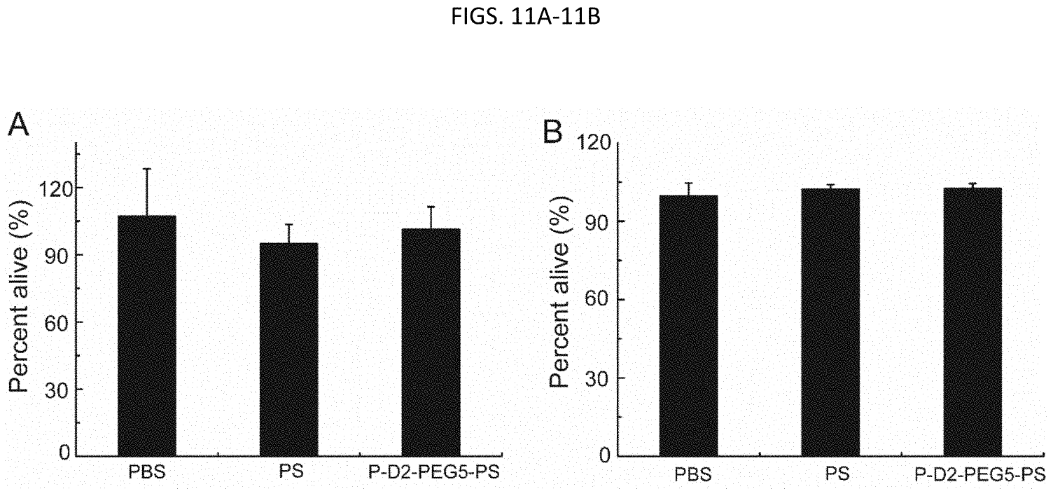

The targeting capacities of P-D2 conjugated PS for DCs were optimized in vitro using bone marrow-derived dendritic cells (BMDCs). Nile red was used as a hydrophobic fluorescent marker and the internalization of PS was evaluated by flow cytometry after 1 h incubations with BMDCs (FIG. 2A,B). In regard to the structure of the peptide display, a significant spacer length dependent enhancement in cellular uptake was observed, with an optimal >3-fold enhancement (p<0.001) identified for the 5 unit PEG spacer compared to PS without the targeting peptide (FIG. 2A). The displayed peptide surface density was optimized for BMDC uptake using P-D2 peptide to copolymer molar ratios of 1%, 2%, and 5%. The PS uptake by BMDCs directly increased with the increasing peptide densities (p<0.001) (FIG. 2B). The loading efficiency of P-D2 peptide on the PS surface was over 87% as determined by the 3-(4-carbpxubemzpul) quinoline-2-carboxaldyhyde (CBQCA) assay, which also confirmed that different densities of P-D2 peptide were stably incorporated onto the PS surfaces after purification by size exclusion chromatography (FIG. 10A). To further optimize the peptide density, DyLight 650-labeled PS incorporating different molar ratios of P-D2-PEG5-Lys-PA (0, 1%, 2%, 3%, 4%, 5%) were investigated. From 1% to 4%, the PS uptake was significantly increased, with a maximum observed at 4% and no further increasing at 5% (FIG. 10B). In addition, no cellular cytotoxicity was observed on BMDCs after the treatment with both PS and P-D2-PEG5-PS (FIG. 11). Given the optimized targeting capacity to BMDCs, P-D2-PEG5-PS with the P-D2 peptide density of 4% was employed as the nanocarrier in subsequent animal studies.

TABLE 3
Physicochemical characteristics of PEG-bl-PPS polymersome
conjugations in PBS solution (pH = 7.4)

| | Average | Polydispersity | Zeta |
|---|---|---|---|
| | diameter | index | potential |
| Samples | (nm) | (PDI) | (mV) |
| PS | 115.2 | 0.24 | −2.2 ± 1.57 |
| P-D2-PS (1%)* | 140.4 | 0.151 | −1.52 ± 0.76 |
| P-D2-PS (2%) | 138.3 | 0.093 | −0.81 ± 1.13 |
| P-D2-PS (5%) | 130.9 | 0.153 | −4.18 ± 0.96 |
| P-D2-PEG5-PS (1%) | 146.3 | 0.162 | −3.13 ± 1.89 |
| P-D2-PEG5-PS (2%) | 141.1 | 0.156 | −1 ± 0.36 |
| P-D2-PEG5-PS (5%) | 142.1 | 0.146 | −1.86 ± 0.74 |
| P-D2-PEG11-PS (1%) | 143.7 | 0.054 | −0.58 ± 1.32 |
| P-D2-PEG11-PS (2%) | 147 | 0.059 | −0.44 ± 1.19 |
| P-D2-PEG11-PS (5%) | 146.7 | 0.482 | −0.87 ± 1.65 |
| P-D2-PEG15-PS (1%) | 146.9 | 0.066 | −5.62 ± 2.33 |
| P-D2-PEG15-PS (2%) | 145.4 | 0.088 | −2.42 ± 1.35 |
| P-D2-PEG15-PS (5%) | 158.3 | 0.277 | −2.92 ± 0.39 |
| P210/P-D2-PEG5-PS (4%) | 143.6 | 0.125 | −5.32 ± 1.24 |

*indicates the molar ratio of P-D2 peptide to copolymer.

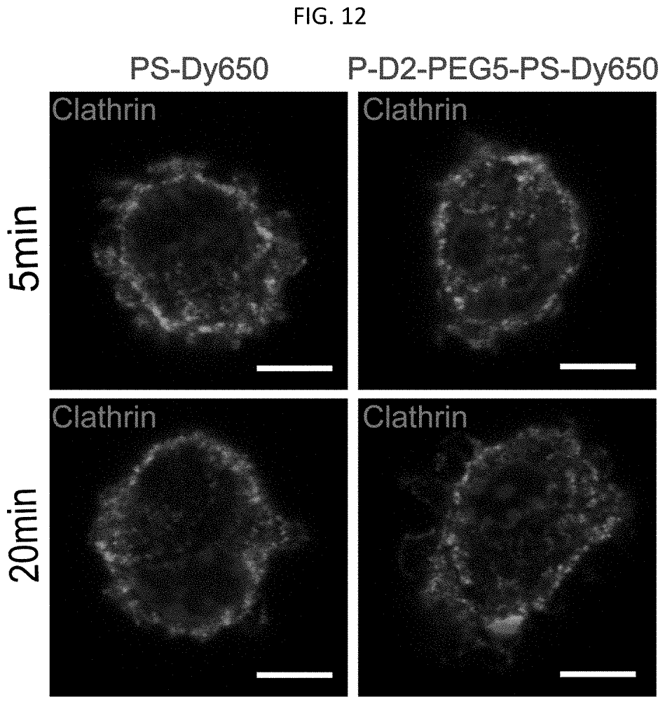

Mechanistic validation of enhanced receptor mediated endocytosis by the optimized P-D2 surface display—To investigate the mechanisms involved in PS uptake by DCs, different inhibitors were employed to interfere with key uptake pathways. Inhibition of micropinocytosis with 5-(N-ethyl-N-isopropyl)amiloride (EIPA) reduced PS but not P-D2-PEG5-PS uptake against BMDCs in a dose dependent manner (FIG. 2C,D). Following incubation with chlorpromazine, which specifically blocks clathrin-dependent receptor mediated endocytosis, fluorescence from DCs treated with either PS or P-D2-PEG5-PS was decreased. However, a significantly larger decrease was observed in P-D2-PEG5-PS treated DCs (1.9 fold, p=0.0002) than PS treated cells (1.27 fold, p=0.06) (FIG. 2E,F). To verify the endocytosis pathway in inhibition studies, BMDCs were incubated with DyLight 650-labeled P-D2-PEG5-PS or PS, and then stained with clathrin heavy chain for imaging by confocal microscopy. Confocal images showed the colocalization of P-D2-PEG5-PS but not PS with clathrin heavy chain especially after incubation at 37° C. for 20 min (FIG. 2G). P-D2-PEG5-PS but not PS were observed to bind BMDC surfaces at 4° C. for 20 min, and neither nanocarrier was internalized at this lowered temperature. (FIG. 12). These studies indicated that PS enter DCs through both micropinocytosis and clathrin-mediated endocytosis and P-D2 decoration significantly increases both the amount and rate of intracellular delivery.

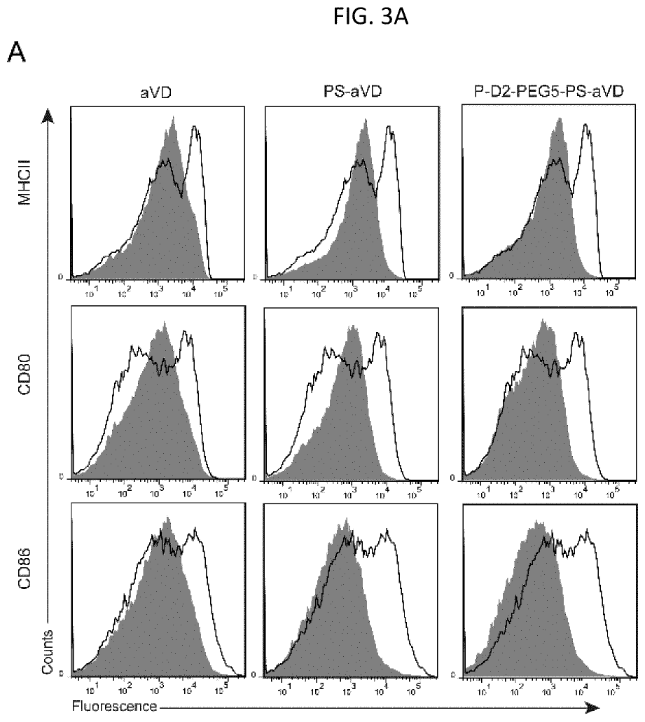

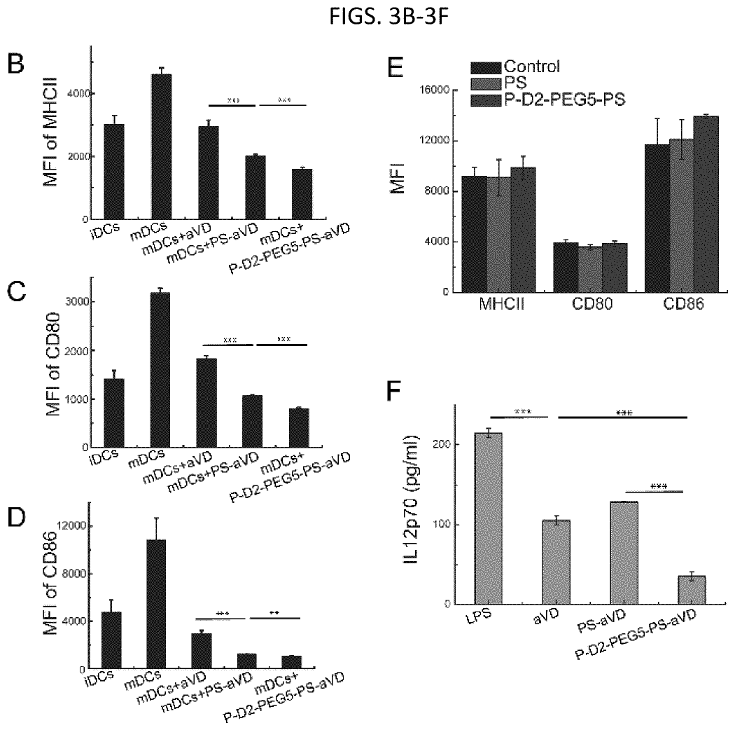

P-D2 decorated PS enhance aVD-dependent inhibition of pro-inflammatory DC activation—DCs play a pivotal role in the stimulation and polarization of T cells in atherosclerosis. aVD has been demonstrated to generate an immature phenotype on DCs with low expression of MHC-II and costimulatory molecules (CD80 and CD86), as well as decreased secretion of proinflammatory cytokines. The immunomodulatory effects of aVD are achieved through its intracellular activation of the VDR, a nuclear hormone receptor that inhibits NF-kB activity by both downregulating gene expression and by physically interacting with IκB kinase β. The ability of PS, P-D2-PEG5-PS, aVD-loaded PS (PS-aVD), and aVD-loaded P-D2-PEG5-PS (P-D2-PEG5-PS-aVD) to modulate costimulatory molecule expression by BMDCs in response to inflammatory stimulation by lipopolysaccharide (LPS) was therefore explored. Cells treated with aVD, PS-aVD and P-D2-PEG5-PS-aVD showed significantly decreased expression of MHC-II, CD80 and CD86 (FIG. 3A-D). Importantly, P-D2-PEG5-PS-aVD significantly reduced the expression of these maturation markers (p<0.001) compared to both PS-aVD and free aVD. This effect was found to be concentration-dependent (FIG. 10C). In the absence of aVD, neither PS nor P-D2-PEG5-PS influenced BMDC surface molecule expression (FIG. 3E). The production of IL-12p70 in cell culture supernatant was also markedly inhibited by P-D2-PEG5-PS-aVD (p<0.001) compared to PS-aVD or free aVD treated groups (FIG. 3F). These results demonstrate that the increased intracellular delivery by P-D2 conjugated PS dramatically enhanced aVD-based suppression of DC pro-inflammatory pathways.

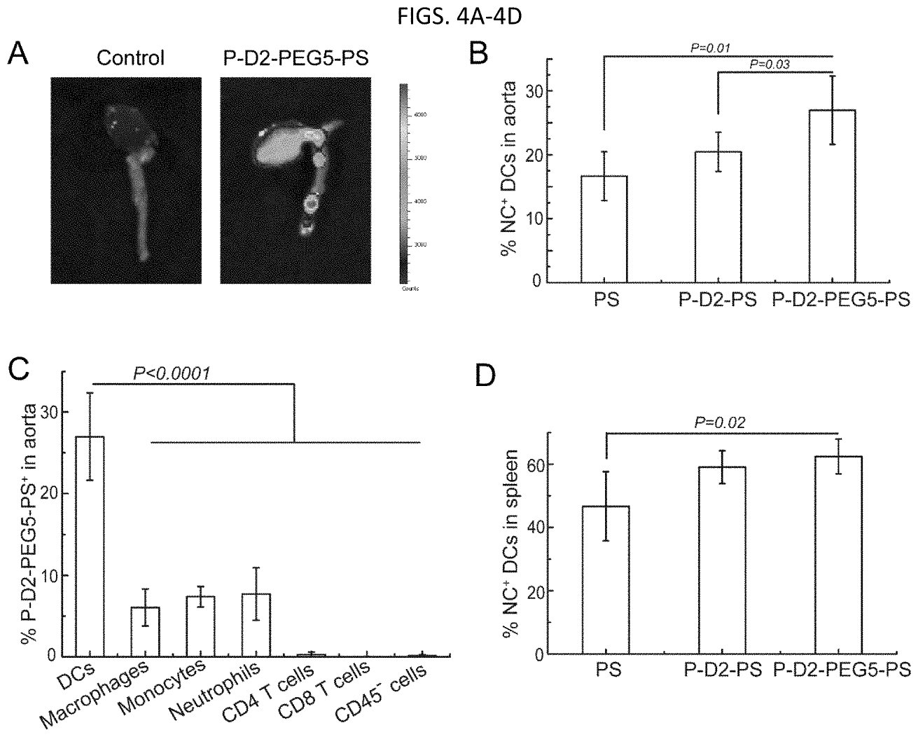

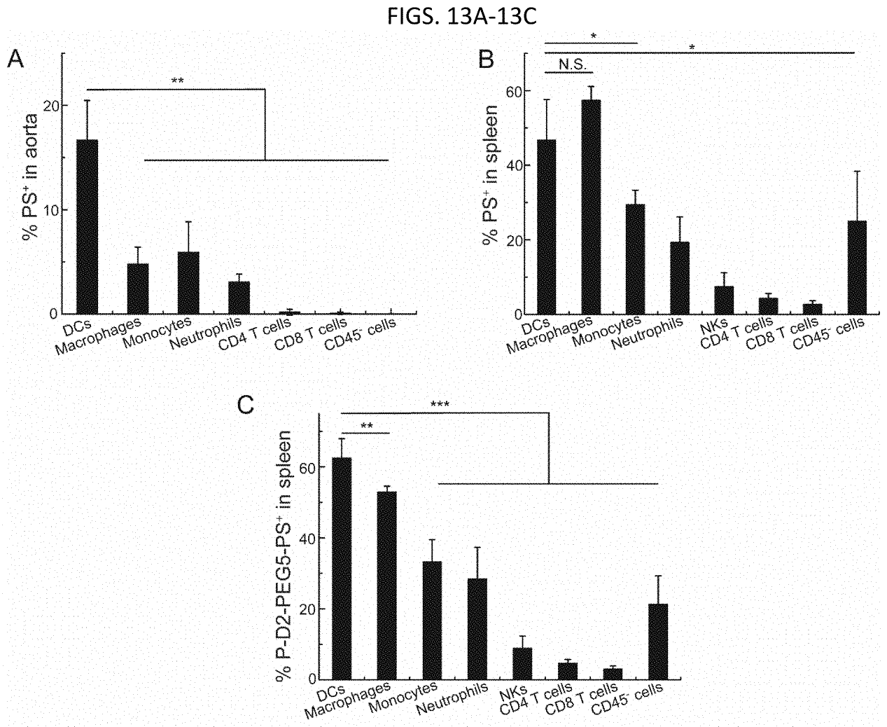

In vivo validation of surface engineered PS with optimized display of P-D2 peptide—To validate the enhanced targeting of DCs in atherosclerosis by combining NSET with P-D2 peptide display, ApoE^{-/-} mice were fed with the high-fat diet for 8 weeks and i.v. injected with PS, P-D2-PS, and P-D2-PEG5-PS covalently labeled with DyLight 680. Peak uptake PS by DCs is suggested to occur at 24 h post i.v. injection; accordingly, spleen and aorta were harvested after 24 hours and analyzed. The biodistribution of the nanocarriers was assessed via an IVIS optical imaging system, revealing the DyLight 680-labeled P-D2-PEG5-PS to accumulate in the pathological lesions and spleen of atherosclerotic mice (FIG. 4A). Flow cytometric analysis indicated that P-D2-PEG5-PS targeted a significantly higher percentage of atheroma-resident DCs than PS and P-D2-PS (FIG. 4B). Moreover, P-D2-PEG5-PS displayed a markedly increased association with DCs than any other cell population in aorta, including macrophages and monocytes, which are the predominant immune cells within the atherosclerotic lesions (FIG. 4C, FIG. 13A). In addition, a significantly higher percentage of P-D2-PEG5-PS⁺ DCs was also observed in spleen of ApoE^{-/-} mice compared to PS⁺ DCs (FIG. 4D). P-D2-PEG5-PS also showed to target significantly higher percentages of DCs than macrophages (p<0.01) and all other isolated cell populations in spleen, including CD45− cells (p<0.001) (FIG. 13B,C).

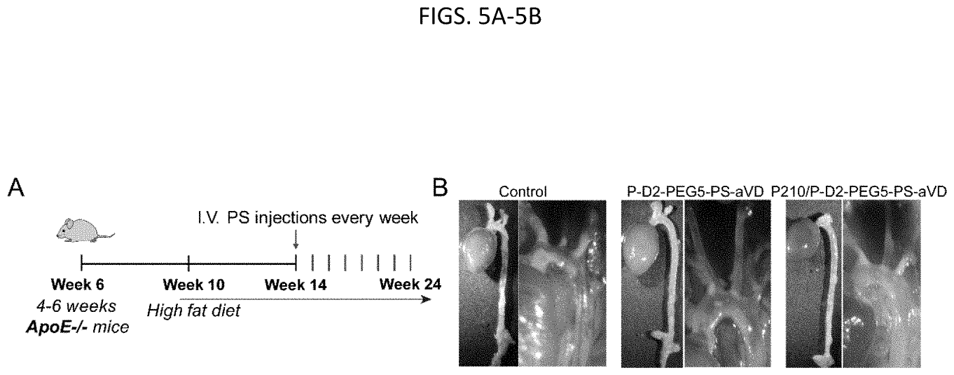

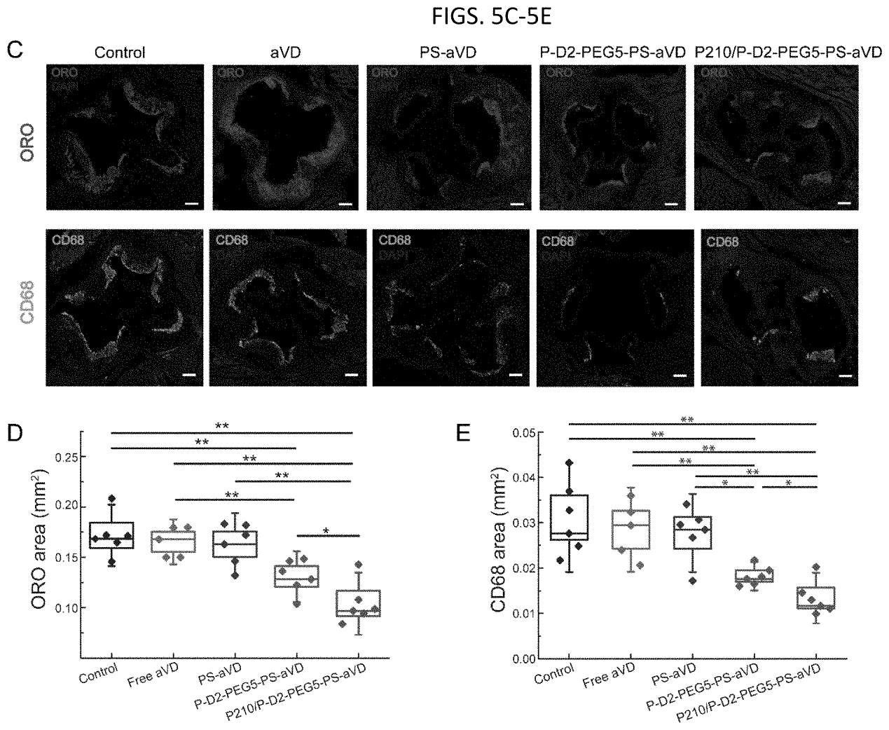

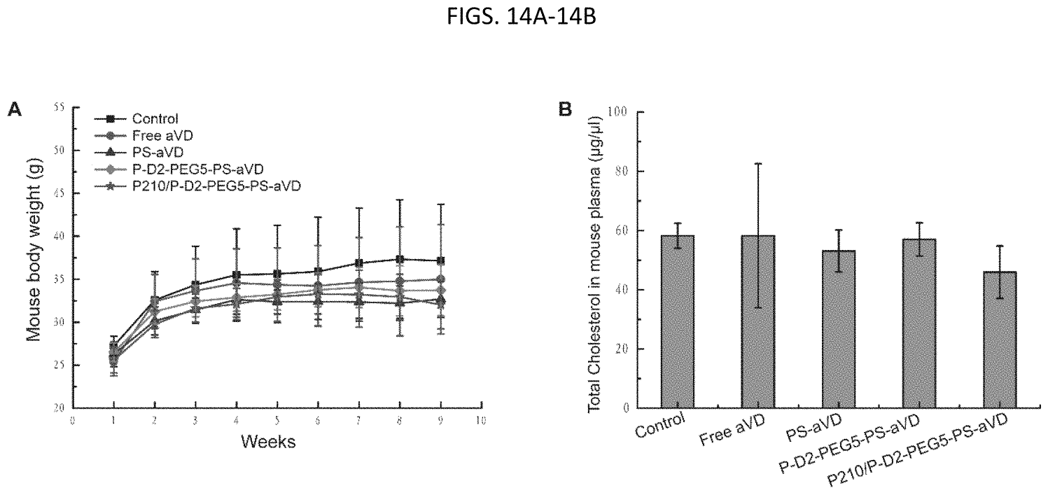

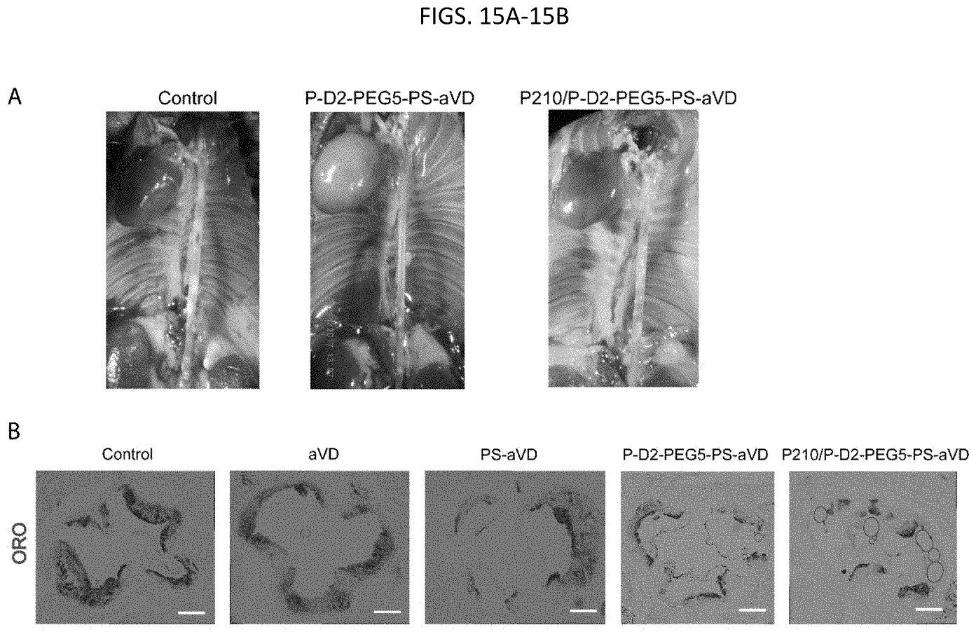

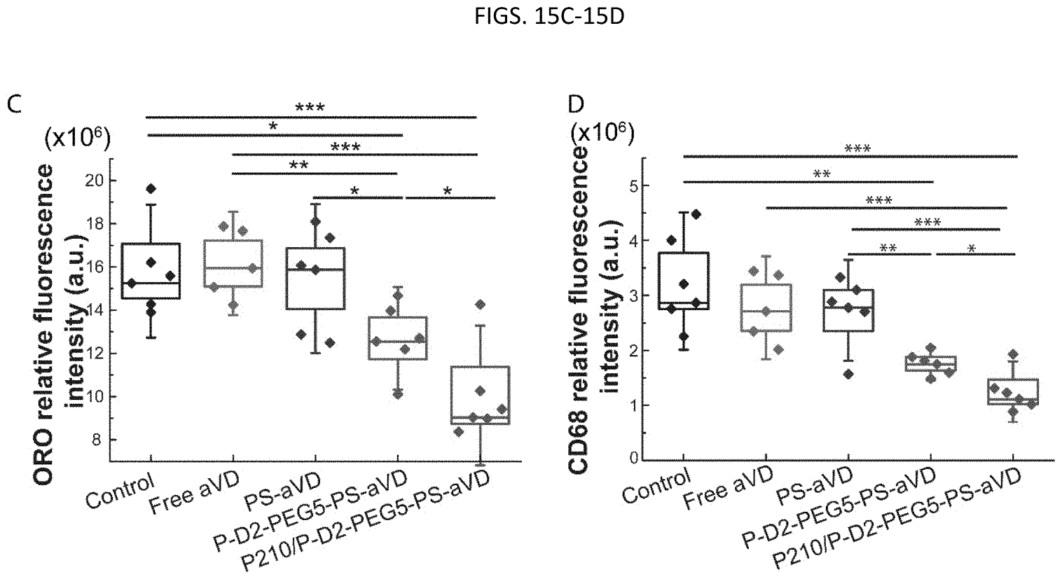

Delivery of P210 to DCs via aVD loaded and P-D2 decorated PS reduces atherosclerosis in ApoE^{-/-} mice—To evaluate the therapeutic potential of the delivery system, 8-10 week old male ApoE^{-/-} mice received a high-fat diet for 4 weeks to establish vascular lesions. The mice were then injected i.v. once per week with one of four therapies: i) free aVD (100 ng aVD/injection); ii) aVD loaded PS (PS-aVD) (100 ng aVD/1.5 mg polymer/injection); iii) aVD loaded and P-D2 decorated PS (P-D2-PEG5-PS-aVD) (100 ng aVD/1.5 mg polymer/injection); iv) aVD and P210 loaded and P-D2 decorated PS (P210/P-D2-PEG5-PS-aVD) (12.5m P210/100 ng aVD/1.5 mg polymer/injection) (FIG. 5A). After 8 weeks of administration, the mice were euthanized. Body weights and mouse activities were monitored every week (FIG. 14A). No significant changes in total cholesterol were found between the 4 groups (FIG. 14B). Different from control mice, the number and size of atherosclerotic lesions, which appeared white, in aorta (i.e. arch of aorta, brachiocephalic arteries, and descending aorta) were clearly reduced in P-D2-PEG5-PS-aVD and P210/P-D2-PEG5-PS-aVD treated mice (FIG. 5B, FIG. 15A). The atherosclerotic lesions from aortic root to arch of aorta were quantitatively analyzed in serial sections stained with Oil Red 0 (ORO), which is a marker of lipid accumulation. A repeatable, unbiased, and quantitative method was developed to analyze the over 200 images by image-processing software written in Python (FIG. 16). The histological analysis also demonstrated that the atherosclerotic lesion formation was significantly reduced in the P210/P-D2-PEG5-PS-aVD treated group compared with control group (p<0.01), aVD group (p<0.01), PS-aVD group (p<0.01), and P-D2-PEG5-PS-aVD group (p<0.05) (FIG. 5C,D, FIG. 15B,C). Macrophages are predominant in symptomatic plaques and can be identified by CD68. The macrophage presence in the aorta was therefore quantified using CD68 immunostaining, which revealed that P-D2-PEG5-PS-aVD and P210/P-D2-PEG5-PS-aVD dramatically decreased macrophage accumulation by 41% (p<0.01) and 57% (p<0.01) on average compared with control mice, by 36% (p<0.01) and 53% (p<0.01) compared with free aVD, and by 34% (p<0.05) and 52% (p<0.01) compared to the PS-aVD group (FIG. 5C,E, FIG. 15D). Additionally, macrophage content in plaques reduced markedly in P210/P-D2-PEG5-PS-aVD treatment group compared to P-D2-PEG5-PS-aVD group (p<0.05) (FIG. 5C,E, FIG. 15D).

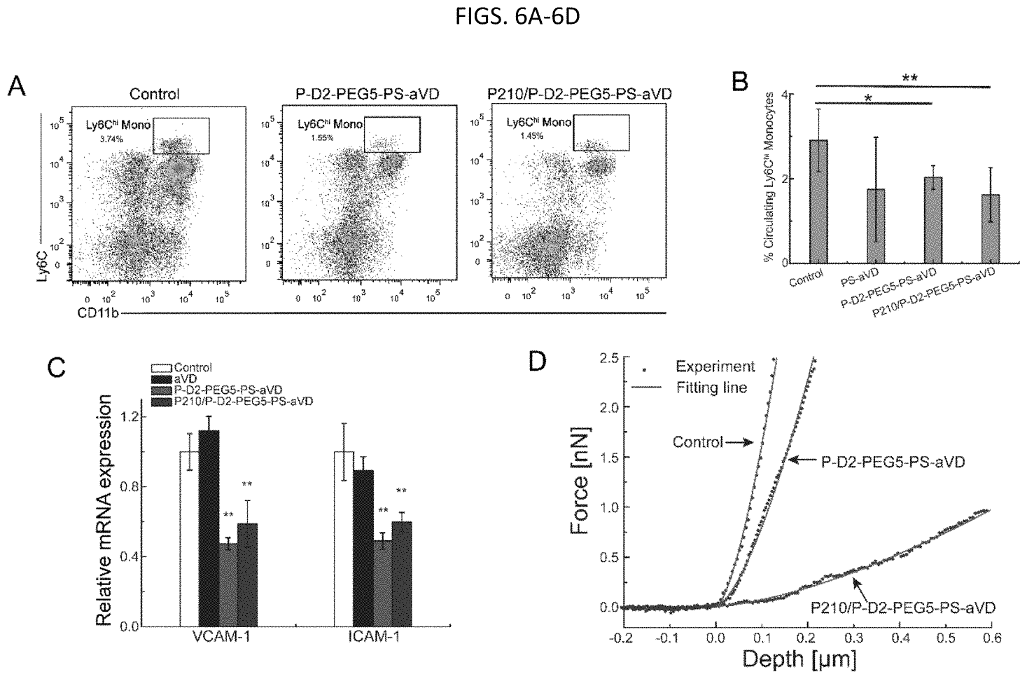

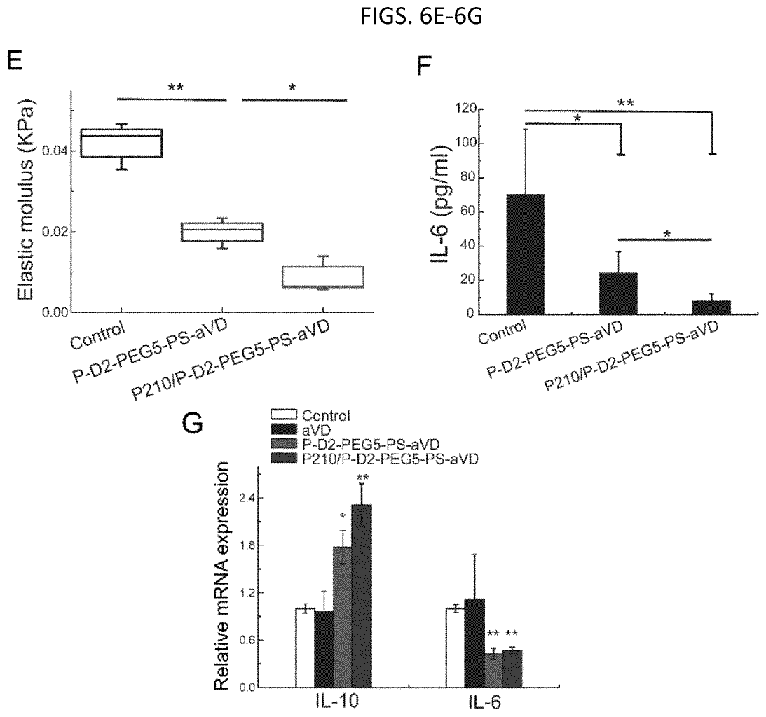

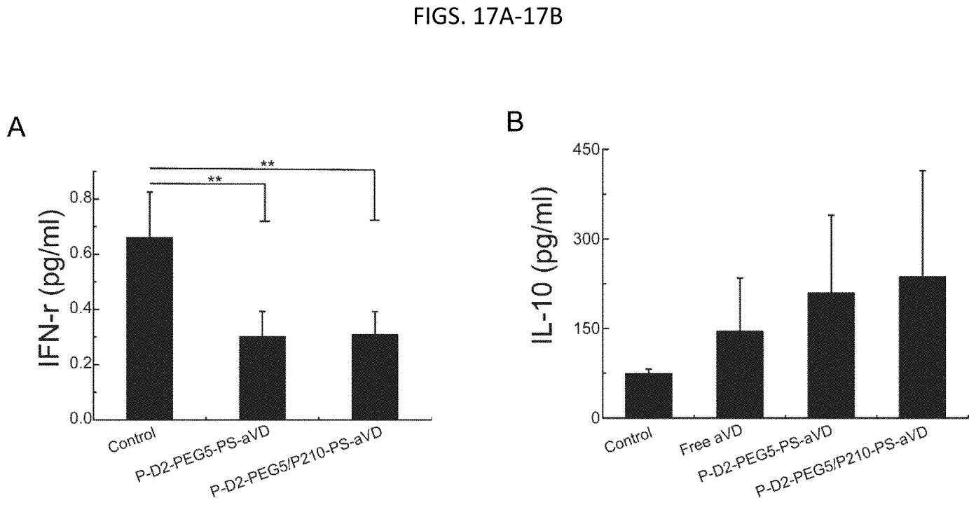

Combined intracellular delivery of aVD and P210 via optimized DC-targeted PS markedly decreases arterial stiffness and inflammation in ApoE^{-/-} A mice—The progressive cell infiltration and structural changes in aortic arteries, especially remodeling and content changes in the ECM, could ultimately lead to rupture of atherosclerotic plaques and subsequent vascular occlusion in humans. Circulating Ly6C^{hi} monocytes are preferentially recruited into atheroma, after which many differentiate into lipid-laden macrophages (foam cells) mediated by adhesion molecules and proinflammatory cytokines. Ly6C^{hi} monocytes, therefore, crucially determines the inflammatory responses in atherosclerosis by fueling lesion cellularity. Flow cytometric analysis showed that both P-D2-PEG5-PS-aVD and P210/P-D2-PEG5-PS-aVD reduced blood Ly6C^{hi} monocytes significantly compared with the control group (FIG. 6A,B). Reduced mRNA expression of adhesion molecules VCAM-1 and ICAM-1 in the artery wall may have contributed to the decreased migration of inflammatory monocytes after P-D2-PEG5-PS-aVD and P210/P-D2-PEG5-PS-aVD treatment (FIG. 6C). Arterial stiffness is a major risk factor for vulnerable plaque rupture, as it has been associated with ECM remodeling, dedifferentiation of vascular smooth muscle cells (SMCs), and systemic and vascular inflammation. Atomic Force Microscopy (AFM) has been extensively used to measure the mechanical properties of living cells and tissue and has been previously employed to assess arterial stiffness in ApoE^{-/-} mice. To further determine the therapeutic effects between P-D2-PEG5-PS-aVD and P210/P-D2-PEG5-PS-aVD treatment on aortic structure, the stiffness of the aortic arch was evaluated ex vivo using AFM in contact mode. As shown in FIG. 6D-E, the Young's modulus of the aortic arch in P210/P-D2-PEG5-PS-aVD was 8.7±4.6 KPa, which was −2.3 fold lower than that in P-D2-PEG5-PS-aVD treated mice (E=20±3.8 KPa, p<0.05) and ˜4.8 fold lower than that in control mice (E=41.9±5.8 KPa, p=0.001). Next, serum cytokine profiles were measured after 8 weeks of treatment, and it was found that IL-6 was significantly decreased in the serum of P210/P-D2-PEG5-PS-aVD treated mice compared to P-D2-PEG5-PS-aVD (p<0.05) and control mice (p<0.01) (FIG. 6F). IFN-γ (p<0.01) was also significantly decreased while IL-10 was elevated in the serum of P210/P-D2-PEG5-PS-aVD and P-D2-PEG5-PS-aVD treated mice compared to control mice (FIG. 17A,B). In addition, the expression of mRNA for anti-inflammatory cytokine IL-10 was elevated significantly, whereas Th1 pro-inflammatory cytokine IL-6 was strikingly reduced in plaques of P210/P-D2-PEG5-PS-aVD treated mice (FIG. 6G). Taken together, these data suggest that the combined delivery of atherosclerotic antigen with aVD enhanced therapeutic decreases in arterial stiffness and chronic inflammation.

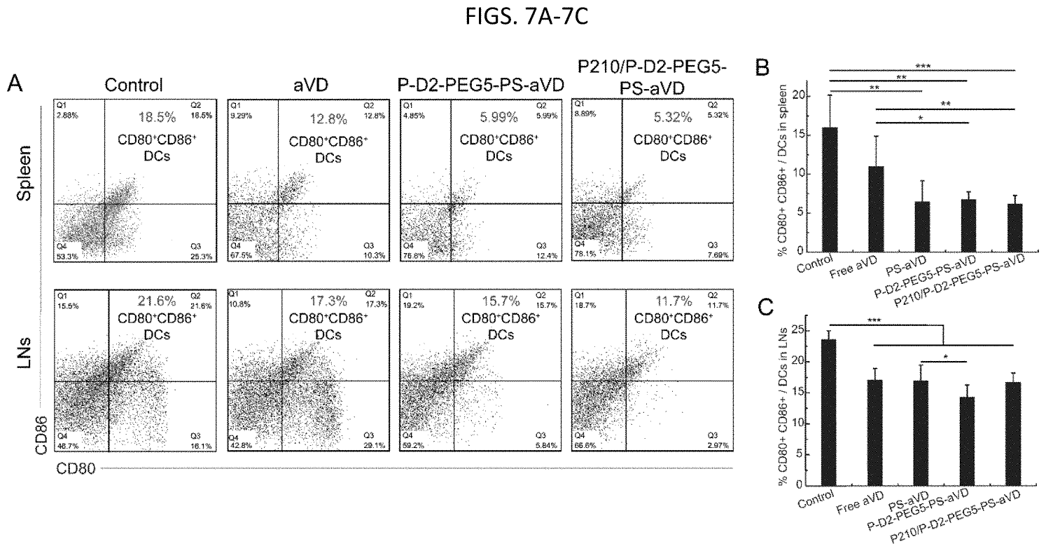

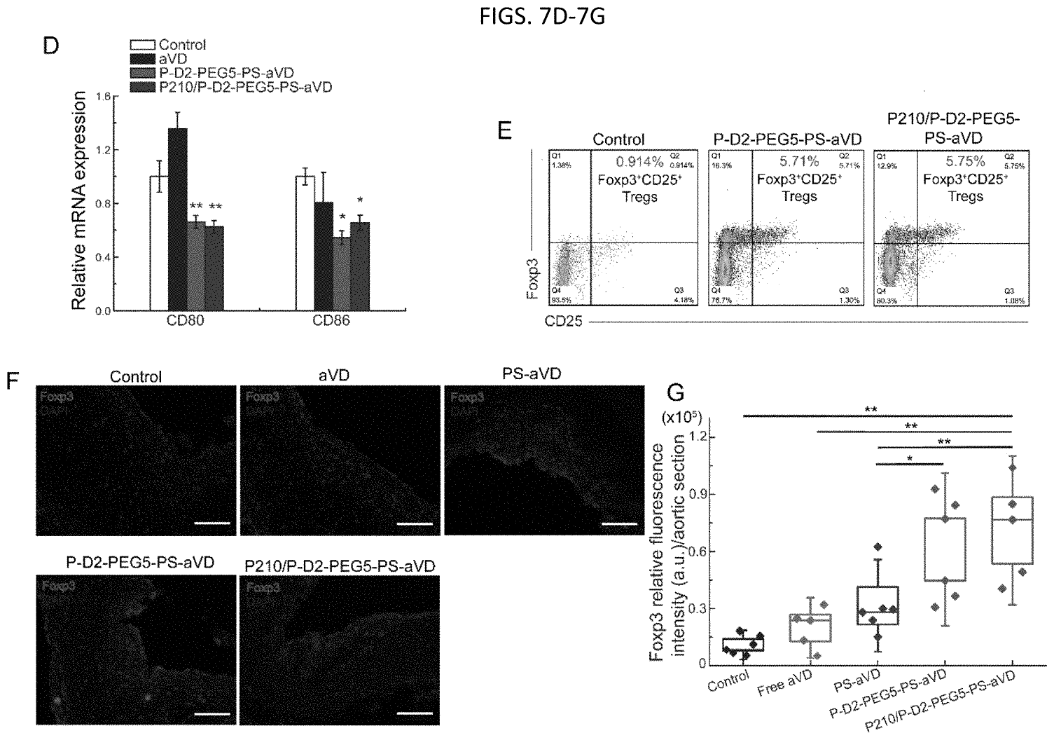

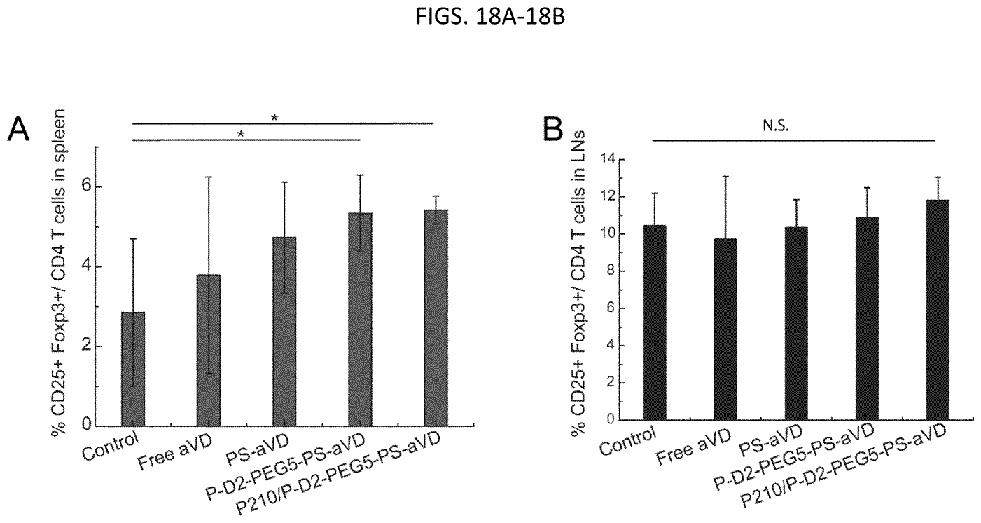

Targeted anti-inflammatory PS inhibit DC maturation and elicit Treg responses—DC-mediated antigen presentation has been suggested to occur within atherosclerotic lesions and in peripheral lymphoid organs, where T cells migrate back to lesions to manipulate local immune responses. While mature DCs can stimulate naïve T cells and initiate antigen-specific immune responses, immature DCs tend to mediate tolerance. Given that P-D2-PEG5-PS-aVD inhibited maturation of BMDCs in vitro, it was hypothesized that the disclosed DC targeting nanocarriers may induce atheroprotective regulatory T cell responses. In the spleen of ApoE^{-/-} mice, significant decreases in CD80⁺CD86⁺ mature DCs in CD11c⁺ populations from both P210/P-D2-PEG5-PS-aVD and P-D2-PEG5-PS-aVD groups were found as compared with control group (p<0.001, p<0.01) and free aVD group (p<0.01, p<0.05) (FIG. 7A-B). In draining lymph nodes (DLNs), it was also observed much lower numbers of CD80⁺CD86⁺ mature DCs from P210/P-D2-PEG5-PS-aVD (p<0.001) and P-D2-PEG5-PS-aVD (p<0.001) groups as compared with control group (FIG. 7A,C). Among those, the P-D2-PEG5-PS-aVD group showed a significantly lower percentage of CD80⁺CD86⁺ CD11c⁺ cells in the DLNs than PS-aVD group (p<0.05) (FIG. 7C). In aortic lesions, P210/P-D2-PEG5-PS-aVD and P-D2-PEG5-PS-aVD treated mice revealed significant decreases in the expression of DC maturation marker CD80 and CD86 genes compared to free aVD (p<0.01 on CD80, p<0.05 on CD86) and control groups (p<0.001 on CD80, p<0.001 on CD86), as determined by quantitative RT-PCR (FIG. 7D). Immature DCs have been shown to induce Foxp3⁺ Treg cells, which could counterbalance pro-inflammatory effector T cells in both mice and humans. Foxp3⁺ Tregs in lymphoid organs and atherosclerotic plaques were evaluated by flow cytometry and immunohistochemistry. The number of Foxp3⁺ CD25⁺ Treg cells was increased significantly in CD4+ T cell populations in the spleen for the P210/P-D2-PEG5-PS-aVD (p<0.05) and P-D2-PEG5-PS-aVD groups (p<0.05) compared to control (FIG. 7E, FIG. 18A), while no statistical difference was observed in DLNs (FIG. 15B). The immunohistochemical studies revealed that the levels of Foxp3⁺ Tregs in the P210/P-D2-PEG5-PS-aVD group significantly increased by 6.5-fold (p=0.004) compared with the control group, 3.6 fold (p=0.008) compared with the free aVD group, and 2.3 fold (p=0.017) compared with PS-aVD (FIG. 7F, G). Taken together, these data indicated that the reduced systemic Th1 responses and local inflammation were in part due to the suppression of DC maturation, which resulted in the induction of atheroprotective Tregs that were recruited to atheroma.

Discussion—Chronic inflammation has been well established as an essential contributing factor to the progression of atherosclerosis, but, despite numerous past and ongoing clinical trials, it has yet to be established as a viable therapeutic target. Current therapeutic interventions for the prevention and treatment of cardiovascular disease focus on lowering serum cholesterol levels, primarily via hydroxymethyl glutaryl coenzyme A (HMG-CoA) reductase inhibitors. High-dose statin therapy may have pleiotropic properties that include reductions in vascular plaque inflammation. Although this anti-inflammatory effect may contribute to statin efficacy, the direct anti-inflammatory effects of statins on atherosclerosis are not fully understood or validated. Importantly, statins are not effective for all patients and frequently result in statin associated muscle symptoms, diabetes mellitus, central nervous system complaints, and other possible side effects. A focused anti-inflammatory treatment may therefore present a viable alternative for the prevention of cardiovascular disease. To address this issue, an immunomodulatory and anti-inflammatory nanocarrier platform is described herein that specifically targets atheroma-resident and splenic DCs (FIG. 4). It is demonstrated herein that low dosage intracellular targeting of aVD via these PS was critical for the induction of tolerogenic DCs, which promotes the differentiation of Foxp3⁺ Tregs, suppresses atherosclerotic inflammation and reduces vascular stiffness (FIGS. 5-7).

Conjugation of targeting ligands, like antibodies and peptides, onto the surfaces of nanocarriers may be used to improve specificity for target cell populations. Both biophysical modeling and cell uptake experiments have demonstrated the complexity of this process, which requires careful consideration of the interface between the surface and the target cell membrane. Self-assembled nanocarriers possess dense hydrophilic coronas that both stabilize the aggregate structure and minimize non-specific protein adsorption. The binding site for the targeting ligand must therefore be at an appropriate distance from this corona in order to efficiently bind its target. Additionally, receptor mediated endocytosis requires a sufficient number of receptor engagements with the ligand to thermodynamically favor changes in curvature required for membrane wrapping and receptor clustering. Thus, an optimal nanocarrier shape, size, aspect ratio, and surface density of the targeting ligand exist to promote endocytosis by specific cell populations, which can differ in receptor expression level and membrane diffusivity and flexibility. In this study, an optimal nanostructure for targeting DCs was synergized with an engineered surface chemistry to further enhance cellular targeting. A simple and controllable approach is described herein to optimize the display of targeting moieties on nanocarrier surfaces using constructs formed with standard Fmoc chemistry. The constructs were efficiently and reproducibly anchored into PS bilayers, allowing the rapid optimization of peptide orientation and surface density for enhanced uptake by specific cell populations. More than 3-fold enhancement in uptake by DCs was achieved both in vitro and in vivo. This methodology may allow the rational engineering of nanocarrier specificity for almost any cell type and can be further improved through the use of multiple targeting peptides, each potentially with different surface densities and degrees of freedom for receptor interactions.

Previous studies have used aVD to inhibit atherosclerotic plaque formation, but immunosuppressive effects were only shown at higher total aVD doses than were required for the disclosed targeted PEG-bl-PPS PS. In a seminal example, Takeda et al. administered aVD orally to mice at a total dose of 4800 ng over the course of 12 weeks to achieve a 39% decrease in plaque area and a 29% reduction in macrophage accumulation (Takeda, M., et al., Oral Administration of an Active Form of Vitamin D<sub>3</sub> (Calcitriol) Decreases Atherosclerosis in Mice by Inducing Regulatory T Cells and Immature Dendritic Cells With Tolerogenic Functions. Arteriosclerosis, Thrombosis, and Vascular Biology, 2010. 30(12): p. 2495-2503). Here it is shown that i.v. administration of P210/P-D2-PEG5-PS-aVD at a total aVD dose of only 800 ng over a period of 8 weeks significantly reduced plaque area by 40% and macrophage content by 57% in high fat diet fed ApoE^{-/-} mice. In comparison, no or limited improvements were detected at the same dosage respectively for the free aVD and PS-aVD treatment groups. The instability of aVD and the broad tissue distribution of VDR may account for prior disappointing therapeutic effects or contradictory results of aVD treatment in the clinic, both of which may be overcome by nanocarriers engineered for targeted delivery to critical immune cell populations.

Tolerogenic DCs with low surface expression of co-stimulatory molecules, reduced expression of Th1-biased cytokines like IL-12p70, and enhanced production of tolerogenic cytokine IL-10 can suppress allogeneic T cells while inducing the generation of regulatory T cells. Tolerogenic DCs have therefore been linked to the treatment of chronic inflammatory conditions. P-D2-PEG5-PS-aVD significantly increased the presence of tolerogenic DCs (CD80/CD86 low) in both the atherosclerotic lesion and spleen compared to free aVD treated and control groups. Significant increases in Foxp3⁺ Treg activation in the aorta and spleen of atherosclerotic mice are also shown herein. Foxp3⁺ Tregs have been demonstrated for the suppression of Th1 immune responses in atherosclerosis, and their induction is highly dependent on DC interactions and activation state. The results presented herein suggest that the therapeutic efficacy of the disclosed DC modulating platform was attributed to cell-mediated anti-inflammatory mechanisms that was enhanced by optimized targeting of DCs.

Complex procedures, side effects and costs associated with the methods for ex vivo modulation of DCs remain considerable challenges. The combination of P210 antigen with the DC modulating platform disclosed herein showed more robust therapeutic effects, including significantly reduced lesion areas and macrophage content. Moreover, the data presented herein demonstrated decreased aortic stiffness as determined by AFM, which was consistent with a studies that indicate arterial softening to be causal for attenuated atherosclerosis. Inflammation has been linked to changes in arterial wall stiffness and extracellular matrix, and notably IL-6 can induce endothelial dysfunction and regulate macrophage differentiation and activation in the aorta for reduced arterial stiffness.

The described DC modulating platform demonstrated specific targeting capacity to DCs and robust immunomodulatory effects in vitro and in vivo, including decreased levels of inflammatory cytokines, increased expression of tolerogenic cytokines, enhanced Treg activation and reduced vascular stiffness. The platform achieves this selectivity by combining NSET [Yi, S., et al., Tailoring Nanostructure Morphology for Enhanced Targeting of Dendritic Cells in Atherosclerosis. ACS Nano, 2016. 10(12): p. 11290-11303, incorporated entirely herein by reference] with an optimized surface display of a targeting ligand, and this combined targeting approach may find utility in a wide range of clinically translatable applications. While in this work aVD and an ApoB-100 peptide were co-delivered to validate the specific atheroprotective anti-inflammatory role of DCs, this platform can support the stable loading and transport of a wide range of additional therapeutic combinations [Allen, S., et al., Facile assembly and loading of theranostic polymersomes via multi-impingement flash nanoprecipitation. J Control Release, 2017. 262: p. 91-103, incorporated herein by reference in its entirety] or be tailored for the targeting of alternative cell populations. Such selective in situ modulation of cell function can allow the probing of the pathological roles of specific cell subsets as well as decrease the effective dosage of a wide range of therapeutics.

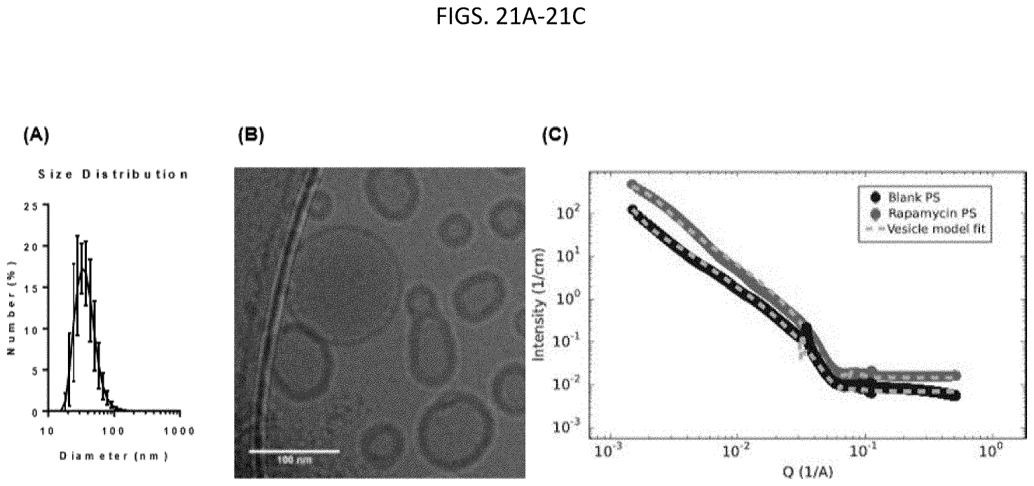

In vivo delivery of low-dose rapamycin-loaded polymersomes prevents rejection of allogenic islet transplantation—Polymersomes were loaded with rapamycin and characterized for size distribution, cryogenic transmission, and small angle x-ray scattering (SAXS). Results are shown in FIG. 21A-C, respectively. Mean diameter of the polymersomes was found to be 91.66 nm with a poly-dispersity index (PDI) of 0.21 (A). CryoTEM and SAXS confirm vesicular morphology (B). Via HPLC rapamycin concentration in rapamycin-loaded polymersomes solution was found to be 0.085 mg/ml, indicating an encapsulation efficiency of 60 to 65% (C).

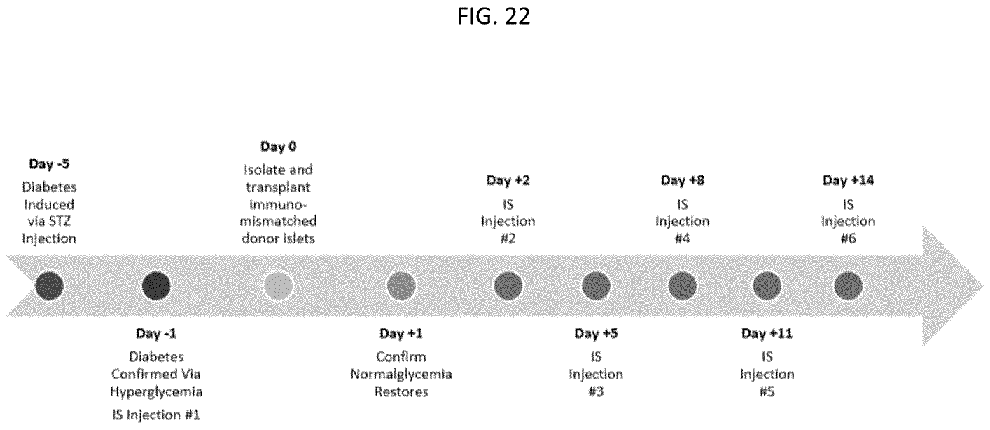

The experimental overview for in vivo allogenic islet transplantation is shown in FIGS. 22. 8 to 14-week-old C57BL/6 mice were induced with type I diabetes via intraperitoneal injection of streptozin (STZ) (190 mg per kg body weight) 5 days prior to islet transplantation. Diabetes was confirmed via hyperglycemia. On the day of transplantation, donor islets were isolated from immunomismatched 8 to 12-week-old Balb/c mice. Islets were transplanted into diabetic C57BL/6 recipients. Two donors were used per recipient (˜200 mouse islets, ˜175 IEQ). Recipient mice were given subcutaneous injections of either free rapamycin solubilized in 0.2% carboxymethylcellulose solution or rapamycin-loaded polymersomes. For the low-dose animals, injections were given every 3 days at a dose of 1 mg per kg body weight starting the day prior to transplantation and terminated 14 days after transplantation (6 injections total). For the high-dose animals, animals were given the day prior to transplant, the day of transplant, and then for 0 days post-transplantation at a dose of 1 mg per kg body weight (11 injections total). Blood glucose and body weight were monitored regularly. One-month post transplantation, an interperitoneally tolerance test was performed to determine islet function. Mice were fasted overnight. Glucose was injected interperitoneally (2 g per kg body weight) and blood glucose concentration was measured as regular intervals.

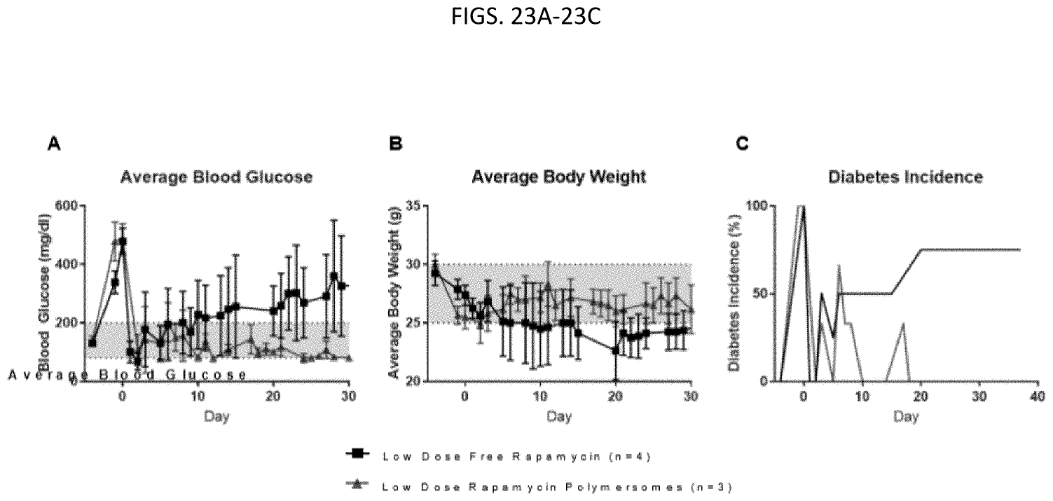

Rapamycin-loaded polymersomes were shown to prevent islet transplantation rejection (FIG. 23A-C). All recipients (3 of 3) treated with low-dose rapamycin-loaded polymersomes experienced sustained normoglycemia and maintained body weight after transplantation, whereas all but one (3 of 4) of the recipients treated with low-dose free rapamycin rejected the islet transplantation prior to day 20, as indicated by the return of hyperglycemia and weight loss.

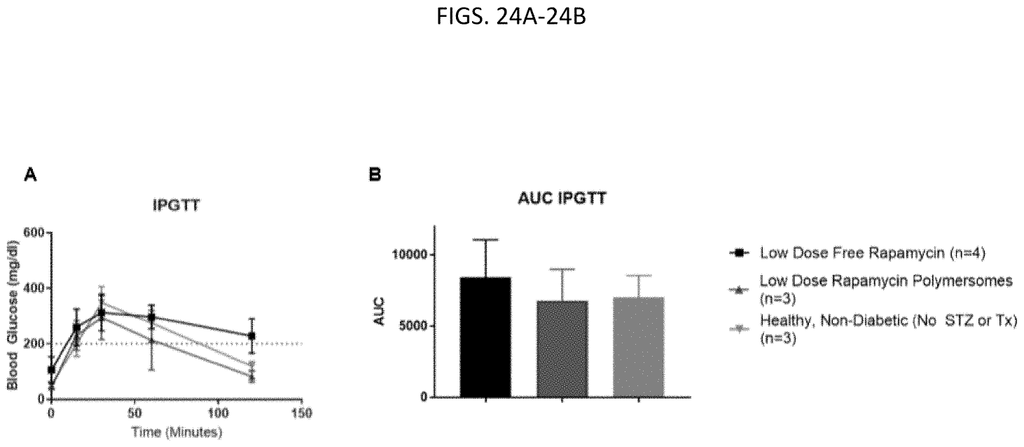

Recipients treated with low dose rapamycin-loaded polymersomes were shown to have improved islet function over those treated with low lose free rapamycin (FIG. 24A-C). Recipients treated with low dose free rapamycin fail to restore normalglycemia after glucose challenge, while recipients treated with low-dose rapamycin-loaded polymersomes show restoration of normalglycemia consistent of that of a non-diabetic animal.

### Example 2

Materials

Unless specified below, all chemicals for polymer synthesis were purchased from Sigma Aldrich (St. Louis, Mo., USA) and all reagents for flow cytometry were purchased from BioLegend (San Diego, Calif., USA).

Animals—Ldlr−/− female mice with a C57Bl/6 background, 4-5 weeks old, were purchased from Jackson Laboratories. All mice were housed and maintained in the Center for Comparative Medicine at Northwestern University. All animal experimental procedures were performed according to protocols approved by the Northwestern University Institutional Animal Care and Use Committee (IACUC). Mice were fed a normal diet until they were 2-3 months old, at which point they were switched to a high-fat diet (Tekklad TD 88137 42% calories from fat). Mice were fed a high-fat diet for 3 months prior to the beginning of treatment.

Polymer synthesis—Poly(ethylene glycol)-block-poly(propylene sulphide) (PEG-b-PPS) was synthesized as described in Example 1. Briefly, commercially available methyl ether PEG (Mₙ 2000) was functionalized with mesylate and subsequently thioacetate groups. Base-deprotection of the PEG-thioacetate afforded a thiolate anion, which was used to perform living ring-opening polymerization of 20 molar equivalents of propylene sulfide, which was end-capped with benzyl bromide (polymer structure schematic in FIG. 25A).

Nanocarrier formulation—PEG-b-PPS micelles were formed via thin film rehydration. 15 mg of PEG45-b-PPS₂₀-Benzyl was weighed into a glass HPLC vial (Thermo Fisher). If the formulation was to contain celastrol, celastrol was added to the vial at this point from a stock solution of 1 mg/mL in tetrahydrofuran (THF). The mixture was dissolved in 1 mL of THF and was left in a vacuum desiccator overnight to remove the THF and coat the walls of the vial in polymer. After desiccation, 1 mL of sterile phosphate buffered saline (1×PBS) was added to each vial. Vials were then shaken for 2 hours at 1000 rpm. Formulations were used immediately or were stored at 4° C.

Nanocarrier characterization—For cryogenic transmission electron microscopy, 4-5 μL of each formulation was applied to a 400-mesh lacy carbon copper grid. Specimens were then plunge-frozen with a Gatan Cryoplunge freezer. These specimens were imaged using a JEOL 3200FS transmission electron microscope operating at 300 keV at 4000× nominal magnification. All images were collected in vitreous ice using a total dose of ˜10 e⁻Å⁻² and a nominal defocus range of 2.0-5.0 μm.

Small angle X-ray scattering (SAXS) studies were performed at the DuPont-Northwestern-Dow Collaborative Access Team (DND-CAT) beamline at Argonne National Laboratory's Advanced Photon Source (Argonne, Ill., USA) with 10 keV (wavelength λ=1.24 Å) collimated X-rays. SAXS was performed on undiluted 15 mg/mL polymer formulations, as described previously. Model fitting was performed using SASView and the built-in polymer micelle model.

Dynamic light scattering measurements (DLS) were performed on 15 μg/mL polymer formulations using a Nano 300 ZS zetasizer (Malvern Panalytical, Malvern, UK), using the number average distribution for calculation of the mean diameter and polydispersity of the formulations.

Celastrol quantification—Celastrol solubility, encapsulation efficiency, and loading capacity were all assessed using high performance liquid chromatography (HPLC) using a Thermo Scientific C18 reverse phase column, with dimethylformamide (DMF) as the mobile phase at 0.5 mL/min. Area under the curve quantification of celastrol absorbance at 280 nm was performed using Thermo Fisher Chromeleon 7 software. A celastrol standard curve was constructed, with good linearity between celastrol concentrations of 2 mg/mL to 12.5 μg/mL, with 6.25 μg/mL being too close to the limit of detection for inclusion in the standard curve.

To determine the loading capacity of celastrol in micelles, defined here as the highest achievable mass of celastrol that can be stably loaded into 1 mg of micelles in 100 μL of 1×PBS, 1 mg of celastrol was added to 1 mg of PEG45-b-PPS₂₀-Benzyl polymer in 500 uL THF in an HPLC vial. THF was removed by vacuum desiccation and micelles were formed via thin film rehydration with 100 μL of 1×PBS. After micelles were formed, the solution was divided in two, with one half being purified via LH-20 lipophilic column filtration to remove unencapsulated celastrol and the other half being left as is. Both samples (column filtered and unfiltered) were then lyophilized and redissolved in 200 μL DMF and celastrol content was quantified via HPLC.

To determine the encapsulation efficiency of celastrol in micelles, defined as the percentage of the originally added celastrol mass that is stably encapsulated in micelles after filtration to remove unencapsulated celastrol, micelles were formed as described above with variable amounts of celastrol, and were filtered using an LH-20 column. Filtered micelles were lyophilized and redissolved in 200 μL DMF and celastrol content was quantified via HPLC.

To determine the solubility of celastrol in aqueous buffer, 1 mg of celastrol was added to a glass vial along with 10 mL of 1×PBS. This solution was heated to 37 C and was stirred using a magnetic stir bar for 1 hour. The PBS solution was centrifuged at 15,000 RCF to pellet insoluble celastrol aggregates and was subsequently lyophilized. The lyophilized powder was resuspended in 200 μL DMF and celastrol was quantified via HPLC.

Celastrol release from micelles into 1×PBS with or without oxidative trigger was determined as follows. Celastrol micelle formulations (500 μL) into Slide-A-Lyzer 10K MWCO MINI dialysis tubes (15 mL tubes, ThermoFisher Scientific) with 13 mL 1×PBS. To each formulation was added either 100 μL of 500 μM H₂O₂ in water (Sigma Aldrich) or 100 uL of water. Tubes were placed on a shaker (250 rpm) and 100 μL of celastrol micelle formulations were taken for absorbance readings and were placed back into the tubes after readings were completed. Absorbance at 424 nm, an absorbance peak for celastrol, were taken using a SpectraMax M3 plate reader using a 96-well plate.

Confocal microscopy—Cel-MC were formed, as described above, using 10 mg polymer, 10 μg celastrol, and 10 μg DiI, a lipophilic dye, rehydrated in 1 mL of 1×PBS. RAW 264.7 cells were added to an 8-chamber coverslip-bottom slide at 20,000 cells per chamber. Cells were either left untreated or were treated with 1 mg/mL micelles (1 μg/mL celastrol) overnight. Cells were then washed twice with 1×PBS and returned to complete media for an additional 24 hours. Cells were incubated with 100 nM LysoTracker Green DND-26 (ThermoFisher Scientific) and 8 μM Hoechst 33342 (ThermoFisher Scientific) in 1×PBS for 30 minutes prior to being washed twice and returned to complete media. Cells were then imaged using an SP5 Leica confocal microscope at 63× objective magnification. Hoechst nuclear staining was detected using a 405 nm laser with emission detected using a HyD detector set to a 440/470 band. Lysotracker Green was detected using a 488 nm laser and a HyD detector set to a 500/530 band. DiI was detected using a 561 nm laser and a HyD detector set to a 570/630 band.

Inhibition assays—NF-κB inhibition by celastrol was assayed using RAW Blue cells (Invivogen), a stably transfected cell line derived from RAW 264.7 macrophage-like cells, which contain the gene for secreted alkaline phosphatase (SEAP) downstream of the NF-κB promoter. Cells were seeded into a 96 well plate at 50,000 cells per well. NF-κB signalling was induced using 100 ng/mL LPS, with celastrol-loaded micelles and free celastrol (0.1% THF in 1×PBS vehicle) added to the cells concurrent with LPS administration. All micelle formulations contained the same amount of polymer (15 mg/mL) but were loaded with variable amounts of celastrol, and free celastrol formulations were prepared to match the concentration of loaded celastrol within Cel-MC formulations. Free celastrol formulation were made by diluting celastrol stock solutions in THF with 1×PBS to reach the appropriate celastrol concentration and 0.1% THF in 1×PBS. Cells were incubated for 16 hours, as per assay instructions, before supernatant was collected for quantification of SEAP activity, as described by the manufacturer. Colorimetric quantification of SEAP activity was performed on an M3 plate reader (SpectraMax) at an absorbance wavelength of 630 nm.

RAW 264.7 cells were plated in 24 well plates at 500,000 cells per well. TNF-α quantification was performed by treating cells with either 10 ng/mL or 1 μg/mL celastrol in either micelle-loaded or free form (in 0.1% THF/1×PBS) with 100 ng/mL LPS for 6 hours, along with positive control wells, in which LPS was added without celastrol. Supernatant was then collected and stored for ELISA quantification of TNF-α secretion (BioLegend), with TNF-α used to generate a standard curve.

Cytotoxicity assays—RAW 264.7 cells were plated into a 96 well black wall plates at 50,000 cells per well. Cells were then treated with Cel-MC or free celastrol, formulated as described above for the inhibition assays. After 16 h of incubation with free celastrol or Cel-MC formulations, cells were washed and incubated with 4 μM calcein-AM and 2 μM ethidium homodimer (Thermo Fisher), as described by the manufacturer. Readings were performed on an M3 plate reader, at excitation/emission wavelengths of 488/530 nm and 488/635 nm for calcein and ethidium homodimer, respectively. Readings were normalized as described by the manufacturer, accounting for background fluorescence and setting 100% viability for untreated cells and 0% viability for cells incubated with 100% methanol for 15 minutes.

RNAseq—RAW 264.7 cells were plated at 1×10⁶ cells per well of 6-well plates. Cells were treated with 100 ng/mL LPS to stimulate NF-κB signalling and were then treated in triplicate with one of the following: 1×PBS, 1 μg/mL celastrol in 0.1% THF/1×PBS, 1 ug/mL celastrol in 1 mg/mL micelle formulation in 1×PBS, 0.1% THF/1×PBS, or unloaded ‘blank’ micelles at 1 mg/mL in 1×PBS. Cells were treated for 2 or 6 hours to capture early and later transcriptional events. Cells were washed three times in 1×PBS before having their RNA extracted using a Qiagen RNeasy Mini Kit, as described by the manufacturer.

RNA samples were sent to Admera Health for RNA quality check using an Agilent Bioanalyzer 2100 Eukaryote Total RNA Pico Series II analysis. RNA samples that passed the quality check were used for library preparation (Lexogen QuantSeq 3′ mRNA-Seq) and were sequenced (Illumina Platform 2×150 6-10M PE reads per sample). The RNA-Seq data was aligned and processed using Lexogen QuantSeq data package. Differential gene and pathway analysis utilized DE-Seq2 (bioconductor.org/packages/release/bioc/html/DESeq2.html) and GSVA (bioconductor.org/packages/release/bioc/html/GSVA.html) using standard default parameters.

In vivo administration of nanocarriers—Four formulations were made for in vivo use: 15 mg/mL polymer blank micelle formulation, 15 mg/mL polymer 100 ng/mL celastrol micelle formulation, 200 ng/mL celastrol in a 1:1 DMSO:1×PBS formulation, and a vehicle control of 1:1 DMSO:1×PBS formulation. Both micelle formulations were injected intravenously (IV) via tail vein injection (100 μL per injection). The free celastrol and vehicle control formulations were injected intraperitoneally (IP) at 50 μL per injection. Injections were performed on high-fat diet mice (3 months on diet before the beginning of treatment) under isoflurane once a week for 18 weeks. Mice remained on high-fat diet for the duration of treatment. Mice were sacked one week after the end of treatment, and organs were harvested for flow cytometry or were mounted for histology.

Flow cytometric analysis of immune cell populations—Organs collected from mice were processed for flow cytometry. Blood was centrifuged to collect all blood cells. Red blood cells were subsequently lysed using ACK lysis buffer, resulting in a single cell suspension of blood immune cells. Spleens and lymph nodes were mechanically disrupted with a 70 μm nylon filter and a syringe plunger, to form a single cell suspension. Splenocytes were additionally treated with ACK lysis buffer to lyse red blood cells. The aortas were sliced into small pieces (˜1 mm²) and incubated at 37° C. at 300 rpm for 30 minutes in an enzyme cocktail to free cells: 125 U/mL collagenase XI, 60 U/mL hyaluronidase I-S, 60 U/mL DNase I (Roche), and 450 U/mL collagenase I in HBSS buffer. The aorta pieces and buffer were then strained and mechanically disrupted through a 70 μm nylon filter with a syringe plunger.

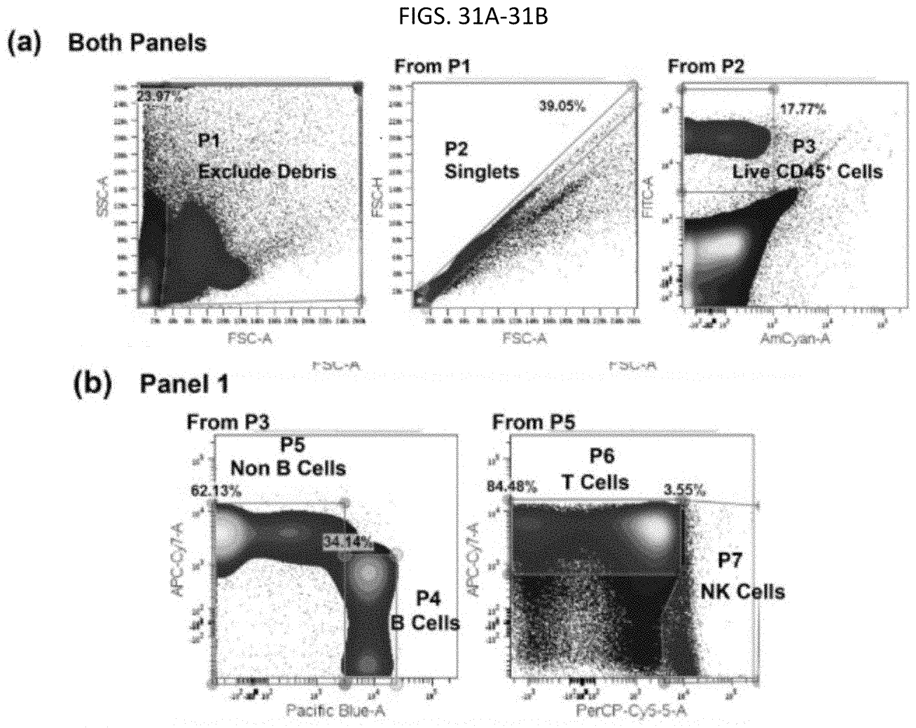

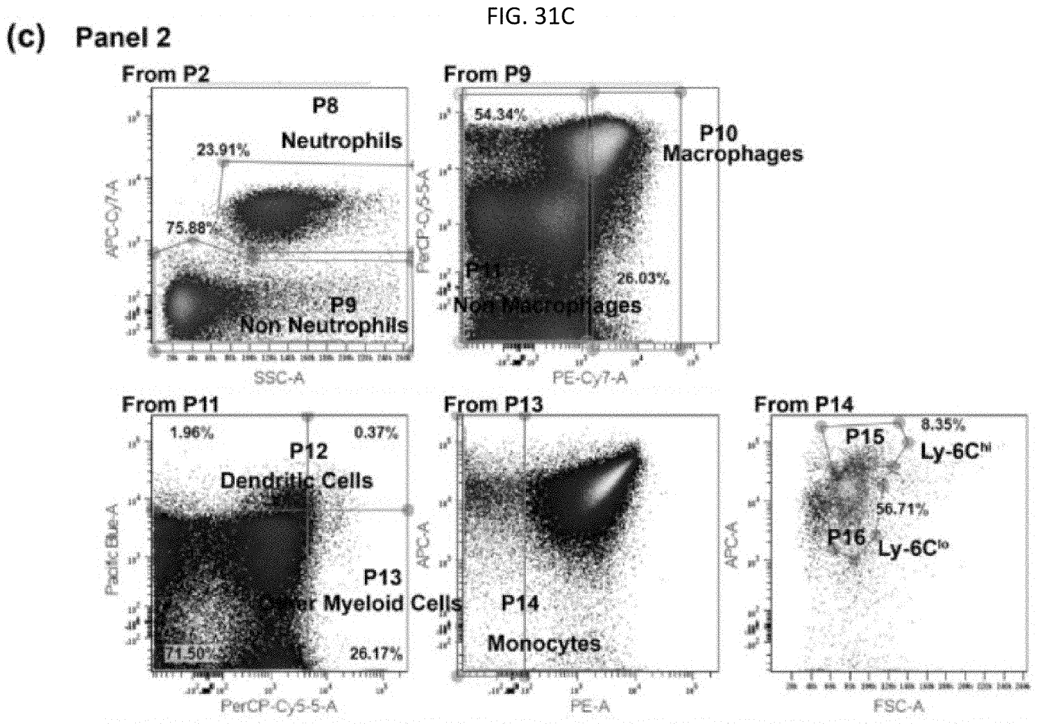

All single cell suspensions were then incubated for 15 minutes in a blocking buffer containing a fixable viability dye, Zombie Aqua, and an FcR blocking antibody anti-CD16/32. Cells were then stained with one of two antibody panels. Panel 1: FITC anti-CD45, APC/Cy7 anti-CD3, PE anti-CD4, APC anti-CD8, Pacific Blue anti-CD19, PerCP/Cy5.5 anti-NK1.1. Panel 2: FITC anti-CD45, PerCP/Cy5.5 anti-CD11b, Pacific Blue anti-CD11c, PE/Cy5 anti-I-A/I-E, PE/Cy7 anti-F4/80, PE CD86, APC anti-Ly6C, APC/Cy7 anti-Ly6G. Cells were washed, fixed, and analyzed using a BD LSR II. Data was analyzed using Cytobank online software. The gating strategy is available in the FIG. 31A-C.

Histological assessment of atherosclerotic plaques—Aortas were carefully dissected from mice to preserve vascular structure and were trimmed and embedded in optimal cutting temperature (OCT) compound for frozen tissue sectioning. Aortas were serially sectioned into 10 μm thick slices, 8-10 sections per slide. Aortic cross sections were stained with Oil Red 0 for fluorescence imaging. Images were taken on a Leica DM6B fluorescent microscope at 20× objective magnification with automated image stitching. Quantification of Oil Red O fluorescent staining was performed using a custom Python script.

Characterization of celastrol-loaded micelles—Micelles formed from PEG-b-PPS typically have a diameter of less than 50 nm and adopt a spherical morphology (FIG. 25A). Size and morphology of nanocarriers can drastically alter their organ and cell-level biodistribution after administration in vivo. Since the loading of cargo may alter the size and morphology of a nanocarrier, the present investigation aimed to confirm the structure of celastrol-loaded micelles (Cel-MC) as compared to unloaded micelles (Blank MC). Cel-MC formulations were prepared at a fixed concentration of polymer (15 mg/mL), but with increasing amounts of celastrol (1 ng/mL, 100 ng/mL, 10 μg/mL). Blank MCs and Cel-MCs share the same aggregate morphology, as demonstrated by the small, spherical micellar dots in the cryoTEM micrographs (FIG. 25B) that represent the hydrophobic poly(propylene sulfide) core. For additional corroboration, SAXS analysis of the formulations was performed and the data was subsequently fitted with a spherical polymer micelle model (FIG. 25C). The modelling parameters indicate a very slight increase in the diameter of micelles upon loading with increasing amounts of celastrol, though it is not statistically significant (Table 4). This is corroborated by DLS data from the same formulations, demonstrating nearly indistinguishable mean diameters, and similarly low polydispersity. Celastrol micelle formulations demonstrated only a slight increase in DLS mean diameter (Table 4), suggesting that all the micelle formulations are of comparable size and that any differences in activity are best explained by the activity of the cargo, celastrol, rather than physical characteristics of the micelles themselves.

TABLE 4
Micelle diameter and polydispersity from dynamic
light scattering and SAXS modeling

| | Celastrol | DLS | | SAXS Model |
|---|---|---|---|---|
| | Loaded | Diameter | | Diameter |
| | (μg) | (nm) | PdI | (nm) |
| Blank MC | 0 | 15.5 | 0.045 | 17.9 |
| Cel-MC | 0.001 | 14.8 | 0.063 | 18.0 |
| | 0.1 | 16.4 | 0.053 | 20.2 |
| | 10 | 16.3 | 0.058 | 23.4 |
| | 1000 | 17.9 | 0.032 | Not Performed |

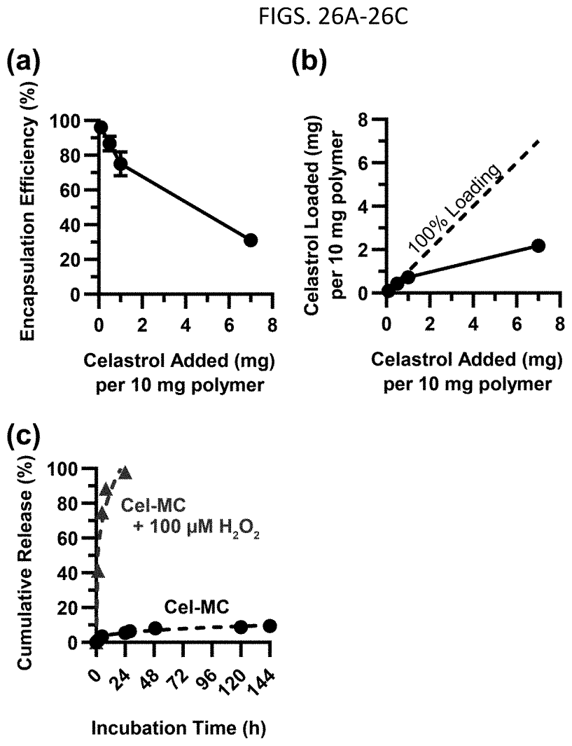

HPLC analysis of formulations before and after removal of unencapsulated celastrol via LH-20 lipophilic column filtration revealed that when celastrol is loaded at 100 μg per 10 mg of polymer the encapsulation efficiency was 96.1±0.8%. This encapsulation efficiency decreased with higher initial amounts of celastrol, suggesting diminishing returns on the amount of celastrol loaded into micelles (FIG. 26A). In order to determine what the maximum loading capacity of celastrol is in micelles, increasing amounts of celastrol were loaded into micelles until visible insoluble aggregates of celastrol were detected during micelle formation. When the amount of celastrol loaded was increased to 7 mg of celastrol per 10 mg of polymer (a theoretical loading capacity of 70%), the encapsulation efficiency dropped to 31.1±3.4%, a loading capacity of 2.2 mg celastrol per 10 mg polymer (22% loading capacity) (FIG. 26B). Quantification of celastrol dissolved in 1×PBS at 37 C found that celastrol is very sparingly soluble in the aqueous buffer, with only 3.5 μg celastrol detected in 1 mL of 1×PBS. The highest concentration of PEG-b-PPS nanocarriers recorded is 200 mg/mL of polymer, which at a celastrol loading capacity of 22% would result in a theoretical celastrol ‘solubility’ of 44 mg/mL in 1×PBS, over 10,000 times higher than unencapsulated celastrol. Perhaps due to this stark difference in solubility, celastrol remains loaded in micelles for days, exhibiting very low release (8.0±0.5%) into a 1×PBS sink over 48 hours (FIG. 26C). Celastrol can, therefore, be stably loaded at very high concentrations into PEG-b-PPS micelles, demonstrating nearly complete loading when loaded at concentrations less than 500 μg celastrol per 10 mg polymer.

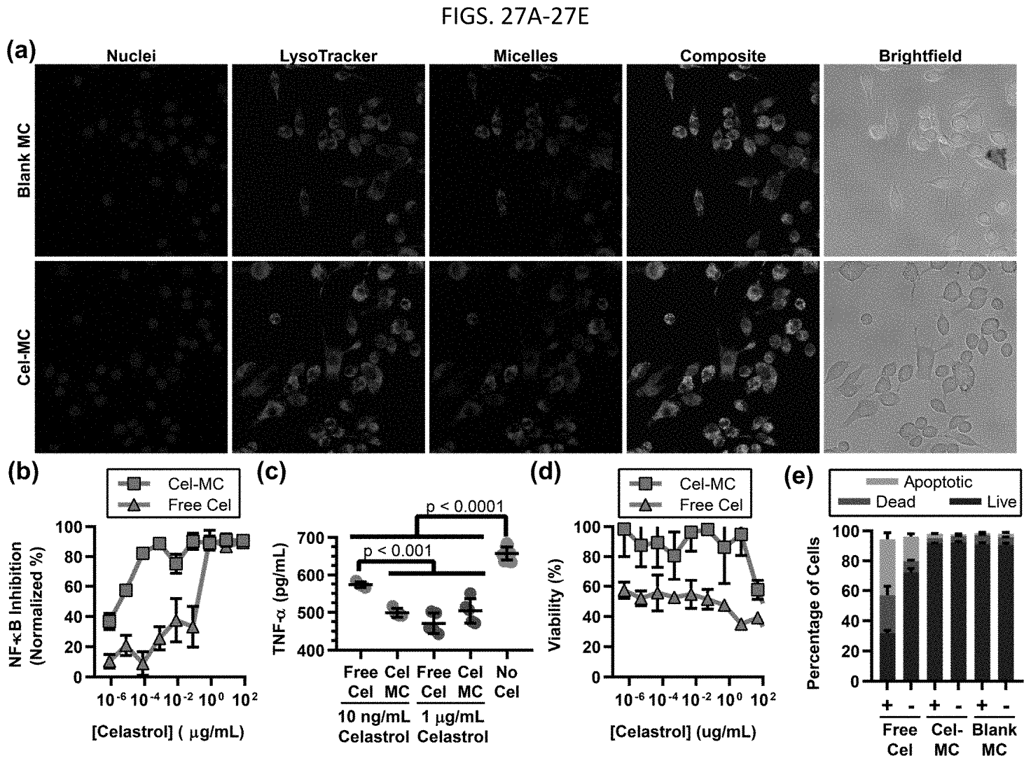

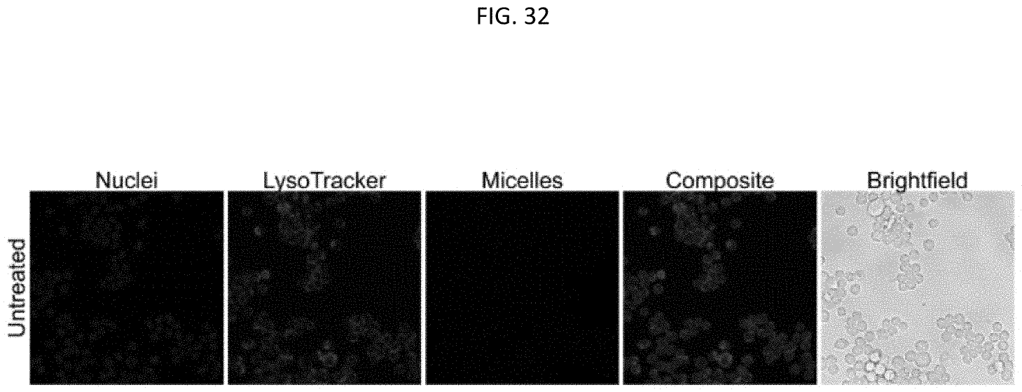

Cel-MC inhibits NF-kB signalling and is less cytotoxic than free celastrol in vitro—Celastrol is a known inhibitor of NF-κB signalling, and the studies described herein aimed to confirm that the encapsulation of celastrol within PEG-b-PPS micelles did not negatively impact its ability to function as an inhibitor. It was confirmed that loading of celastrol into micelles does not aberrantly affect their uptake and subcellular localization. Confocal images of Cel-MC were formed and labelled with DiI, a lipophilic dye with spectral properties similar to that of tetramethylrhodamine, which remains associated with PEG-b-PPS nanocarriers for nanocarrier tracking purposes. To ensure that LysoTracker signal is not collected in both the green and red filter sets, leading to an overestimation of colocalization, one well of cells were imaged in the absence of DiI-labeled micelles at the same laser power and detector sensitivity as the micelle-treated cells. These cells showed negligible bleed through into the red channel, ensuring that colocalization observed between the green and red channels accurately reflects the presence of micelles in lysosomes (FIG. 32). Confocal images demonstrate that internalized Cel-MC show strong colocalization with lysosomes, stained with LysoTracker Green (FIG. 27A), and this colocalization does not differ between unloaded micelles and Cel-MC.

Next, it was confirmed that encapsulated celastrol is able to maintain its function as an NF-κB inhibitor, as it is possible that encapsulation could diminish the ability of celastrol to be released and reach its binding targets. To do so, a reporter cell line, RAW Blue macrophages, was used, in which an NF-κB responsive promoter drives the expression of secreted alkaline phosphatase (SEAP). Upon induction of NF-κB signalling, the cells produce and export SEAP into the supernatant, which can be collected to quantify NF-κB activity using a colorimetric assay of SEAP activity. Both free (solubilized) celastrol and Cel-MCs were able to inhibit NF-κB signalling in RAW Blue cells treated with LPS (FIG. 27B). However, free celastrol demonstrates a steep decline in its efficacy between 1 μg/mL and 0.1 μg/mL concentrations, with a half maximal effective concentration (EC₅₀) of 0.2m/mL. In comparison, Cel-MC has an estimated EC₅₀ of 4.2 pg/mL, a concentration nearly 50,000 times lower. This expansion of the inhibitory concentration range of celastrol is best explained by the increased efficiency of delivery of an inhibitory dose to each cell.

While the RAW Blue cell line functions well as a transcriptional reporter, the activity of celastrol was also assessed an enzyme linked immunosorbent assay (ELISA) for TNF-α, a cytokine produced and secreted as a consequence of NF-κB activation. TNF-α plays a key role in both cell survival, apoptosis, stress response, and immune cell recruitment, making its modulation an important part of a potential anti-inflammatory strategy. The RAW Blue assay suggested a drop in inhibitory efficacy for free celastrol between 0.01-1 μg/mL celastrol, which was not seen for Cel-MC (FIG. 27B). To confirm this difference, inhibition of RAW 264.7 cells was performed with 10 ng/mL or 1 μg/mL celastrol in either free or Cel-MC form for 6 hours, with simultaneous LPS treatment of cells. This resulted in a decrease in TNF-α secretion (FIG. 27C). At 1 μg/mL celastrol, both free celastrol and Cel-MC significantly decreased TNF-α levels in the supernatant compared to control cells treated only with LPS. Treatment with free celastrol and Cel-MC at this concentration were not significantly different from one another. At the 10 ng/mL celastrol concentration, however, Cel-MC treatment significantly outperforms free celastrol inhibition, which only partially (but still significantly) inhibits TNF-α production. This corroborates the data from the RAW Blue assay and highlights the finding that Cel-MC remains an effective inhibitor of NF-κB at concentrations of celastrol that are not as inhibitory in free form. Unlike the nearly complete inhibition of SEAP activity demonstrated in the RAW Blue assay in FIG. 27B, TNF-α was still at detectable levels in the supernatant of cells after both celastrol treatments. This suggests that either not all cells had their NF-κB signaling completely inhibited, or TNF-α expression was induced by an alternative NF-kB independent mechanism. The amount of LPS used in these in vitro experiments likely dwarfs the amount of inflammatory stimuli in most in vivo contexts, but serves to illustrate that encapsulated celastrol is able to function efficiently as an inhibitor of NF-κB signaling.

Although beneficial for chemotherapeutic applications, the high cytotoxicity of celastrol hinders its use as an anti-inflammatory agent. Since Cel-MC demonstrated high NF-κB inhibition at significantly lower celastrol concentrations than free form celastrol (FIG. 27B), it was hypothesized that Cel-MC could serve as a potent anti-inflammatory at nontoxic concentrations of celastrol. It was found that at very high doses (0.8 mg/mL celastrol), both free celastrol and Cel-MC are highly cytotoxic to RAW 264.7 cells (FIG. 27D). However, as the dosage of celastrol is decreased, there is a marked increase in cell viability for Cel-MC, but not for free celastrol. When comparing the concentrations that are relevant for successful inhibition of NF-κB, it is apparent that there is only a very narrow range of concentrations for free celastrol that are both effective at inhibiting NF-κB and moderately tolerated by cells. In contrast, Cel-MC has a very broad range of concentrations of loaded celastrol that demonstrate high NF-κB inhibition along with high cell viability.

Celastrol has been investigated as a potential anti-cancer therapeutic due to its ability to induce cell death, and potentially binds to a number of proteins involved in apoptosis. As the manner of cell death can influence the downstream immune response, the onset of apoptosis upon treatment with celastrol was evaluated. Since NF-κB signalling in RAW 264.7 cells results in the secretion of TNF-α (FIG. 27C), an inducer of apoptosis in some contexts as well as cell survival and proliferation in others, the effect of the presence or absence of LPS during celastrol treatment on the induction of apoptosis or necrosis was assessed. 1 μg/mL of celastrol was used for testing, which is near the lowest concentration at which both free and micelle-loaded celastrol strongly inhibit NF-κB (FIG. 27B). For cells treated with free celastrol for 4 h, 37.5±4.4% of cells were found to be apoptotic in the presence of LPS, which significantly reduced to 16.5±1.8% (p<0.0001) in the absence of LPS (FIG. 27E). These results likely reflects a synergy between celastrol-induced and LPS-dependent TNF-induced apoptosis. Strikingly, there was no significant difference observed when comparing Cel-MC to blank controls with or without LPS, both of which induced less than 4% of cells to be apoptotic and were significantly (p<0.0001) lower than cells treated with free form celastrol (FIG. 27E).

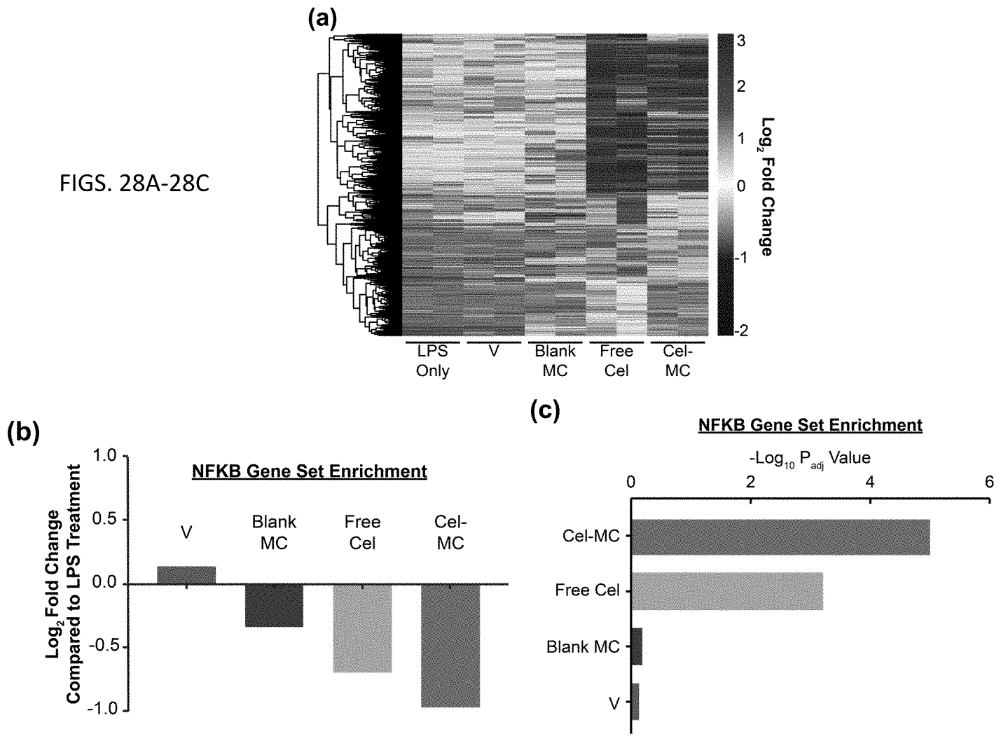

As celastrol has been shown to target a number of different pathways in different cell types it was confirmed on a transcriptional level that free celastrol and Cel-MC treatments do not have strikingly different transcriptional profiles in an inflammatory cell. LPS-treated RAW 264.7 cells as our model inflammatory cell type was treated with 1 μg/mL celastrol in free or Cel-MC form, a concentration shown in FIG. 27 to inhibit NF-κB. RNA was extracted from the cells and RNAseq was performed on the mRNA. Heatmap analysis of the 2084 genes significantly altered by free celastrol treatment of LPS-treated RAW 264.7 cells after 2 hours (FIG. 28A). Notably, pro-inflammatory genes such as illb⁵⁶, tnf⁵⁷, and nfatc1⁵⁸ were significantly downregulated in both groups and anti-inflammatory genes such as lrp1⁵⁹, irf7⁶⁰, and slpi⁶¹, were significantly upregulated in both groups (P_{adj}<0.1; DE-Seq2). Downregulation of illb and tnf is notable as their products, IL-1β and TNF-α, are highly implicated in the pathogenesis of atherosclerosis. IL-1β, a product of inflammasome signaling, and TNF-α, a product of TLR engagement, are both induced by oxLDL and result in the promotion of foam cell formation and the enrichment of other pro-inflammatory cells in the developing atherosclerotic plaque. Pathway analysis confirmed that NF-κB target genes were significantly downregulated under both conditions (P_{adj}<0.1; FIG. 28B,C). This analysis confirms that the encapsulation of celastrol in PEG-b-PPS micelles does not adversely affect its ability to function as a small molecular inhibitor. To the contrary, encapsulation of celastrol results in a lower EC₅₀ and better cell viability.

Cel-MC treatment reduces inflammatory immune cell populations in atherosclerotic plaques—Two additional hinderances to the therapeutic use of celastrol are poor solubility/bioavailability and signalling promiscuity in a wide range of cells and tissues. Translation from in vitro to in vivo work highlights these difficulties, as they are difficult to assess in solely mammalian cell culture experiments. Having found non-cytotoxic doses of Cel-MC and tolerable doses of free celastrol (100 ng/mL, FIG. 27), it was examined whether controlled delivery of encapsulated celastrol via PEG-b-PPS micelles could ameliorate inflammation within atherosclerotic plaques.

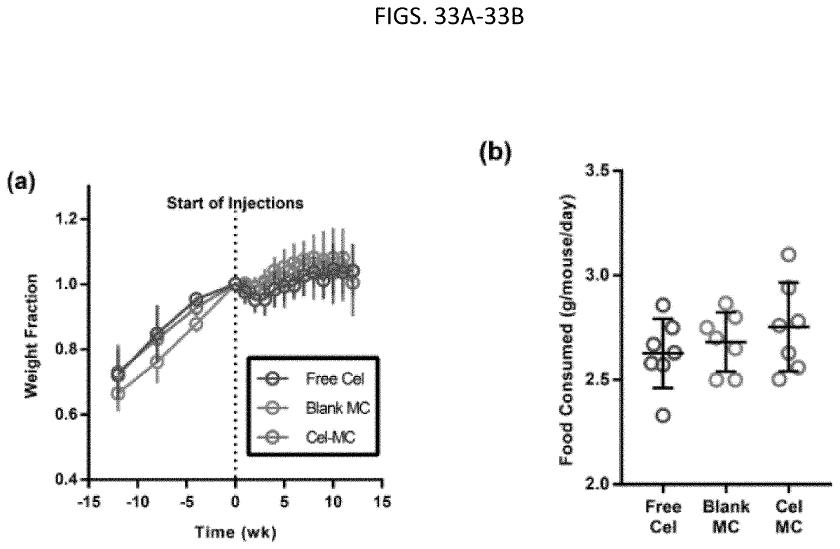

Celastrol is typically administered to humans orally and to mice intraperitoneally (IP). IP injections have limited applicability to humans, so the free celastrol IP injections were used as a control and used the more relevant intravenous (IV) route of administration was used for Cel-MC formulations. As the goal was to alleviate inflammatory signaling in atheromas, early stage atherosclerotic lesions were first established through the feeding of a high fat diet to ldlr−/− mice for 3 months. Subsequently, weekly administrations of the treatments were administered for 3 additional months. Mice were monitored and weighed to discern any changes in appetence or weight due to treatment toxicity, of which neither was detected (FIG. 33A-B). At the end of the experiment, mice had been on high fat diet for 6 months, which typically results in the development of late stage plaques in ldlr−/− mice. Mice were sacked, and major lymphoid organs (spleen, lymph nodes, blood) and aorta were collected and processed for flow cytometry to characterize the immune cell population profile of the different tissues.

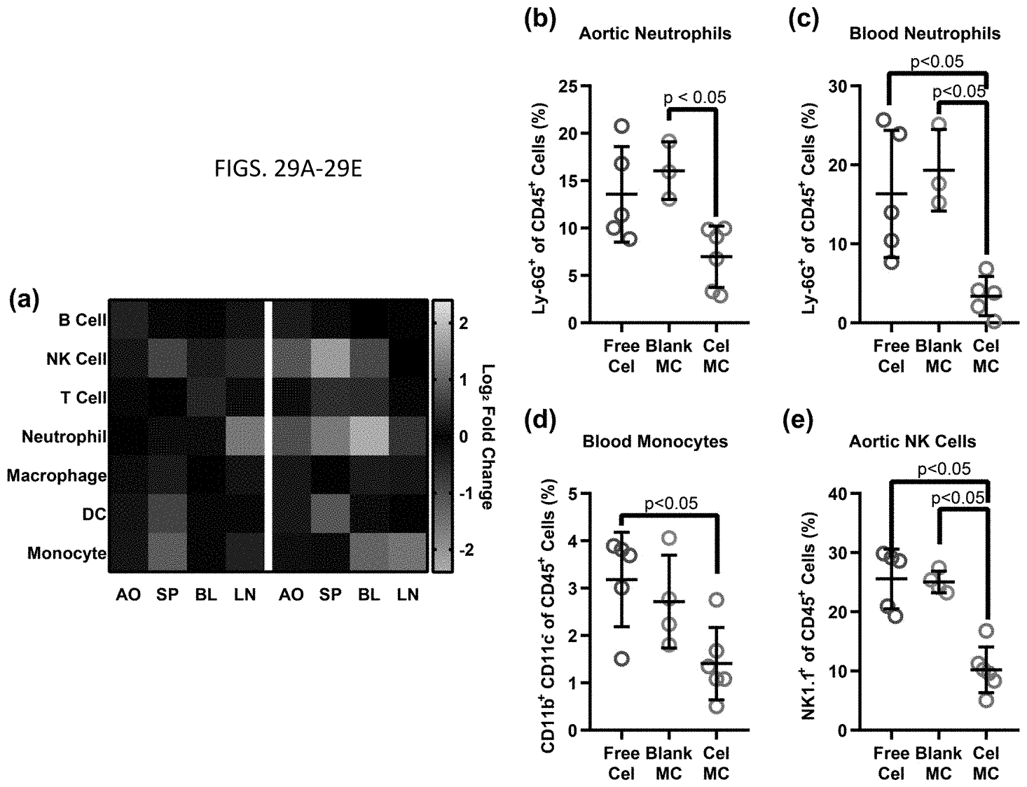

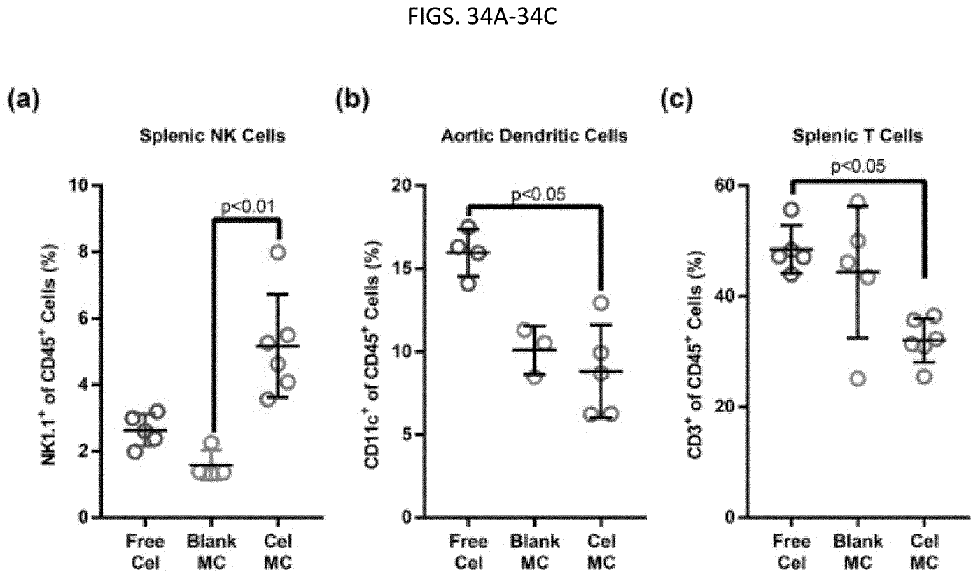

Changes in cell population were compared between celastrol treatments and the blank micelle treatment control, resulting in a log₂ fold change heatmap (FIG. 29A). Free celastrol at 33 μg/kg/week had muted effects, with a mixture of cell population increases and decreases compared to blank MC. In contrast, Cel-MC resulted in statistically significant decreases for several key inflammatory cell populations compared to free celastrol and blank MC (FIG. 29A-E, FIG. 34A-C). Neutrophils in both the blood (FIG. 29B) and atherosclerotic plaque (FIG. 29C) saw a significant reduction in their share of the immune cell population upon Cel-MC treatment. Neutrophils are highly pro-inflammatory and secrete a network of proteins and DNA called neutrophil extracellular traps, a process known as NETosis. NETosis has been implicated in the progression of atherosclerosis by licensing macrophages to secrete pro-inflammatory cytokines and by inducing the cell death of vascular endothelial cells. As such, this reduction in the neutrophil population could have therapeutic relevance. Similarly, monocytes in the blood are often pro-inflammatory, and their reduction during the course of Cel-MC treatment (FIG. 29D) could help ameliorate the inflammatory state in the plaque, where monocytes are often recruited and induced to differentiate into macrophages and foam cells. NK cells in atherosclerotic plaques were also reduced (FIG. 29E). Intriguingly, NK cells significantly increased in the spleen of Cel-MC treated mice (FIG. 34A), potentially suggesting an alteration in their trafficking.

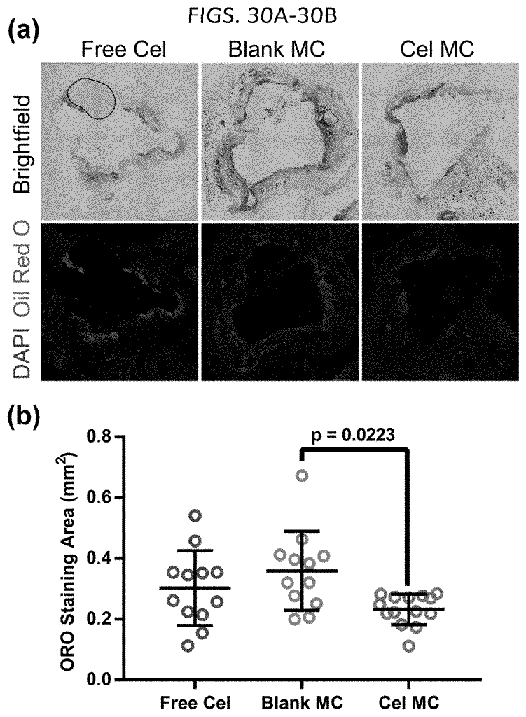

Cel-MC treatment reduces plaque area—One proxy for plaque progression in mice is plaque area. To determine whether Cel-MC reduced plaque area, Oil Red O (ORO) staining on frozen histology cross sections of mouse aorta was performed. ORO is a fluorescent stain for lipid rich regions of atherosclerotic plaques. Representative sections for Cel-MC and Blank MC treated mouse aortas are shown in FIG. 30A. A quantitative comparison between the ORO staining of different treatment groups was achieved using an automated script written in Python (FIG. 30B), which revealed a significant decrease in plaque area between Cel-MC and Blank-MC control treatments. The administration of free celastrol at this low dosage did not significantly reduce the plaque area compared to the Blank MC control. These results illustrate the benefit of encapsulation within PEG-b-PPS nanocarriers to adjust the therapeutic window of celastrol, achieving targeted therapeutically relevant modulation of key inflammatory cell populations at a sufficiently low dosage to avoid toxicity.

Encapsulation of celastrol into PEG-b-PPS micelles resulted in significant decreases in both effective dose required to inhibit NF-κB as well as cytotoxicity in vitro. In vivo, Cel-MC modulated the proportional makeup of immune cell populations within atherosclerotic plaques and systemically, both of which contribute to the development and progression of atherosclerosis. As a demonstration of therapeutic efficacy, Cel-MC reduced plaque area compared to Blank MC controls in high fat diet fed ldlr−/− mice. Together, these findings provide proof of concept that PEG-b-PPS nanocarriers can drastically enhance the therapeutic utility of celastrol both in vitro and in vivo. With regards to atherosclerosis, it is demonstrated herein that targeted delivery of an anti-inflammatory small molecule inhibitor to immune cells results in a significant reduction of a marker for plaque progression.

### Example 3

Nanodrugs are defined as nanocarrier formulation of currently used drugs. Nanodrugs have rapidly emerged due to the convergence of biomedical engineering, pharmacology, and nanotechnology. An important feature of nanocarriers is their ability to dictate to which cells a drug is delivered. Rapamycin, a known immunosuppressive mTOR inhibitor, directly acts on T cells to inhibit their proliferation and secretion of IL-2. Because of rapamycin's wide biodistribution it also arrests the cell cycle of non-immune cells, causing side effects. However, when rapamycin is delivered via poly(ethylene glycol)-b-poly(propylene sulfide) (PEG-b-PPS) polymersome nanocarriers (rPS), the drug is primarily taken up by antigen presenting cells (APCs), completely changing the drug's mechanism of action. Uptake of rapamycin by APCs, induces anti-inflammatory Ly-6C^{low} monocytes and tolerogenic semi-mature dendritic cells with high presentation of MHC II and low levels of costimulatory molecules. The presentation of “signal 1 in the absence of signal 2” by these tolerogenic APCs cause anergy of acute rejection causing CD4+ effector T cells and promotes proliferation of tolerance inducing CD8+ regulatory T cells. Subcutaneous injection of rPS is used to target the lymph nodes. Furthermore, we demonstrate rPS can be used for enhanced fully major histocompatibility complex (MHC)-mismatched allogeneic islet transplantation to the clinically relevant intraportal (liver) transplantation site with reduced side effects such as weakened immune defenses and alopecia.

### Materials and Methods

Animals—8 to 12-week-old, male C57BL/6J and Balb/c mice were purchased from Jackson Labs. Mice were housed in the Center for Comparative Medicine at Northwestern University. All animal protocols were approved by Northwestern University's Institutional Animal Care and Use Committee (IACUC).

Materials—Unless explicitly stated below, all reagents and chemicals were purchased from Sigma-Aldrich.

Polymer Synthesis—Poly(ethylene glycol)-block-poly(propylene sulfide) (PEG-b-PPS) was synthesized as previously described by us¹⁶. In brief, methyl ether PEG (MW 750) was functionalized with mesylate. The mesylate was reacted with thioacetic acid to form PEG-thioacetate and then base activating the thioacetate to form a thiolate anion and initiate ring opening polymerization of propylene sulfide. Benzyl bromide was used as an end-capping agent to form PEG17-b-PPS₃₀-Bz or the thiolate anion was protonated to form PEG17-b-PPS₃₀-SH. The polymer was characterized by H-NMR and gel permeation chromatography (GPC).

Nanocarrier Formulation—Polymersomes (PS) were formed via thin film rehydration, as previously described. In brief, 20 mg of PEG₁₇-b-PPS₃₀-Bz was weighted in a sterilized 1.8 ml glass HPLC vial. 750 ul of dichloromethane (DCM) was added to the vial. To form, rPS 0.5 mg of rapamycin, dissolved at 25 mg/ml in ethanol, was also added. The vial was desiccated to remove the DCM. Next, 1 ml of phosphate-buffered saline (PBS) was added to the vial. The vials were shaken at 1500 rpm overnight. PS were extruded multiple times first via 0.2 um and then 0.1 um syringe filters. Excess rapamycin was removed via size exclusion chromatography using a Sephadex LH-20 column with 1×PBS.

Poly(lactide-co-glycolide) nanoparticles (PLGA) were prepared using an Oil-in-Water (O/W) single emulsion method. Briefly, organic phase containing PLGA (Polyscitech) (60 mg in 1 mL dichloromethane) was added to 6 mL of aqueous phase containing 2.5% (w/v) of polyvinyl alcohol (PVA). The resultant mixture was emulsified on ice using an ultrasonic processor to form an O/W emulsion. This emulsion was then added drop wise into 5 mL of stirring 0.25% PVA solution at room temperature to evaporate the organic solvent. Nanoparticles were collected after 6 hours of stirring followed by centrifugation at 17,000×g for 10 minutes. After centrifugation nanoparticles were washed twice with cold water to remove residual PVA and redispersed in phosphate buffered saline (PBS). The rapamycin loaded PLGA nanocarriers (rPLGA) were prepared by adding rapamycin (3 mg) to the organic phase.

Nanocarrier Characterization—Dynamic Light Scattering (DLS): DLS measurements were performed on a Nano 300 ZS Zetasizer (Malvern) and were used to determine nanocarrier diameter distribution and corresponding polydispersity index.

Cryogenic transmission electron microscopy (cryoTEM): 200-mesh lacey carbon grids were glow-discharged for 30 seconds in a Pelco easiGlow glow-discharger at 15 mA with a chamber pressure of 0.24 mBar. 4 μL of sample was then pipetted onto the grid and plunge-frozen into liquid ethane in a FEI Vitrobot Mark III cryo plunge freezing device for 5 seconds with a blot offset of 0.5 mm. Grids were then loaded into a Gatan 626.5 cryo transfer holder, imaged at −172° C. in a JEOL JEM1230 LaB6 emission TEM at 100 kV, and the data was collected on a Gatan Orius 2k×2k camera.

Small angle x-ray scattering (SAXS): SAXS was performed at Argonne National Laboratory's Advanced Photo Source with collimated X-rays (10 keV; 1.24 Å). Data reduction was performed using Primus software and modeling was performed using SASView.

Quantification of Rapamycin Loading¹⁶—rPS (50 ul) were lyophilized and re-dissolved in HPLC grade DMF. Salts were removed via centrifugation at 17,000 g for 10 minutes. Rapamycin content of rPS was characterized via HPLC (Thermo Fisher Dionex UltiMate 3000) using an Agilent Polypore 7.5×300 mm column and an Agilent Polypore 7.5×50 mm guard column. The system was housed at 60° C. DMF (0.5 ml/minute) was used as the mobile phase. Rapamycin was detected at 270 nm. Thermo Scientific Chromeleon software was used for analysis. The concentration of rapamycin was characterized via the area under the curve in comparison to a standard curve of rapamycin concentrations.

Immunomodulation Study—Healthy C57BL/6J mice were subjected to a “standard dosage regime.” Animals were injected subcutaneously for 11 days with rapamycin (in 0.2% CMC) or rPS at a dose of 1 mg/kg. Equivalent dose of 1×PBS or PS were injected as controls. After 11 days, the mice were sacrificed. Blood, lymph nodes (axial, brachial, and inguinal), liver and spleen were collected and processed for flow cytometry.

Flow cytometry—Blood was spun down at 3000 g for 25 minutes to separate the plasma and blood cells. The blood cells were treated with 1× red blood cell lysis buffer (Fisher) for 5 minutes on ice, washed with 1×PBS and spun down, thrice. The liver was minced, treated with collagenase for 45 minutes at 37° C., processed through a 70 nm filter, and then treated with 1× red blood cell lysis buffer (Fisher) for 5 minutes on ice, washed with 1×PBS and spun down. The spleen was processed through a 70 nm filter and treated with 1× red blood cell lysis buffer (Fisher) for 5 minutes on ice, washed with 1×PBS and spun down. Lymph nodes were passed through a 70 nm filter, washed with 1×PBS and spun down. All cells were resuspended in a cocktail of Zombie Near Infrared (BioLegend) for viability and anti-mouse CD16/CD32 for FcR blocking with BD Brilliant Violet cell staining buffer and incubated at 4° C. for 15 minutes. Next, an antibody cocktail consisting of Pacific Blue anti-mouse CD11c (BioLegend), BV480 anti-mouse NK1.1 (BD), BV510 anti-mouse CD19 (BioLegend), BV570 anti-mouse CD3 (BioLegend), BV650 anti-mouse F4/80 (BioLegend), BV650 anti-mouse MEW II (IA-IE) (BioLegend), BV711 anti-mouse Ly-6C (BioLegend), BV750 anti-mouse CD45R/B220 (BioLegend), BV785 anti-mouse CD11b (BioLegend), AF532 anti-mouse CD8a (Invitrogen), PerCP-Cy5.5 anti-mouse CD45 (BioLegend), PerCp-eFluor711 anti-mouse CD80 (Invitrogen), PE-Dazzle594 anti-mouse CD25 (BioLegend), PE-Cy5 anti-mouse CD4 (BioLegend), PE-Cy7 anti-mouse CD169 (BioLegend), APC anti-mouse FoxP3 (Invitrogen), AF647 anti-mouse CD40 (BioLegend), APC-R700 anti-mouse Ly-6G (BioLegend), and APC/Fire 750 anti-mouse CD86 (BioLegend) was added to the cells and incubated for 20 minutes at 4° C. The cells were washed with 1×PBS, fixed and permeabilized using a FoxP3 Fix/Perm Kit (BioLegend), according to the manufacturer's protocol. Next, anti-mouse FoxP3 was added and incubated for 30 minutes in the dark at room temperature. Finally, cells were washed twice with 1×PBS and resuspended in cell buffer. The cells were analyzed on an Aurora flow cytometer (CyTek). Spectral unmixing was performed using SpectroFlo (CyTek) and analysis was performed using FloJo software. Gating was performed as outlined in FIG. S57^{44,45.}

T-distributed stochastic neighbor embedding (t-SNE)—For each analyses, FlowJo's DownSample plugin was used to randomly select an equal number of events from each cell population (CD45+, CD3+, CD19+, CD11b+, or CD11c+) of every sample. The purpose of DownSample was to both normalize the contribution of each mouse replicate and reduce computational burden. Next, samples from mice that underwent the same treatment and same cell population were concatenated. The tSNE plugin was run on concatenated samples using the Auto opt-SNE learning configuration with 3000 iterations, a perplexity of 50 and a learning rate equivalent to 7% of the number of events⁴⁶. The KNN algorithm was set to exact (vantage point tree) and the Barnes-Hut gradient algorithm was employed.

Indocyanine Green Biodistribution—Indocyanine green (ICG) polymersomes were formed using thin film rehydration, as previously described¹³. In brief, 20 mg of PEG₁₇-b-PPS₃₀-Bz was weighted in a sterilized 1.8 ml glass HPLC vial. 750 ul of dichloromethane (DCM) was added to the vial. The vial was desiccated to remove the DCM. Next, 1 ml of 0.258 mM ICG in 1×PBS was added to the vial. The vials were shaken at 1500 rpm overnight. PS were extruded multiple times first via 0.2 um and then 0.1 um syringe filters. Float-A Lyzer G2 Dialysis devices (Fisher) were used to remove unloaded ICG. ICG loading was quantified relative to standards composed of known amounts of polymer and ICG in a 1:33 molar ratio using absorbance at 820 nm as previously described by our group¹³. C57BL/6J mice received subcutaneous injections of either free ICG (in 1×PBS) or ICG-PS. ICG concentration was matched at 50 ug/ml. The injection volume was 150 ul. At 2, 24- and 48-hours post-injection, the mice were sacrificed, blood was collected via cardiac puncture, and perfusion was performed using heparinized 1×PBS. Liver, spleen, kidneys, heart and lung were harvested and imaged via IVIS Lumina with an excitation wavelength of 745 nm, an emission wavelength of 810 nm, an exposure time of 2 seconds and a f/stop of 2.

Rapamycin Biodistribution—Mice were injected with rapamycin (in 0.2% CMC) or rPS at 1 mg/ml and sacrificed at the following time points: 0.5, 2, 8, 16, 24, and 48 hours. Urine was collected via metabolic cages during the duration between injection and sacrifice for the 8, 16, 24 and 48-hour timepoints. The following tissues and/or organs were collected: blood, spleen, liver, kidneys, heart, brain, lungs, lymph nodes (axial and brachial), and fat pad. Rapamycin was extracted from blood and urine using a solution of methanol and acetonitrile (50:50 v/v) doped with rapamycin-D3 (Cambridge Isotope Laboratories) as an internal standard. Tissue samples were homogenized in homogenization tubes prefilled with stainless steel ball bearings (Sigma) using a solution of phosphoric acid (8%), acetonitrile and acetic acid (30:67.2:2.8 v/v/v). After homogenization, tissue samples were also doped with rapamycin-D3. All samples were precipitated via incubation at −20° C., followed by centrifugation. The supernatant was collected and LC-MS/MS (Shimadzu LC-30AD pumps; SIL-30ACMP autosampler; CBM-20A oven; Sciex Qtrap 6500) was used to determine rapamycin concentration. Rapamycin had a retention time of 2.7 minutes. Rapamycin-D3 had a retention time of 3.0 minutes.

Allogeneic Islet Transplantation—Diabetes was induced via streptozotocin (IP; 190 mg/kg) injection five days prior to transplantation and confirmed via hyperglycemia (blood glucose>400 mg/dl). Starting the day prior to transplantation, mice were injected with PBS, PS, rapamycin, or rPS (N=3 per group) at 1 mg/kg (or equivalent) in accordance with a standard dosage (11 doses, given daily) or a low dosage (6 doses, given every 3^{rd} day). On the day of transplantation, islets were isolated from Balb/c mice via common bile duct cannulation and pancreas distension with collagenase. Islets isolated from two donors (˜200 mouse islets, ˜175 IEQ) were transplanted to C57B6/J recipients via the portal vein. Body weight and blood glucose concentration were monitored closely for 100 days post-transplantation. Intraperitoneal glucose tolerance test (IPGTT) was performed one-month post transplantation. The animals were fasted for 16 hours before being injected intraperitoneally with 2 g dextrose (200 g/L; Gibco) per kg body weight. Blood glucose concentrations were measured at 0, 15, 30, 60- and 120-minutes post-injection.

Alopecia Assessment—Dorsal photos were taken weekly to assess for alopecia. At 100-days post-transplantation, the mice were euthanized and skin samples were excised in the dorsal region at the subcutaneous injection site. Skin samples were placed in cassettes, fixed in 4% paraformaldehyde, and embedded in paraffin. Tissue blocks were sectioned at a thickness of 5 nm and stained with hematoxylin and eosin (H&E). Digital images were taken on a Nikon microscope.

Single Cell RNA Sequencing—Healthy C57BL/6J mice were subjected to a “standard dosage regime.” Animals were injected subcutaneously for 11 days with rapamycin (in 0.2% CMC) or rPS at a dose of 1 mg/kg. Equivalent dose of 1×PBS or PS were injected as controls. After 11 days, the mice were sacrificed, and the liver and spleen were excised. The organs were processed as was done for flow cytometry. CD4+ regulatory T cells and macrophages were isolated using magnetic sorting (MojoSort; BioLegend). Briefly, cells were first incubated in a cocktail of PE anti-mouse CD169 and PE anti-mouse F4/80 antibodies (BioLegend). After washing, incubation in anti-PE nanobeads (BioLegend) occurred. Macrophages were magnetically sorted from non-macrophages. The non-macrophages cell fraction was then incubated in mouse CD4+ T cell isolation biotin-antibody cocktail (BioLegend) and sorted. The CD4+ T cell fraction was then incubated in APC anti-mouse CD25 antibody (BioLegend), followed by washing, incubation in anti-APC nanobeads (BioLegend) and sorting. RNA was isolated from separated macrophages and CD4+ regulatory T cells using R-Neasy Mini Kit with DNAse digestion (Qiagen), Samples were frozen and shipped to Admera Health where they underwent library preparation using the Lexogen 3′ mRNA-Seq Library Prep Kit FWD HT (Lexogen) and were sequenced on an Illumina sequencer (HiSeq 2500 2×150 bp). For each pair, Read 2 was discarded and only Read 1 was used for downstream data analysis. Sequencing quality was analyzed with FastQC v0.11.5⁴⁷ and reads were trimmed and filtered with Trimmomatic v0.3948. One sample from the spleen T cell PBS treatment group and one sample from the spleen T cell rapamycin treatment group were discarded due to low sequencing quality. Reads were aligned with STAR v2.6.0a 49 to the GRCm38.p6 mouse reference genome primary assembly using the GRCm38.p6 mouse reference primary comprehensive gene annotation (https://www.gencodegenes.org/mouse/). Quantification and differential expression was performed with Cuffdiff from Cufflinks v2.2.150-52 again using the GRCm38.p6 mouse reference primary comprehensive gene annotation and a 0.05 FDR. Detailed settings for each software are included in Table S1. The raw data displayed in FIG. 4e broken down by cell type is in Table S2.

### Results

There is an unmet need for targeted immunosuppressive therapies that have reduced off-target effects, as 64% of transplant recipients report that these side effects significantly lower their quality of life^{1,2}. Due to the undesirable accumulation and action of immunosuppressive drugs in organs and cells, these drugs tend to have many off-target effects causing negative side effects for patients^{2,3}. Commonly prescribed immunosuppressive drugs tend to have nonspecific biodistributions—meaning that they indiscriminately affect target and non-target tissues²⁻⁴. For example, rapamycin, a maintenance immunosuppressive drug, primarily partitions into red blood cells (95%) and then eventually accumulates in organs that do not aid in immunosuppressive functions, including the heart, kidneys, intestines and testes⁵⁻⁸. Furthermore, immunosuppressive drugs tend to act on pathways that have many downstream effects². Rapamycin inhibits the mammalian target of rapamycin (mTOR) pathway; halting the cycle of T cells in the G1 phase and thus inhibiting proliferation^{2,3}. However, due to the ubiquitous nature of mTOR, other cell types also experience cell cycle arrest and inhibited proliferation^{2,3}. Clinically, this can cause patients taking rapamycin to experience malignancy, enhanced susceptibility to infection, impaired wound healing, thrombopenia, alopecia, gastrointestinal issues, gonadal dysfunction, hypertension, hyperlipidemia, nephrotoxicity and peripheral edema^{3,9}. In many cases, these off-target effects cause side effects that negatively impact the transplanted organ or tissue. For example, tacrolimus, which is commonly given for kidney transplantation is nephrotoxic, and rapamycin, which is often given for pancreas and islet transplantation is diabetogenic^{2,3}. Thus, the drug that is intended to protect the transplanted graft from the body's immune system can actually be damaging the graft itself. Finally, many of these drugs, including rapamycin, tacrolimus and cyclosporine, are highly hydrophobic and have poor bioavailability. In some cases, toxic solubilizing agents, such as polyethoxylated castor oil, have been used to make these drugs more bioavailable for parenteral administration, however this is associated with hypersensitivity reactions, such as anaphylaxis^{10,11} Clinicians indicate a need for targeted therapies that lead to fewer graft rejections and adverse effects,¹² which are objectives that can be achieved via nanomedicine, wherein synthetic nanoscale materials are employed to target specific cells and tissues to reduce side effects.^{10,12-15}

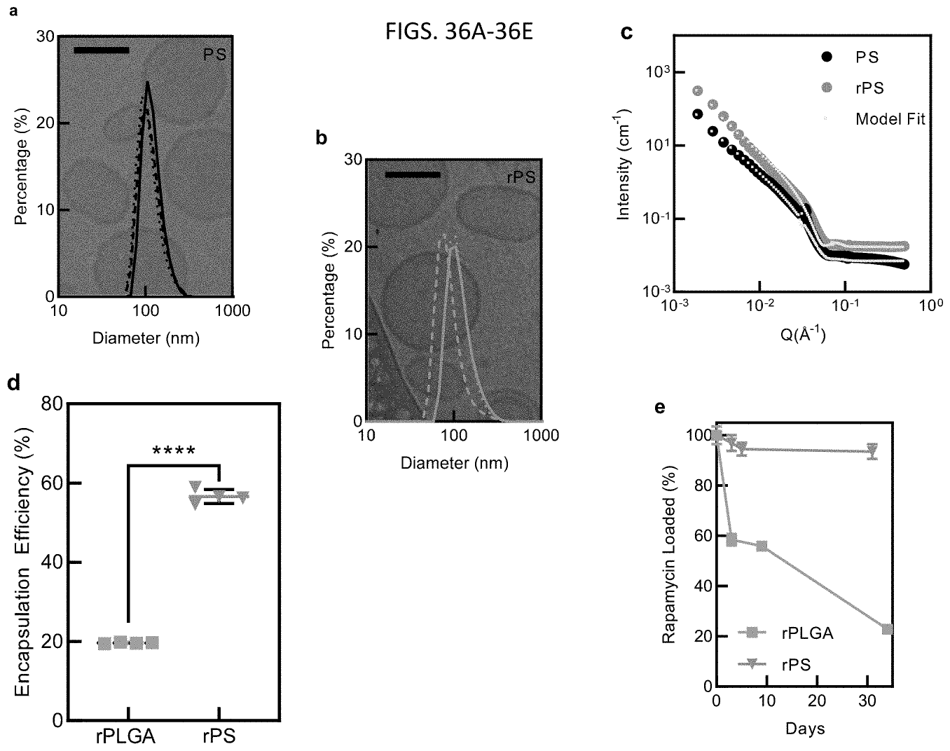

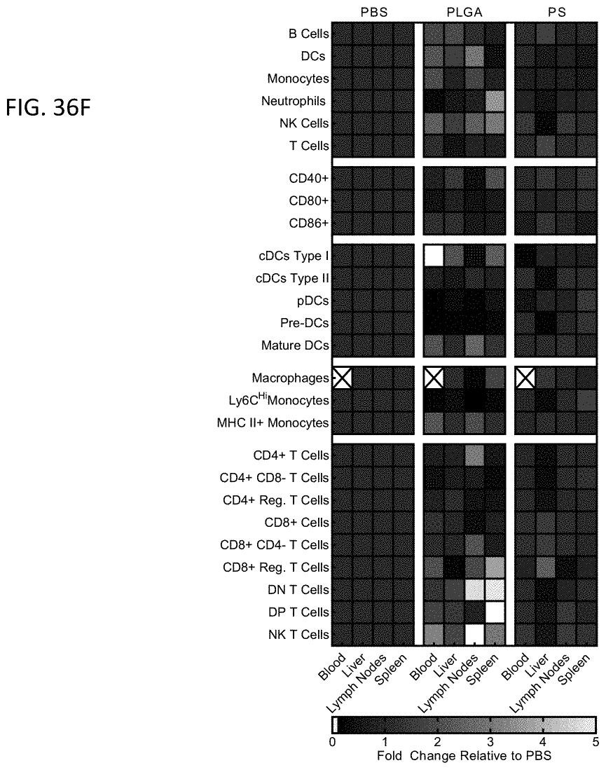

‘Nanodrugs’ are a result of recent advances in nanotechnology, wherein drugs are loaded into ‘nanocarriers’. Nanocarriers can be thought of as safe, nontoxic synthetic viruses that are composed of man-made or natural polymers. Like viruses, these nanocarriers are designed to transport through the body to target specific cells and tissues. When loaded with drugs, nanocarriers can better control where the drugs go within the body and even change how the encapsulated drugs function. Self-assembling nanostructures fabricated from the amphiphilic, diblock, copolymer poly(ethylene glycol)-b-poly (propylene sulfide) (PEG-b-PPS) provide a potential tool to address this challenge because they can be engineered to target their cargo. We have shown that by varying the length of the hydrophilic PEG block, a variety of nanocarrier morphologies can be formed¹⁶. Each morphology has a unique biodistribution and is preferentially uptaken by specific antigen presenting immune cells (APCs)^{13,17}. These PEG-b-PPS nanostructures are capable of loading both hydrophobic and hydrophilic molecules as payloads¹⁶. These payloads can be released when nanostructures are endocytosed by APCs¹⁸. The hydrophobic portion of the polymer is oxidation sensitive and will degrade under the oxidative conditions of an APC's endocytosis¹⁸. Herein, we show that the hydrophobic mTOR inhibitor rapamycin can be loaded into the polymersome (PS) nanocarrier via thin film rehydration without altering the PS morphology as assessed by dynamic light scattering (DLS), cryogenic transmission electron micrograph (cryoTEM) (FIGS. 36A and 36B), and small angle x-ray scattering (SAXS) (FIG. 36C). The structures were found to have an average diameter of 102.8±1.2 nm for blank PS and 105.1±2.6 nm for rPS with a polydispersity index of less than 0.2 (0.180 for blank PS and 0.168 for rPS) (FIGS. 36A and 36B).

Unlike other nanocarrier systems, such as liposomes and poly(lactic-co-glycolic acid) (PLGA)-based nanocarriers that cause an intrinsic inflammatory response, PEG-b-PPS nanocarriers have a high payload encapsulation efficiency, are shelf-stable and are nonimmunogenic. In the case of rapamycin, the encapsulation efficiency was greater than 55% for PS as compared to less than 20% for comparable PLGA nanocarriers (FIG. 36D). Thus, less drug is wasted in the fabrication process. Furthermore, the loaded drug remains stability inside the rPS particles for over one month, while loaded rapamycin escapes PLGA nanocarriers within a matter of days (FIG. 36E). Unloaded PS cause minimal immunomodulatory activity whereas PLGA nanocarriers cause an extensive immunomodulatory response (FIG. 26F). Thus, PEG-b-PPS nanocarriers allow for greater precision and control of immunomodulatory strategies by significantly reducing background, non-specific inflammatory responses to the nanodrug. Of importance, PS are nontoxic to both mice and non-human primates¹⁹⁻²¹.

Here, we demonstrate that PEG-b-PPS PS loaded with the hydrophobic mTOR inhibitor rapamycin (rPS) redirect the delivery of rapamycin to APCs to induce an anti-inflammatory phenotype and modulate tolerogenic T cell responses. The subcutaneous route of administration provides the advantage of targeted lymphatic drainage²², avoidance of first past metabolism²³ and a path for translation from mice to humans. We demonstrate the utility of rPS induced immunomodulation for fully major histocompatibility complex (MHC)-mismatched allogeneic islet transplantation to both the clinically relevant intraportal (liver) transplantation site. Furthermore, as compared to rapamycin, reduced off-target effects are observed on both the physical and transcriptional level with rPS treatment.

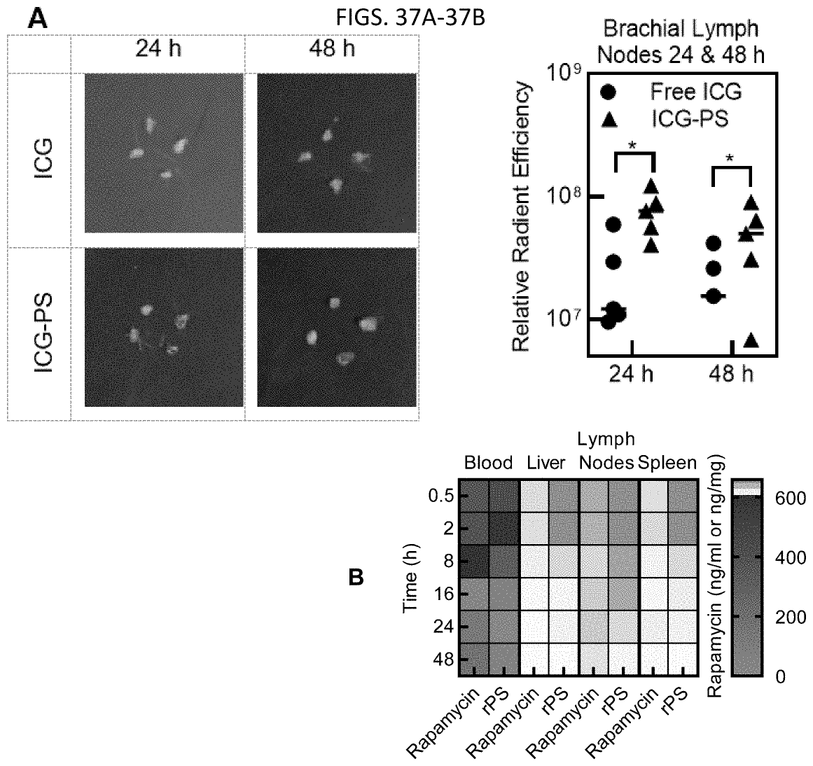

Polymersome delivery alters organ-level biodistribution (FIGS. 37A-37B)—A targeted and sustained biodistribution is necessary for immunosuppressive drugs to achieve their intended effect, while mitigating side effects. This is particularly important for islet transplantation. To demonstrate that PEG-b-PPS PS can alter the biodistribution of a compound, indocyanine green dye (ICG-PS), a drug mimic, was loaded into PS. We subcutaneously injected C57BL/6J mice with ICG-PS or free ICG and sacrificed animals at 2, 24, and 48 hours post injection and analyzed organs via IVIS (SUPPLEMENT). We show that ICG-PS allows for sustained drainage to the brachial lymph nodes at 24 and 48 hours post-injection (FIG. 37A). To confirm that this effect holds true for rapamycin, rapamycin (in 0.2% carboxymethyl cellulose (CMC)) or rPS were subcutaneously injected into C57BL/6J mice and the animals were sacrificed at various time points to assess rapamycin content in their various organs. We show that delivery of rapamycin via rPS increase rapamycin concentration in immune cell-rich tissues, such as the blood, liver, axial and brachial lymph nodes and spleen (FIG. 37B).

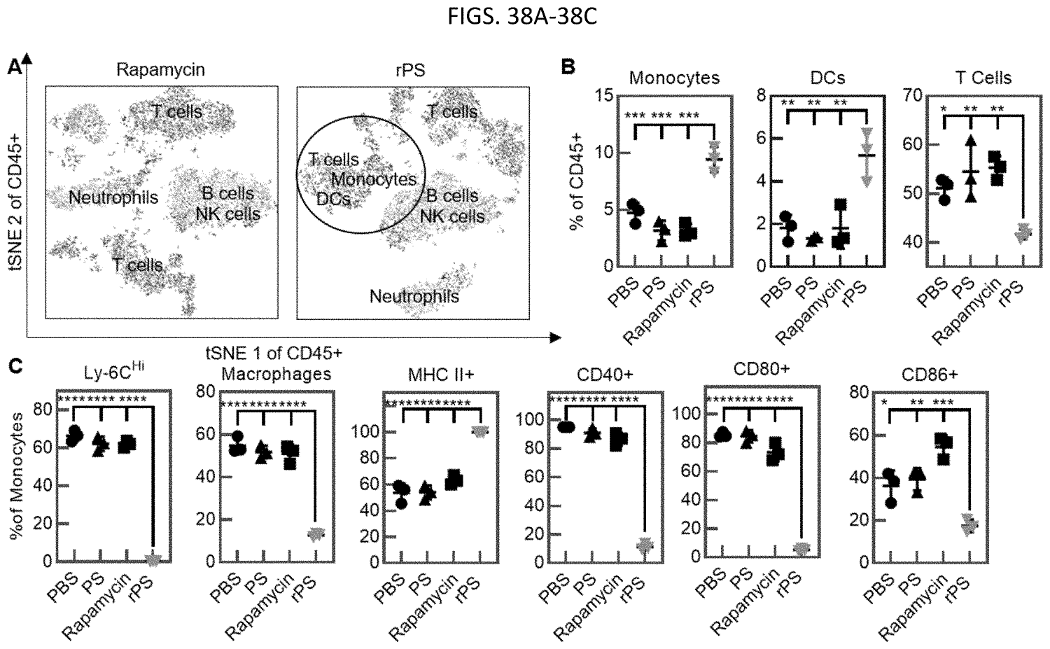

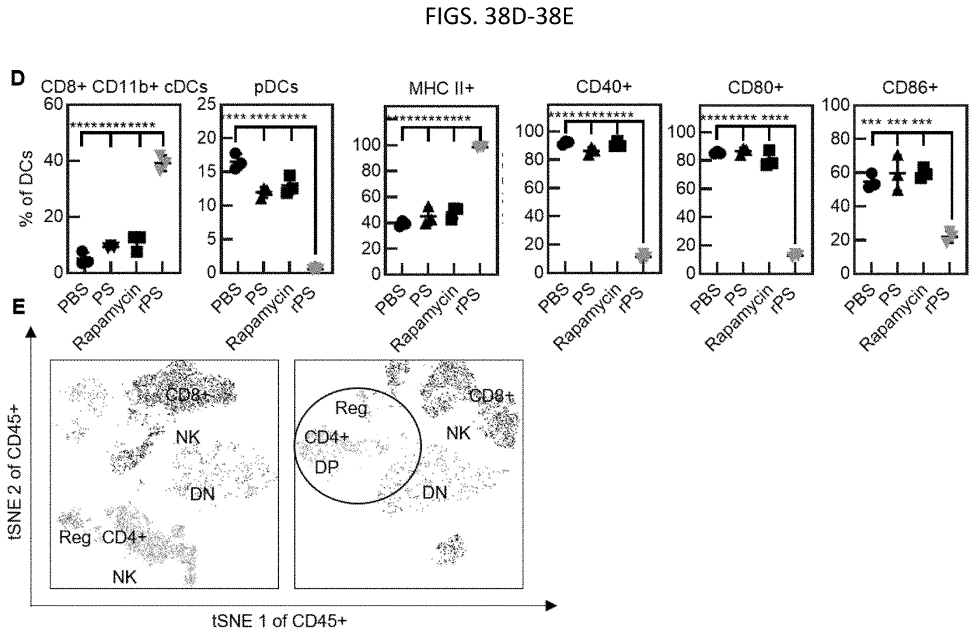

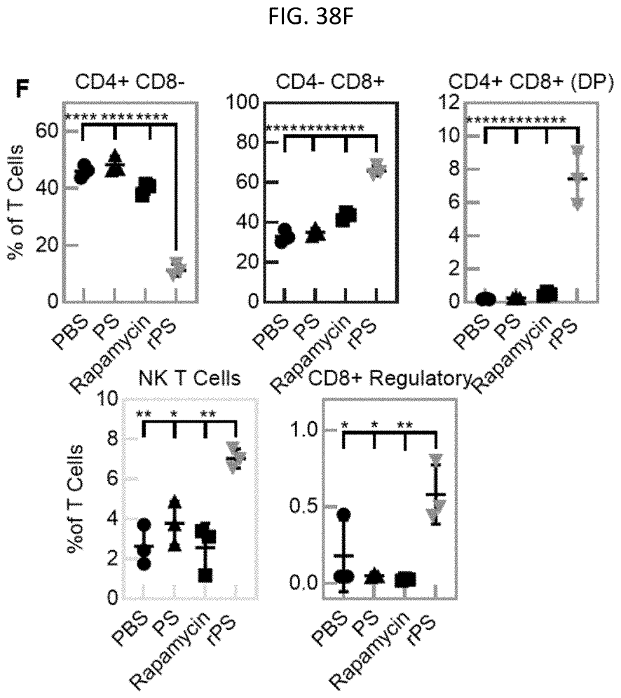

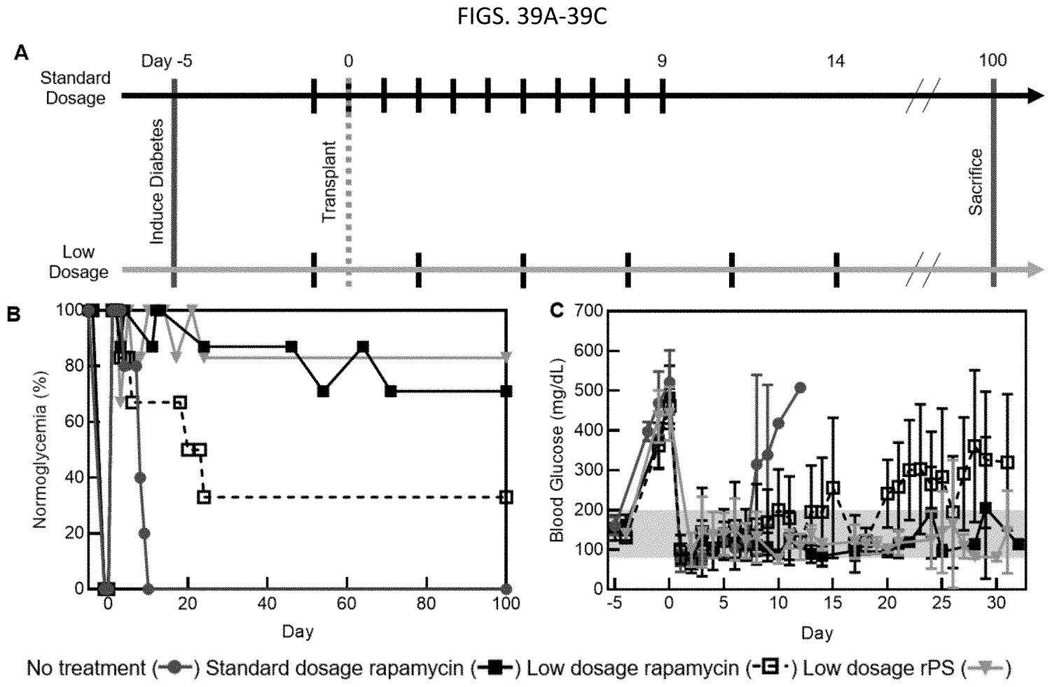

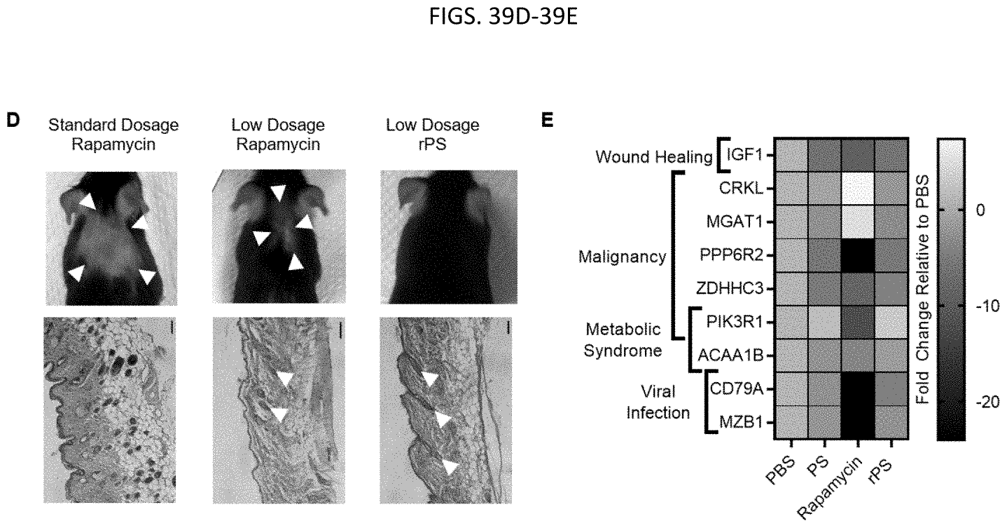

Polymersome delivery alters mechanism of action (FIGS. 38A-38F)—As an mTOR inhibitor, rapamycin is known to have its immunosuppressive effect via inhibition of T cell proliferation². Specifically, to mediate immunosuppression, rapamycin acts primarily on the intracellular FKBP12 receptor of these T cells with some immunomodulatory activity in mediated via APCs. Delivery of rapamycin via rPS shifts all dosed rapamycin to act on the FKBP12 receptors in APCs. In order to investigate if the immunomodulatory mechanism remained the same when rapamycin was targeted specifically to APCs, we subcutaneously injected healthy mice with either rapamycin or rPS using a standard immunosuppressive dosing protocol for islet transplantation (FIG. 39A) and then assessed immune cell populations via flow cytometry. First off, we show enhanced rPS-induced costimulation blockade of CD40, CD80 and CD86 in APCs (FIGS. 38C and 38D). Without the expression of costimulatory membrane proteins on antigen-presenting cells, T cells are unable to become activated to mount an immune response against a given transplanted graft. Costimulation blockade has been shown to enhance graft survival²⁴. Furthermore, rPS induce Ly-6C depletion of monocytic cell populations (FIG. 38C). This non-classical monocyte population has a dual-fold advantage for transplantation applications, in which it supports an anti-inflammatory phenotype amenable to graft tolerance²⁵ and it has been shown to aid in the prevention of viral infections²⁶. While these monocytes lack Ly-6C, they express mature levels of MEW II (FIG. 38C). Elevated MEW II levels are also observed in DCs. It has been previously shown that Ly-6C low, MEW II high monocytes differentiate into MEW II high DCs, which together confer CD8+ T cell antigen-specific tolerance²⁷ (FIG. 38F). Specifically, the DCs have a very unique CD8⁺ CD11b+ cDC presentation. CD8+ DCs are known to cross-tolerize CD8+ T cells and CD11b+ DCs are known to cross-tolerize CD4+ T cells. We hypothesize with this rare phenotype of cDCs tolerization of both populations may be achieved. Specifically, the high expression levels of MHC II on monocytes and DCs will provide CD4+ T cells will signal 1 for activation (FIGS. 38C and 38D). However, the lack of costimulatory molecules on these APCs (FIGS. 38C and 38D) will fail to provide CD4+ T cells with signal 2 for activation. As a result, the CD4+ T cells go into a state of anergy. We believe this is what is occurring in the clusters observed on the tSNE plots (FIGS. 38A and 38E) Specifically, CD4+ T cell populations are reduced in favor of CD8+ T cell populations, including regulatory CD8+ T cells (FIG. 38F). CD8+ CD25+ FoxP3+ regulatory T cells have enhanced suppressor capabilities relative to their CD4+ counterparts. The reduction of recipient CD4+ T cells is favorable, as this cell type has been associated with acute graft failure²⁸. The tolerogenic properties of CD8+ regulatory T cells are thought to prevent graft-versus-host disease and autoimmune diseases²⁹ Despite their tolerogenic properties, CD8+ T cells confer immunoprotection against pathogens³⁰. This protective effect is augmented by the upregulation NK T cells (FIG. 38F). In addition, fittingly, the upregulation of double positive CD4⁺ CD8+ T cells (DP T cells) has a dual function (FIG. 38F). DP T cells show suppressive functions, such as secreting anti-inflammatory cytokines under normal conditions, but enhanced responsiveness during infection, for example activating effector cells in the case of human immunodeficiency virus³¹.

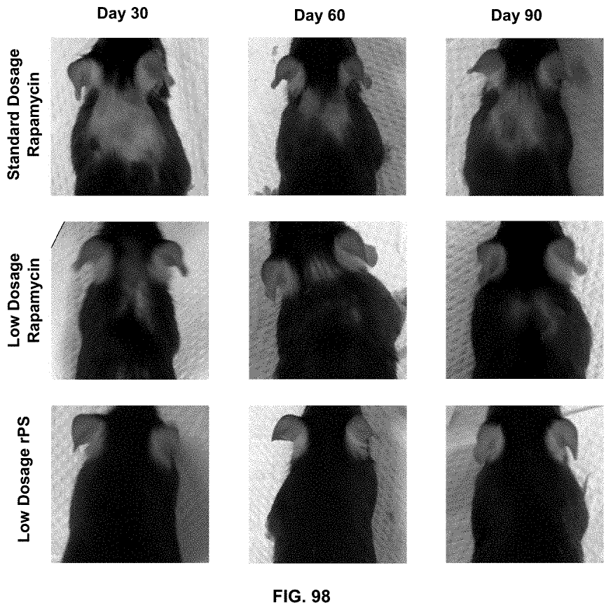

Polymersome delivery reduces the effective dose and reduces deleterious effects in vivo (FIGS. 39B and 39C)—In vivo assessment was conducted using a clinically relevant intraportal (liver) fully-MHC mismatched allogeneic islet transplantation model. C57BL/6J mice were induced with diabetes via streptozotocin injection. A standard dosage protocol known to allow for fully-MHC mismatched allogeneic islet graft viability for more than 100 days was compared to a low dosage protocol (FIG. 39B). The standard dosage protocol consisted of 11 injections, given daily. The low dosage protocol consisted of 6 doses given every 3 days (FIG. 39A). All doses were equivalent (1 mg rapamycin per kg body weight) (FIG. 39A). Diabetic C57BL/6J mice were transplanted via the portal vein with islets from fully MHC mismatched Balb/c mice. The efficacy success of the dosing regimen was confirmed by the restoration and maintenance of normoglycemia, confirming survival of the islet graft. Mice not treated with the drug all experienced graft rejection within 10 days of transplantation (FIGS. 39B and 39C). 71% of mice treated with the standard rapamycin protocol remained normoglycemic 100 days post transplantation (FIG. 39B). When the low dosage protocol was used, only a third of the mice treated with rapamycin remained normoglycemic 100 days post-transplantation, whereas 83% of mice treated with low dosage rPS had normal blood glucose concentrations (FIG. 39B). Furthermore, intraperitoneal glucose tolerance test (IPGTT), conducted at 30 days post-transplantation shows no difference in islet responsiveness with low-dosage rPS treatment as compared to standard dosage rapamycin (FIG. 98).

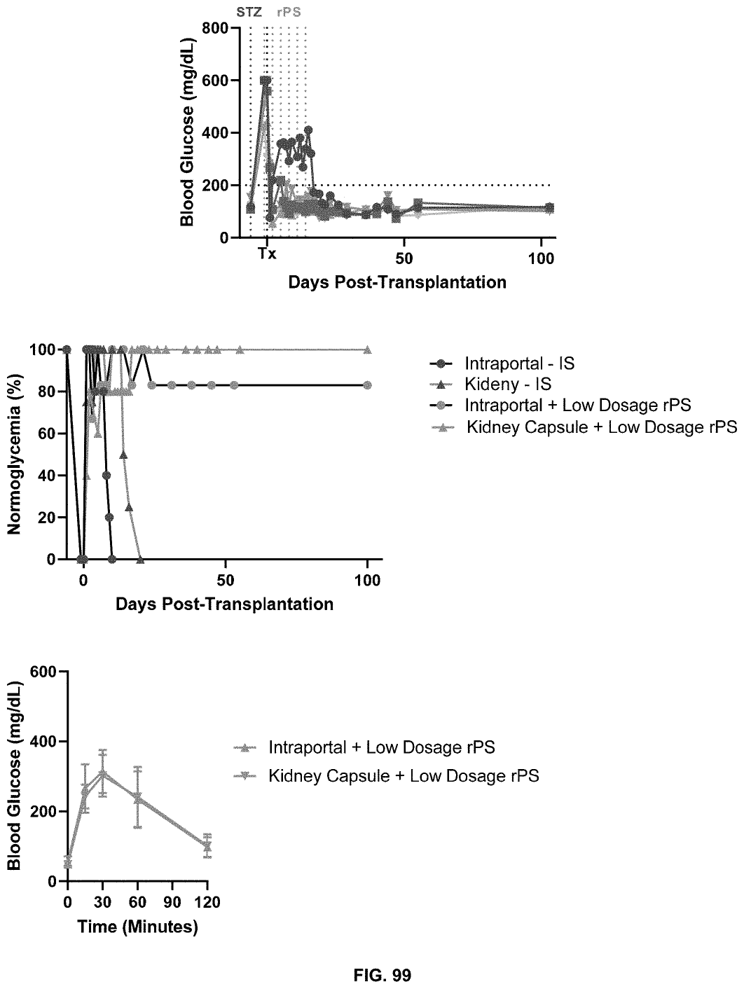

We observed that mice treated with free rapamycin experienced injection site alopecia (FIG. 39D). Alopecia is a known side effect of rapamycin, impacting approximately 10% of patients³². While alopecia was reduced in the low dosage free rapamycin group (FIG. 39D, FIG. 99), no alopecia was observed in the low dosage rPS group (FIG. 39D). Histological analysis confirms our gross observations (FIG. 39D, FIG. 99). Only immature follicles were identified in the standard rapamycin group (FIG. 39D, FIG. 99) with some mature follicles present in the low dosage free rapamycin group (FIG. 39D, FIG. 99). Organized mature follicles were identified in the low dosage rPS group (FIG. 39D, FIG. 99). Furthermore, single cell RNA sequencing analysis of macrophages and CD4+ regulatory T cells from the spleen and liver demonstrates that rPS mitigate expression of genes associated with rapamycin side effects. Specifically, rPS causes less inhibition of insulin-like growth factor 1 (IGF1), which is associated with impaired wound healing (FIG. 39E, Tables 5 and 6). Oncogenes CRKL (V-Crk Avian Aarcoma Virus CT10) is downregulated with rPS treatment, whereas it is upregulated with free rapamycin treatment³³(FIG. 39E, Tables 5 and 6). Tumor suppressor genes known to be downregulated by rapamycin, including MGAT1 (Mannosyl Glycoprotein Acetylglucosaminyl-Transferase 1)³⁴, PIK3R1 (Phosphoinositide-3-Kinase Regulatory Subunit 1)^{35,36}, PPP6R2 (Protein Phosphatase 6 Regulatory Subunit 2)³⁷, and ZDHHC3 (Zinc Finger DHHC-Type Palmitoyltransferase 3)³⁸ (FIG. 39E, Tables 5 and 6). Oncogenes CRKL (V-Crk Avian Aarcoma Virus CT10) is downregulated with rPS treatment, whereas it is upregulated with rapamycin treatment³³ (FIG. 39E, Tables 5 and 6). Tumor suppressor genes known to be downregulated by rapamycin, including MGAT1 (Mannosyl Glycoprotein Acetylglucosaminyl-Transferase 1)³⁴, PIK3R1 (Phosphoinositide-3-Kinase Regulatory Subunit 1)^{35,36}, PPP6R2 (Protein Phosphatase 6 Regulatory Subunit 2)³⁷, and ZDHHC3 (Zinc Finger DHHC-Type Palmitoyltransferase 3)³⁸ are less inhibited with rPS (FIG. 39E, Tables 5 and 6). Furthermore, inhibition of genes associated with the regulation of metabolic processes caused by rapamycin, including ACAA1 (Acetyl-CoA Acyltransferase 1)³⁹ and PIK3R14⁴⁰ is reduced when rapamycin is given in rPS form (FIG. 39E, Tables 5 and 6). Rapamycin causes downregulation of rPS genes associated with the protective response to viral infection, including CD79A⁴¹ and MZB1 (Marginal Zone B And B1 Cell Specific Protein)⁴² (FIG. 39E, Tables 5 and 6). The inhibition of these viral response genes is not seen with rPS treatment (FIG. 39E, Tables 5 and 6).

TABLE 5
Single-cell RNA sequencing analysis workflow

| Workflow | Program | Command Line |
|---|---|---|
| Quality Control | FastQC | fastqc <input_path_to/untrimmed.fq.gz> |
| | v0.11.5 | |
| Trimming and | Trimmomatic | java -jar ./Trimmomatic-0.39/trimmomatic-0.39.jar SE -threads 16 -phred33 |
| Filtering | v0.39 | <input_path_to/untrimmed.fq.gz> <output_path_to/trimmed.fq.gz> ILLUMINACLIP:TruSeq3- |
| | | SE.fa:2:30:10 LEADING:30 TRAILING:30 MINLEN:36 |
| Alignment | STAR | STAR --genomeDir ../star_index/ --readFilesCommand zcat -readFilesIn <input_path_to/trimmed.fq.gz>-- |
| | v2.6.0a | outFilterType BySJout --runThreadN 16 --outFilterMultimapNmax 100 --alignSJoverhangMin 8 -- |
| | | alignSJDBoverhangMin 1 --outFilterMismatchNoverLmax 0.05 --alignIntronMin 20 --alignIntronMax |
| | | 1000000 --alignMatesGapMax 1000000 --outSAMattributes NH HI NM MD --outSAMstrandField |
| | | intronMotif --outSAMmapqUnique 60 --outSAMtype BAM SortedByCoordinate --outReadsUnmapped |
| | | Fastx --limitBAMsortRAM 30000000000 --outFileNamePrefix <output_path_to/sorted_bam> |
| Quantification | Cufflinks | cuffdiff -L PBS,PS,R,RPS -o &lt;path_to/output_dir/> -p 16 -b |
| and Differential | v2.2.1 | ../genome_fasta/GRCm38.primary_assembly.genome.fa -u |
| Expression | | ../GTF/gencode.vM24.primary_assembly.annotation.gtf |
| Analysis | | treatment_1_sample_1.bam,treatment_1_sample_2.bam,treatment_1_sample_3.bam |
| | | treatment_2_sample_1.bam,treatment_2_sample_2.bam,treatment_2_sample_3.bam |
| | | treatment_3_sample_1.bam,treatment_3_sample_2.bam,treatment_3_sample_3.bam |
| | | treatment_4_sample_1.bam,treatment_4_sample_2.bam,treatment_4_sample_3.bam |

TABLE 6
Single-cell RNA sequencing raw data

| | | | PS | | Rapamycin | | rPS | | Rapamycin vs rPS | |
|---|---|---|---|---|---|---|---|---|---|---|
| | | Geneset | Fold | P | Fold | P | Fold | P | Fold | P |
| | Ensembl Code | Name | Change | Value | Change | Value | Change | Value | Change | Value |
| Spleen Macrophages | | | | | | | | | | |
| 2279 | ENSMUSG00000020053 | IGF1 | −6.39305 | 0.01815 | −9.10778 | 0.00425 | −6.03822 | 0.02255 | 3.06956 | 0.02915 |
| 10384 | ENSMUSG00000036561 | PPP6R2 | −5.69947 | 0.011 | −24 | 0.00005 | −5.5058 | 0.01425 | 24 | 0.00005 |
| 5190 | ENSMUSG00000025786 | ZDHHC3 | −5.15342 | 0.02015 | −8.14259 | 0.03895 | −4.23154 | 0.04065 | 3.91105 | 0.0462 |
| 2460 | ENSMUSG00000020346 | MGAT1 | −2.54064 | 0.0472 | 5.0747 | 0.0171 | −3.04016 | 0.01425 | −8.11486 | 0.001 |
| Spleen CD4+ | | | | | | | | | | |
| Regulatory T Cells | | | | | | | | | | |
| 573 | ENSMUSG00000003379 | CD79A | −2.52198 | 0.0419 | −24 | 0.00005 | −4.38266 | 0.00455 | 24 | 0.00005 |
| 4444 | ENSMUSG00000024353 | MZB1 | −2.06991 | 0.0297 | −24 | 0.00005 | −2.50151 | 0.0115 | 24 | 0.00005 |
| 947 | ENSMUSG00000006134 | CRKL | −0.689499 | 0.4598 | 7.38707 | 0.02155 | −1.97122 | 0.04935 | −9.3583 | 0.00525 |
| 12235 | ENSMUSG00000041417 | PIK3R1 | 1.96275 | 0.0626 | −12 | 0.00005 | 3.12528 | 0.07685 | 12 | 0.00005 |
| 1331 | ENSMUSG00000010651 | ACAA1 | −2.44129 | 0.0089 | −3.83338 | 0.0077 | −1.23789 | 0.03675 | 2.5955 | 0.0395 |

### DISCUSSION

While the immunosuppressive agents on the market currently fail to meet the needs of patients there has been as substantial slowdown in regulatory approval of new immunosuppressive drugs since the 1990s². This is because it has proven to be challenging to find new compounds that show improvement in regard to efficiency and safety over approved drugs' Nanomedicine has the potential to harness the effective properties of existing therapies, while mitigating undesirable effects, thus, overcoming the shortcomings of today's immunosuppressive drugs^{10,43}. As we have demonstrated herein, loading an off-the-shelf drug into inert nanocarriers engineered to stability deliver payloads to APCs, not only alters the biodistribution and effective dose of the drug, but also the mechanism of action. We show that PS enhance immune cell uptake of loaded drugs, thus partitioning the biodistribution of the drugs to immune-rich tissues. With these properties, we have demonstrated that rapamycin delivery via rPS enhances potency as only about half of the dosage (55%) is needed to effectively maintain allogeneic islet graft survival and can mitigate side effects, such as injection site alopecia and damaging transplantation site free radicals. Most importantly, we demonstrate that by delivering an existing drug to a different cell type when can completely change that drug's mechanism of action. Specifically, shuttling rapamycin to APCs via rPS significantly enhanced immunosuppressive effects via costimulation blockade (FIG. 37A) and an increased CD8+ regulatory T cell population (FIG. 37D), while preventing systemic immune compromise via enhanced CD8+ T cell populations (FIG. 37C) and rapamycin-associated side effects (FIGS. 39D and 39E). In many ways, the effects of rPS are similar to that of the biologic drugs belatacept, as this CTLA-IgG protein based drug binds to CD80 and CD86 on antigen-presenting cells to induced costimulation blockade, thus inducing reductions in T cell proliferation, specifically reducing CD4+ T cells more than CD8+ T cells. While the mechanisms of action may be similar, as a small molecule formulation rPS comes with a much smaller price tag and is administered subcutaneously, as opposed to intravenously, thus patients can take their medication in the comfort of their home instead of traveling to their doctor's office. In future studies, a head to head comparison of rPS and belatacept should be conducted to determine relative efficiency.

### REFERENCES

1 Kory, L. Nonadherence to immunosuppressive medications: a pilot survey of members of the transplant recipients international organization. Transplant Proc 31, 14S-15S, doi:10.1016/s0041-1345(99)00114-1 (1999).
2 Wiseman, A. C. Immunosuppressive Medications. Clin J Am Soc Nephrol 11, 332-343, doi:10.2215/CJN.08570814 (2016).
3 Berney, T. & Secchi, A. Rapamycin in islet transplantation: friend or foe? Transpl Int 22, 153-161, doi:10.1111/j.1432-2277.2008.00743.x (2009).
4 Trepanier, D. J., Gallant, H., Legatt, D. F. & Yatscoff, R. W. Rapamycin: distribution, pharmacokinetics and therapeutic range investigations: an update. Clin Biochem 31, 345-351 (1998).
5 Yatscoff, R. W., Wang, P., Chan, K., Hicks, D. & Zimmerman, J. Rapamycin: distribution, pharmacokinetics, and therapeutic range investigations. Ther Drug Monit 17, 666-671 (1995).
6 Napoli, K. L., Wang, M. E., Stepkowski, S. M. & Kahan, B. D. Distribution of sirolimus in rat tissue. Clin Biochem 30, 135-142 (1997).
7 Ferron, G. M., Mishina, E. V., Zimmerman, J. J. & Jusko, W. J. Population pharmacokinetics of sirolimus in kidney transplant patients. Clin Pharmacol Ther 61, 416-428, doi:10.1016/S0009-9236(97)90192-2 (1997).
8 Meier-Kriesche, H. U. & Kaplan, B. Toxicity and efficacy of sirolimus: relationship to whole-blood concentrations. Clin Ther 22 Suppl B, B93-100 (2000).
9 Hafiz, M. M. et al. Immunosuppression and procedure-related complications in 26 patients with type 1 diabetes mellitus receiving allogeneic islet cell transplantation. Transplantation 80, 1718-1728 (2005).
10 Ventola, C. L. Progress in Nanomedicine: Approved and Investigational Nanodrugs. P T 42, 742-755 (2017).
11 Kang, S. Y. et al. Intravenous tacrolimus and cyclosporine induced anaphylaxis: what is next? Asia Pac Allergy 5, 181-186, doi:10.5415/apallergy.2015.5.3.181 (2015).
12 Halloran, P. F. Immunosuppressive drugs for kidney transplantation. N Engl J Med 351, 2715-2729, doi:10.1056/NEJMra033540 (2004).
13 Yi, S. et al. Tailoring Nanostructure Morphology for Enhanced Targeting of Dendritic Cells in Atherosclerosis. ACS Nano 10, 11290-11303, doi:10.1021/acsnano.6b06451 (2016).
14 Yi, S. et al. Surface Engineered Polymersomes for Enhanced Modulation of Dendritic Cells During Cardiovascular Immunotherapy. Advanced Functional Materials 29, 1904399, doi:10.1002/adfm.201904399 (2019).
15 Al-Lawati, H., Aliabadi, H. M., Makhmalzadeh, B. S. & Lavasanifar, A. Nanomedicine for immunosuppressive therapy: achievements in pre-clinical and clinical research. Expert Opin Drug Deliv 15, 397-418, doi:10.1080/17425247.2018.1420053 (2018).
16 Allen, S., Osorio, O., Liu, Y. G. & Scott, E. Facile assembly and loading of theranostic polymersomes via multi-impingement flash nanoprecipitation. J Control Release 262, 91-103, doi:10.1016/j.jconrel.2017.07.026 (2017).
17 Dowling, D. J. et al. Toll-like receptor 8 agonist nanoparticles mimic immunomodulating effects of the live BCG vaccine and enhance neonatal innate and adaptive immune responses. J Allergy Clin Immunol 140, 1339-1350, doi:10.1016/j.jaci.2016.12.985 (2017).
18 Napoli, A., Valentini, M., Tirelli, N., Muller, M. & Hubbell, J. A. Oxidation-responsive polymeric vesicles. Nat Mater 3, 183-189, doi:10.1038/nmat1081 (2004).
19 Allen S D, L. Y., Bobbala S, Cai L, Hecker P I, Temel R, Scott E A. Polymersomes scalably fabricated via flash nanoprecipitation are non-toxic in non-human primates and associate with leukocytes in the spleen and kidney following intravenous administration. Nano Research, doi:10.1007/s12274-018-2069-x (2018).
20 Stano, A., Scott, E. A., Dane, K. Y., Swartz, M. A. & Hubbell, J. A. Tunable T cell immunity towards a protein antigen using polymersomes vs. solid-core nanoparticles. Biomaterials 34, 4339-4346, doi:10.1016/j.biomaterials.2013.02.024 (2013).
21 Scott, E. A. et al. Dendritic cell activation and T cell priming with adjuvant- and antigen-loaded oxidation-sensitive polymersomes. Biomaterials 33, 6211-6219, doi:10.1016/j.biomaterials.2012.04.060 (2012).
22 Stabler, C. L., Li, Y., Stewart, J. M. & Keselowsky, B. C. Engineering immunomodulatory biomaterials for type 1 diabetes. Nat Rev Mater 4, 429-450 (2019).
23 Zorec, B., Preat, V., Miklavcic, D. & Payselj, N. Active enhancement methods for intra- and transdermal drug delivery: a review. Zdr Vestn 82, 339-356 (2013).
24 Malm, H., Pahlman, C., Veress, B., Corbascio, M. & Ekberg, H. Combined costimulation blockade prevents rejection of allogeneic islets in mice. Scandinavian Journal of Immunology 64, 398-403 (2006).
25 Kawamura, S. & Ohteki, T. Monopoiesis in humans and mice. Int Immunol 30, 503-509, doi:10.1093/intimm/dxy063 (2018).
26 Zhu, J., Chen, H., Huang, X., Jiang, S. & Yang, Y. Ly6C(hi) monocytes regulate T cell responses in viral hepatitis. JCI Insight 1, e89880, doi:10.1172/jci.insight.89880 (2016).
27 Peng, Y., Latchman, Y. & Elkon, K. B. Ly6C(low) monocytes differentiate into dendritic cells and cross-tolerize T cells through PDL-1. J Immunol 182, 2777-2785, doi:10.4049/jimmunol.0803172 (2009).
28 Pietra, B. A., Wiseman, A., Bolwerk, A., Rizeq, M. & Gill, R. G. CD4 T cell-mediated cardiac allograft rejection requires donor but not host MHC class II. Journal of Clinical Investigation 106, 1003-1010 (2000).
29 Vieyra-Lobato, M. R., Vela-Ojeda, J., Montiel-Cervantes, L., Lopez-Santiago, R. & Moreno-Lafont, M. C. Description of CD8(+) Regulatory T Lymphocytes and Their Specific Intervention in Graft-versus-Host and Infectious Diseases, Autoimmunity, and Cancer. J Immunol Res 2018, 3758713, doi:10.1155/2018/3758713 (2018).
30 Zhang, N. & Bevan, M. J. CD8(+) T cells: foot soldiers of the immune system. Immunity 35, 161-168, doi:10.1016/j.immuni.2011.07.010 (2011).
31 Overgaard, N. H., Jung, J. W., Steptoe, R. J. & Wells, J. W. CD4+/CD8+ double-positive T cells: more than just a developmental stage? J Leukoc Biol 97, 31-38, doi:10.1189/jlb.1RU0814-382 (2015).
32 Mahe, E. et al. Cutaneous adverse events in renal transplant recipients receiving sirolimus-based therapy. Transplantation 79, 476-482 (2005).
33 Song, Q. et al. CRKL regulates alternative splicing of cancer-related genes in cervical cancer samples and HeLa cell. BMC Cancer 19, 499, doi:10.1186/s12885-019-5671-8 (2019).
34 Akiva, I. & Birgul Iyison, N. MGAT1 is a novel transcriptional target of Wnt/beta-catenin signaling pathway. BMC Cancer 18, 60, doi:10.1186/s12885-017-3960-7 (2018).
35 Lin, Y. et al. PIK3R1 negatively regulates the epithelial-mesenchymal transition and stem-like phenotype of renal cancer cells through the AKT/GSK3beta/CTNNB1 signaling pathway. Sci Rep 5, 8997, doi:10.1038/srep08997 (2015).
36 Vallejo-Diaz, J., Chagoyen, M., Olazabal-Moran, M., Gonzalez-Garcia, A. & Carrera, A. C. The Opposing Roles of PIK3R1/p85alpha and PIK3R2/p85beta in Cancer. Trends Cancer 5, 233-244, doi:10.1016/j.trecan.2019.02.009 (2019).
37 Zhong, J. et al. Protein phosphatase PP6 is required for homology-directed repair of DNA double-strand breaks. Cell Cycle 10, 1411-1419, doi:10.4161/cc.10.9.15479 (2011).
38 Kinney, N., Varghese, R. T., Anandakrishnan, R. & Garner, H. R. S. ZDHHC3 as a Risk and Mortality Marker for Breast Cancer in African American Women. Cancer Inform 16, 1176935117746644, doi:10.1177/1176935117746644 (2017).
39 Cazanave, S. et al. The Transcriptomic Signature Of Disease Development And Progression Of Nonalcoholic Fatty Liver Disease. Sci Rep 7, 17193, doi:10.1038/s41598-017-17370-6 (2017).
40 Thauvin-Robinet, C. et al. PIK3R1 mutations cause syndromic insulin resistance with lipoatrophy. Am J Hum Genet 93, 141-149, doi:10.1016/j.ajhg.2013.05.019 (2013).
41 Havranek, O. et al. Tonic B-cell receptor signaling in diffuse large B-cell lymphoma. Blood 130, 995-1006, doi:10.1182/blood-2016-10-747303 (2017).
42 Andreani, V. et al. Cochaperone Mzb1 is a key effector of Blimp 1 in plasma cell differentiation and beta1-integrin function. Proc Natl Acad Sci USA 115, E9630-E9639, doi:10.1073/pnas.1809739115 (2018).
43 Allen, S. D. et al. Celastrol-loaded PEG-b-PPS nanocarriers as an anti-inflammatory treatment for atherosclerosis. Biomater Sci 7, 657-668, doi:10.1039/c8bm01224e (2019).
44 Allen, S. D., Bobbala, S., Karabin, N. B., Modak, M. & Scott, E. A. Benchmarking Bicontinuous Nanospheres against Polymersomes for in Vivo Biodistribution and Dual Intracellular Delivery of Lipophilic and Water-Soluble Payloads. ACS Appl Mater Interfaces 10, 33857-33866, doi:10.1021/acsami.8b09906 (2018).
45 Yu, Y. R. et al. A Protocol for the Comprehensive Flow Cytometric Analysis of Immune Cells in Normal and Inflamed Murine Non-Lymphoid Tissues. Plos One 11, e0150606, doi:10.1371/journal.pone.0150606 (2016).
46 Belkina, A. C. et al. Automated optimized parameters for T-distributed stochastic neighbor embedding improve visualization and analysis of large datasets. Nat Commun 10,5415, doi:10.1038/s41467-019-13055-y (2019).
47 Andrews, S. FastQC: a quality control tool for high throughput sequence data. Available online at: bioinformatics.babraham.ac.uk/projects/fastqc (2010).
48 Bolger, A. M., Lohse, M. & Usadel, B. Trimmomatic: a flexible trimmer for Illumina sequence data. Bioinformatics 30, 2114-2120, doi:10.1093/bioinformatics/btu170 (2014).
49 Dobin, A. et al. STAR: ultrafast universal RNA-seq aligner. Bioinformatics 29, 15-21, doi:10.1093/bioinformatics/bts635 (2013).
50 Trapnell, C. et al. Transcript assembly and quantification by RNA-Seq reveals unannotated transcripts and isoform switching during cell differentiation. Nat Biotechnol 28, 511-515, doi:10.1038/nbt.1621 (2010).
51 Trapnell, C. et al. Differential analysis of gene regulation at transcript resolution with RNA-seq. Nat Biotechnol 31, 46-53, doi:10.1038/nbt.2450 (2013).
52 Roberts, A., Trapnell, C., Donaghey, J., Rinn, J. L. & Pachter, L. Improving RNA-Seq expression estimates by correcting for fragment bias. Genome Biol 12, R22, doi:10.1186/gb-2011-12-3-r22 (2011).

We claim:

1. A method of treating an inflammatory condition in a subject in need thereof, the method comprising administering to the subject a therapeutically effective amount of a nanocarrier comprising:
a poly(ethylene glycol)-block-poly(propylene sulfide) copolymer; and
rapamycin.

2. The method of claim 1, wherein the inflammatory condition is at least one of atherosclerosis, arthritis, organ transplantation, cell transplantation, or inflammatory bowel disease.

3. The method of claim 1, wherein the transplantation rejection is islet transplantation rejection.

4. The method of claim 1, wherein the therapeutically effective amount of the nanocarrier is about 1 μg/kg to about 400 μg/kg.

5. The method of claim 1, wherein the nanocarrier is administered parenterally to the subject.

6. The method of claim 1, wherein the nanocarrier is an aqueous core polymersome or a bicontinuous nanosphere.

7. The method of claim 1, wherein the nanocarrier is a micelle or a filomicelle.

8. The method of claim 1, wherein the poly(ethylene glycol)-block-poly(propylene sulfide) copolymer has a PEG weight fraction of 0.12 to 0.69.

9. The method of claim 8, wherein the poly(ethylene glycol)-block-poly(propylene sulfide) copolymer has a PEG weight fraction of 0.25.

10. The method of claim 1, wherein the nanocarrier is incorporated into a pharmaceutical composition with one or more pharmaceutically acceptable excipients.

11. A method of preventing transplantation rejection in a patient in need thereof, the method comprising administering to the subject a therapeutically effective amount of a nanocarrier comprising:
a poly(ethylene glycol)-block-poly(propylene sulfide) copolymer; and
rapamycin.

12. The method of claim 11, wherein the transplantation rejection is cell transplantation rejection, tissue transplantation rejection, or organ transplantation rejection.

13. The method of claim 11, wherein the transplantation rejection is islet transplantation rejection.

14. The method of claim 11, wherein the therapeutically effective amount of the nanocarrier is about 1 μg/kg to about 400 μg/kg.

15. The method of claim 11, wherein the nanocarrier is administered parenterally to the subject.

16. The method of claim 11, wherein the nanocarrier is an aqueous core polymersome or a bicontinuous nanosphere.

17. The method of claim 11, wherein the nanocarrier is a micelle or a filomicelle.

18. The method of claim 11, wherein the poly(ethylene glycol)-block-poly(propylene sulfide) copolymer has a PEG weight fraction of 0.12 to 0.69.

19. The method of claim 18, wherein the poly(ethylene glycol)-block-poly(propylene sulfide) copolymer has a PEG weight fraction of 0.25.

20. The method of claim 11, wherein the nanocarrier is incorporated into a pharmaceutical composition with one or more pharmaceutically acceptable excipients.


SEQUENCE LISTING (19 sequences)

SEQ ID NO: 1 - protein, 16 aa, synthetic construct
  REGION 1..16: note = synthetic - P-D2 peptide
GGVTLTYQFAAGPRDK

SEQ ID NO: 2 - protein, 22 aa, synthetic construct
  REGION 1..22: note = synthetic - P210 peptide
KTTKQSFDLSVKAQYKKKNKHK

SEQ ID NO: 3 - DNA, 23 bp, synthetic construct
  misc_feature 1..23: note = synthetic - primer
  source 1..23: mol_type = other DNA
ggcacagtcaaggccgagaatgg

SEQ ID NO: 4 - DNA, 25 bp, synthetic construct
  misc_feature 1..25: note = synthetic - primer
  source 1..25: mol_type = other DNA
gatgttagtggggtctcgctcctgg

SEQ ID NO: 5 - DNA, 25 bp, synthetic construct
  misc_feature 1..25: note = synthetic - primer
  source 1..25: mol_type = other DNA
ctcatgatagtgcctgtgtcctcaa

SEQ ID NO: 6 - DNA, 20 bp, synthetic construct
  misc_feature 1..20: note = synthetic - primer
  source 1..20: mol_type = other DNA
agggccagcataggtgcaag

SEQ ID NO: 7 - DNA, 24 bp, synthetic construct
  misc_feature 1..24: note = synthetic
  source 1..24: mol_type = other DNA
agtttccatgtccaaggctcattc

SEQ ID NO: 8 - DNA, 22 bp, synthetic construct
  misc_feature 1..22: note = synthetic - primer
  source 1..22: mol_type = other DNA
ttgtaacggcaaggcagcaata

SEQ ID NO: 9 - DNA, 24 bp, synthetic construct
  misc_feature 1..24: note = synthetic - primer
  source 1..24: mol_type = other DNA
gttctgtacgagcactatttgggc

SEQ ID NO: 10 - DNA, 26 bp, synthetic construct
  misc_feature 1..26: note = synthetic - primer
  source 1..26: mol_type = other DNA
gtgaagtcgtagagtccagttgttcc

SEQ ID NO: 11 - DNA, 19 bp, synthetic construct
  misc_feature 1..19: note = synthetic - primer
  source 1..19: mol_type = other DNA
gggttgccaagccttatcg

SEQ ID NO: 12 - DNA, 24 bp, synthetic construct
  misc_feature 1..24: note = synthetic - primer
  source 1..24: mol_type = other DNA
cttctcacccagggaattcaaatg

SEQ ID NO: 13 - DNA, 23 bp, synthetic construct
  misc_feature 1..23: note = synthetic - primer
  source 1..23: mol_type = other DNA
ttccatccagttgccttcttggg

SEQ ID NO: 14 - DNA, 24 bp, synthetic construct
  misc_feature 1..24: note = synthetic - primer
  source 1..24: mol_type = other DNA
gggagtggtatcctctgtgaagtc

SEQ ID NO: 15 - DNA, 23 bp, synthetic construct
  misc_feature 1..23: note = synthetic - primer
  source 1..23: mol_type = other DNA
caattcacactgaatgccagctc

SEQ ID NO: 16 - DNA, 21 bp, synthetic construct
  misc_feature 1..21: note = synthetic - primer
  source 1..21: mol_type = other DNA
caagcaagtccgtctcgtcca

SEQ ID NO: 17 - DNA, 21 bp, synthetic construct
  misc_feature 1..21: note = synthetic - primer
  source 1..21: mol_type = other DNA
caagcaagtccgtctcgtcca

SEQ ID NO: 18 - DNA, 21 bp, synthetic construct
  misc_feature 1..21: note = synthetic - primer
  source 1..21: mol_type = other DNA
tgccggcatatacgagtgtga

SEQ ID NO: 19 - DNA, 22 bp, synthetic construct
  misc_feature 1..22: note = synthetic - primer
  source 1..22: mol_type = other DNA
cccgatggcaggtattaccaag